SEQUENCE LISTING (177 sequences)

SEQ ID NO: 1 - protein, 292 aa, Homo sapiens
  REGION 1..292: note = FcgammaRI binding domain
  SIGNAL 1..15
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFH

SEQ ID NO: 2 - protein, 290 aa, Homo sapiens
  REGION 1..290: note = Tim1 binding domain
  SIGNAL 1..20
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKG

SEQ ID NO: 3 - protein, 314 aa, Homo sapiens
  REGION 1..314: note = Tim4 binding domain
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQ

SEQ ID NO: 4 - protein, 202 aa, Homo sapiens
  REGION 1..202: note = Tim3 binding domain
  SIGNAL 1..21
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 5 - protein, 161 aa, Homo sapiens
  REGION 1..161: note = FA58C2 binding domain
LNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 6 - protein, 94 aa, Homo sapiens
  REGION 1..94: note = GAS6 binding domain
  SIGNAL 1..30
MAPSLSPGPAALRRAPQLLLLLLAAECALAALLPAREATQFLRPRQRRAFQVFEEAKQGHLERECVEELCSREEAREVFENDPETDYFYPRYLD

SEQ ID NO: 7 - protein, 87 aa, Homo sapiens
  REGION 1..87: note = protein S binding domain
  SIGNAL 1..24
MRVLGGRCGALLACLLLVLPVSEANFLSKQQASQVLVRKRRANSLLEETKQGNLERECIEELCNKEEAREVFENDPETDYFYPKYLV

SEQ ID NO: 8 - protein, 918 aa, Homo sapiens
  REGION 1..918: note = BAI1 binding domain
AAGADAGPGPEPCATLVQGKFFGYFSAAAVFPANASRCSWTLRNPDPRRYTLYMKVAKAPVPCSGPGRVRTYQFDSFLESTRTYLGVESFDEVLRLCDPSAPLAFLQASKQFLQMRRQQPPQHDGLRPRAGPPGPTDDFSVEYLVVGNRNPSRAACQMLCRWLDACLAGSRSSHPCGIMQTPCACLGGEAGGPAAGPLAPRGDVCLRDAVAGGPENCLTSLTQDRGGHGATGGWKLWSLWGECTRDCGGGLQTRTRTCLPAPGVEGGGCEGVLEEGRQCNREACGPAGRTSSRSQSLRSTDARRREELGDELQQFGFPAPQTGDPAAEEWSPWSVCSSTCGEGWQTRTRFCVSSSYSTQCSGPLREQRLCNNSAVCPVHGAWDEWSPWSLCSSTCGRGFRDRTRTCRPPQFGGNPCEGPEKQTKFCNIALCPGRAVDGNWNEWSSWSACSASCSQGRQQRTRECNGPSYGGAECQGHWVETRDCFLQQCPVDGKWQAWASWGSCSVTCGAGSQRRERVCSGPFFGGAACQGPQDEYRQCGTQRCPEPHEICDEDNFGAVIWKETPAGEVAAVRCPRNATGLILRRCELDEEGIAYWEPPTYIRCVSIDYRNIQMMTREHLAKAQRGLPGEGVSEVIQTLVEISQDGTSYSGDLLSTIDVLRNMTEIFRRAYYSPTPGDVQNFVQILSNLLAEENRDKWEEAQLAGPNAKELFRLVEDFVDVIGFRMKDLRDAYQVTDNLVLSIHKLPASGATDISFPMKGWRATGDWAKVPEDRVTVSKSVFSTGLTEADEASVFVVGTVLYRNLGSFLALQRNTTVLNSKVISVTVKPPPRSLRTPLEIEFAHMYNGTTNQTCILWDETDVPSSSAPPQLGPWSWRGCRTVPLDALRTRCLCDRLSTFAILAQLSADANMEKATLPS

SEQ ID NO: 9 - DNA, 876 bp, Homo sapiens
  misc_feature 1..876: note = FcgammaRI binding domain
  source 1..876: mol_type = other DNA
atgtggttcctgactacgttgttgctgtgggtccctgtagacggccaagtagacacaacgaaagcagtgatcacgctccaaccgccttgggtgtctgtgttccaagaagaaacagttacactgcactgtgaggtcctccacctgcctggttcttcatctactcaatggtttctcaacggaacagcaacacaaacaagtaccccttcctacagaattacgagtgcatctgttaacgattcaggagagtataggtgccagcgagggctttcaggccggtccgaccccattcaactcgaaattcaccgcggttggcttctgctgcaagtatcctctcgggtcttcacggaaggtgaaccacttgccttgcgctgtcacgcatggaaagataagctcgtctacaacgttttgtattatcggaatggaaaggcatttaagttttttcattggaactcaaaccttacgatcctcaaaaccaatatcagtcataacggtacgtaccactgctcaggcatgggcaagcatcgctatacgtccgcagggattagcgtgacagttaaggagctcttccccgcgcctgtgctgaatgcgagcgtaacttcaccccttctggagggcaacttggtgaccctctcttgtgagacgaaacttctccttcagaggccgggcctgcaactctatttcagcttttatatgggttctaaaactcttcgaggcagaaacacgagcagcgaatatcagatactgactgcccggcgggaagacagtggcctttattggtgcgaggctgcaacagaagatggcaatgtccttaaaaggtctcccgaattggagctccaagtgcttggcttgcaactccctacacccgtatggttccac

SEQ ID NO: 10 - DNA, 870 bp, Homo sapiens
  misc_feature 1..870: note = Tim1 binding domain
  source 1..870: mol_type = other DNA
atgcacccccaggttgttatactttcattgatcctgcatttggccgactccgtggcgggttccgtaaaagttggaggggaagctggaccaagcgtcaccttgccttgccactactctggagccgtgacgagtatgtgctggaatcgaggatcctgtagtcttttcacatgccaaaatggcatagtttggaccaatgggacgcacgtcacctaccgaaaagacactagatacaaactcctgggtgacctcagcaggagagatgtgtctctgactattgaaaacactgctgtttctgactctggagtctactgttgccgggtcgagcaccgaggatggttcaatgacatgaagatcacggtcagcttggaaatcgtcccgcccaaggtaaccactacaccaatagttactacggttcccacggtaaccacggttcgaaccagcaccacagtacccacaactacgaccgttccaaccactacagtccccacaaccatgagtatccctacgacaactacggtcctgacaaccatgaccgtcagcactaccacgagtgtgcctacgactactagcataccgacgactacttcagttccagtcaccaccacggtgagtacattcgtgcctccaatgccattgccgaggcaaaaccacgaacccgtggcgacatctccgtctagtccgcaaccagcagagacccatcccaccacgcttcagggggcaatcaggagagaacctacaagttcacccctctacagctatacaaccgatggaaacgacacagttacagaaagtagtgacggtttgtggaataacaaccaaacacaattgttcctggagcacagtctgttgacagccaacactacaaaggga

SEQ ID NO: 11 - DNA, 942 bp, Homo sapiens
  misc_feature 1..942: note = Tim4 binding domain
  source 1..942: mol_type = other DNA
atgagtaaagagccgcttatcctgtggcttatgatagagttttggtggttgtatttgaccccggtcacgagcgaaacggtagtgactgaagtattgggtcatcgggtaaccttgccttgcttgtatagctcctggtctcataatagtaatagcatgtgctggggcaaggaccaatgcccctatagcggatgcaaggaggcgctcattcgcacagacggaatgcgggtgacatcaaggaagagtgctaagtaccggcttcagggcacaatcccacgcggcgacgtgtcactgactatccttaatccatccgagagcgactctggtgtctattgttgcaggatcgaggttccgggatggttcaatgatgtaaagatcaatgtaagactcaatctgcaacgggcatctacaaccacgcatcggacagccactactaccacaaggagaacaactactacgtcacccacgactactcgacagatgaccactacacctgcggccctgccaactacggttgtaactactccggatctgacaaccgggacaccgttgcaaatgacaaccattgcagtatttaccacggcaaacacgtgtctctctctgaccccatctactcttccggaggaggccaccgggctccttacaccggagccgtctaaggaaggcccaatcttgaccgcagagagtgagaccgtacttccgagcgattcatggtccagtgtcgagagcacatccgctgacaccgtccttcttacgtccaaagaaagtaaagtttgggacctcccgtccacgagccacgtttctatgtggaagacctcagatagcgttagctccccacagccaggagcaagcgacaccgcagtaccggagcaaaacaagacgactaagactggccagatggatggtatcccaatgtcaatgaaaaatgagatgcccatatcacaa

SEQ ID NO: 12 - DNA, 606 bp, Homo sapiens
  misc_feature 1..606: note = Tim3 binding domain
  source 1..606: mol_type = other DNA
atgtttagccatctcccttttgattgcgtcttgttgcttcttcttctccttctgacgagatcatctgaagttgaatatcgcgcggaagtcggccaaaacgcatatctgccgtgtttttacaccccggctgcaccggggaacttggttcccgtttgttggggtaagggggcgtgtcccgtttttgagtgcggtaacgtagtgctccgaactgatgaaagagatgtaaattactggacgagccggtactggttgaatggggattttaggaagggcgacgtttcccttaccatagaaaacgtaactcttgcggattctgggatttattgttgcaggatacaaatccccggaataatgaacgatgagaaattcaatttgaagctcgtaataaaaccggcaaaagtaactccagctcccaccaggcagcgagattttacggcagcatttcccaggatgctcactactcgcggtcatggccctgccgagactcagaccctcggtagtcttcctgatatcaatctcacgcaaattagtacattggcgaatgaattgagggattcaagactcgccaatgatctgcgcgacagtggagcgactattaggataggg

SEQ ID NO: 13 - DNA, 483 bp, Homo sapiens
  misc_feature 1..483: note = FA58C2 binding domain
  source 1..483: mol_type = other DNA
ctcaacgggtgtgctaatccccttggcctgaagaataacagcatacctgacaagcaaataacagcgtcaagttcttataaaacttgggggctgcatctgttctcctggaaccccagttacgctagactcgacaaacaaggcaattttaacgcatgggtggcaggctcttacgggaacgatcagtggctgcaagtagacttgggaagtagtaaggaggtgactgggatcattacccagggggcacgaaatttcggttccgttcagttcgttgcatcttataaggtagcgtattcaaatgactccgcgaattggaccgaatatcaggacccgcgaaccggatcaagcaagatttttccggggaattgggacaaccactctcacaaaaaaaatttgtttgaaacacctatactggcgcggtacgttagaatcctcccagttgcctggcacaaccggatagcgcttagactggaattgttggggtgc

SEQ ID NO: 14 - DNA, 282 bp, Homo sapiens
  misc_feature 1..282: note = Gas6 binding domain
  source 1..282: mol_type = other DNA
atggctccctctttgtcaccaggacctgcggctcttaggcgagccccgcagctgctgcttctcctgctcgctgcagaatgcgctctcgctgcactcttgcccgcgagggaggcgactcagttcttgcgcccccggcagagacgagcattccaagtctttgaggaagcgaaacaaggtcatctcgagcgagaatgcgtggaggagctgtgttctagggaggaagcacgcgaagtctttgagaatgacccggaaacggactacttttacccccggtatcttgat

SEQ ID NO: 15 - DNA, 261 bp, Homo sapiens
  misc_feature 1..261: note = Protein S binding domain
  source 1..261: mol_type = other DNA
atgcgcgtgttggggggtcgctgtggtgcgctccttgcttgtctccttttggttcttcccgtctccgaggctaatttcctgtcaaaacaacaggctagtcaagtcttggtgcgcaagaggagagctaacagccttctggaagagaccaagcaaggtaatctggagagagagtgtatcgaggaactttgtaacaaagaggaagcacgcgaagtatttgaaaatgacccggaaaccgattatttttacccaaaatatctcgta

SEQ ID NO: 16 - protein, 12 aa, synthetic construct
  REGION 1..12: note = modified IgG4 hinge
ESKYGPPCPPCP

SEQ ID NO: 17 - protein, 13 aa, Homo sapiens
  REGION 1..13: note = TLR4 juxtamembrane domain
PVLSLNITCQMNK

SEQ ID NO: 18 - protein, 21 aa, Homo sapiens
  REGION 1..21: note = Tim1 transmembrane domain
IYAGVCISVLVLLALLGVIIA

SEQ ID NO: 19 - protein, 21 aa, Homo sapiens
  REGION 1..21: note = Tim4 transmembrane domain
LLMIIAPSLGFVLFALFVAFL

SEQ ID NO: 20 - protein, 21 aa, Homo sapiens
  REGION 1..21: note = FcgammaRI transmembrane domain
VLFYLAVGIMFLVNTVLWVTI

SEQ ID NO: 21 - protein, 21 aa, Homo sapiens
  REGION 1..21: note = FcepsilonRIgamma transmembrane domain
LCYILDAILFLYGIVLTLLYC

SEQ ID NO: 22 - protein, 21 aa, Homo sapiens
  REGION 1..21: note = CD8a transmembrane domain
IYIWAPLAGTCGVLLLSLVIT

SEQ ID NO: 23 - protein, 21 aa, Homo sapiens
  REGION 1..21: note = MERTK transmembrane domain
FGCFCGFILIGLILYISLAIR

SEQ ID NO: 24 - protein, 21 aa, Homo sapiens
  REGION 1..21: note = Axl transmembrane domain
YVLLGAVVAAACVLILALFLV

SEQ ID NO: 25 - protein, 21 aa, Homo sapiens
  REGION 1..21: note = Tyro3 transmembrane domain
VPVVLGVLTALVTAAALALIL

SEQ ID NO: 26 - protein, 27 aa, Homo sapiens
  REGION 1..27: note = CD28 transmembrane domain
FWVLVVVGGVLACYSLLVTVAFIIFWV

SEQ ID NO: 27 - protein, 22 aa, Homo sapiens
  REGION 1..22: note = CD4 transmembrane domain
MALIVLGGVAGLLLFIGLGIFF

SEQ ID NO: 28 - protein, 21 aa, Homo sapiens
  REGION 1..21: note = DAP12 transmembrane domain
GVLAGIVMGDLVLTVLIALAV

SEQ ID NO: 29 - protein, 21 aa, Homo sapiens
  REGION 1..21: note = BAI1 transmembrane domain
VTLIVGCGVSSLTLLMLVIIY

SEQ ID NO: 30 - protein, 21 aa, Homo sapiens
  REGION 1..21: note = MRC1 transmembrane domain
GVVIIVILLILTGAGLAAYFF

SEQ ID NO: 31 - protein, 21 aa, Homo sapiens
  REGION 1..21: note = TLR1 transmembrane domain
LLIVTIVATMLVLAVTVTSLC

SEQ ID NO: 32 - protein, 21 aa, Homo sapiens
  REGION 1..21: note = TLR2 transmembrane domain
ALVSGMCCALFLLILLTGVLC

SEQ ID NO: 33 - protein, 21 aa, Homo sapiens
  REGION 1..21: note = TLR3 Transmembrane domain
FFMINTSILLIFIFIVLLIHF

SEQ ID NO: 34 - protein, 21 aa, Homo sapiens
  REGION 1..21: note = TLR4 transmembrane domain
TIIGVSVLSVLVVSVVAVLVY

SEQ ID NO: 35 - protein, 21 aa, Homo sapiens
  REGION 1..21: note = TLR5 transmembrane domain
FSLFIVCTVTLTLFLMTILTV

SEQ ID NO: 36 - protein, 21 aa, Homo sapiens
  REGION 1..21: note = TLR6 transmembrane domain
ALVSGMCCALFLLILLTGVLC

SEQ ID NO: 37 - protein, 21 aa, Homo sapiens
  REGION 1..21: note = TLR7 transmembrane domain
LILFSLSISVSLFLMVMMTAS

SEQ ID NO: 38 - protein, 21 aa, Homo sapiens
  REGION 1..21: note = TLR8 transmembrane domain
AVILFFFTFFITTMVMLAALA

SEQ ID NO: 39 - protein, 21 aa, Homo sapiens
  REGION 1..21: note = TLR9 transmembrane domain
FALSLLAVALGLGVPMLHHLC

SEQ ID NO: 40 - DNA, 63 bp, Homo sapiens
  misc_feature 1..63: note = Tim1 transmembrane domain
  source 1..63: mol_type = other DNA
atatacgctggagtctgtataagtgtccttgtactgttggcgttgctgggggtcattattgcc

SEQ ID NO: 41 - DNA, 63 bp, Homo sapiens
  misc_feature 1..63: note = Tim4 transmembrane domain
  source 1..63: mol_type = other DNA
ttgcttatgattattgcgccaagccttggatttgtgctgttcgcactcttcgtagcttttctc

SEQ ID NO: 42 - DNA, 63 bp, Homo sapiens
  misc_feature 1..63: note = FcgammaRI transmembrane domain
  source 1..63: mol_type = other DNA
gtactgttttatctcgccgtagggataatgttcctcgtgaacaccgtactgtgggtaacaata

SEQ ID NO: 43 - DNA, 63 bp, Homo sapiens
  misc_feature 1..63: note = CD8a transmembrane domain
  source 1..63: mol_type = other DNA
atatacatttgggcaccgctggctggaacttgcggcgttctcttgttgagtctggtgattact

SEQ ID NO: 44 - DNA, 63 bp, Homo sapiens
  misc_feature 1..63: note = MERTK transmembrane domain
  source 1..63: mol_type = other DNA
tttggctgtttctgtggatttattctgattggtcttatcctctatatttccttggcgatcaga

SEQ ID NO: 45 - DNA, 63 bp, Homo sapiens
  misc_feature 1..63: note = Axl transmembrane domain
  source 1..63: mol_type = other DNA
tatgtcttgcttggtgccgtcgttgctgccgcctgtgtgttgatactcgcacttttcttggtg

SEQ ID NO: 46 - DNA, 63 bp, Homo sapiens
  misc_feature 1..63: note = Tyro3 transmembrane domain
  source 1..63: mol_type = other DNA
gtacccgtcgttttgggggtcctgaccgcgctcgttactgcggcagcactcgcactgatactt

SEQ ID NO: 47 - DNA, 66 bp, Homo sapiens
  misc_feature 1..66: note = CD4 transmembrane domain
  source 1..66: mol_type = other DNA
atggctctgatcgtactgggcggagtggcaggattgctgctctttattggactgggcattttcttc

SEQ ID NO: 48 - protein, 185 aa, Homo sapiens
  REGION 1..185: note = TLR1 signaling domain
SYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 49 - protein, 175 aa, Homo sapiens
  REGION 1..175: note = TLR2 signaling domain
HRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 50 - protein, 179 aa, Homo sapiens
  REGION 1..179: note = TLR3 signaling domain
EGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 51 - protein, 187 aa, Homo sapiens
  REGION 1..187: note = TLR4 signaling domain
KFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 52 - protein, 198 aa, Homo sapiens
  REGION 1..198: note = TLR5 signaling domain
TKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 53 - protein, 189 aa, Homo sapiens
  REGION 1..189: note = TLR6 signaling domain
YLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 54 - protein, 189 aa, Homo sapiens
  REGION 1..189: note = TLR7 signaling domain
HLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 55 - protein, 193 aa, Homo sapiens
  REGION 1..193: note = TLR8 signaling domain
HHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 56 - protein, 193 aa, Homo sapiens
  REGION 1..193: note = TLR9 signaling domain
GWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 57 - protein, 522 aa, Homo sapiens
  REGION 1..522: note = Traf6 signaling domain - full length
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV

SEQ ID NO: 58 - protein, 274 aa, Homo sapiens
  REGION 1..274: note = truncated TRAF6 signaling domain
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 59 - protein, 473 aa, Homo sapiens
  REGION 1..473: note = MERTK signaling domain
KRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 60-61 - skipped (2 entries)

SEQ ID NO: 62 - protein, 42 aa, Homo sapiens
  REGION 1..42: note = FcepsilonRIgamma signaling domain
RLKIQVRKAAITSYEKSDGVYTGLSTRNQETYETLKHEKPPQ

SEQ ID NO: 63 - protein, 61 aa, Homo sapiens
  REGION 1..61: note = FcgammaR1 signaling domain
RKELKRKKKWDLEISLDSGHEKKVISSLQEDRHLEEELKCQEQKEEQLQEGVHRKEPQGAT

SEQ ID NO: 64 - protein, 77 aa, Homo sapiens
  REGION 1..77: note = FcgammaR2A signaling domain
CRKKRISANSTDPVKAAQFEPPGRQMIAIRKRQLEETNNDYETADGGYMTLNPRAPTDDDKNIYLTLPPNDHVNSNN

SEQ ID NO: 65 - protein, 77 aa, Homo sapiens
  REGION 1..77: note = FcgammaR2c signaling domain
CRKKRISANSTDPVKAAQFEPPGRQMIAIRKRQPEETNNDYETADGGYMTLNPRAPTDDDKNIYLTLPPNDHVNSNN

SEQ ID NO: 66 - protein, 25 aa, Homo sapiens
  REGION 1..25: note = FcgammaR3A signaling domain
KTNIRSSTRDWKDHKFKWRKDPQDK

SEQ ID NO: 67 - protein, 85 aa, Homo sapiens
  REGION 1..85: note = BAFF-R signaling domain
SWRRRQRRLRGASSAEAPDGDKDAPEPLDKVIILSPGISDATAPAWPPPGEDPGTTPPGHSVPVPATELGSTELVTTKTAGPEQQ

SEQ ID NO: 68 - protein, 52 aa, Homo sapiens
  REGION 1..52: note = DAP12 signaling domain
YFLGRLVPRGRGAAEAATRKQRITETESPYQELQGQRSDVYSDLNTQRPYYK

SEQ ID NO: 69 - protein, 86 aa, Homo sapiens
  REGION 1..86: note = NFAM1 signaling domain
LWNKKRMRGPGKDPTRKCPDPRSASSPKQHPSESVYTALQRRETEVYACIENEDGSSPTAKQSPLSQERPHRFEDDGELNLVYENL

SEQ ID NO: 70 - protein, 29 aa, Homo sapiens
  REGION 1..29: note = truncated NFAM1 signaling domain
SSPKQHPSESVYTALQRRETEVYACIENE

SEQ ID NO: 71 - protein, 29 aa, Homo sapiens
  REGION 1..29: note = CD79b truncated signaling domain (185-213)
DSKAGMEEDHTYEGLDIDQTATYEDIVTL

SEQ ID NO: 72 - protein, 303 aa, Homo sapiens
  REGION 1..303: note = TRAF2 signaling domain
MAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 73 - protein, 274 aa, Homo sapiens
  REGION 1..274: note = TRAF3 signaling domain
MESSKKMDSPGALQTNPPLKLHTDRSAGTPVFVPEQGGYKEKFVKTVEDKYKCEKCHLVLCSPKQTECGHRFCESCMAALLSSSSPKCTACQESIVKDKVFKDNCCKREILALQIYCRNESRGCAEQLMLGHLLVHLKNDCHFEELPCVRPDCKEKVLRKDLRDHVEKACKYREATCSHCKSQVPMIALQKHEDTDCPCVVVSCPHKCSVQTLLRSELSAHLSECVNAPSTCSFKRYGCVFQGTNQQIKAHEASSAVQHVNLLKEWSNSLEKKV

SEQ ID NO: 74 - DNA, 1566 bp, Homo sapiens
  misc_feature 1..1566: note = Traf6 signaling domain - full length
  source 1..1566: mol_type = other DNA
atgtcactccttaactgcgaaaacagttgtgggagttcacaatccgaaagtgattgttgcgtggcgatggcgtcttcatgctctgcggttaccaaggatgactctgtgggaggcaccgcatctaccggaaatctgagctcttcttttatggaggaaattcagggctacgacgttgagtttgatcctcctctcgaatctaagtatgagtgccccatatgtctcatggcgttgagagaagcagtgcagactccgtgcggacatcgcttctgcaaggcgtgtattataaagagtatacgcgatgcgggtcacaaatgtccagtggacaacgagatactgcttgaaaatcaacttttccccgacaattttgcaaagagagagatactgtctttgatggttaagtgtccaaacgagggctgcttgcacaaaatggaactccgacaccttgaagaccaccaggcacactgcgagttcgccctcatggattgcccacaatgccagcgcccgttccaaaagtttcacataaacatccacatactgaaggactgtcctaggagacaagtaagctgtgacaattgcgcagcgtcaatggcgttcgaggacaaggagatacacgatcaaaactgtcctctggcgaatgtgatctgcgaatattgcaatacgatcttgatccgcgaacagatgcctaatcattacgacctcgattgtccgaccgcgccaattccttgtactttttctaccttcggatgtcatgagaaaatgcaacgaaatcacctggctcgccatcttcaggagaatactcagagccacatgcgcatgttggctcaagccgtacatagccttagcgtaataccggactcaggttatatatccgaagtacggaattttcaagaaaccatacatcaacttgaaggaaggttggtacgacaggatcatcagatacgcgaattgacggccaagatggaaacccagagcatgtatgtcagtgagcttaagcgcactatccgaaccctggaggataaagttgccgaaatcgaagctcaacaatgcaacgggatatacatttggaaaataggtaacttcggaatgcacctgaagtgtcaagaagaagaaaaacctgtcgttattcattcccccggcttttatacagggaagcctgggtataagctttgcatgaggctccacctccaattgccgacggcgcaaaggtgcgcaaattacatttctctgtttgtccatactatgcagggtgagtacgatagtcacttgccgtggcccttccagggtaccatacgattgaccatcctggatcagagcgaggcccccgtgcgacagaatcatgaagaaataatggatgctaagccggaactgctcgctttccagagacctacaattccgcgaaatcctaagggttttggctatgttacgttcatgcatctggaagcactcagacaaagaacattcattaaagatgacaccttgcttgtgcggtgtgaggtgtcaaccaggttcgacatgggatctctcagacgggaggggttccaaccgcgctctacagacgctggagtg

SEQ ID NO: 75 - protein, 45 aa, Homo sapiens
  REGION 1..45: note = CD79b signaling domain (185-229)
DSKAGMEEDHTYEGLDIDQTATYEDIVTLRTGEVKWSVGEHPGQE

SEQ ID NO: 76 - protein, 141 aa, Homo sapiens
  REGION 1..141: note = MyD88 TIR domain
HMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 77 - DNA, 183 bp, Homo sapiens
  misc_feature 1..183: note = FcgammaR1 signaling domain
  source 1..183: mol_type = other DNA
agaaaggaactcaagcgcaagaagaagtgggacctggagatttctctcgactccggtcacgaaaagaaggtcatcagtagcttgcaagaggaccgacacttggaagaagaacttaaatgccaggaacagaaagaggagcagctccaggagggagtccaccggaaagaaccacagggagcaact

SEQ ID NO: 78 - DNA, 231 bp, Homo sapiens
  misc_feature 1..231: note = FcgammaR2A signaling domain
  source 1..231: mol_type = other DNA
tgtcgaaagaagcggatttcagccaatagtacagacccagtgaaagccgctcaatttgagccacccggtcgacagatgatcgcaattaggaaacgccaactggaggaaacgaataatgattacgaaacggcagatgggggctacatgacgctcaatcctagagctccgaccgacgacgacaagaatatatatctgactctccctcccaacgaccacgtaaacagtaataac

SEQ ID NO: 79 - DNA, 231 bp, Homo sapiens
  misc_feature 1..231: note = FcgammaR2C signaling domain
  source 1..231: mol_type = other DNA
tgcagaaagaagcggataagtgcaaatagtactgatcccgttaaagcagcacaatttgagccgccaggacggcaaatgattgcaatcagaaaacgacaacccgaggaaaccaataatgactacgagaccgctgacggagggtatatgacgttgaatccccgcgcaccaacggatgacgataagaacatttatcttacgctgccccctaacgatcatgtgaatagcaataac

SEQ ID NO: 80 - DNA, 75 bp, Homo sapiens
  misc_feature 1..75: note = FcgammaR3A signaling domain
  source 1..75: mol_type = other DNA
aaaacaaatatccggtcctctacgagggactggaaagatcataaattcaagtggagaaaagatcctcaggataaa

SEQ ID NO: 81 - protein, 487 aa, synthetic construct
  REGION 1..487: note = CER05 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 82 - protein, 487 aa, synthetic construct
  REGION 1..487: note = CER06 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 83 - protein, 500 aa, synthetic construct
  REGION 1..500: note = CER07 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 84 - protein, 479 aa, synthetic construct
  REGION 1..479: note = CER17 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 85 - protein, 479 aa, synthetic construct
  REGION 1..479: note = CER18 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 86 - protein, 498 aa, synthetic construct
  REGION 1..498: note = CER19 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 87 - protein, 498 aa, synthetic construct
  REGION 1..498: note = CER20 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 88 - protein, 493 aa, synthetic construct
  REGION 1..493: note = CER21 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 89 - protein, 493 aa, synthetic construct
  REGION 1..493: note = CER22 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 90 - protein, 493 aa, synthetic construct
  REGION 1..493: note = CER23 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 91 - protein, 493 aa, synthetic construct
  REGION 1..493: note = CER24 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 92 - protein, 485 aa, synthetic construct
  REGION 1..485: note = CER26 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 93 - protein, 475 aa, synthetic construct
  REGION 1..475: note = CER27 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 94 - protein, 489 aa, synthetic construct
  REGION 1..489: note = CER28 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 95 - skipped

SEQ ID NO: 96 - protein, 603 aa, synthetic construct
  REGION 1..603: note = CER30 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 97 - protein, 574 aa, synthetic construct
  REGION 1..574: note = CER31 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMESSKKMDSPGALQTNPPLKLHTDRSAGTPVFVPEQGGYKEKFVKTVEDKYKCEKCHLVLCSPKQTECGHRFCESCMAALLSSSSPKCTACQESIVKDKVFKDNCCKREILALQIYCRNESRGCAEQLMLGHLLVHLKNDCHFEELPCVRPDCKEKVLRKDLRDHVEKACKYREATCSHCKSQVPMIALQKHEDTDCPCVVVSCPHKCSVQTLLRSELSAHLSECVNAPSTCSFKRYGCVFQGTNQQIKAHEASSAVQHVNLLKEWSNSLEKKV

SEQ ID NO: 98 - protein, 478 aa, synthetic construct
  REGION 1..478: note = CER42 chimeric engulfment receptor
  SIGNAL 1..22
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKESKYGPPCPPCPTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 99 - protein, 22 aa, Homo sapiens
  REGION 1..22: note = GM-CSF signal peptide sequence
MLLLVTSLLLCELPHPAFLLIP

SEQ ID NO: 100 - protein, 22 aa, Mus musculus
  REGION 1..22: note = Tim4 signal peptide sequence
MSKGLLLLWLVTELWWLYLTPA

SEQ ID NO: 101 - protein, 19 aa, synthetic construct
  REGION 1..19: note = P2A self-cleaving peptide
ATNFSLLKQAGDVEENPGP

SEQ ID NO: 102 - protein, 18 aa, synthetic construct
  REGION 1..18: note = T2A self-cleaving peptide
EGRGSLLTCGDVEENPGP

SEQ ID NO: 103 - protein, 20 aa, synthetic construct
  REGION 1..20: note = E2A self-cleaving peptide
QCTNYALLKLAGDVESNPGP

SEQ ID NO: 104 - protein, 22 aa, synthetic construct
  REGION 1..22: note = F2A self-cleaving peptide
VKQTLNFDLLKLAGDVESNPGP

SEQ ID NO: 105 - protein, 285 aa, Homo sapiens
  REGION 1..285: note = truncated EGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCH

SEQ ID NO: 106 - protein, 279 aa, Mus musculus
  REGION 1..279: note = Tim4 binding domain
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQT

SEQ ID NO: 107 - protein, 257 aa, Mus musculus
  REGION 1..257: note = Tim4 binding domain without signal peptide
ASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQT

SEQ ID NO: 108 - protein, 21 aa, Mus musculus
  REGION 1..21: note = Tim4 transmembrane domain
ILIIACCVGFVLMVLLFLAFL

SEQ ID NO: 109 - protein, 267 aa, synthetic construct
  REGION 1..267: note = FMC63 scFv
  SIGNAL 1..22
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 110 - DNA, 81 bp, Homo sapiens
  misc_feature 1..81: note = CD28 transmembrane domain
  source 1..81: mol_type = other DNA
ttttgggtgctggtggtggttggtggagtcctggcttgctatagcttgctagtaacagtggcctttattattttctgggtg

SEQ ID NO: 111 - DNA, 63 bp, Homo sapiens
  misc_feature 1..63: note = DAP12 transmembrane domain
  source 1..63: mol_type = other DNA
ggcgtgctggcagggatcgtgatgggagacctggtgctgacagtgctcattgccctggccgtg

SEQ ID NO: 112 - DNA, 63 bp, Homo sapiens
  misc_feature 1..63: note = TLR4 transmembrane domain
  source 1..63: mol_type = other DNA
accatcattggtgtgtcggtcctcagtgtgcttgtagtatctgttgtagcagttctggtctat

SEQ ID NO: 113 - DNA, 63 bp, Homo sapiens
  misc_feature 1..63: note = BAI1 transmembrane domain
  source 1..63: mol_type = other DNA
gtgacgctcatcgtgggctgtggcgtgtcctctctcaccctgctcatgctggtcatcatctac

SEQ ID NO: 114 - protein, 24 aa, Homo sapiens
  VARIANT 1..24: note = Tim4 signal peptide
MSKEPLILWLMIEFWWLYLTPVTS

SEQ ID NO: 115 - DNA, 126 bp, Homo sapiens
  misc_feature 1..126: note = FcepsilonRIgamma signaling domain
  source 1..126: mol_type = other DNA
cgactgaagatccaagtgcgaaaggcagctataaccagctatgagaaatcagatggtgtttacacgggcctgagcaccaggaaccaggagacttacgagactctgaagcatgagaaaccaccacag

SEQ ID NO: 116 - DNA, 156 bp, Homo sapiens
  misc_feature 1..156: note = DAP12 signaling domain
  source 1..156: mol_type = other DNA
tattttctgggaaggctcgttcctagaggtagaggtgctgccgaagcagcgacgcgcaaacagaggattactgaaacggagtctccctaccaagagctgcaaggccagaggtcagatgtctattcagacttgaacacacaaaggccatactacaaa

SEQ ID NO: 117 - DNA, 255 bp, Homo sapiens
  misc_feature 1..255: note = BAFFR signaling domain
  source 1..255: mol_type = other DNA
tcctggagacggcgacaaaggcgcttgcgcggcgcatcatccgcagaggcgcccgacggcgataaggacgcgcccgaaccccttgataaagttattatcttgtcaccgggaatttctgacgctacggcacccgcgtggcctcctccgggcgaagatcctggtacgacaccccctggacacagtgttcccgtgcccgcgacagagctcggtagcacagaactggtgaccacaaagacggcgggaccggaacagcaa

SEQ ID NO: 118 - DNA, 135 bp, Homo sapiens
  misc_feature 1..135: note = CD79b signaling domain
  source 1..135: mol_type = other DNA
gacagtaaagccgggatggaagaggaccacacatacgaggggcttgacatagatcaaacagcgacatacgaagacatcgtaaccttgcggactggagaggttaaatggtcagtcggagaacaccccggccaagaa

SEQ ID NO: 119 - DNA, 258 bp, Homo sapiens
  misc_feature 1..258: note = NFAM1 signaling domain
  source 1..258: mol_type = other DNA
ctctggaataaaaagaggatgcgcggcccgggaaaagacccaacgagaaagtgtcccgatccccgcagtgcgtcaagccccaagcagcatccttccgaaagcgtatatacggcacttcaacgccgggaaacggaggtatatgcgtgtattgagaacgaggacgggtcatccccgaccgccaaacagtcccctctcagccaagagcgacctcacaggtttgaggacgatggtgaactcaatctggtctacgaaaacctg

SEQ ID NO: 120 - DNA, 2754 bp, Homo sapiens
  misc_feature 1..2754: note = BAI1 binding domain
  source 1..2754: mol_type = other DNA
gccgccggagcagacgcggggcccgggcccgagccgtgcgccacgctggtgcagggaaagttcttcggctacttctccgcggccgccgtgttcccggccaacgcctcgcgctgctcctggacgctacgcaacccggacccgcggcgctacactctctacatgaaggtggccaaggcgcccgtgccctgcagcggccccggccgcgtgcgcacctaccagttcgactccttcctcgagtccacgcgcacctacctgggcgtggagagcttcgacgaggtgctgcggctctgcgacccctccgcacccctggccttcctgcaggccagcaagcagttcctgcagatgcggcgccagcagccgccccagcacgacgggctccggccccgggccgggccgccgggccccaccgacgacttctccgtggagtacctggtggtggggaaccgcaaccccagccgtgccgcctgccagatgctgtgccgctggctggacgcgtgtctggccggtagtcgcagctcgcacccctgcgggatcatgcagaccccctgcgcctgcctgggcggcgaggcgggcggccctgccgcgggacccctggccccccgcggggatgtctgcttgagagatgcggtggctggtggccctgaaaactgcctcaccagcctgacccaggaccggggcgggcacggcgccacaggcggctggaagctgtggtccctgtggggcgaatgcacgcgggactgcgggggaggcctccagacgcggacgcgcacctgcctgcccgcgccgggcgtggagggcggcggctgcgagggggtgctggaggagggtcgccagtgcaaccgcgaggcctgcggccccgctgggcgcaccagctcccggagccagtccctgcggtccacagatgcccggcggcgcgaggagctgggggacgagctgcagcagtttgggttcccagccccccagaccggtgacccagcagccgaggagtggtccccgtggagcgtgtgctccagcacctgcggcgagggctggcagacccgcacgcgcttctgcgtgtcctcctcctacagcacgcagtgcagcggacccctgcgcgagcagcggctgtgcaacaactctgccgtgtgcccagtgcatggtgcctgggatgagtggtcgccctggagcctctgctccagcacctgtggccgtggctttcgggatcgcacgcgcacctgcaggcccccccagtttgggggcaacccctgtgagggccctgagaagcaaaccaagttctgcaacattgccctgtgccctggccgggcagtggatggaaactggaatgagtggtcgagctggagcgcctgctccgccagctgctcccagggccgacagcagcgcacgcgtgaatgcaacgggccttcctacgggggtgcggagtgccagggccactgggtggagacccgagactgcttcctgcagcagtgcccagtggatggcaagtggcaggcctgggcgtcatggggcagttgcagcgtcacgtgtggggctggcagccagcgacgggagcgtgtctgctctgggcccttcttcgggggagcagcctgccagggcccccaggatgagtaccggcagtgcggcacccagcggtgtcccgagccccatgagatctgtgatgaggacaactttggtgctgtgatctggaaggagaccccagcgggagaggtggctgctgtccggtgtccccgcaacgccacaggactcatcctgcgacggtgtgagctggacgaggaaggcatcgcctactgggagccccccacctacatccgctgtgtttccattgactacagaaacatccagatgatgacccgggagcacctggccaaggctcagcgagggctgcctggggagggggtctcggaggtcatccagacactggtggagatctctcaggacgggaccagctacagtggggacctgctgtccaccatcgatgtcctgaggaacatgacagagattttccggagagcgtactacagccccacccctggggacgtacagaactttgtccagatccttagcaacctgttggcagaggagaatcgggacaagtgggaggaggcccagctggcgggccccaacgccaaggagctgttccggctggtggaggactttgtggacgtcatcggcttccgcatgaaggacctgagggatgcataccaggtgacagacaacctggttctcagcatccataagctcccagccagcggagccactgacatcagcttccccatgaagggctggcgggccacgggtgactgggccaaggtgccagaggacagggtcactgtgtccaagagtgtcttctccacggggctgacagaggccgatgaagcatccgtgtttgtggtgggcaccgtgctctacaggaacctgggcagcttcctggccctgcagaggaacacgaccgtcctgaattctaaggtgatctccgtgactgtgaaacccccgcctcgctccctgcgcacacccttggagatcgagtttgcccacatgtataatggcaccaccaaccagacctgtatcctgtgggatgagacggatgtaccctcctcctccgcccccccgcagctcgggccctggtcgtggcgcggctgccgcacggtgcccctcgacgccctccggacgcgctgcctctgtgaccggctctccaccttcgccatcttagcccagctcagcgccgacgcgaacatggagaaggcgactctgccgtcg

SEQ ID NO: 121 - DNA, 63 bp, Homo sapiens
  misc_feature 1..63: note = FcepsilonRIgamma transmembrane domain
  source 1..63: mol_type = other DNA
ctttgttacattctcgacgcgatattgttcctttatggaatagttttgacgctcctttattgc

SEQ ID NO: 122 - protein, 488 aa, synthetic construct
  REGION 1..488: note = CER43 chimeric engulfment receptor
  SIGNAL 1..22
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 123 - protein, 487 aa, synthetic construct
  REGION 1..487: note = CER44 chimeric engulfment receptor
  SIGNAL 1..22
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 124 - protein, 574 aa, synthetic construct
  REGION 1..574: note = CER29 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 125 - protein, 626 aa, synthetic construct
  REGION 1..626: note = CER110 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAYFLGRLVPRGRGAAEAATRKQRITETESPYQELQGQRSDVYSDLNTQRPYYK

SEQ ID NO: 126 - protein, 574 aa, synthetic construct; identical to SEQ ID NO: 124
  REGION 1..574: note = CER111B chimeric engulfment receptor
  SIGNAL 1..22

SEQ ID NO: 127 - protein, 638 aa, synthetic construct
  REGION 1..638: note = CER113 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLASWRRRQRRLRGASSAEAPDGDKDAPEPLDKVIILSPGISDATAPAWPPPGEDPGTTPPGHSVPVPATELGSTELVTTKTAGPEQQ

SEQ ID NO: 128 - protein, 660 aa, synthetic construct
  REGION 1..660: note = CER112 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLALWNKKRMRGPGKDPTRKCPDPRSASSPKQHPSESVYTALQRRETEVYACIENEDGSSPTAKQSPLSQERPHRFEDDGELNLVYENL

SEQ ID NO: 129 - skipped

SEQ ID NO: 130 - protein, 579 aa, synthetic construct
  REGION 1..579: note = CER102 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYLWNKKRMRGPGKDPTRKCPDPRSASSPKQHPSESVYTALQRRETEVYACIENEDGSSPTAKQSPLSQERPHRFEDDGELNLVYENL

SEQ ID NO: 131 - protein, 538 aa, synthetic construct
  REGION 1..538: note = CER103A protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYDSKAGMEEDHTYEGLDIDQTATYEDIVTLRTGEVKWSVGEHPGQE

SEQ ID NO: 132 - protein, 522 aa, synthetic construct
  REGION 1..522: note = CER103B protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYDSKAGMEEDHTYEGLDIDQTATYEDIVTL

SEQ ID NO: 133 - protein, 545 aa, synthetic construct
  REGION 1..545: note = CER104 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYYFLGRLVPRGRGAAEAATRKQRITETESPYQELQGQRSDVYSDLNTQRPYYK

SEQ ID NO: 134 - protein, 578 aa, synthetic construct
  REGION 1..578: note = CER105 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 135 - protein, 579 aa, synthetic construct
  REGION 1..579: note = CER106 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLWNKKRMRGPGKDPTRKCPDPRSASSPKQHPSESVYTALQRRETEVYACIENEDGSSPTAKQSPLSQERPHRFEDDGELNLVYENLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 136 - protein, 522 aa, synthetic construct
  REGION 1..522: note = CER107 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLDSKAGMEEDHTYEGLDIDQTATYEDIVTLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 137 - protein, 545 aa, synthetic construct
  REGION 1..545: note = CER108 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLYFLGRLVPRGRGAAEAATRKQRITETESPYQELQGQRSDVYSDLNTQRPYYKHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 138 - protein, 578 aa, synthetic construct
  REGION 1..578: note = CER109 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSWRRRQRRLRGASSAEAPDGDKDAPEPLDKVIILSPGISDATAPAWPPPGEDPGTTPPGHSVPVPATELGSTELVTTKTAGPEQQHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 139 - protein, 619 aa, synthetic construct
  REGION 1..619: note = CER111A protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLADSKAGMEEDHTYEGLDIDQTATYEDIVTLRTGEVKWSVGEHPGQE

SEQ ID NO: 140 - protein, 659 aa, synthetic construct
  REGION 1..659: note = CER113 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLASWRRRQRRLRGASSAEAPDGDKDAPEPLDKVIILSPGISDATAPAWPPPGEDPGTTPPGHSVPVPATELGSTELVTTKTAGPEQQ

SEQ ID NO: 141 - protein, 1050 aa, synthetic construct
  REGION 1..1050: note = CER114 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 142 - protein, 1050 aa, synthetic construct
  REGION 1..1050: note = CER115 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 143 - protein, 767 aa, synthetic construct
  REGION 1..767: note = CER116 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 144 - protein, 767 aa, synthetic construct
  REGION 1..767: note = CER117 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 145 - protein, 571 aa, synthetic construct
  REGION 1..571: note = CER118 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKLWNKKRMRGPGKDPTRKCPDPRSASSPKQHPSESVYTALQRRETEVYACIENEDGSSPTAKQSPLSQERPHRFEDDGELNLVYENL

SEQ ID NO: 146 - protein, 514 aa, synthetic construct
  REGION 1..514: note = CER119B protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKDSKAGMEEDHTYEGLDIDQTATYEDIVTL

SEQ ID NO: 147 - protein, 537 aa, synthetic construct
  REGION 1..537: note = CER120 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKYFLGRLVPRGRGAAEAATRKQRITETESPYQELQGQRSDVYSDLNTQRPYYK

SEQ ID NO: 148 - protein, 759 aa, synthetic construct
  REGION 1..759: note = CER121 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 149 - protein, 527 aa, synthetic construct
  REGION 1..527: note = CER122 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSYFLGRLVPRGRGAAEAATRKQRITETESPYQELQGQRSDVYSDLNTQRPYYK

SEQ ID NO: 150 - protein, 749 aa, synthetic construct
  REGION 1..749: note = CER123 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 151 - protein, 561 aa, synthetic construct
  REGION 1..561: note = CER124 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSLWNKKRMRGPGKDPTRKCPDPRSASSPKQHPSESVYTALQRRETEVYACIENEDGSSPTAKQSPLSQERPHRFEDDGELNLVYENL

SEQ ID NO: 152 - protein, 520 aa, synthetic construct
  REGION 1..520: note = CER125A protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSDSKAGMEEDHTYEGLDIDQTATYEDIVTLRTGEVKWSVGEHPGQE

SEQ ID NO: 153 - protein, 504 aa, synthetic construct
  REGION 1..504: note = CER125B protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSDSKAGMEEDHTYEGLDIDQTATYEDIVTL

SEQ ID NO: 154 - protein, 24 aa, synthetic construct
  REGION 1..24: note = T2A self-cleaving peptide variant
LEGGGEGRGSLLTCGDVEENPGPR

SEQ ID NO: 155 - protein, 19 aa, synthetic construct
  REGION 1..19: note = T2A self-cleaving peptide variant
EGRGSLLTCGDVEENPGPR

SEQ ID NO: 156 - protein, 23 aa, synthetic construct
  REGION 1..23: note = T2A self-cleaving peptide variant
LEGGGEGRGSLLTCGDVEENPGP

SEQ ID NO: 157 - protein, 27 aa, synthetic construct
  REGION 1..27: note = P2A self-cleaving peptide variant
RAKRSGSGATNFSLLKQAGDVEENPGP

SEQ ID NO: 158 - protein, 598 aa, synthetic construct
  REGION 1..598: note = HPV16 E7 TCRbeta chain-P2A-TCRalpha chain
MAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 159 - protein, 386 aa, synthetic construct
  REGION 1..386: note = CER25 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLWNKKRMRGPGKDPTRKCPDPRSASSPKQHPSESVYTALQRRETEVYACIENEDGSSPTAKQSPLSQERPHRFEDDGELNLVYENL

SEQ ID NO: 160 - protein, 135 aa, synthetic construct
  REGION 1..135: note = HPV16 E7 TCR Vbeta region
MAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVL

SEQ ID NO: 161 - protein, 173 aa, synthetic construct
  REGION 1..173: note = TCR Cbeta region, Cys-substituted
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 162 - protein, 126 aa, synthetic construct
  REGION 1..126: note = HPV16 E7TCR Valpha region
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIP

SEQ ID NO: 163 - protein, 137 aa, synthetic construct
  REGION 1..137: note = TCR Calpha region, Cys-substituted, LVL substituted
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 164 - protein, 1108 aa, synthetic construct
  REGION 1..1108: note = CER5_T2A_HPV16_E7_TCR tandem cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSILEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 165 - protein, 1119 aa, synthetic construct
  REGION 1..1119: note = CER19_T2A_HPV16_E7_TCR tandem cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATISLEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 166 - protein, 1114 aa, synthetic construct
  REGION 1..1114: note = CER21_T2A_HPV16_E7_TCR tandem cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYLEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 167 - protein, 1007 aa, synthetic construct
  REGION 1..1007: note = CER25_T2A_HPV16_E7_TCR tandem cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLWNKKRMRGPGKDPTRKCPDPRSASSPKQHPSESVYTALQRRETEVYACIENEDGSSPTAKQSPLSQERPHRFEDDGELNLVYENLLEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 168 - protein, 1096 aa, synthetic construct
  REGION 1..1096: note = CER27_T2A_HPV16_E7_TCR tandem cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSLEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 169 - protein, 1195 aa, synthetic construct
  REGION 1..1195: note = CER29_T2A_HPV16_E7_TCR tandem cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLALEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 170 - protein, 1195 aa, synthetic construct
  REGION 1..1195: note = CER31_T2A_HPV16_E7_TCR tandem cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMESSKKMDSPGALQTNPPLKLHTDRSAGTPVFVPEQGGYKEKFVKTVEDKYKCEKCHLVLCSPKQTECGHRFCESCMAALLSSSSPKCTACQESIVKDKVFKDNCCKREILALQIYCRNESRGCAEQLMLGHLLVHLKNDCHFEELPCVRPDCKEKVLRKDLRDHVEKACKYREATCSHCKSQVPMIALQKHEDTDCPCVVVSCPHKCSVQTLLRSELSAHLSECVNAPSTCSFKRYGCVFQGTNQQIKAHEASSAVQHVNLLKEWSNSLEKKVLEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 171 - protein, 155 aa, Homo sapiens
  REGION 1..155: note = truncated MyD88 signaling domain without TIR domain
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 172 - protein, 296 aa, Homo sapiens
  REGION 1..296: note = MyD88 signaling domain
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 173 - protein, 530 aa, synthetic construct
  REGION 1..530: note = CER119A protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKDSKAGMEEDHTYEGLDIDQTATYEDIVTLRTGEVKWSVGEHPGQE

SEQ ID NO: 174 - protein, 778 aa, synthetic construct
  REGION 1..778: note = CER126 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 175 - protein, 778 aa, synthetic construct
  REGION 1..778: note = CER127 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEACHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 176 - protein, 796 aa, synthetic construct
  REGION 1..796: note = CER128 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEACHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 177 - protein, 796 aa, synthetic construct
  REGION 1..796: note = CER129 protein
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

(19) United States
(12) Patent Application Publication
(10) Pub. No.: US 2024/0174766 A1
(43) Pub. Date: May 30, 2024

(54) CHIMERIC ENGULFMENT RECEPTOR MOLECULES AND METHODS OF USE

(71) Applicant: CERO THERAPEUTICS, INC., South San Francisco, CA (US)

(72) Inventor: Daniel Mark COREY, Menlo Park, CA (US)

(21) Appl. No.: 18/322,450

(22) Filed: May 23, 2023

Related U.S. Application Data
(62) Division of application No. 16/646,530, filed on Mar. 11, 2020, now Pat. No. 11,708,423.
(60) Provisional application No. 62/652,822, filed on Apr. 4, 2018.
(60) Provisional application No. 62/649,529, filed on Mar. 28, 2018.
(60) Provisional application No. 62/563,615, filed on Sep. 26, 2017.

(51) Int. Cl. C07K 16/44; A61K 35/17; A61P 35/00; C07K 14/705; C07K 14/735; C07K 16/28

(52) U.S. Cl. CPC C07K 16/44; A61K 35/17; A61P 35/00; C07K 14/70503; C07K 14/70535; C07K 14/70596; C07K 16/2803; C07K 2317/53; C07K 2317/622; C07K 2319/02; C07K 2319/03

(57) ABSTRACT

The present disclosure relates to chimeric engulfment receptor molecules, host cells modified to include the phagocytic engulfment molecules, and methods of making and using such receptor molecules and modified cells.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 200265_402 WO_SEQUENCE_LISTING.txt. The text file is 407 KB, was created on Sep. 19, 2018, and is being submitted electronically via EFS-Web.

### BACKGROUND

There are two principle types of phagocytosis, which are influenced by the target, cell-type and surrounding milieu. Anti-microbe phagocytosis clears and degrades disease-causing microbes, induces pro-inflammatory signaling through cytokine and chemokine secretion, and recruits immune cells to mount an effective inflammatory response. This type of phagocytosis is often referred to as “inflammatory phagocytosis” (or “immunogenic phagocytosis”). However, in some instances, such as with certain persistent infections, anti-inflammatory responses may follow microbial uptake. Anti-microbe phagocytosis is commonly performed by professional phagocytes of the myeloid lineage, such as immature dendritic cells (DCs) and macrophages and by tissue-resident immune cells.

Phagocytosis of damaged, self-derived apoptotic cells or cell debris (e.g., efferocytosis), in contrast, is typically a non-inflammatory (also referred to as a “non-immunogenic”) process. Billions of damaged, dying, and unwanted cells undergo apoptosis each day. Unwanted cells include, for example, excess cells generated during development, senescent cells, infected cells (intracellular bacteria or viruses), transformed or malignant cells, and cells irreversibly damaged by cytotoxic agents. Phagocytes execute specific, swift removal of apoptotic cells without causing damage to the surrounding tissues or inducing a pro-inflammatory immune response. Steps for apoptotic cell clearance include: (1) release of “find me” signals from apoptotic cells to recruit phagocytes to the location of apoptotic cells; (2) “eat me” signals exposed on the surface of apoptotic cells are bound by phagocytes via specific receptors; (3) cytoskeletal rearrangement to engulf the apoptotic cell; and (4) the ingested apoptotic cell is digested and specific phagocytic responses are elicited (e.g., secretion of anti-inflammatory cytokines).

There is an ongoing need for new compositions and methods of treating infections, inflammatory diseases, immune diseases, and various cancers. The methods and compositions disclosed herein meets such needs by enhancing the removal of infected, transformed, malignant, apoptotic, damaged or necrotic cells from the body in treatment of various cancers, acute and chronic infections, inflammatory, immune and selected neurological diseases.

### BRIEF SUMMARY

Chimeric, engulfment receptors are described herein. In certain embodiments, the chimeric engulfment receptors (“CER” in the singular and “CERs” in the plural) include an extracellular domain, a transmembrane domain, and an intracellular engulfment signaling domain. The transmembrane domain is positioned between and connects the extracellular domain and the engulfment signaling domain. The extracellular domain comprises a binding domain and an optional extracellular spacer domain positioned between and connecting the binding domain and transmembrane domain. In certain embodiments, the chimeric engulfment receptors described herein are chimeric proteins having (a) and extracellular domain that targets a pro-engulfment marker or a target antigen associated with a disease, disorder, condition, or infection, (b) a transmembrane domain, and (c) an engulfment signaling domain that comprises a toll-like receptor (TLR) signaling domain, a Traf6 signaling domain, aTraf2 signaling domain, or a Traf3 signaling domain. In certain embodiments, the engulfment signaling domain comprises a primary engulfment signaling domain and a secondary engulfment signaling domain. In particular embodiments, the chimeric engulfment receptors are single chain chimeric proteins. Chimeric engulfment receptors may be designed to generate an inflammatory response to a target cell/organ/tissue/area. While apoptotic cell clearance is typically a non-inflammatory process, inflammation can be beneficial to the host in certain contexts, such as, for example, in the context of clearance of apoptotic tumor cells to induce an immune response to residual tumor cells.

In some embodiments, the extracellular domain of the CER includes a binding domain specific to a pro-engulfment marker. In certain such embodiments, the extracellular domain includes a phosphatidylserine (PtdSer) binding domain. In embodiments of the CERs described herein, a PtdSer binding domain can include all or a portion of the extracellular domain of T cell immunoglobulin and mucin domain 1 (Tim1), T cell immunoglobulin and mucin domain 4 (Tim4), or T cell immunoglobulin and mucin domain 3 (Tim3). In other embodiments a PtdSer binding domain can include all or a portion of a binding domain derived from FA58C2, GAS6, protein S, Factor VII, Factor IX, Factor X, or prothrombin PS.

In further embodiments, the extracellular domain binds to a target antigen. In certain such embodiments, the extracellular domain includes all or part of the extracellular domain of an Fc receptor (FcR), such as, for example, FcγR1, FcγR2A, FcγR2B2, FcγR2C, FcγR3A, FcεR1, and FcαR1. In still other embodiments where the extracellular domain binds a target antigen, the extracellular domain can include an antibody or an antigen-binding domain thereof. For example, the extracellular domain can include an antibody or an antigen-binding domain selected from intrabodies, peptibodies, nanobodies, single domain antibodies, SMIPs, and multispecific antibodies. In certain such embodiments, the extracellular domain includes a Fab binding domain. In yet other such embodiments, the extracellular domain includes a scFv.

Upon binding of the extracellular domain of the CER to the pro-engulfment marker or targeted antigen, the engulfment signaling domain stimulates engulfment signaling activity. Thus, upon activation, the engulfment signaling domain included in the CER transduces effector functional signals that direct the host cell to engulf. In certain embodiments, the engulfment signaling domain comprises: a primary engulfment signaling domain comprising a TLR signaling domain, a Traf6 signaling domain, a Traf2 signaling domain, or a Traf3 signaling domain; and a secondary engulfment signaling domain. Examples of secondary engulfment signaling domains include FcγR1, FcγR2A, FcγR2B2, FcγR2C, FcγR3A, FcεR1, FcαR1, BAFF-R, NFAM1, DAP12, MERTK, CD79b, TLR, Traf2, Traf3, and Traf6 signaling domains.

In further aspects, the present disclosure is directed to cells genetically modified to express a CER. In specific embodiments, the CER confers an engulfment phenotype not exhibited by a single, naturally-occurring receptor protein. In other embodiments, a CER according to the present description confers an engulfment phenotype to a cell that does not naturally exhibit engulfment activity. In one embodiment, antigen binding by a CER induces phagocytic signal transduction cascade in a cell that does not naturally exhibit phagocytic signal transduction activity. In another embodiment, a CER-expressing cell that does not naturally exhibit engulfment activity and that engulfs a target cell is capable of degrading the target cell. In other embodiments, a CER according to the present disclosure further confers a phenotype to the host cell, such as enhanced proliferative activity, expansion activity, activation, cytolytic activity, antigen presentation activity, memory formation, persistence, or a combination thereof that may otherwise not be present in a host cell that does not express the CER. In certain embodiments, cells are genetically modified to express a CER that targets a pro-engulfment marker associated with dead, dying, damaged, infected, or necrotic cells. In other embodiments, cells are genetically modified to express a CER that targets a marker, such as an antibody, associated with an infectious microbe or molecule induced by an infectious particle. In such embodiments, the genetically modified cells promote clearance or degradation of the targeted cells or microbes upon binding by the CER of the marker associated with the targeted infectious microbe or the targeted molecule induced by an infectious particle. In other specific embodiments, cells are genetically modified to express a CER that targets an antigenic marker that does not normally trigger engulfment. For example, in such embodiments, the extracellular domain of the CER can include an antibody or antigen-binding portion of an antibody, such as a Fab binding domain or a scFv specific to an antigenic marker. In certain such embodiments, the antigenic marker can be, a surface protein, glycoprotein, or glycolipid characteristic of aberrant cells associated with a disease, disorder, or other undesirable condition. In such embodiments, the genetically modified cells promote clearance or degradation of the aberrant cells upon binding of the antigenic marker by the CER. In certain embodiments, cells that are genetically modified to express a CER that targets an antigenic marker that does not normally trigger engulfment are B cells.

In yet further aspects, the present disclosure is directed to a method treating a subject suffering from a disease, disorder or undesired condition. Embodiments of these methods include administering to a subject a therapeutically effective amount of a pharmaceutical composition including one or more CERs or a population of cells genetically modified to express one or more CERs according to the present description.

In other aspects, the present disclosure provides methods for altering the engulfment phenotype of a host cell. In certain embodiments, such methods include one or more of the following: methods for producing a population of cells exhibiting an engulfment phenotype by introducing into and expressing a CER in host cells that do not naturally exhibit an engulfment phenotype; methods for altering the engulfment phenotype of a population of cells by introducing into and expressing a CER in the host cells, wherein the CER confers an engulfment phenotype specific to a pro-engulfment marker or antigenic marker not naturally targeted by the host cells; and methods for enhancing the engulfment phenotype of a population of cells by introducing into and expressing a CER in the host cells, wherein the CER is specific to a pro-engulfment marker or antigenic marker naturally targeted by the host cells and expression of the CER by the host cells enhances the engulfment by the host cells of cells, microbes, or particles exhibiting the targeted pro-engulfment or antigenic marker.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIGS. 1A-B show illustrative schematics of chimeric engulfment receptors (CERs). FIG. 1A shows an illustrative CER having an extracellular domain specific for a pro-engulfment marker (e.g., Tim4 binding domain for phosphatidylserine), a transmembrane domain, an engulfment signaling domain comprising a TLR signaling domain, Traf6 signaling domain, Traf2 signaling domain, or Traf3 signaling domain, and optionally, a secondary engulfment signaling domain. FIG. 1B shows an illustrative CER having an extracellular domain comprising a scFv binding domain, a transmembrane domain, an engulfment signaling domain comprising a TLR signaling domain, Traf6 signaling domain, Traf2 signaling domain, or Traf3 signaling domain, and optionally, a secondary engulfment signaling domain.

FIGS. 2A-2B show a comparison of a natural lymphocyte and a lymphocyte modified with a CER of the present disclosure. FIG. 2A shows an endogenous lymphocyte. FIG. 2B shows a lymphocyte modified with a CER of the present disclosure.

FIG. 3 shows an illustrative method of administration of the CERs of the present disclosure.

FIGS. 4A-4C show illustrative treatment timelines. FIG. 4A shows a treatment scheme for therapy with cells modified with a CER. FIG. 4B shows a treatment scheme for CER-modified cells used in combination with non-phagocytic T cellular immune therapies. FIG. 4C shows a treatment scheme for CER-modified cells used in combination with monoclonal antibodies, conventional chemotherapy, or radiation therapy.

FIG. 5 shows an illustrative triple combination treatment timeline comprising radiation therapy, CER immunotherapy (e.g., targeting phosphatidylserine expressing cells), followed by TCR or CAR immunotherapy.

FIG. 6 shows a vector map for a lentiviral vector comprising “CER05” chimeric engulfment receptor having an amino acid sequence of SEQ ID NO:81. CER05 comprises a Tim4 binding domain, a Tim4 transmembrane domain, and a TLR4 signaling domain. The lentiviral vector also comprises a sequence encoding truncated EGFR (SEQ ID NO:105), which is separated from the CER05 sequence by a viral T2A sequence.

FIG. 7 show fluorescence microscope images of in vitro engulfment of dexamethasone treated thymocytes by CER05+ Ba/F3 cells. White arrows indicate phagocytosis events. The image on the right is was created by automated software using the image on the left, with the CER05+ Ba/F3 cells outlined in blue and the engulfed target cells shown in white.

FIG. 8 shows a bar graph of phagocytic index of CER05+, CER07+, or control EGFRt+ Ba/F3 cells that were co-cultured with dexamethasone treated thymocytes.

FIG. 9 shows fluorescence microscope image of co-localization of engulfed thymocytes and Lysotracker green signal in CER05+ Ba/F3 cells. White arrows indicate co-localization of pHrodo red labeled thymocytes and acidic compartments stained with LysoTracker green.

FIG. 10 shows fluorescence microscope image of co-localization of engulfed pHrodo red labeled thymocytes and acidic compartments stained with Lysotracker green signal in control tEGFR+ Ba/F3 cells. No co-localization is observed in this image.

FIG. 11 shows fluorescence microscope image of in vitro engulfment of staurosporine treated, pHrodo red stained CT26 colon cancer cells by CELLTRACE Violet stained CER05+ Ba/F3 cells. White arrows indicate phagocytosis events.

FIG. 12 shows fluorescence microscope images of in vitro engulfment of staurosporine treated, pHrodo red stained CT26 colon cancer cells by CELLTRACE Violet stained CER05+ Ba/F3 cells (left) compared to control tEGFR+ Ba/F3 cells (right). White arrows indicate phagocytosis events.

FIG. 13 shows a scatterplot of hybrid cell counts extracting CT26 target cell area from CER05+ Ba/F3 cells, CER07+ Ba/F3 cells, or EGFRt+ control Ba/F3 cells. The area ratio represents the overlay area of CT26 cells within Ba/F3 cells.

FIG. 14 a bar graph of phagocytic index of CER05+, CER07+, or control EGFRt+ Ba/F3 cells that were co-cultured with staurosporine treated CT26 colon cancer cells.

FIG. 15 shows a vector map for a lentiviral vector comprising “CER07” chimeric engulfment receptor having an amino acid sequence of SEQ ID NO:83. CER07 comprises a Tim4 binding domain, a TLR4 juxtamembrane domain, a TLR4 transmembrane domain, and a TLR4 signaling domain. The lentiviral vector also comprises a sequence encoding truncated EGFR (SEQ ID NO:105), which is separated from the CER07 sequence by a viral T2A sequence.

FIG. 16 shows fluorescence microscope image of in vitro engulfment of staurosporine treated CT26 colon cancer cells by CER07+ Ba/F3 cells. White arrows indicate phagocytosis events.

FIG. 17 shows fluorescence microscope images of in vitro engulfment of staurosporine treated, pHrodo red stained CT26 colon cancer cells by CELLTRACE Violet stained CER07+ Ba/F3 cells (left) compared to control tEGFR+ Ba/F3 cells (right). White arrows indicate phagocytosis events.

FIG. 18 shows a vector map for a lentiviral vector comprising “CER21” chimeric engulfment receptor having an amino acid sequence of SEQ ID NO:88. CER21 comprises a Tim4 binding domain, a Tim4 transmembrane domain, and a TLR8 signaling domain. The lentiviral vector also comprises a sequence encoding truncated EGFR (SEQ ID NO:105), which is separated from the CER21 sequence by a viral T2A sequence.

FIG. 19 shows fluorescence microscope images of in vitro phagocytosis (FIG. 19A) and cytolysis (FIG. 19B) of staurosporine treated Jurkat cells by CER21+ human primary B cells.

FIGS. 20A-20B show graphs measuring apoptotic object count (FIG. 20A) and apoptotic fluorescent count (FIG. 20B) for CER21+ human primary B cells co-cultured with Jurkat cells (+ and − staurosporine (STS)) as a measure of cytolytic activity. Human primary B cells transduced with truncated EGFR was used as controls (+ and − STS).

FIG. 21 shows fluorescence microscope images of in vitro apoptosis in co-culture of CER21+ human primary B cells or control EGFRt+ human primary B cells with paclitaxel treated H1703 non-small cell lung cancer cells. Cells undergoing apoptosis fluoresce red (top row). Automated software outlined the red fluorescent objects (bottom row).

FIG. 22 shows a graph of apoptotic object count area for CER21+ human primary B cells or control EGFRt+ human primary B cells co-cultured with paclitaxel treated H1703 cells.

FIG. 23 shows FACS plots (left) and histograms (right) of CER19+, CER21+, or control human primary B cell proliferation following co-culture with paclitaxel treated Jurkat lymphoma cells in the absence of exogenous cytokines.

FIG. 24 shows enhanced activation state of CER21+ human primary B cells as measured by increased expression of pro-inflammatory IL-1 cytokines (graph on upper left), costimulatory molecules (graph on upper right), B cell activation and survival molecules (graph on upper right), lymphocyte chemoattractants (graph on lower left) and molecules involved in lymph node tissue remodeling (graph on lower right). Expression levels were compared to control vector transduced B cells.

FIG. 25 shows fluorescence microscope images of in vitro phagocytosis of CD19+ Raji cells by CD19 specific CER44+ Ba/F3 cells. White arrows indicate engulfment events. Four enlargements of the fluorescent microscope images showing phagocytosis are shown on the right.

FIG. 26 shows FACS analysis of CER43+ Ba/F3 cells, CER44+ Ba/F3 cells, or control EGFRt Ba/F3 cells that were co-cultured with CD19+ Raji lymphoma cells. Engulfment of Raji cells by CER43+, CER44+, or EGFRt+ Ba/F3 cells was measured by cell population that stained double positive for pHrodo Red and CELLTRACE Violet.

FIG. 27 shows a graph of frequency of phagocytosis for CER43+, CER44+, or control EGFRt+ Ba/F3 cells co-cultured with Raji cells.

FIG. 28 shows a vector map for a lentiviral vector comprising “CER43” chimeric engulfment receptor having an amino acid sequence of SEQ ID NO: 122. CER43 comprises an extracellular domain comprising a CD19 specific FMC63 scFv, an extracellular spacer region comprising a TLR4 juxtamembrane domain, a TLR4 transmembrane domain, and an engulfment signaling domain comprising a TLR4 signaling domain. The lentiviral vector also comprises a sequence encoding truncated EGFR (SEQ ID NO:105), which is separated from the CER43 sequence by a viral T2A sequence.

FIG. 29 shows a vector map for a lentiviral vector comprising “CER44” chimeric engulfment receptor having an amino acid sequence of SEQ ID NO:123. CER44 comprises an extracellular domain comprising a CD19 specific FMC63 scFv, an extracellular spacer region comprising a modified IgG4 hinge region, a TLR4 transmembrane domain, and an engulfment signaling domain comprising a TLR4 signaling domain. The lentiviral vector also comprises a sequence encoding truncated EGFR (SEQ ID NO:105), which is separated from the CER44 sequence by a viral T2A sequence.

FIGS. 30A-30B show in vitro co-culture assay schematic and data from CD4+ T cells transduced with selected CER+CD8+ T cells transduced with HPV16 E7 specific TCR. FIG. 30A is a schematic for exemplary in vitro co-culture experiments. CD8 T cells were activated and transduced with a lentivirus cassette encoding a human papilloma virus 16 (HPV16) E7 protein-specific TCR, while CD4 T cells from the same graft were activated and transduced with a lentivirus encoding a CER. Both sets of cells were expanded ex vivo and combined at a 1:1 ratio and co-cultured with HPV16 E7+ head and neck squamous cell carcinoma cells (SCC152). FIG. 30B is a bar graph showing that combination of CD4 T cell/CER of the present disclosure with a CD8+/HPV16 E7 TCR (see, PCT Published Application No. WO2015/184228; SEQ ID NO:158) enhances cytolysis of target cells as measured by caspase induction compared to administration of the HPV16 E7 TCR alone. SCC152 is a head and neck squamous carcinoma that is HPV16+. Human primary CD8+ cells transduced with a HPV16 E7 TCR were co-cultured alone with SCC152 cells or in combination with CD4+ T cells transduced with various CERs of the present disclosure (CER5, CER17, CER19, CER21, CER23, CER26, CER27, CER103B, CER104, CER105, CER106, or CER116) at a 1:1 ratio. The number of caspase positive SCC152 target cells in the co-culture assay was measured by quantifying the intensity of red fluorescence from a caspase 3/7 apoptosis reagent that couples the activated caspase 3/7 recognition motif with a red reagent that fluoresces upon cleavage. The caspase 3/7 apoptosis reagent was added to the co-culture assay after 6 hours, and fluorescence was detected using BZ-X710 Keyence microscope and using hybrid capture software. The target SCC152 cells (transduced with green fluorescent protein (GFP)) were determined similarly. The Y-axis represents % caspase positive targets (#of caspase events/#of GFP target cells)*100.

FIG. 31 is bar graph showing that combination of CD4 T cell/CER of the present disclosure with a CD8+/HPV16 E7 TCR enhances cytolysis of target cells as measured by caspase induction compared to administration of the HPV16 E7 TCR alone. Human primary CD8+ cells transduced with a HPV16 E7 TCR were co-cultured alone with SCC152 cells or in combination with CD4+ T cells transduced with various CERs of the present disclosure (CER5, CER17, CER19, CER21, CER23, CER26, CER27, CER103B, CER104, CER105, CER106, or CER116) at a 1:1 ratio. The number of caspase positive SCC152 target cells in the co-culture assay was measured by quantifying the intensity of red fluorescence from a caspase 3/7 apoptosis reagent that couples the activated caspase 3/7 recognition motif with a red reagent that fluoresces upon cleavage. The caspase 3/7 apoptosis reagent was added to the co-culture assay after 6 hours, and fluorescence was detected using BZ-X710 Keyence microscope and using hybrid capture software. The Y-axis represent the intensity of caspase reagent in arbitrary units (a.u.)

FIG. 32 is a bar graph showing that addition of CD4+ T cell/CER104 (Tim4-TLR8) to co-culture experiments with CD8+ T cell/HPV16 E7 TCR and SCC152 target cells enhances cytotoxicity as measured by lactate dehydrogenase (LDH) cytotoxicity assay. Presence of LDH was assayed 4 hours after co-culture of CD8 T cells transduced with HPV16 E7 TCR and CD4 T cells transduced with CER104 at a 1:1 ratio with SCC152 target cells at varying target cell:effector cell ratios (0.5:1, 1:1, 1:2.5, 1:5, 1:10, 1:20).

FIG. 33 is a bar graph of the quantification of SCC152 HPV⁺ head and neck squamous carcinoma cells over time. Target cells were co-cultured with CD8 T cells transduced with HPV16 E7 TCR+CD4 T cells transduced with a selected CER, or controls (CD8 T cell transduced with HPV16 E7 TCR+CD4 transduced control) at a 1:1:1 ratio. The number of target cells were quantified using imaging software. Addition of CD4+ T cells transduced with various CERs of the present disclosure to CD8+ T cell/HPV16 E7 TCRs enhanced clearance of SCC152 target cells.

FIG. 34 is a line graph showing caspase 3/7 induction over time in co-culture experiments. The graph shows the number of caspase positive SCC152 target cells in a co-culture assay containing CD8 T cells transduced with HPV16 E7 TCR and CD4 T cells transduced with either control or a selected CER. The intensity of caspase was measured by quantifying the intensity of red fluorescence from a caspase 3/7 apoptosis reagent that couples the activated caspase 3/7 recognition motif with a red reagent that fluoresces upon cleavage. Measurements were taken at 2, 6, 8, and 10 hours of the co-culture assay.

FIG. 35 is a bar graph showing enhanced effector cytokine profile elicited upon co-culture of SCC152 cells with CD8 T cells transduced with HPV16 E7 TCR+CD4 T cells transduced with selected CERs of the present disclosure. CD8 T cells transduced with HPV16 E7 TCRs were co-administered with CD4 T cells transduced selected CERs at a 1:1 ratio to SCC152 target cells for an effector:target cell ratio of 1:1. Antigen-specific cytokine secretion was determined by measuring cytokine concentrations in the cell supernatants from each co-culture experiment using a mesoscale multi-array cytokine plate. The addition of a CD4 T cell/CER to CD8 T cell/HPV16 E7 TCR enhances IFN-γ, IL-2, TNFa, and IL-13 responses over CD8 T cell/HPV16 E7 TCR alone or combined with CD4 T cell transduced with truncated EGFR. The following cytokines were measured in the assay: IFN-γ, IL-2, TNFα, IL-4, IL-6, IL-12b, IL-13, IL-1b, and IL-10.

FIG. 36 is a bar graph representing quantification of CD4 T cell-CER mediated phagocytosis of SCC152 target cells. Results calculated as ((number of phagocytic target events)/(total number of effectors))*100 from 3×3 40× images, 4 hours after initiation of co-culture assay. CD8 T cells transduced with HPV16 E7 TCR and CD4 T cells transduced with selected CERs (CER5, CER17, CER19, CER21, CER23, CER26, CER27, CER103B, CER104, CER105, CER106, or CER116) were co-cultured with SCC152 squamous head and neck carcinoma target cells at a 1:1:0.5 ratio for 4 hours and imaged. CD8 T cell/HPV16 E7 TCR+CD4 T cell/CER displayed enhanced SCC152 engulfment activity as compared to CD8 T cell/HPV16 E7 TCR alone.

FIG. 37 is a bar graph representing quantification of CD4 T cell-CER mediated phagocytosis of SCC152 target cells. Results calculated as (median area ratio of target events in effector cells*% phagocytosis) from 3×3 40× images, 4 hours after initiation of co-culture assay. CD8 T cells transduced with HPV16 E7 TCR and CD4 T cells transduced with selected CERs (CER5, CER17, CER19, CER21, CER23, CER26, CER27, CER103B, CER104, CER105, CER106, or CER116) were co-cultured with SCC152 squamous head and neck carcinoma target cells at a 1:1:0.5 ratio for 4 hours and imaged. CD8 T cell/HPV16 E7 TCR+CD4 T cell/CER displayed enhanced SCC152 engulfment activity as compared to CD8 T cell/HPV16 E7 TCR alone.

FIGS. 38A-38F show vector maps for exemplary tandem expression cassettes. The tandem expression cassettes harbor both a HPV16 E7 specific TCR to induce a tumor (e.g., cervical) specific cytolytic response and a phosphatidylserine specific CER to elicit tumor specific phagocytic activity upon cytolysis-induced phosphatidylserine exposure. FIG. 38A shows an exemplary tandem expression cassette comprising a polynucleotide encoding a chimeric engulfment receptor (CER) 5 construct and a polynucleotide encoding a HPV16 E7 specific TCR. CER5 is positioned upstream of the HPV16 E7 specific TCR. The sequences encoding CER5 and HPV16 E7 TCR are operably linked to a EF-1α promoter and separated by a T2A peptide. CER5 comprises a Tim4 binding domain, a Tim4 transmembrane domain, and a TLR4 engulfment signaling domain. FIG. 38B shows an exemplary tandem expression cassette comprising a polynucleotide encoding a chimeric engulfment receptor (CER) 19 construct and a polynucleotide encoding an HPV16 E7 specific TCR. CER19 is positioned upstream of the HPV16 E7 specific TCR. The sequences encoding CER19 and HPV16 E7 TCR are operably linked to a EF-1α promoter and separated by a T2A peptide. CER19 comprises a Tim4 binding domain, a Tim4 transmembrane domain, and a TLR5 signaling domain. FIG. 38C shows an exemplary tandem expression cassette comprising a polynucleotide encoding a CER21 construct and a polynucleotide encoding an HPV16 E7 specific TCR. CER21 is positioned upstream of the HPV16 E7 specific TCR. The sequences encoding CER21 and HPV16 E7 TCR are operably linked to a EF-1α promoter and separated by a T2A peptide. CER21 comprises a Tim4 binding domain, a Tim4 transmembrane domain, and a TLR8 signaling domain. FIG. 38D shows an exemplary tandem expression cassette comprising a polynucleotide encoding CER27 construct and a polynucleotide encoding an HPV16 E7 specific TCR. CER27 is positioned upstream of the HPV16 E7 specific TCR. The sequences encoding CER27 and HPV16 E7 TCR are operably linked to a EF-1α promoter and separated by a T2A peptide. CER27 comprises a Tim4 binding domain, a Tim4 transmembrane domain, and a TLR2 signaling domain. FIG. 38E shows an exemplary tandem expression cassette comprising a polynucleotide encoding CER29 construct and a polynucleotide encoding an HPV16 E7 specific TCR. CER29 is positioned upstream of the HPV16 E7 specific TCR. The sequences encoding CER29 and HPV16 E7 TCR are operably linked to a EF-1α promoter and separated by a T2A peptide. CER29 comprises a Tim4 binding domain, a Tim4 transmembrane domain, and a Traf6 signaling domain. FIG. 38F shows an exemplary tandem expression cassette comprising a polynucleotide encoding CER31 construct and a polynucleotide encoding an HPV16 E7 specific TCR. CER31 is positioned upstream of the HPV16 E7 specific TCR. The sequences encoding CER31 and HPV16 E7 TCR are operably linked to a EF-1α promoter and separated by a T2A peptide. CER31 comprises a Tim4 binding domain, a Tim4 transmembrane domain, and a Traf3 signaling domain.

FIG. 39 is a line graph showing Caspase 3/7 induction over time in HPV16 E7+ SCC152 cells following co-culture with human primary CD8 T cells transduced with a lentiviral vector comprising HPV16 E7 TCR and Chimeric Engulfment Receptor 21 (CER21) separated by a T2A sequence. The HPV16 E7 TCR and CER21 confers enhanced target cell killing capacity to host CD8 T cells as compared to host CD8 T cells comprising HPV16 E7 TCR alone. Effector CD8+ T cells were incubated with target SCC152 cells at a 1:1 ratio. Total caspase 3/7 fluorescence was quantified over time.

FIG. 40 is a bar graph showing caspase 3/7 induction in HPV16 E7+ SCC152 cells upon co-culture with CD8 T cells transduced with a lentiviral vector comprising HPV16 E7 TCR and CER separated by a T2A sequence, Mock transduced cells were used as a negative control. Labeling of the IncuCyte® caspase 3/7 red apoptosis reagent enables detection of cells undergoing apoptosis (red fluorescence). Measurements were taken over time from co-culture experiments comparing CD8 transduced with a tandem CER− HPV16 E7 TCR cassette to HPV16 E7 TCR control.

FIG. 41 is a bar graph showing quantification of phagocytosis after 6 hours of co-culture of CD8+ T cells transduced with HPV16 E7 TCR, CER21-HPV16 E7 TCR expression cassette, CER29-HPV16 E7 TCR expression cassette, or CER31-HPV16 E7 TCR expression cassette. Quantification of phagocytosis was performed by the hybrid capture software in Keyence BZ-X710 imaging system wherein % phagocytosis was determined by identifying the number of red fluorescent targets (SCC152 cells) inside blue stained effector cells (cell trace violet labeled CD8+ T cells transduced with CER-HPV16 E7 TCR expression cassette—#of red internalized/#of blue)×100.

FIG. 42 is a 3D bar graph showing cytokine secretion patterns of CD8 T cells transduced with CER21-HPV16 E7 TCR expression cassette or HPV16 E7 TCR alone and co-cultured with SCC152 target cells. To determine cytokine secretion patterns, CER21-HPV16 E7 TCR modified CD8 T cells were co-cultured with SCC152 target cells. Antigen-specific cytokine secretion was determined by measuring cytokine concentrations in the cell supernatants from each co-culture using a mesoscale multi-array cytokine plate. The following cytokines were measured in the assay: IFNγ, IL-2, TNFα, IL-4, IL-6, IL-12b, IL-13, IL-1b, and IL-10. CD8 T cells transduced with CER21-HPV16 E7 TCR expression cassette exhibit antigen specific effector function as shown by cytokine secretion, e.g., IFNγ.

FIGS. 43A-43B show that EGFR-kinase inhibitors (FIG. 43A) Osimeritinib and (FIG. 43B) Brigatinib elicit a secondary, pro-engulfment marker on HCC159 cells upon drug exposure as detected by a Tim4-IgG1 Fc recombinant fusion protein.

FIGS. 44A-44B show that EGFR-kinase inhibitors (FIG. 44A) Erlotinib and (FIG. 44B) Gefitinib elicit a secondary, pro-engulfment marker on HCC159 cells upon drug exposure as detected by a Tim4-IgG1 Fc recombinant fusion protein.

FIGS. 45A-45B show that when EGFR inhibitor Osimeritinib (250 nM, 500 nM, and 1000 nM) was combined with phosphatidylserine-specific CER123- or CER126-expressing cells, growth of NSCLC cells harboring EGFR rearrangements was synergistically suppressed in vitro as measured by MTT assay (FIG. 45A) or microscopy (500 nM osimeritinib+CER126) (FIG. 45B). In FIG. 45A, the left bar graph shows data using an effector:target cell ratio of 1:1, the middle bar graph shows data using an effector:target ratio of 2:1, and the right bar graph shows data using an effector:target ratio of 5:1.

FIG. 46 shows that in the presence of osimeritinib (0, 500, or 1000 nM), CER123- or CER126-expressing cells demonstrate inducible, dose-dependent killing of NSCLC cells. The left bar graph shows data using an effector:target cell ratio of 5:1, and the right bar graph shows data using an effector:target ratio of 2:1.

FIG. 47 shows fluorescent micrographs of phagocytic elimination of EGFR-mutated NSCLC cells by CER122-modified cells following treatment with 500 nM osimeritinib (left panel) and lack of phagocytosis of NSCLC cells by mock transduced cells following treatment with 500 nM osimeritinib (right panel). The lower panel shows an enlargement of phagocytosis of NSCLC cells by CER122-modified cells following osimeritinib treatment, with white arrows indicating phagocytic events (pHrodo red targets within CT-violet-labeled T cells).

FIGS. 48A-48B show that when EGFR inhibitor Osimeritinib (0.1 nM, 1 nM, and 5 nM) was combined with phosphatidylserine-specific CER123- or CER126-expressing cells, growth of NSCLC cells harboring EGFR rearrangements was synergistically suppressed in vitro as measured by MTT assay (FIG. 48A) or microscopy (1 nM osimeritinib+CER123) (FIG. 48B). In FIG. 48A, the left bar graph shows data using an effector:target cell ratio of 1:1, the middle bar graph shows data using an effector:target ratio of 2:1, and the right bar graph shows data using an effector:target ratio of 5:1.

FIG. 49 shows that in the presence of osimeritinib (0, 1 nM, and 5 nM), CER123- or CER126-expressing cells demonstrate inducible, dose-dependent killing of NSCLC cells. The left bar graph shows data using an effector:target cell ratio of 5:1, and the right bar graph shows data using an effector:target ratio of 2:1.

FIG. 50 shows IFN-γ secretion levels in CER123- and CER126-expressing cells co-cultured with HCC159 cells with varying levels of Osimeritinib (0, 1 nM and 5 nM).

FIGS. 51A-51B: CER-expressing T cells in combination with Osimeritinib (1 nM) synergistically kill HCC827 NSCLC cells harboring EGFR mutations in vitro. FIG. 51A shows % viability for HCC827 cells incubated with Osimeritinib+CER21, CER108, CER104, or CER129-expressing T cells. FIG. 51B shows % viability for HCC827 cells incubated with Osimeritinib+CER27, CER120, CER122, CER123, CER124, or CER126-expressing T cells.

FIG. 52 shows % cell killing HCC159 NSCLC cells harboring EGFR mutation by CER-expressing CD4+ T cells in combination with +Osimeritinib (1 nM) as measured by LDH cytotoxicity assay.

FIG. 53 shows bright field microscopy images from co-culture experiments of HCC827+ cells. Cells were treated with Osimeritinib (1 nM) for 48 hours (right image) or without (left image).

FIG. 54 shows bright field microscopy images from co-culture experiments of HCC827+ cells. HCC827 cells were treated with CER104-transduced CD4+ T cells with Osimeritinib (1 nM) for 48 hours (right image) or without Osimeritinib (left image). Arrow indicates cluster of dead HCC827 EGFR⁺ cells surrounded by phagocytic CER104+ T cells.

FIG. 55 shows bright field microscopy images from co-culture experiments of HCC827+ cells. HCC827 cells were treated with CER21-transduced CD4+T cells with Osimeritinib (1 nM) for 48 hours (right image) or without Osimeritinib (left image). Arrow indicates cluster of dead HCC827 EGFR⁺ cells surrounded by phagocytic CER21⁺ T cells.

FIG. 56 shows bright field microscopy images from co-culture experiments of HCC827+ cells. HCC827 cells were treated with CER122-transduced CD4+T cells with Osimeritinib (1 nM) for 48 hours (right image) or without Osimeritinib (left image). Arrow indicates cluster of dead HCC827 EGFR cells surrounded by phagocytic CER122 T cells.

FIG. 57 shows % killing of H1975 NSCLC cells harboring EGFR mutations by CER-expressing T cells in combination with 1 μM Osimeritinib as measured by LDH cytotoxicity assay.

FIG. 58 shows bright field microscopy images from co-culture experiments of H1975 cells. H1975 cells were treated with CER126-transduced T cells+Osimeritinib (500 nM) for 48 hours (right image) or mock-transduced (vector only) T cells+Osimeritinib (500 nM) (left image).

FIGS. 59A-59B show that ALK inhibitors (FIG. 59A) Alectinib and (FIG. 59B) Crizotinib elicit a secondary, pro-engulfment marker on A549 cells upon drug exposure as detected by a Tim4-IgG1 Fc recombinant fusion protein.

FIGS. 60A-60D show that ALK inhibitors Crizotinib or Alectinib combined with CER104- or CER122-expressing T cells synergistically suppress in vitro growth of NSCLC cells harboring ALK rearrangements. FIG. 60A and FIG. 60B show effects of Alectinib (250 nM, 500 nM, 1 μM, 2.5 μM, 3.7 μM, 7 μM, or 10 μM)+CER104- or CER122-modified T cells on A549 cell viability after 48 hours co-culture at effector:target cell ratio of 2:1 (FIG. 60A) and 5:1 (FIG. 60B). FIG. 60C and FIG. 60D show effects of Crizotinib (250 nM, 500 nM, 1 μM, 2.5 μM, 3.7 μM, 7 μM, or 10 μM)+CER104- or CER122-modified T cells on A549 cell viability after 48 hours co-culture at effector:target cell ratio of 2:1 (FIG. 60C) and 5:1 (FIG. 60D). Mock transduced (vector only) T cells were used as control.

FIGS. 61A-61D show that ALK inhibitors Crizotinib or Alectinib combined with CER104- or CER122-expressing T cells synergistically suppress in vitro growth of NSCLC cells harboring ALK rearrangements. FIG. 61A and FIG. 61B show effects of Alectinib (250 nM, 500 nM, 1 μM, 2.5 μM, 3.7 μM, 7 μM, or 10 μM)+CER104- or CER122-modified T cells on A549 cell viability after 72 hours co-culture at effector:target cell ratio of 2:1 (FIG. 61A) and 5:1 (FIG. 61B). FIG. 61C and FIG. 61D show effects of Crizotinib (250 nM, 500 nM, 1 μM, 2.5 μM, 3.7 μM, 7 μM, or 10 μM)+CER104- or CER122-modified T cells on A549 cell viability after 72 hours co-culture at effector:target cell ratio of 2:1 (FIG. 61C) and 5:1 (FIG. 61D). Mock transduced (vector only) T cells were used as control.

FIGS. 62A-62E show fluorescent micrographs (40× magnification) of phagocytic elimination of ALK-positive A549 NSCLC cells treated with ALK inhibitor (alectinib or crizotinib). A549 cells were labeled with pHrodo red dye, a pH sensing dye to indicate localization in low-PH retaining endosomes. CD4 T cells were labeled with CT-violet. FIG. 62A shows mock-transduced (vector only) T cells do not exhibit phagocytosis of A549 cells. FIGS. 62B-C show that CER104-expressing T cells phagocytosed A549 cells when co-cultured with 1 μM Alectinib (FIG. 62B) or 1 μM Crizotinib (FIG. 62C). FIGS. 62D-E show that CER117-expressing T cells phagocytosed A549 cells when co-cultured with 1 μM Alectinib (FIG. 62D) or 1 μM Crizotinib (FIG. 62E). White arrows indicate examples of phagocytic events (pHrodo red target cells within CT-violet labeled CD4 T cells).

FIGS. 63A-63F show fluorescent micrographs (63× magnification) of phagocytic elimination of ALK-positive A549 NSCLC cells treated with ALK inhibitor (alectinib). A549 cells were labeled with pHrodo red dye, a pH sensing dye to indicate localization in low-PH retaining endosomes. CD4 T cells were labeled with CT-violet. FIG. 63A shows mock-transduced (vector only) T cells do not exhibit phagocytosis of A549 cells. FIG. 63D is an enlarged view of the area in FIG. 63A outlined by the white square. FIG. 63B shows that CER123-expressing T cells phagocytosed A549 cells when co-cultured with 2.5 μM Alectinib (FIG. 63B). FIG. 63E is an enlarged view of the area in FIG. 63B outlined by the white square. FIG. 63C shows that CER126-expressing T cells phagocytosed A549 cells when co-cultured with 2.5 μM Alectinib (FIG. 63C). FIG. 63F is an enlarged view of the area in FIG. 63C outlined by the white square. White arrows indicate examples of phagocytic events (pHrodo red target cells within CELLTRACE-violet labeled CD4 T cells).

FIGS. 64A-64B are bar graphs representing % phagocytosis (FIG. 64A) and phagocytic index (FIG. 64B) of Alectinib (1 μM) treated A549 cells by CER123- or CER126-expressing T cells.

FIGS. 65A-65C show that CER-expressing T cells demonstrate dose-dependent, inducible cell killing responses in the presence of ALK inhibitors Crizotinib and Alectinib. FIG. 65A shows that in the presence of Crizotinib (0, 2500, or 3700 nM), CER123- or CER126-expressing cells demonstrate inducible, dose-dependent killing of A549 cells. The left bar graph shows data using an effector:target cell ratio of 5:1, and the right bar graph shows data using an effector:target ratio of 2:1. FIG. 65B shows that in the presence of Alectinib (0, 2500, or 3700 nM), CER123- or CER126-expressing cells demonstrate inducible, dose-dependent killing of A549 cells. The left bar graph shows data using an effector:target cell ratio of 5:1, and the right bar graph shows data using an effector:target ratio of 2:1. FIG. 65C shows micrograph images from co-culture experiments with near complete loss of Crizotinib (3700 nM) treated A549 cells in the presence of CER126-transduced T cells (left panel) as compared to control (right panel).

FIGS. 66A-66B show IFN-γ secretion levels in CER123- and CER126-expressing cells co-cultured with A549 cells with Alectinib (3,700 nM) (FIG. 66A) or Crizotinib (3,700 nM) (FIG. 66B).

FIGS. 67A-67B show that various CER-expressing T cells in combination with Alectinib synergistically suppress growth of NSCLC cells harboring ALK rearrangements in vitro. FIG. 67A shows % cell viability of A549 ALK+ cells co-cultured with Alectinib+CER21-, CER108-, CER104-, or CER129-expressing T cells. FIG. 67B shows % cell viability of A549 ALK+ cells co-cultured with Alectinib+CER27-, CER120-, CER122-, CER123-, CER124-, or CER126-expressing T-cells.

FIG. 68 shows bright field microscopy images from co-culture experiments of A549 ALK+ cells. A549 ALK+ cells were treated with CER104-transduced T cells+Alectinib (3.7 μM) for 48 hours (right image) or mock-transduced (vector only) T cells+Alectinib (3.7 μM) (left image). Arrow indicates cluster of dead A549 ALK+ cells surrounded by phagocytic CER104 T cells.

FIG. 69 shows bright field microscopy images from co-culture experiments of A549 ALK+ cells. A549 ALK+ cells were treated with CER126-transduced T cells+Alectinib (3.7 μM) for 48 hours (right image) or mock-transduced (vector only) T cells+Alectinib (3.7 μM) (left image). Arrow indicates cluster of dead A549 ALK+ cells surrounded by phagocytic CER126 T cells.

FIG. 70 shows a bar graph representing quantification of adjusted phagocytic index of Alectinib-treated A549 cells+various CER-expressing T cells (CER21, CER27, CER30, CER108, CER110, CER112, CER120, CER122, CER123, CER124, CER126, CER127, or CER104).

FIG. 71 shows fluorescent micrograph images (40×) of phagocytic elimination of ALK+ NSCLC cells co-cultured with various CER-modified T-cells+Alectinib. Yellow triangles indicate phagocytic events (pH rodo red target cells within CT-violet-labeled CD4 T cells). Mock-transduced (vector only) T cells do not exhibit phagocytic activity.

FIGS. 72A-72C show time course (4 hours, 8 hours, and 12 hours) of phagocytic uptake of Alectinib-treated A549 ALK+ cells by CER122-expressing T cells as % phagocytosis (FIG. 72A) and phagocytic index (FIG. 72B). FIG. 72C shows fluorescent micrograph images obtained at 16 hours co-culture. Yellow arrows indicate phagocytic events (pHrodo red targets within CT-violet-labeled CD4 T cells) (FIG. 72C right image). Mock-transduced (vector only) controls exhibit no phagocytic activity (FIG. 72C left image).

FIG. 73 shows a bar graph of % killing by CER-expressing T cells co-cultured with A549 ALK+ cells+Alectinib (3.7 μM) as measured by LDH cytoxicity assay. Mock-transduced (vector only) T cells were used as a control.

FIG. 74 is a schematic of an exemplary treatment regimen of adoptive transfer of CER-transduced T cells in combination with ALK inhibitor therapy.

FIGS. 75A-75D show that generation of CER-expressing T cells can be expanded. FIG. 75A: T cells were enriched, activated, and transduced with a CER122-T2A-tEGFR lentiviral construct and phenotyped for surface tEGFR and T cell markers CD4 and CD8 by FACS. FIG. 75B: The total number of transduced and control T cells in unselected cultures were determined after CD3 and CD28 bead activation. FIG. 75C: 2D fluorescence droplet digital PCR plot from DNA of CER122-expressing T cell demonstrates amplification of a region from the CER cassette. The blue cluster on the plot (upper left) represents droplets that are positive for CER122 only, and the orange cluster (top right) represents clusters that are positive for both CER122 and RPP30. FIG. 75D: Table showing copy number value (CNV) for CER122-transduced T cells determined by droplet digital PCR.

FIGS. 76A-76C show that CER122 expressing T cells enhance anti-tumor responses to ALK inhibitor (15 mg/kg Alectinib) in vivo. FIG. 76A shows tumor volume measurements post-adoptive transfer in untreated, Alectinib only+mock transduced T cells (vector only), and Alectinib+CER122-transduced T cells (n=5/treatment group) NSG mice. FIG. 76B shows growth of A549/luciferase+/ALK+ cells in NSG mice, as evaluated by bioluminescence imaging. FIG. 76C shows bioluminescence imaging of A549-positive tumor burden at day 8 post-adoptive transfer.

FIGS. 77A-77B show FACS plots (FIG. 77A) and bar graph (FIG. 77B) showing early expansion of CD45+/CER122+ human T cells in peripheral blood post-adoptive cell treatment. FIG. 77B shows expansion of CER122 transduced T cells post-adoptive transfer as measured by an APC-conjugated, anti-human CD45 antibody at days 4, 8, 16, and 25 post-transfer.

FIG. 78 is a schematic of an exemplary treatment regimen of adoptive transfer of CER-transduced T cells in combination with EGFR inhibitor therapy.

FIG. 79 is a line graph showing tumor volume measurements post-adoptive transfer in untreated, Osimeritinib only, and Osimeritinib+CER122-transduced cells (n=5 per group).

FIGS. 80A-80B shows analysis of phagocytosis of HPV+ SCC152 cells by CER-expressing CD4+ T cells. FIG. 80A shows a magnitude breadth curve of CD4+ T cell phagocytosis by CER type. FIG. 80B shows fluorescent micrograph images of SCC152 target cells engulfed by CD4+ CER126-transduced T cells. Top image is an enlargement of a cell in the lower left image showing a SCC152 cell engulfed by CER126-transduced CD4+ T cell. Lower left image shows SCC152 cells (stained with pHrodo red) engulfed by CE126R-transduced CD4+ T cells; lower right image is the same micrograph, showing CER126-transduced CD4+ T cells illuminated with CELLTRACE violet. White arrow indicates CD8+ T cell transduced with E7-specific TCR and that is pHrodo Red negative (lower left panel of FIG. 80B). Software rendition of phagocytosis (lower right panel of FIG. 80B).

FIG. 81 shows cytokine secretion from CER-expressing CD4+ T cell+E7-specific TCR CD8+ T cell co-culture experiments. The addition of a CER-expressing CD4+ T cell to E7-specific TCR expressing CD8+ T cell enhanced levels of IFNγ secretion.

FIGS. 82A-82B show viSNE maps of mass cytometry data of CER-transduced CD4+ T cells upon antigen encounter. CER-transduced CD4+ T cells were co-cultured with E7-specific TCR-transduced CD8+ T cells and HPV+ SCC152 target cells and then interrogated by mass cytometry (CyTof). Intact CER-CD4+ T cells are shown in plots displaying tSNE1 and tSNE2 axes. Nine intracellular markers were used for the viSNE analysis. Each dot represents a single cell. FIG. 82A: Coloring the plots by a few of the measured markers (GM-CSF, MIP1b, Perforin, TNF, IL-17, Granzyme B, IL-4, IL-2, and IFNγ) shows the phenotype across viSNE ‘islands.’ Red represents high expression and blue represents low expression for each marker. FIG. 82B: Populations of CD4⁺ T cells were generated using a clustering algorithm from all 32 markers and overlaid onto the viSNE map. Arrows indicate enrichment of islands expressing the intracellular marker IFNγ in samples containing CER104, CER116, and CER117.

FIGS. 83A-83B show viSNE maps of mass cytometry data of CER-expressing CD4+ T cells upon antigen encounter. CER-transduced CD4+ T cells were co-cultured with E7-specific TCR-transduced CD8+ T cells and HPV+ SCC152 target cells and then interrogated by mass cytometry (CyTof). Intact CER-CD4+ T cells are shown in plots displaying tSNE1 and tSNE2 axes. Eighteen cell surface markers were used for the viSNE analysis. Each dot represents a single cell. FIG. 83A: Populations of CD4⁺ T cells were generated using a clustering algorithm from all 18 markers and overlaid onto the viSNE map. Arrows indicate enrichment of islands expressing the T cell activation marker CD69 in samples containing CER104 and CER116. FIG. 83B: Color plots show the phenotype across viSNE ‘islands.’ Red represents high expression and blue represents low expression for each marker. Highlighted region indicates cells expressing T cell activation marker CD69.

FIGS. 84A-84B show viSNE maps of mass cytometry data of CER-expressing CD4+ T cells upon antigen encounter. CER-transduced CD4+ T cells were co-cultured with E7-specific TCR-transduced CD8+ T cells and HPV+ SCC152 target cells and then interrogated by mass cytometry (CyTof). Intact CER-CD4+ T cells are shown in plots displaying tSNE1 and tSNE2 axes. Eighteen cell surface markers were used for the viSNE analysis. Each dot represents a single cell. FIG. 84A: Populations of CD4⁺ T cells were generated using a clustering algorithm from all 18 markers and overlaid onto the viSNE map. Arrows indicate loss of islands expressing the naïve T cell marker CD45RA within the CCR7 population among CER104 and CER116 samples compared to controls. FIG. 84B: Color plots show the phenotype across viSNE ‘islands.’ Red represents high expression and blue represents low expression for each marker. Highlighted region indicates cells the naïve T cell marker CD45RA. CER104 and CER116-transduced CD4 T cells were associated with memory formation after antigen encounter.

FIGS. 85A-85G show induction of phagocytic signal transduction cascades in CER-expressing cells and luminal content degradation. FIG. 85A: Ba/F3 cell lines harboring CER21, CER116, or an empty plasmid (mock) were co-cultured with dexamethasone pre-treated thymocytes for 2 hours. The Rac1 inhibitor NSC23766 (Selleck Chem) was added during the co-culture in appropriate wells. Cells were then collected, solubilized in lysis buffer, and protein lysates underwent immuno-precipitation for phospho-Rac1 using PAK-PBD beads (Cytoskeleton Inc.). Immunoprecipitates were eluted and 25 ug of protein was loaded onto SDS-PAGE gradient gels and then probed with monoclonal Rac-1 primary antibody (Cytoskeleton Inc.) overnight at 4° C., washed, and hybridized with anti-mouse HRP (Jackson Labs). Prior to immunoprecipitation, some sample was retained for protein estimation and total Rac1 estimation. Basal samples indicate CER-expressing cell lines cultured without target cells. FIG. 85B: Gel bands were quantified using ImageJ and the proportion of activated Rac1 quantified. FIG. 85C: Representative FACs profiles for pHrodo cells in Ba/f3 cell lines harboring CER21 after 6 hour co-culture. The addition of a specific Rac1 small molecule inhibitor abolishes phagocytosis (right). The numbers indicates the percentage (phagocytosis) of pHrodo cells in CER21 Ba/f3 cells. FIG. 85D: Phagocytic indices were calculated from fluorescent imaging (FIG. 85E). FIG. 85E: Representative visualization of phagocytosis assays of CER-harboring Ba/f3 cell lines in the presence or absence of Rac1 inhibition. FIG. 85F: Ba/f3 cell lines were co-cultured with pHrodo-red labeled-target cells (prey) overnight and subsequently purified by FACS. Target cell destruction was visualized by time-lapse imaging and quantified over time. The addition of a TRAF6 signaling to CER116 domain enhances CER116 luminal content degradation over time, with near complete resolution of luminal contents by 36 hours. FIG. 85G: Time lapse imaging demonstrates destruction of CER116-expressing Ba/F3 cells luminal contents. pHrodo-red labeled contents are broken down over-time; CER116-harboring Ba/F3 cells (top panels) catabolize target cells, allowing cells to return to homeostasis and resume immune responsiveness.

FIG. 86 shows a schematic of an exemplary antigen presentation assay. In a phagocytic assay step, CD4 and CD8+ T cell lines expressing CERs were co-cultured with the CD4 and CD8 T cells expressing HPV E7 specific TCR and SCC152 (HPV+) cells overnight. The following day CER+ T cells were subsequently FACS-sorted. FACS plot depicts CT violet CERs. Following FACS purification, antigen presentation of HPV oncoproteins was evaluated. CER-expressing cells were co-cultured at a 1:2 ratio with E6 & E7-specific TCR/NFAT reporter cell lines, and NFAT activation measured over time using a plate reader.

FIG. 87 shows a line graph of NFAT activation in E6/E7 TCR-transduced T cells comprising an NFAT reporter gene following co-culture with CD4+ and CD8+ CER123-transduced T cells that have been co-cultured with HPV⁺ tumor cells and CD4+/CD8+ E7 TCR transduced T cells as shown in the schematic in FIG. 86. CER-expressing CD4⁺ and CD8⁺ T cell lines, after phagocytosing HPV⁺ tumor cells, are capable of cross-presentation of E7 HPV oncoproteins to E7 TCR/NFAT reporter-expressing T cells as measured by NFAT activation.

### DETAILED DESCRIPTION

Chimeric proteins including (a) an extracellular domain comprising an extracellular binding domain and, optionally, an extracellular spacer domain, (b) a transmembrane domain, and (c) an engulfment signaling domain comprising a toll-like receptor (TLR) signaling domain, a Traf6 signaling domain, a Traf2 signaling domain, or a Traf3 signaling domain, and nucleic acid molecules encoding said chimeric proteins are described herein. Additionally, cells modified to express these chimeric proteins and methods and compositions for delivery of such modified cells to a subject in need thereof are provided. The chimeric proteins are referred to herein as a “chimeric engulfment receptor” or “chimeric engulfment receptors” (“CER” in the singular and “CERs” in the plural). Chimeric engulfment receptors described herein are capable of conferring an engulfment phenotype to a host cell that is genetically modified to express said chimeric engulfment receptor. In such certain embodiments, expression of a CER as described herein confers an engulfment phenotype to a host cell that does not naturally exhibit an engulfment phenotype. In other such embodiments, expression of a CER as described herein by a host cell confers an engulfment phenotype specific to a pro-engulfment marker or antigenic marker not naturally targeted by the host cell. In still other such embodiments, expression of a CER as described herein by a host cell confers an engulfment phenotype specific to a pro-engulfment marker or antigenic marker naturally targeted by the host cell and expression of the CER by the host cell enhances engulfment by the host cell of cells, microbes, or particles exhibiting the targeted pro-engulfment or antigenic marker.

In certain embodiments, the CER targets an engulfment marker associated with apoptotic, dead, dying, damaged, infected, or necrotic cells. In other embodiments, the CER targets an antibody bound cell associated with an infectious microbe or particle. In still other embodiments, the CER targets an antigenic marker displayed by aberrant cells or misfolded proteins associated with a disease, disorder, or other undesired condition.

One or more CERs according to the present description can be transduced into and expressed in cells, such as T cells, Natural Killer Cells, Natural Killer T cells, B cells, lymphoid precursor cells, dendritic cells, Langerhans cells, and myeloid cells. In certain embodiments, in addition to engineering the CER to bind to a specified target molecule (e.g., an engulfment marker or an antigenic marker), the engulfment signaling domain of the CER is selected to provide desired engulfment activity. In another embodiment, the CER comprises a primary engulfment signaling domain and a secondary engulfment signaling domain.

Host cells that are genetically modified to express one or more CERs according to the present description can be used for specific engulfment of a target cell or particle expressing a target molecule to which the extracellular domain of the CER binds. In certain embodiments, the target cell or particle may be a tumor cell, a cancer cell, a microbe (e.g., bacteria, fungus, virus), a protozoan parasite, an aberrant cell, or a misfolded protein associated with an infection, disease, disorder, or other undesired condition. In further embodiments, host cells that are genetically modified to express one or more CERs according to the present description are used to treat cancer, an infectious disease (viral, bacterial, fungal, protozoan), an inflammatory disease, an immune disease (e.g., autoimmune disease) in a subject either as a primary therapy or as an adjunct or combination therapy. The CER of the present disclosure may confer pro-inflammatory (immunogenic) phenotype to a host cell expressing the CER via the TLR, Traf6, Traf2, or Traf3 engulfment signaling domain. In certain embodiments, the CER modified host cell further exhibits enhanced proliferative activity, expansion activity, activation, memory formation, cytolytic activity, antigen presentation activity, phagocytic signaling activity, luminal degradation, or any combination thereof that may otherwise not be present in a host cell that does not express the CER.

### Definitions

Prior to setting forth this disclosure in more detail, it may be helpful to an understanding thereof to provide definitions of certain terms to be used herein.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

Terms understood by those in the art of antibody technology are each given the meaning acquired in the art, unless expressly defined differently herein. The term “antibody” is used in the broadest sense and includes polyclonal and monoclonal antibodies. An “antibody” may refer to an intact antibody comprising at least two heavy (H) chains and two light (L) chains inter-connected by disulfide bonds, as well as an antigen-binding portion (or antigen-binding domain) of an intact antibody that has or retains the capacity to bind a target molecule. An antibody may be naturally occurring, recombinantly produced, genetically engineered, or modified forms of immunoglobulins, for example intrabodies, peptibodies, nanobodies, single domain antibodies, SMIPs, multispecific antibodies (e.g., bispecific antibodies, diabodies, triabodies, tetrabodies, tandem di-scFV, tandem tri-scFv, ADAPTIR). A monoclonal antibody or antigen-binding portion thereof may be non-human, chimeric, humanized, or human, preferably humanized or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988). “Antigen-binding portion” or “antigen-binding domain” of an intact antibody is meant to encompass an “antibody fragment,” which indicates a portion of an intact antibody and refers to the antigenic determining variable regions or complementary determining regions of an intact antibody. Examples of antibody fragments include, but are not limited to, Fab, Fab′, F(ab′)2, and Fv fragments, Fab′-SH, F(ab′)₂, diabodies, linear antibodies, scFv antibodies, VH, and multispecific antibodies formed from antibody fragments. A “Fab” (fragment antigen binding) is a portion of an antibody that binds to antigens and includes the variable region and CH1 of the heavy chain linked to the light chain via an inter-chain disulfide bond. An antibody may be of any class or subclass, including IgG and subclasses thereof (IgG₁, IgG₂, IgG₃, IgG₄), IgM, IgE, IgA, and IgD.

The term “variable region” or “variable domain” refers to the domain of an antibody heavy or light chain that is involved in binding of the antibody to antigen. The variable domains of the heavy chain and light chain (VH and VL, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (HCDR1, HCDR2, HCDR3) and three CDRs in each light chain variable region (LCDR1, LCDR2, LCDR3).

The terms “antigen” and “Ag” refer to a molecule that provokes an immune response. The immune response provoked may involve antibody production, the activation of specific immunologically-competent cells, or both. Macromolecules, including proteins, glycoproteins, and glycolipids, can serve as an antigen. Antigens can be derived from recombinant or genomic DNA. As contemplated herein, an antigen need not be encoded (i) solely by a full length nucleotide sequence of a gene or (ii) by a “gene” at all. An antigen can be generated or synthesized, or an antigen can be derived from a biological sample. Such a biological sample can include, but is not limited, to a tissue sample, a tumor sample, a cell, or a biological fluid.

The term “epitope” or “antigenic epitope” includes any molecule, structure, amino acid sequence or protein determinant within an antigen that is specifically bound by a cognate immune binding molecule, such as an antibody or fragment thereof (e.g., scFv), T cell receptor (TCR), chimeric engulfment receptor, or other binding molecule, domain or protein. Epitopic determinants generally contain chemically active surface groupings of molecules, such as amino acids or sugar side chains, and can have specific three dimensional structural characteristics, as well as specific charge characteristics. An epitope may be a linear epitope or a conformational epitope. The term “anti-tumor effect” refers to a biological effect which can be manifested by a decrease in tumor volume, a decrease in the number of tumor cells, a decrease in the number of metastases, an increase in life expectancy, or amelioration of various physiological symptoms associated with a cancerous condition. An “anti-tumor effect” can also be manifested by prevention of a hematological malignancy or tumor formation.

“Autoimmune disease” refers to a disorder that results from an autoimmune response. An autoimmune disease is the result of an inappropriately excessive response to a self-antigen. An autoimmune response may involve self-reactive B-cells that produce autoantibodies, self-reactive T-cells, or both. An “autoantibody” as used herein is an antibody produced by a subject that binds to a self-antigen also produced by the subject.

“Autologous” refers to any material derived from the same subject to which it is later to be re-introduced.

“Allogeneic” refers to a graft derived from a different subject of the same species.

As used herein, the terms “binding domain,” “binding region,” and “binding moiety” refer to a molecule, such as a peptide, oligopeptide, polypeptide, or protein that possesses the ability to specifically and non-covalently bind, associate, unite, recognize, or combine with a target molecule (e.g., PtdSer, an IgG antibody, an IgE antibody, an IgA antibody, CD138, CD38, CD33, CD123, CD79b, mesothelin, PSMA, BCMA, ROR1, MUC-16, L1CAM, CD22, CD19, EGFRviii, VEGFR-2, or GD2). A binding domain includes any naturally occurring, synthetic, semi-synthetic, or recombinantly produced binding partner for a biological molecule or other target of interest. In some embodiments, the binding domain is an antigen-binding domain, such as an antibody or functional binding domain or antigen-binding portion thereof. Exemplary binding domains include single chain antibody variable regions (e.g., domain antibodies, sFv, scFv, Fab), receptor ectodomains (e.g., TNF-α), ligands (e.g., cytokines, chemokines), or synthetic polypeptides selected for the specific ability to bind to a biological molecule.

A variety of assays are known for identifying binding domains of the present disclosure that specifically bind a particular target, as well as determining binding domain affinities, such as Western blot, ELISA, and BIACORE® analysis (see also, e.g., Scatchard et al., Ann. N.Y. Acad. Sci. 51:660, 1949; and U.S. Pat. Nos. 5,283,173, 5,468,614, or the equivalent). As used herein, “specifically binds” refers to an association or union of a binding domain, or a fusion protein thereof, to a target molecule with an affinity or Kₐ (i.e., an equilibrium association constant of a particular binding interaction with units of 1/M) equal to or greater than 10⁵ M⁻¹, while not significantly associating or uniting with any other molecules or components in a sample.

The term “cancer” as used herein is defined as disease characterized by the rapid and uncontrolled growth of aberrant cells. The aberrant cells may form solid tumors or constitute a hematological malignancy. Cancer cells can spread locally or through the bloodstream and lymphatic system to other parts of the body. Examples of various cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

A “disease” is a state of health of a subject wherein the subject cannot maintain homeostasis, and wherein, if the disease is not ameliorated, then the subject's health continues to deteriorate. In contrast, a “disorder” or “undesirable condition” in a subject is a state of health in which the subject is able to maintain homeostasis, but in which the subject's state of health is less favorable than it would be in the absence of the disorder or undesirable condition. Left untreated, a disorder or undesirable condition does not necessarily result in a further decrease in the subject's state of health.

A “microbe” or “microorganism” refers to any species of bacteria, virus, archaea, or fungi.

A “particle” refers to a fragment of a cell or a small object of at least 100 nm and up to 6 μm in diameter and that is derived from a living cell or organism. A particle can be a viral particle, small mineral particle, cellular debris, or a synthetic particle.

“Encoding” refers to the inherent property of specific polynucleotide sequences, such as DNA, cDNA, and mRNA sequences, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Thus, a polynucleotide encodes a protein if transcription and translation of mRNA corresponding to that polynucleotide produces the protein in a cell or other biological system. Both a coding strand and a non-coding strand can be referred to as encoding a protein or other product of the polynucleotide.

Unless otherwise specified, a “nucleotide sequence encoding an amino acid sequence” includes all nucleotide sequences that are degenerate versions of each other and that encode the same amino acid sequence.

As used herein, the term “endogenous” or “native” refers to a gene, protein, compound, molecule or activity that is normally present in a host or host cell.

As used herein, the term “engulfment” refers to a receptor-mediated process wherein endogenous or exogenous cells or particles greater than 100 nm in diameter are internalized by a phagocyte or host cell of the present disclosure. Engulfment is typically composed of multiple steps: (1) tethering of the target cell or particle via binding of an engulfment receptor to a pro-engulfment marker or antigenic marker directly or indirectly (via a bridging molecule) on a target cell or particle; and (2) internalization or engulfment of the whole target cell or particle, or a portion thereof. In certain embodiments, internalization may occur via cytoskeletal rearrangement of a phagocyte or host cell to form a phagosome, a membrane-bound compartment containing the internalized target. Engulfment may further include maturation of the phagosome, wherein the phagosome becomes increasingly acidic and fuses with lysosomes (to form a phagolysosome), whereupon the engulfed target is degraded (e.g. “phagocytosis”). Alternatively, phagosome-lysosome fusion may not be observed in engulfment. In yet another embodiment, a phagosome may regurgitate or discharge its contents to the extracellular environment before complete degradation. In some embodiments, engulfment refers to phagocytosis. In some embodiments, engulfment includes tethering of the target cell or particle by the phagocyte of host cell of the present disclosure, but not internalization. In some embodiments, engulfment includes tethering of the target cell or particle by the phagocyte of host cell of the present disclosure and internalization of part of the target cell or particle.

As used herein, the term “phagocytosis” refers to an engulfment process of cells or large particles (≥100 nm) wherein tethering of a target cell or particle, engulfment of the target cell or particle, and degradation of the internalized target cell or particle occurs. In certain embodiments, phagocytosis comprises formation of a phagosome that encompasses the internalized target cell or particle and phagosome fusion with a lysosome to form a phagolysosome, wherein the contents therein are degraded. In certain embodiments, during phagocytosis, following binding of a CER expressed on a phagocyte or a host cell of the present disclosure to an engulfment marker expressed by a target cell or particle, a phagocytic synapse is formed; an actin-rich phagocytic cup is generated at the phagocytic synapse; phagocytic arms are extended around the target cell or particle through cytoskeletal rearrangements; and ultimately, the target cell or particle is pulled into the phagocyte or host cell through force generated by motor proteins. As used herein, “phagocytosis” includes the process of “efferocytosis”, which specifically refers to the phagocytosis of apoptotic or necrotic cells in a non-inflammatory manner.

As used herein, the term “pro-engulfment marker” refers to a moiety (e.g., protein, lipid, or polysaccharide) that an apoptotic, necrotic, pyroptotic, or infected cell exhibits on its surface that distinguishes it from a non-apoptotic, non-necrotic, non-pyroptotic, oncotic, or uninfected cell, respectively. A pro-engulfment marker can be an intracellular moiety that is surface exposed on an apoptotic or necrotic cell, a moiety that has altered glycosylation or altered surface charge on an apoptotic or necrotic cell, or a serum moiety that is bound to an apoptotic, necrotic, pyroptotic, or oncotic cell. Examples of pro-engulfment markers for apoptotic cells include phosphatidylserine (PtdSer), ICAM-3, oxidized low density lipoprotein, calreticulin, annexin I, complement C1q, and thrombospondin. Necrotic, oncotic, and pyroptotic cells also expose PtdSer pro-engulfment markers on the cell surface. Engulfment receptors can detect (or bind) a pro-engulfment marker on a target cell (e.g., a damaged, infected, apoptotic, necrotic, pyroptotic, or oncotic cell) directly or indirectly using soluble bridging molecules as intermediaries that bind to the pro-engulfment marker.

A “toll-like receptor” refers to a member of a family of conserved immune receptors that are pattern recognition receptors (PRR) that recognize molecules that are conserved in pathogens but distinguishable from host molecules (e.g., pathogen-associated molecular patterns (PAMPs)) as well as endogenous molecules released from necrotic or dying cells (danger-associated molecular patterns (DAMPs)). Examples of TLR PAMP ligands include bacterial lipoprotein, bacterial peptidoglycans, double-stranded RNA, lipopolysaccharides, bacterial flagella, single-stranded RNA, and CpG DNA. DAMPs include heat shock proteins and protein fragments from the extracellular matrix. TLRs are type I transmembrane proteins characterized by an extracellular domain containing leucine-rich repeats (LRRs), a juxtamembrane domain comprising acidic amino acids lying between the LRRs and the transmembrane domain, and a cytoplasmic signaling domain that contains a conserved region called the Toll/IL-1 receptor (TIR) domain. TLRs are expressed on the membranes of leukocytes including dendritic cells, macrophages, natural killer cells, cells of the adaptive immunity (T and B cells) and non-immune cells (epithelial and endothelial cells, and fibroblasts). Ligand binding by TLRs initiates signaling cascades leading to the activation of transcription factors, such as AP-1, NF-κB and interferon regulatory factors (IRFs), resulting in production of interferons, pro-inflammatory cytokines, and effector cytokines that direct the adaptive immune response. A TLR may be from any mammal, e.g., humans, primates, cows, horses, sheep, dogs, cats, mice, rats, rabbits, guinea pigs, or pigs. A TLR may be any one of the ten TLRs (TLR1-TLR10) that have been identified in humans or any one of the thirteen TLRs have been identified in mice (TLR1-13). TLRs are located on the plasma membrane, except for TLR3, TLR7, TLR8, and TLR9, which are endosomal TLRs.

A “TLR signaling domain” refers to the cytoplasmic domain of a TLR molecule comprising a TIR domain or a functional fragment thereof. In certain embodiments, a TLR signaling domain may be a signaling domain or functional fragment thereof of any one of TLR1, TLR2, TLR3, TLR4, TLR5, TLR6, TLR7, TLR8, or TLR9.

An “engulfment signaling domain” refers to an intracellular effector domain, which upon binding of the target molecule (e.g., pro-engulfment marker or antigenic marker) targeted by the extracellular domain of a CER expressed by a host cell, activates one or more signaling pathways in the host cell resulting in engulfment, including, in specific embodiments, cytoskeletal rearrangement of the host cell and internalization of the target cell, microbe, or particle associated with the marker or antigen. In certain embodiments, an engulfment signaling domain activates one or more signaling pathways resulting in phagocytosis of the target cell, microbe, or particle. In certain embodiments, the engulfment signaling domain includes an engulfment signaling domain comprising a TLR signaling domain, Traf6 signaling domain, Traf2 signaling domain, or Traf3 signaling domain. In certain other embodiments, the engulfment signaling domain includes a primary engulfment signaling domain and a secondary engulfment signaling domain. An engulfment signaling domain may comprise the full length intracellular component of an engulfment signaling molecule or a functional fragment thereof.

A “pro-inflammatory engulfment signaling domain” refers to an effector domain that (i) stimulates engulfment of the targeted cell, microbe, or particle and (ii) is derived from an endogenous receptor or signaling molecule that typically stimulates one or more of (a) host cell secretion of inflammatory cytokines, such as, for example, TNFα, IL-1, IL-6, IL-12, and IL-23, (b) host cell secretion of inflammatory chemokines, such as, for example, CCL5 (RANTES), CXCL9, and CXCL10, (c) upregulation of cell surface co-stimulatory markers, such as, for example, CD80, CD86, HLA-DR, CD40, HVEM, and 4-1BBL, and (d) activation of one or more signaling cascades, such as NF-κB, that induce, potentiate, or complement chemotherapies, antibody-based immune therapies, or cellular therapies, such as, for example, T cell targeted therapies. In certain embodiments, stimulation of pro-inflammatory engulfment signaling promotes inflammation in the local tissue milieu. A pro-inflammatory engulfment signaling domain can also be referred to as an “immunogenic” engulfment signaling domain or an “inflammatory” engulfment signaling domain.

As used herein, an “effector domain” is an intracellular portion of a fusion protein or receptor that can directly or indirectly promote a biological or physiological response in a cell expressing the effector domain when receiving the appropriate signal. In certain embodiments, an effector domain is part of a protein or protein complex that receives a signal when bound, or it binds directly to a target molecule, which triggers a signal from the effector domain. For example, in response to binding of the CER to a target molecule, the effector domain may transduce a signal to the interior of the host cell, eliciting an effector function, e.g., engulfment, phagolysosome maturation, secretion of inflammatory cytokines and/or chemokines. An effector domain may directly promote a cellular response when it contains one or more signaling domains or motifs. In other embodiments, an effector domain will indirectly promote a cellular response by associating with one or more other proteins that directly promote a cellular response.

As used herein, “heterologous” or “non-endogenous” or “exogenous” refers to any gene, protein, compound, molecule or activity that is not native to a host cell or a subject, or is any gene, protein, compound, molecule, or activity native to a host or host cell but has been altered or mutated such that the structure, activity or both is different as between the native and mutated molecules. In certain embodiments, heterologous, non-endogenous or exogenous molecules (e.g., receptors, ligands) may not be endogenous to a host cell or subject, but instead nucleic acids encoding such molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a molecule or activity found in or derived from a host cell, species or strain. For example, a heterologous or exogenous molecule or gene encoding the molecule may be homologous to a native host or host cell molecule or gene that encodes the molecule, respectively, but may have an altered structure, sequence, expression level or combinations thereof. A non-endogenous molecule may be from the same species, a different species or a combination thereof.

“Junction amino acids” or “junction amino acid residues” refer to one or more (e.g., about 2-20) amino acid residues between two adjacent motifs, regions or domains of a polypeptide. Junction amino acids may result from the construct design of a chimeric protein (e.g., amino acid residues resulting from the use of a restriction enzyme site during the construction of a nucleic acid molecule encoding a fusion protein).

“Nucleic acid molecule” and “polynucleotide” can be in the form of RNA or DNA, which includes cDNA, genomic DNA, and synthetic DNA. A nucleic acid molecule may be double stranded or single stranded, and if single stranded, may be the coding strand or non-coding (anti-sense strand). A coding molecule may have a coding sequence identical to a coding sequence known in the art or may have a different coding sequence, which, as the result of the redundancy or degeneracy of the genetic code, or by splicing, can encode the same polypeptide.

The term “overexpressed” or “overexpression” of an antigen refers to an abnormally high level of antigen expression in a cell. Overexpressed antigen or overexpression of antigen is often associated with a disease state, such as in hematological malignancies and cells forming a solid tumor within a specific tissue or organ of a subject. Solid tumors or hematological malignancies characterized by overexpression of a tumor antigen can be determined by standard assays known in the art.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “mature polypeptide” or “mature protein” refers to a protein or polypeptide that is secreted or localized in the cell membrane or inside certain cell organelles (e.g., the endoplasmic reticulum, golgi, or endosome) and does not include an N-terminal signal peptide.

A “signal peptide”, also referred to as “signal sequence”, “leader sequence”, “leader peptide”, “localization signal” or “localization sequence”, is a short peptide (usually 15-30 amino acids in length) present at the N-terminus of newly synthesized proteins that are destined for the secretory pathway. A signal peptide typically comprises a short stretch of hydrophilic, positively charged amino acids at the N-terminus, a central hydrophobic domain of 5-15 residues, and a C-terminal region with a cleavage site for a signal peptidase. In eukaryotes, a signal peptide prompts translocation of the newly synthesized protein to the endoplasmic reticulum where it is cleaved by the signal peptidase, creating a mature protein that then proceeds to its appropriate destination.

The “percent identity” between two or more nucleic acid or amino acid sequences is a function of the number of identical positions shared by the sequences (i.e., % identity=number of identical positions/total number of positions×100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST).

A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:NY (1975), pp. 71-77; Lewin, Genes IV, Oxford University Press, NY and Cell Press, Cambridge, MA (1990), p. 8).

The term “chimeric” refers to any nucleic acid molecule or protein that is not endogenous and comprises sequences joined or linked together that are not normally found joined or linked together in nature. For example, a chimeric nucleic acid molecule may comprise regulatory sequences and coding sequences that are derived from different sources, or regulatory sequences and coding sequences that are derived from the same source but arranged in a manner different than that found in nature.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “subject,” “patient” and “individual” are used interchangeably herein and are intended to include living organisms in which an immune response can be elicited (e.g., mammals). Examples of subjects include humans, primates, cows, horses, sheep, dogs, cats, mice, rats, rabbits, guinea pigs, pigs, and transgenic species thereof

The term “T cells” refers to cells of T cell lineage. “Cells of T cell lineage” refers to cells that show at least one phenotypic characteristic of a T cell or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺CD8⁺ double positive; single positive CD4⁺ or CD8⁺; TCRαβ or TCR γδ; or mature and functional or activated T cells. The term “T cells” encompasses naïve T cells (CD45 RA+, CCR7+, CD62L+, CD27+, CD45RO−), central memory T cells (CD45RO⁺, CD62L⁺, CD8⁺), effector memory T cells (CD45RA+, CD45RO−, CCR7−, CD62L−, CD27−), mucosal-associated invariant T cells, natural killer T cells, and tissue resident T cells.

The term “B cells” refers to cells of the B cell lineage. “Cells of T cell lineage” refers to cells that show at least one phenotypic characteristic of a B cell or a precursor or progenitor thereof that distinguishes the cells from other lymphoid cells, and cells of the erythroid or myeloid lineages. Such phenotypic characteristics can include expression of one or more proteins specific for B cells (e.g., CD19⁺, CD72+, CD24+, CD20⁺), or a physiological, morphological, functional, or immunological feature specific for a B cell. For example, cells of the B cell lineage may be progenitor or precursor cells committed to the B cell lineage (e.g., pre-pro-B cells, pro-B cells, and pre-B cells); immature and inactivated B cells or mature and functional or activated B cells. Thus, “B cells” encompass naïve B cells, plasma cells, regulatory B cells, marginal zone B cells, follicular B cells, lymphoplasmacytoid cells, plasmablast cells, and memory B cells (e.g., CD27⁺, IgD⁻).

A “therapeutically effective amount” or “effective amount” of a chimeric protein or cell expressing a chimeric protein of this disclosure (e.g., a CER or a cell expressing a CER) refers to that amount of protein or cells sufficient to result in amelioration of one or more symptoms of the disease, disorder, or undesired condition being treated. When referring to an individual active ingredient or a cell expressing a single active ingredient, administered alone, a therapeutically effective dose refers to the effects of that ingredient or cell expressing that ingredient alone. When referring to a combination, a therapeutically effective dose refers to the combined amounts of active ingredients or combined adjunctive active ingredient with a cell expressing an active ingredient that results in a therapeutic effect, whether administered serially or simultaneously.

“Treat” or “treatment” or “ameliorate” refers to medical management of a disease, disorder, or undesired condition of a subject. In general, an appropriate dose or treatment regimen comprising a host cell expressing a CER of this disclosure is administered in an amount sufficient to elicit a therapeutic or prophylactic benefit. Therapeutic or prophylactic/preventive benefit includes improved clinical outcome; lessening or alleviation of symptoms associated with a disease, disorder, or undesired condition; decreased occurrence of symptoms; improved quality of life; longer disease-free status; diminishment of extent of disease, disorder, or undesired condition; stabilization of disease state; delay of disease progression; remission; survival; prolonged survival; or any combination thereof.

The phrase “under transcriptional control” or “operatively linked” as used herein means that a promoter is in the correct location and orientation in relation to a polynucleotide to control the initiation of transcription by RNA polymerase and expression of the polynucleotide.

A “vector” is a nucleic acid molecule that is capable of transporting another nucleic acid. Vectors may be, for example, plasmids, cosmids, viruses, or phage. The term should also be construed to include non-plasmid and non-viral compounds which facilitate transfer of nucleic acid into cells. An “expression vector” is a vector that is capable of directing the expression of a protein encoded by one or more genes carried by the vector when it is present in the appropriate environment.

In certain embodiments, the vector is a viral vector. Examples of viral vectors include, but are not limited to, adenovirus vectors, adeno-associated virus vectors, retrovirus vectors, gammaretrovirus vectors, and lentivirus vectors. “Retroviruses” are viruses having an RNA genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses. “Lentivirus” refers to a genus of retroviruses that are capable of infecting dividing and non-dividing cells. Examples of lentiviruses include, but are not limited to HIV (human immunodeficiency virus, including HIV type 1 and HIV type 2, equine infectious anemia virus, feline immunodeficiency virus (FIV), bovine immune deficiency virus (BIV), and simian immunodeficiency virus (SIV).

In other embodiments, the vector is a non-viral vector. Examples of non-viral vectors include lipid-based DNA vectors, modified mRNA (modRNA), self-amplifying mRNA, closed-ended linear duplex (CELiD) DNA, and transposon-mediated gene transfer (PiggyBac, Sleeping Beauty). Where a non-viral delivery system is used, the delivery vehicle can be a liposome. Lipid formulations can be used to introduce nucleic acids into a host cell in vitro, ex vivo, or in vivo. The nucleic acid may be encapsulated in the interior of a liposome, interspersed within the lipid bilayer of a liposome, attached to a liposome via a linking molecule that is associated with both the liposome and the nucleic acid, contained or complexed with a micelle, or otherwise associated with a lipid.

Additional definitions are provided throughout the present disclosure.

### Chimeric Engulfment Receptors (CERs)

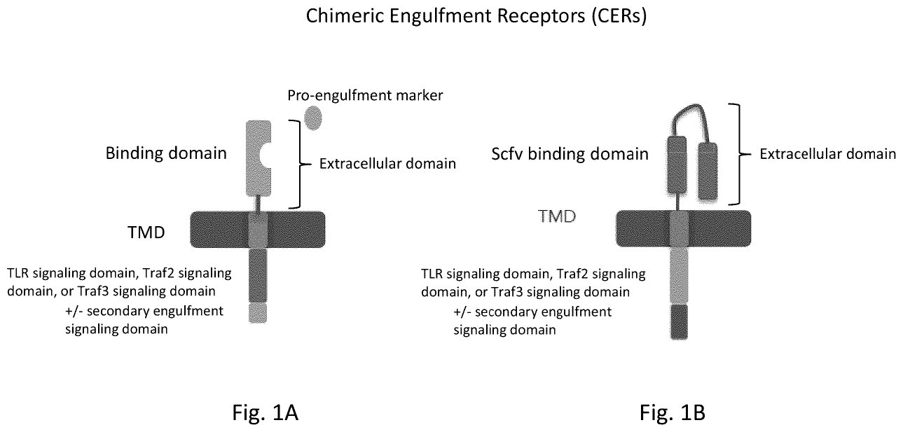

Chimeric engulfment receptors (CERs) are described herein. In particular embodiments, the CER is a chimeric, single chain protein, which comprises an extracellular domain and an engulfment signaling domain comprising a TLR signaling domain, a Traf6 signaling domain, a Traf2 signaling domain, or a Traf3 signaling domain, which are connected by a transmembrane domain. The extracellular domain includes an extracellular binding domain and, optionally, an extracellular spacer domain. When expressed in a host cell, a CER confers an engulfment phenotype to the modified host cell (the host cell is “switched” to an engulfment phenotype), specific to a selected pro-engulfment marker or antigenic marker present on or expressed by target cells, microbes, particles, or other materials. In particular CER embodiments, the chimeric protein comprises, from amino-terminus to carboxy-terminus: an extracellular domain having a binding domain specific for a target molecule and an optional extracellular spacer domain; a transmembrane domain; and an engulfment signaling domain comprising a TLR signaling domain, Traf6 signaling domain, Traf2 signaling domain, or Traf3 signaling domain. In further embodiments, the engulfment signaling domain comprises a primary engulfment signaling domain and a secondary engulfment signaling domain (see, e.g., FIGS. 1A and 1B).

The component parts of a CER as disclosed herein can be selected and arranged to provide a desired engulfment phenotype. For example, in certain embodiments, the extracellular domain can include a binding domain specific to: (i) a pro-engulfment marker associated with apoptotic, dead, dying, damaged, or necrotic cells; or (ii) an antigenic marker displayed by foreign (e.g., a microbe), infected, or aberrant cells associated with an infection, disease, disorder, or other undesired condition.

The engulfment signaling domain can include one or more effector (also referred to as “signaling”) domains that drive engulfment of the targeted cell. Signaling by the engulfment signaling domain is triggered by binding of the extracellular domain to the targeted pro-engulfment or antigenic marker. In certain embodiments, the engulfment signaling domain comprises an engulfment signaling domain comprising a TLR signaling domain, a Traf6 signaling domain, a Traf2 signaling domain, or a Traf3 signaling domain. In particular embodiments, a TLR signaling domain comprises a TLR1 signaling domain, a TLR 2 signaling domain, a TLR3 signaling domain, a TLR4 signaling domain, a TLR5 signaling domain, a TLR6 signaling domain, a TLR7 signaling domain, a TLR8 signaling domain, or a TLR9 signaling domain. In other embodiments, the engulfment signaling domain comprises a primary engulfment signaling domain comprising a TLR signaling domain, a Traf6 signaling domain, a Traf2 signaling domain, or a Traf3 signaling domain and a secondary engulfment signaling domain. In particular embodiments, the secondary engulfment signaling domain comprises a TLR1, TLR2, TLR3, TLR4, TLR5, TLR6, TLR7, TLR8, TLR9, TRAF6, TRAF2, TRAF3, FcγR1, FcγR2A, FcγR2B2, FcγR2C, FcγR3A, FcεR1, FcαR1, BAFFR, NFAM1, Dap12, MERTK, or CD79b signaling domain. A CER according to the present disclosure can be engineered for application in a variety of therapeutic contexts (e.g., clearance of apoptotic, dead, dying, damaged, infected, or necrotic cells, clearance of microbes responsible for infectious disease, and clearance of aberrant cells associated with a disease, disorder or undesired condition), while providing engulfment signaling that complements the desired therapeutic outcome (e.g., pro-inflammatory engulfment signaling).

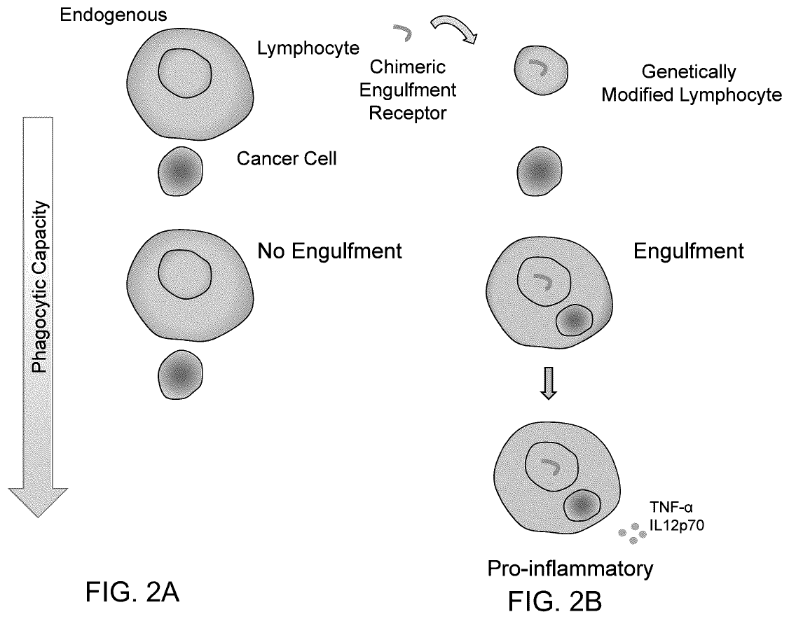

FIGS. 2A and 2B provide a functional comparison of a natural lymphocyte with a lymphocyte modified with an embodiment of a CER of the present disclosure. FIG. 2A shows an endogenous lymphocyte, and as is represented in the figure, the natural lymphocyte does not exhibit an engulfment phenotype. However, as is illustrated in FIG. 2B, a lymphocyte modified to express a CER as described herein exhibits an engulfment phenotype specific to the targeted cancer cell, leading to engulfment (e.g., phagocytosis) and elimination of the targeted cancer cell. In particular embodiments, the engulfment signaling domains included in CERs according to the present description can drive pro-inflammatory engulfment signaling.

Component parts of the fusion proteins of the present disclosure are further described in detail herein.

### I. Extracellular Domain

As described herein, a CER comprises an extracellular domain specific to a target molecule. In certain embodiments, the extracellular domain includes an extracellular binding domain that specifically binds a targeted pro-engulfment marker or antigen. Binding of a target molecule by the binding domain may block the interaction between the target molecule (e.g., a receptor or a ligand) and another molecule and, for example, interfere with, reduce or eliminate certain functions of the target molecule (e.g., signal transduction). In some embodiments, the binding of a target molecule may induce certain biological pathways or identify the target molecule or cell expressing the target molecule or cell expressing the target molecule for elimination.

A binding domain may be any polypeptide or peptide that specifically binds a target molecule of interest. Sources of binding domains include receptor binding domains, ligand binding domains, and antibodies or antigen binding portions, such as antibody variable regions from various species (which can be in the form of antibodies, sFvs, scFvs, Fabs, scFv-based grababody, or soluble VH domain or domain antibodies), including human, rodent, avian, or ovine. Additional sources of binding domains include variable regions of antibodies from other species, such as camelid (from camels, dromedaries, or llamas; Ghahroudi et al., FEBS Lett. 414:521, 1997; Vincke et al., J. Biol. Chem. 284:3273, 2009; Hamers-Casterman et al., Nature 363:446, 1993 and Nguyen et al., J. Mol. Biol. 275:413, 1998), nurse sharks (Roux et al., Proc. Nat'l. Acad. Sci. (USA) 95:11804, 1998), spotted ratfish (Nguyen et al., Immunogen. 54:39, 2002), or lamprey (Herrin et al., Proc. Nat'l. Acad. Sci. (USA) 105:2040, 2008 and Alder et al. Nat. Immunol. 9:319, 2008). These antibodies can form antigen-binding regions using only a heavy chain variable region, i.e., these functional antibodies are homodimers of heavy chains only (referred to as “heavy chain antibodies”) (Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008).

In some embodiments, the extracellular domain binds to a pro-engulfment marker. In certain such embodiments, the pro-engulfment marker targeted by the extracellular domain is phosphatidylserine (PtdSer), ICAM-3, oxidized low density lipoprotein, calreticulin, annexin I, complement C1q, or thrombospondin. In further embodiments, the extracellular domain that binds to a pro-engulfment marker is derived from an endogenous engulfment receptor or a soluble bridging molecule for an engulfment receptor (e.g., GAS6, Protein S, MFG-E8). In some embodiments, the entire extracellular portion (for membrane spanning molecules), the entire bridging molecule, or a truncated portion of an engulfment receptor or bridging molecule is used, provided that the truncated portion retains sufficient binding activity to the pro-engulfment marker (i.e., is a functional variant). In further embodiments, the extracellular portion of an engulfment receptor or bridging molecule used for the extracellular domain is a variant of the entire extracellular portion (for membrane spanning molecules), the entire bridging molecule, or a truncated portion of the engulfment receptor or bridging molecule, provided that the variant retains sufficient binding activity to the pro-engulfment marker (i.e., is a functional variant).

In some embodiments, the extracellular domain includes a T-cell immunoglobulin and mucin domain 1 (Tim1), T-cell immunoglobulin and mucin domain 4 (Tim4), T-cell immunoglobulin and mucin domain 3 (Tim3), stabilin-2, RAGE, or Fc receptor (FcR) extracellular domain. In specific embodiments, an FcR extracellular domain can include a binding domain from FcγR1, FcγR2A, FcγR2B2, FcγR2C, FcγR3A, FcεR1, or FcαR1. In further embodiments, the extracellular domain can include a PtdSer binding domain from Tim1, Tim4, Tim3, stabilin-2, receptor for advanced glycation end products (RAGE), brain-specific angiogenesis inhibitor 1 (BAI1), Milk Fat Globule-EGF Factor 8 Protein (MFG-E8) (e.g., a FA58C2 domain that mediates high affinity binding to PtdSer), Growth Arrest Specific 6 (GAS6), protein S, protein C, Factor II, Factor VII, Factor IX, Factor X, Beta 2-glycoprotein I, α5β3 integrin and other integrins, CR3 complement receptor, CR4 complement receptor, CD14, CD93, annexin V, phosphatidylserine receptor (PSr), prothrombin, or scavenger receptors such as scavenger receptor B (SRB) (e.g., SRB1 (CD36)), scavenger receptor C (SRC) (e.g., LOX-1, SRCL), scavenger receptor D (SRD) (e.g., CD68, macrosialin), and PSOX.

In some embodiments, the extracellular domain comprises or is a sequence that is at least 90%, at least 91%, at least 92%, at least 93%, at least 94%, at least 95%, at least 96%, at least 97%, at least 98%, at least 99%, at least 99.5%, or 100% identical to a FcγRI binding domain comprising an amino acid sequence of SEQ ID NO: 1 or amino acids 16-292 of SEQ ID NO:1, TIM1 binding domain comprising an amino acid sequence of SEQ ID NO:2 or amino acids 21-290 of SEQ ID NO:2, a TIM4 binding domain comprising an amino acid sequence of SEQ ID NO:3 or amino acids 25-314 of SEQ ID NO:3, a TIM3 binding domain comprising an amino acid sequence of SEQ ID NO:4 or amino acids 22-202 of SEQ ID NO:4, a FA58C2 binding domain comprising an amino acid sequence of SEQ ID NO:5, a GAS6 binding domain comprising an amino acid sequence of SEQ ID NO:6 or amino acids 31-94 of SEQ ID NO:6, a BAI1 binding domain comprising an amino acid sequence of SEQ ID NO:8 or a protein S binding domain comprising an amino acid sequence of SEQ ID NO:7 or amino acids 25-87 of SEQ ID NO:7. In certain other embodiments, the extracellular domain is encoded by a polynucleotide sequence that comprises or is a sequence that is at least 90%, at least 91%, at least 92%, at least 93%, at least 94%, at least 95%, at least 96%, at least 97%, at least 98%, at least 99%, at least 99.5%, or 100% identical to a polynucleotide encoding FcγRI binding domain according to SEQ ID NO:9, a polynucleotide encoding a TIM1 binding domain according to SEQ ID NO:10, a polynucleotide encoding a TIM4 binding domain according to SEQ ID NO:11, a polynucleotide encoding a TIM3 binding domain according to SEQ ID NO: 12, a polynucleotide encoding FA58C2 binding domain according to SEQ ID NO: 13, a polynucleotide encoding a GAS6 binding domain according to SEQ ID NO:14, a polynucleotide encoding a BAI1 binding domain according to SEQ ID NO:120, or a polynucleotide sequence encoding a protein S binding domain according to SEQ ID NO:15.

In other embodiments, the extracellular domain is derived from least one of the following: CD14, which binds to ICAM3; a scavenger receptor extracellular domain, which binds to oxidized LDL; a lectin, which binds to altered sugars; CD36, which binds to thrombospondin; or LRP1/CD91 or a lectin moiety, which binds to calreticulin.

In still other embodiments, the extracellular domain includes an antibody or antigen binding fragment thereof, such as a single chain Fv fragment (scFv) that comprises VH and VL regions, specific for a target molecule of interest. In certain embodiments, the antibody is chimeric, human, or humanized. In further embodiments, the V_{H} and V_{L} regions are human or humanized. In particular embodiments, the extracellular domain is an antibody or antigen binding portion thereof that is specific for a pro-engulfment marker. Antibodies specific for phosphatidylserine are known in the art (see, U.S. Pat. No. 7,247,303; Khogeer et al., 2015, Lupus 24:186-90; Gerber et al., 2015, Am. J. Nucl. Med. Mol. Imaging, 5:493-503, each of which is incorporated by reference in its entirety). In particular embodiments, a target molecule of interest is a tumor antigen, for example CD138, CD38, CD33, CD123, CD72, CD79a, CD79b, mesothelin, PSMA, BCMA, ROR1, MUC-16, L1CAM, CD22, CD19, CD20, CD23, CD24, CD37, CD30, CA125, CD56, c-Met, EGFR, GD-3, HPV E6, HPV E7, MUC-1, HER2, folate receptor α, CD97, CD171, CD179a, CD44v6, WT1, VEGF-α, VEGFR1, IL-13Rα1, IL-13Rα2, IL-11Rα, PSA, FcRH5, NKG2D ligand, NY-ESO-1, TAG-72, CEA, ephrin A2, ephrin B2, Lewis A antigen, Lewis Y antigen, MAGE, MAGE-A1, RAGE-1, folate receptor β, EGFRviii, VEGFR-2, LGR5, SSX2, AKAP-4, FLT3, fucosyl GM1, GM3, o-acetyl-GD2, and GD2, and exemplary V_{H} and V_{L} regions include the segments of anti-CD138, -CD38, -CD33, -CD123, -CD72, -CD79a-CD79b, -mesothelin, -PSMA, -BCMA, -ROR1, -MUC-16, -L1CAM, -CD22, -CD19, -CD20, -CD23, -CD24, -CD37, -CD30, -CA125, -CD56, -c-Met, -EGFR, -GD-3, -HPV E6, -HPV E7, -MUC-1, -HER2, -folate receptor α, -CD97, -CD171, -CD179a, -CD44v6, -WT1, -VEGF-α, -VEGFR1, -IL-13Rα1, -IL-13Rα2, -IL-11Rα, -PSA, -FcRH5, -NKG2D ligand, -NY-ESO-1, -TAG-72, -CEA, -ephrin A2, -ephrin B2, -Lewis A antigen, -Lewis Y antigen, -MAGE, -MAGE-A1, -RAGE-1, -folate receptor β, -EGFRviii, -VEGFR-2, -LGR5, -SSX2, -AKAP-4, -FLT3, -fucosyl GM1, -GM3, -o-acetyl-GD2, and -GD2 specific monoclonal antibodies, respectively.

In further embodiments, the extracellular domain includes a Fab specific for a target of interest. In such embodiments, targets of interest include CD138, CD38, CD33, CD123, CD72, CD79a, CD79b, mesothelin, PSMA, BCMA, ROR1, MUC-16, L1CAM, CD22, CD19, CD20, CD23, CD24, CD37, CD30, CA125, CD56, c-Met, EGFR, GD-3, HPV E6, HPV E7, MUC-1, HER2, folate receptor α, CD97, CD171, CD179a, CD44v6, WT1, VEGF-α, VEGFR1, IL-13Rα1, IL-13Rα2, IL-11Rα, PSA, FcRH5, NKG2D ligand, NY-ESO-1, TAG-72, CEA, ephrin A2, ephrin B2, Lewis A antigen, Lewis Y antigen, MAGE, MAGE-A1, RAGE-1, folate receptor β, EGFRviii, VEGFR-2, LGR5, SSX2, AKAP-4, FLT3, fucosyl GM1, GM3, o-acetyl-GD2, and GD2, and Fab regions include portions of anti-CD138, -CD38, -CD33, -CD123, -CD72, -CD79a, -CD79b, -mesothelin, -PSMA, -BCMA, -ROR1, -MUC-16, -L1CAM, -CD22, -CD19, -CD20, -CD23, -CD24, -CD37, -CD30, -CA125, -CD56, -c-Met, -EGFR, -GD-3, -HPV E6, -HPV E7, -MUC-1, -HER2, -folate receptor α, -CD97, -CD171, -CD179a, -CD44v6, -WT1, -VEGF-α, -VEGFR1, -IL-13Rα1, -IL-13Rα2, -IL-11Rα, -PSA, -FcRH5, -NKG2D ligand, -NY-ESO-1, -TAG-72, -CEA, -ephrin A2, -ephrin B2, -Lewis A antigen, -Lewis Y antigen, -MAGE, MAGE-A1, -RAGE-1, -folate receptor β, -EGFRviii, -VEGFR-2, -LGR5, -SSX2, AKAP-4, -FLT3, -fucosyl GM1, -GM3, -o-acetyl-GD2, and -GD2 specific monoclonal antibodies, respectively.

A target molecule, which is specifically bound by an extracellular domain of a CER of the present disclosure, may be found on or in association with a cell of interest (“target cell”). Exemplary target cells include a cancer cell, a cell associated with an autoimmune disease or disorder or with an inflammatory disease or disorder, and an infectious microbe (e.g., bacteria, virus, or fungi), or infected cell (e.g., virus-infected cell). A cell of an infectious organism, such as a mammalian parasite, is also contemplated as a target cell.

In some embodiments, the extracellular domain optionally comprises an extracellular, non-signaling spacer or linker domain. Where included, such a spacer or linker domain may position the binding domain away from the host cell surface to further enable proper cell/cell contact, binding, activation, and expansion. An extracellular spacer domain is generally located between the extracellular binding domain and the transmembrane domain. The length of the extracellular spacer may be varied to optimize target molecule binding based on the selected target molecule, selected binding epitope, binding domain size and affinity (see, e.g., Guest et al., J. Immunother. 28:203-11, 2005; Hudecek et al., Clin. Cancer Res. 19:3153-64, 2013; Hudecek et al., Cancer Immunol. Res. 3:125-35, 2015; PCT Publication No. WO 2014/031687; each of which is incorporated by reference in its entirety). In certain embodiments, an extracellular spacer domain comprises a TLR juxtamembrane domain (e.g., TLR1, TLR2, TLR3, TLR4, TLR5, TLR6, TLR7, TLR8, or TLR9 juxtamembrane domain). In a particular embodiment, an extracellular spacer domain comprises a TLR4 juxtamembrane domain comprising an amino acid sequence of SEQ ID NO:17. In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region. An altered IgG₄ hinge region is described in PCT Publication No. WO 2014/031687, which hinge region is incorporated herein by reference in its entirety. In a particular embodiment, an extracellular spacer domain comprises a modified IgG₄ hinge region having an amino acid sequence of ESKYGPPCPPCP (SEQ ID NO:16). Other examples of hinge regions that may be used in the CERs described herein include the hinge region present in the extracellular regions of type 1 membrane proteins, such as CD8a, CD4, CD28 and CD7, which may be wild-type or variants thereof. In further embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In a particular embodiment, the Fc domain is modified to prevent in vivo interactions with cells expressing FcγRs that may result in off-target activation of CER-modified cells. In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D. In yet further embodiments, an extracellular spacer domain may be derived from MERTK.

### II. Transmembrane Domains

The transmembrane domain connects and is positioned between the extracellular domain and the engulfment signaling domain. The transmembrane domain is a hydrophobic alpha helix that transverses the host cell membrane. The transmembrane domain may be directly fused to the binding domain or to the extracellular spacer domain if present. In certain embodiments, the transmembrane domain is derived from an integral membrane protein (e.g., receptor, cluster of differentiation (CD) molecule, enzyme, transporter, cell adhesion molecule, or the like). The transmembrane domain can be naturally associated with either the extracellular domain or the engulfment signaling domain included in the CER (e.g., a CER comprises a Tim4 binding domain and a Tim4 transmembrane domain). In certain embodiments, the transmembrane domain and the extracellular domain are derived from different molecules, the transmembrane domain and the engulfment signaling domain are derived from different molecules, or the transmembrane domain, extracellular domain, and engulfment signaling domain are all derived from different molecules.

In certain embodiments, the transmembrane domain is a TLR transmembrane domain (e.g., TLR1, TLR2, TLR3, TLR4, TLR5, TLR6, TLR7, TLR8, or TLR9 transmembrane domain), a Tim1 transmembrane domain, a Tim4 transmembrane domain, an FcR transmembrane domain (e.g., FcγR1, FcγR2A, FcγR2B2, FcγR2C, FcγR3A, FcεR1, or FcαR1 transmembrane domain), a CD8a transmembrane domain, a MERTK transmembrane domain, an Axl transmembrane domain, a Tyro3 transmembrane domain, a BAI1 transmembrane domain, a CD4 transmembrane domain, a CD28 transmembrane domain a MRC1 transmembrane domain, or a DAP12 transmembrane domain.

In specific embodiments, the transmembrane domain comprises or is a sequence that is at least 90%, at least 91%, at least 92%, at least 93%, at least 94%, at least 95%, at least 96%, at least 97%, at least 98%, at least 99%, at least 99.5%, or 100% identical to a TLR1 transmembrane domain comprising an amino acid sequence of SEQ ID NO:31, a TLR2 transmembrane domain comprising an amino acid sequence of SEQ ID NO:32, a TLR3 transmembrane domain comprising an amino acid sequence of SEQ ID NO:33, a TLR4 transmembrane domain comprising an amino acid sequence of SEQ ID NO:34, a TLR5 transmembrane domain comprising an amino acid sequence of SEQ ID NO:35, a TLR6 transmembrane domain comprising an amino acid sequence of SEQ ID NO:36, a TLR7 transmembrane domain comprising an amino acid sequence of SEQ ID NO:37, a TLR8 transmembrane domain comprising an amino acid sequence of SEQ ID NO:38, a TLR9 transmembrane domain comprising an amino acid sequence of SEQ ID NO:39, a Tim1 transmembrane domain comprising an amino acid sequence of SEQ ID NO:18, a Tim4 transmembrane domain comprising an amino acid sequence of SEQ ID NO:19, an FcγRI transmembrane domain comprising an amino acid sequence of SEQ ID NO:20, a FcεRIγ transmembrane domain comprising an amino acid sequence of SEQ ID NO:21, a CD8a transmembrane domain comprising an amino acid sequence of SEQ ID NO:22, a MERTK transmembrane domain comprising an amino acid sequence of SEQ ID NO:23, an Axl transmembrane domain comprising an amino acid sequence of SEQ ID NO:24, a Tyro3 transmembrane domain comprising an amino acid sequence of SEQ ID NO:25, a BAI1 transmembrane domain comprising an amino acid sequence of SEQ ID NO:29, a CD28 transmembrane domain as set forth in an amino acid sequence of SEQ ID NO:26, a CD4 transmembrane domain comprising an amino acid sequence of SEQ ID NO:27, a MRC1 transmembrane domain comprising an amino acid sequence of SEQ ID NO:30, or a DAP12 transmembrane domain comprising an amino acid sequence of SEQ ID NO:28.

In other embodiments, the transmembrane domain is provided by a polynucleotide sequence that comprises or is a sequence that is at least 90%, at least 91%, at least 92%, at least 93%, at least 94%, at least 95%, at least 96%, at least 97%, at least 98%, at least 99%, at least 99.5%, or 100% identical to a polynucleotide sequence encoding a Tim1 transmembrane domain according to SEQ ID NO:40, a polynucleotide sequence encoding a Tim4 transmembrane domain according to SEQ ID NO:41, a polynucleotide sequence encoding a FcεRIγ transmembrane domain according to SEQ ID NO: 121, a polynucleotide sequence encoding an FcγRI transmembrane domain according to SEQ ID NO:42, a polynucleotide sequence encoding a CD8a transmembrane domain according to SEQ ID NO:43, a polynucleotide sequence encoding MERTK transmembrane domain according to SEQ ID NO:44, a polynucleotide sequence encoding an Axl transmembrane domain according to SEQ ID NO:45, a polynucleotide sequence encoding a Tyro3 transmembrane domain according to SEQ ID NO:46, a polynucleotide sequence encoding a CD28 transmembrane domain according to SEQ ID NO:110, a polynucleotide sequence encoding a BAIL transmembrane domain according to SEQ ID NO:113, a polynucleotide sequence encoding a CD4 transmembrane domain according to SEQ ID NO:47, a polynucleotide sequence encoding a DAP12 transmembrane domain according to SEQ ID NO:111, or a polynucleotide sequence encoding a TLR4 transmembrane domain according to SEQ ID NO:112.

It is understood that direct fusion of one domain to another domain of a CER described herein does not preclude the presence of intervening junction amino acids. Junction amino acids may be natural or non-natural (e.g., resulting from the construct design of a chimeric protein).

### III. Engulfment Signaling Domains

The engulfment signaling domain of a CER is an intracellular effector domain and is capable of transmitting functional signals to a cell in response to binding of the extracellular domain of the CER to a target molecule. CERs of the present disclosure may include one or more engulfment signaling domains as described herein.

In certain embodiments, an engulfment signaling domain is an intracellular signaling domain of an endogenous toll-like (TLR) receptor or an intracellular signaling domain of an endogenous signal transduction protein that is involved in TLR signaling. Ten TLRs have been identified in humans. Examples of endogenous TLRs from which engulfment signaling domains can be derived include TLR1, TLR2, TLR3, TLR4, TLR5, TLR6, TLR7, TLR8, and TLR9. Examples of endogenous signal transduction proteins that are involved in TLR signaling that may be used to derive engulfment signaling domains include Traf6, Traf2, and Traf3.

In particular embodiments, the primary engulfment signaling domain comprises or is a sequence that is at least 90%, at least 91%, at least 92%, at least 93%, at least 94%, at least 95%, at least 96%, at least 97%, at least 98%, at least 99%, at least 99.5%, or 100% identical to a TLR1 signaling domain comprising an amino acid sequence of SEQ ID NO:48, a TLR2 signaling domain comprising an amino acid sequence of SEQ ID NO:49, a TLR3 signaling domain comprising an amino acid sequence of SEQ ID NO:50, a TLR4 signaling domain comprising an amino acid sequence of SEQ ID NO:51, a TLR5 signaling domain comprising an amino acid sequence of SEQ ID NO:52, a TLR6 signaling domain comprising an amino acid sequence of SEQ ID NO:53, a TLR7 signaling domain comprising an amino acid sequence of SEQ ID NO:54, a TLR8 signaling domain comprising an amino acid sequence of SEQ ID NO:55, a TLR9 signaling domain comprising an amino acid sequence of SEQ ID NO:56, a Traf6 signaling domain comprising an amino acid sequence of SEQ ID NO:57, a truncated Traf6 signaling domain comprising an amino acid sequence of SEQ ID NO:58, a Traf2 signaling domain comprising an amino acid sequence of SEQ ID NO:72, or a Traf3 signaling domain comprising an amino acid sequence of SEQ ID NO:73.

The engulfment signaling domain may be any portion of an engulfment signaling molecule that retains sufficient signaling activity. In some embodiments, a full length or full length intracellular component of an engulfment signaling molecule is used. In some embodiments, a truncated portion of an engulfment signaling molecule or intracellular component of an engulfment signaling molecule is used, provided that the truncated portion retains sufficient signal transduction activity. In further embodiments, an engulfment signaling domain is a variant of an entire or truncated portion of an engulfment signaling molecule, provided that the variant retains sufficient signal transduction activity (i.e., is a functional variant).

In certain embodiments, the engulfment signaling domain comprises: a primary engulfment signaling domain comprising a TLR signaling domain, a Traf6 signaling domain, a Traf2 signaling domain, or a Traf3 signaling domain; and a secondary engulfment signaling domain. A secondary engulfment signaling domain may comprise an FcR signaling domain (including an FcγR1 signaling domain, an FcγR2A signaling domain, an FcγR2C signaling domain, FcγR2B2 signaling domain, an FcγR3A signaling domain, FcγR2C signaling domain, FcγR3A signaling domain, FcεR1 signaling domain, and FcαR1 signaling domain), a B-cell activating factor receptor (BAFF-R) signaling domain, a DAP12 (also referred to as TYRO Protein Tyrosine Kinase Binding Protein (TYROBP)) signaling domain, an NFAT Activating Protein With ITAM Motif 1 (NFAM1) signaling domain, a MERTK signaling domain, a TLR1 signaling domain, a TLR2 signaling domain, a TLR3 signaling domain, a TLR4 signaling domain, a TLR5 signaling domain, a TLR6 signaling domain, a TLR7 signaling domain, a TLR8 signaling domain, a TLR9 signaling domain, a Traf6 signaling domain, a Traf2 signaling domain, or a Traf3 signaling domain, or a CD79b signaling domain. In particular embodiments, the secondary engulfment signaling domain comprises or is a sequence that is at least 90%, at least 91%, at least 92%, at least 93%, at least 94%, at least 95%, at least 96%, at least 97%, at least 98%, at least 99%, at least 99.5%, or 100% identical to a FcεRIγ signaling domain comprising an amino acid sequence of SEQ ID NO:62, an FcγRI signaling domain comprising an amino acid sequence of SEQ ID NO:63, an FcγR2A signaling domain comprising an amino acid sequence of SEQ ID NO:64, an FcγR2C signaling domain comprising an amino acid sequence of SEQ ID NO:65, an FcγR3A signaling domain comprising an amino acid sequence of SEQ ID NO:66, a BAFF-R signaling domain comprising an amino acid sequence of SEQ ID NO:67, a DAP12 signaling domain comprising an amino acid sequence of SEQ ID NO:68, a NFAM1 signaling domain comprising an amino acid sequence of SEQ ID NO:69, a truncated NFAM1 signaling domain comprising an amino acid sequence of SEQ ID NO:70, a CD79b signaling domain comprising an amino acid sequence of SEQ ID NO:75, a truncated CD79b signaling domain comprising an amino acid sequence of SEQ ID NO:71, a MERTK signaling domain comprising an amino acid sequence of SEQ ID NO:59, a TLR1 signaling domain comprising an amino acid sequence of SEQ ID NO:48, a TLR2 signaling domain comprising an amino acid sequence of SEQ ID NO:49, a TLR3 signaling domain comprising an amino acid sequence of SEQ ID NO:50, a TLR4 signaling domain comprising an amino acid sequence of SEQ ID NO:51, a TLR5 signaling domain comprising an amino acid sequence of SEQ ID NO:52, a TLR6 signaling domain comprising an amino acid sequence of SEQ ID NO:53, a TLR7 signaling domain comprising an amino acid sequence of SEQ ID NO:54, a TLR8 signaling domain comprising an amino acid sequence of SEQ ID NO:55, a TLR9 signaling domain comprising an amino acid sequence of SEQ ID NO:56, a Traf6 signaling domain comprising an amino acid sequence of SEQ ID NO:57, a truncated Traf6 signaling domain comprising an amino acid sequence of SEQ ID NO:58, a Traf2 signaling domain comprising an amino acid sequence of SEQ ID NO:72, or a Traf3 signaling domain comprising an amino acid sequence of SEQ ID NO:73.

In other embodiments, the secondary engulfment signaling domain is provided by a polynucleotide sequence that comprises or is a sequence that is at least 90%, at least 91%, at least 92%, at least 93%, at least 94%, at least 95%, at least 96%, at least 97%, at least 98%, at least 99%, at least 99.5%, or 100% identical to a polynucleotide encoding a FcεRIγ signaling domain according to SEQ ID NO:115, a polynucleotide encoding an FcγR1 signaling domain according to SEQ ID NO:77, a polynucleotide encoding an FcγR2A signaling domain according to SEQ ID NO:78, a polynucleotide encoding an FcγR2C signaling domain according to SEQ ID NO:79, a polynucleotide encoding an FcγR3A signaling domain according to SEQ ID NO:80, a polynucleotide encoding a BAFF-R signaling domain according to SEQ ID NO: 117, a polynucleotide encoding a DAP12 signaling domain according to SEQ ID NO:116, a polynucleotide encoding a NFAM1 signaling domain according to SEQ ID NO:119, or a polynucleotide encoding a CD79b signaling domain according to SEQ ID NO:118.

In certain embodiments, signaling by the engulfment signaling domain results in expression of at least one of an inflammatory cytokine, an inflammatory chemokine, or a co-stimulatory cell surface marker. In yet further embodiments, the inflammatory cytokine is TNFα, IL-1, IL-6, IL-12, or IL-23; the inflammatory chemokine is CCL5 (RANTES), CXCL9, or CXCL10; and the co-stimulatory cell surface marker is CD80, CD86, HLA-DR, CD40, HVEM, or 4-1BBL; or any combination thereof.

In certain embodiments, the presence of a secondary engulfment signaling domain enhances engulfment activity of the CER, persistence of the CER modified host cell, expansion of the CER modified host cell, or a combination thereof. In a particular embodiment, inclusion of a secondary engulfment signaling domain with a primary engulfment signaling domain enhances engulfment activity of the CER, phagocytic signaling activity of the CER modified host cell, degradation of luminal contents by the CER modified host cell, activation of the CER modified host cell, persistence of the CER modified host cell, memory formation of the CER modified host cell, expansion of the CER modified host cell, antigen presenting activity by the CER modified host cell, or any combination thereof.

In certain embodiments, the presence of a TLR signaling domain with a Traf2, Traf3, or Traf6 signaling domain in a CER provides enhanced functionality to the CER and/or CER-modified host cell. In one embodiment, a primary engulfment signaling domain comprises a TLR1, TLR2, TLR3, TLR4, TLR5, TLR6, TLR7, TLR8, or TLR9 signaling domain and a secondary engulfment signaling domain comprises a Traf6, Traf2, or Traf3 signaling domain. In another embodiment, a primary engulfment signaling domain comprises a Traf6, Traf2, or Traf3 signaling domain, and a secondary engulfment signaling domain comprises a TLR1, TLR2, TLR3, TLR4, TLR5, TLR6, TLR7, TLR8, or TLR9 signaling domain. In certain embodiments, CERs comprising both a TLR signaling domain and a Traf2, Traf3, or Traf6 signaling domain exhibits enhanced activation, persistence, memory formation, antigen presentation, or any combination thereof.

It is understood that in embodiments where a CER comprises a primary engulfment signaling domain and a secondary engulfment signaling domain, the positions of the engulfment signaling domains may be exchanged. For example, in a CER comprising a primary engulfment signaling domain and a secondary engulfment signaling domain, the secondary engulfment signaling domain may comprise a TLR signaling domain (e.g., TLR1, TLR2, TLR3, TLR4, TLR5, TLR6, TLR7, TLR8 or TLR9), a Traf2 signaling domain, or a Traf3 signaling domain, and the primary signaling domain may comprise an FcR signaling domain (including an FcγR1 signaling domain, an FcγR2A signaling domain, an FcγR2C signaling domain, FcγR2B2 signaling domain, an FcγR3A signaling domain, FcγR2C signaling domain, FcγR3A signaling domain, FcεR1 signaling domain, and FcαR1 signaling domain), a BAFF-R signaling domain, a DAP12 signaling domain, a NFAM1 signaling domain, MERTK signaling domain, a CD79b signaling domain, a TLR signaling domain, a Traf2 signaling domain, a Traf3 signaling domain, or a Traf6 signaling domain.

### IV. Examples of CERs

The component parts of a CER as disclosed herein can be selected and arranged in various combinations to provide a desired engulfment phenotype to a host cell. In addition to inducing engulfment of a cell, microbe, or particle expressing or characterized by a molecule targeted by a CER-modified host cell, a CER as described herein may be designed to initiate a pro-inflammatory engulfment response, enhance engulfment activity, degradation of luminal contents, cytolytic activity, cell activation, cell expansion, cell memory, cell persistence, antigen presentation, or cell proliferation, depending upon the target cell or particle, disease state, and desired therapeutic outcome.

In one aspect, the present disclosure provides a chimeric engulfment receptor (CER) comprising a single chain chimeric protein, the single chain chimeric protein comprising: an extracellular domain comprising a binding domain that binds to phosphatidylserine (PtdSer); an engulfment signaling domain comprising a TLR signaling domain, a Traf6 signaling domain, a Traf2 signaling domain, or a Traf3 signaling domain; and a transmembrane domain positioned between and connecting the extracellular domain and the engulfment signaling domain.

In certain embodiments, the extracellular domain further comprises an extracellular spacer domain positioned between the binding domain and the transmembrane domain.

In certain embodiments, the CER further comprises a secondary engulfment signaling domain. A secondary engulfment signaling domain may comprise an FcR signaling domain (including an FcγR1 signaling domain, an FcγR2A signaling domain, an FcγR2C signaling domain, FcγR2B2 signaling domain, an FcγR3A signaling domain, FcγR2C signaling domain, FcγR3A signaling domain, FcεR1 signaling domain, and FcαR1 signaling domain), a BAFF-R signaling domain, a DAP12 signaling domain, a NFAM1 signaling domain, a CD79b signaling domain, a MERTK signaling domain, a TLR signaling domain, a Traf6 signaling domain, a Traf2 signaling domain, or a Traf3 signaling domain.

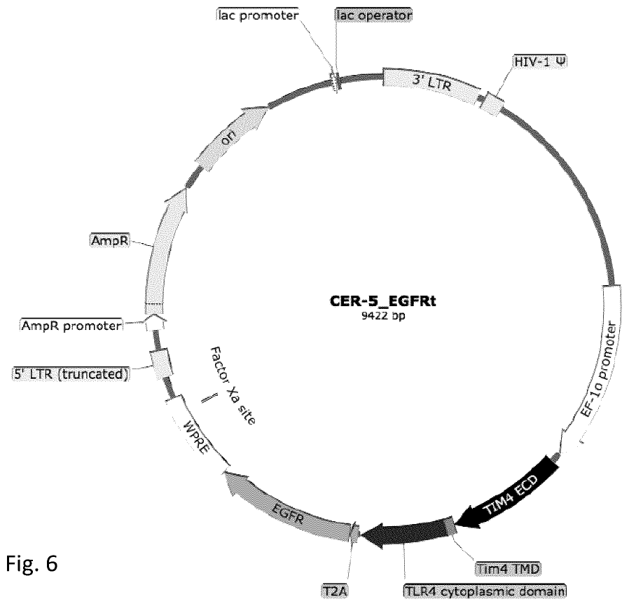

An embodiment of a CER including an extracellular domain comprising a binding domain that binds to PtdSer comprises: an extracellular domain comprising a TIM4 PtdSer binding domain; a transmembrane domain comprising a TIM4 transmembrane domain, and an engulfment signaling domain comprising a TLR4 signaling domain (also referred to herein as “CER05”) (see, e.g., FIG. 6). In certain embodiments, such a CER comprises an amino acid sequence of SEQ ID NO:81. In some embodiments, the CER mature polypeptide sequence comprises an amino acid sequence of SEQ ID NO:81 without the signal peptide sequence (amino acids 1-22 of SEQ ID NO:81).

Another embodiment of a CER including an extracellular domain comprising a binding domain that binds to PtdSer comprises: an extracellular domain comprising a TIM4 PtdSer binding domain; a transmembrane domain comprising a TLR4 transmembrane domain, and an engulfment signaling domain comprising a TLR4 signaling domain (also referred to herein as “CER06”). In certain embodiments, such a CER comprises an amino acid sequence of SEQ ID NO:82. In some embodiments, the CER mature polypeptide sequence comprises an amino acid sequence of SEQ ID NO:82 without the signal peptide sequence (amino acids 1-22 of SEQ ID NO:82).

Another embodiment of a CER including an extracellular domain comprising a binding domain that binds to PtdSer comprises: an extracellular domain comprising a TIM4 PtdSer binding domain; an extracellular spacer domain comprising a TLR4 juxtamembrane domain, a transmembrane domain comprising a TLR4 transmembrane domain, and an engulfment signaling domain comprising a TLR4 signaling domain (also referred to herein as “CER07”). In certain embodiments, such a CER comprises an amino acid sequence of SEQ ID NO:83. In some embodiments, the CER mature polypeptide sequence comprises an amino acid sequence of SEQ ID NO:83 without the signal peptide sequence (amino acids 1-22 of SEQ ID NO:83).

Another embodiment of a CER including an extracellular domain comprising a binding domain that binds to PtdSer comprises: an extracellular domain comprising a TIM4 PtdSer binding domain; a transmembrane domain comprising a Tim4 transmembrane domain, and an engulfment signaling domain comprising a TLR3 signaling domain (also referred to herein as “CER17”). In certain embodiments, such a CER comprises an amino acid sequence of SEQ ID NO:84. In some embodiments, the CER mature polypeptide sequence comprises an amino acid sequence of SEQ ID NO:84 without the signal peptide sequence (amino acids 1-22 of SEQ ID NO:84).

Another embodiment of a CER including an extracellular domain comprising a binding domain that binds to PtdSer comprises: an extracellular domain comprising a TIM4 PtdSer binding domain; a transmembrane domain comprising a TLR3 transmembrane domain, and an engulfment signaling domain comprising a TLR3 signaling domain (also referred to herein as “CER18”). In certain embodiments, such a CER comprises an amino acid sequence of SEQ ID NO:85. In some embodiments, the CER mature polypeptide sequence comprises an amino acid sequence of SEQ ID NO:85 without the signal peptide sequence (amino acids 1-22 of SEQ ID NO:85).

Another embodiment of a CER including an extracellular domain comprising a binding domain that binds to PtdSer comprises: an extracellular domain comprising a TIM4 PtdSer binding domain; a transmembrane domain comprising a Tim4 transmembrane domain, and an engulfment signaling domain comprising a TLR5 signaling domain (also referred to herein as “CER19”). In certain embodiments, such a CER comprises an amino acid sequence of SEQ ID NO:86. In some embodiments, the CER mature polypeptide comprises an amino acid sequence of SEQ ID NO:86 without the signal peptide sequence (amino acids 1-22 of SEQ ID NO:86).

Yet another embodiment of a CER including an extracellular domain comprising a binding domain that binds to PtdSer comprises: an extracellular domain comprising a TIM4 PtdSer binding domain; a transmembrane domain comprising a TLR5 transmembrane domain, and an engulfment signaling domain comprising a TLR5 signaling domain (also referred to herein as “CER20”). In certain embodiments, such a CER comprises an amino acid sequence of SEQ ID NO:87. In some embodiments, the CER mature polypeptide sequence comprises an amino acid sequence of SEQ ID NO:87 without the signal peptide sequence (amino acids 1-22 of SEQ ID NO:87).

Another embodiment of a CER including an extracellular domain comprising a binding domain that binds to PtdSer comprises: an extracellular domain comprising a TIM4 PtdSer binding domain; a transmembrane domain comprising a Tim4 transmembrane domain, and an engulfment signaling domain comprising a TLR8 signaling domain (also referred to herein as “CER21”). In certain embodiments, such a CER comprises an amino acid sequence of SEQ ID NO:88. In some embodiments, the CER mature polypeptide sequence comprises an amino acid sequence of SEQ ID NO:88 without the signal peptide sequence (amino acids 1-22 of SEQ ID NO:88).

Another embodiment of a CER including an extracellular domain comprising a binding domain that binds to PtdSer comprises: an extracellular domain comprising a TIM4 PtdSer binding domain; a transmembrane domain comprising a TLR8 transmembrane domain, and an engulfment signaling domain comprising a TLR8 signaling domain (also referred to herein as “CER22”). In certain embodiments, such a CER comprises an amino acid sequence of SEQ ID NO:89. In some embodiments, the CER mature polypeptide sequence comprises an amino acid sequence of SEQ ID NO:89 without the signal peptide sequence (amino acids 1-22 of SEQ ID NO:89).

Another embodiment of a CER including an extracellular domain comprising a binding domain that binds to PtdSer comprises: an extracellular domain comprising a TIM4 PtdSer binding domain; a transmembrane domain comprising a Tim4 transmembrane domain, and an engulfment signaling domain comprising a TLR9 signaling domain (also referred to herein as “CER23”). In certain embodiments, such a CER comprises an amino acid sequence of SEQ ID NO:90. In some embodiments, the CER mature polypeptide sequence comprises an amino acid sequence of SEQ ID NO:90 without the signal peptide sequence (amino acids 1-22 of SEQ ID NO:90).

Another embodiment of a CER including an extracellular domain comprising a binding domain that binds to PtdSer comprises: an extracellular domain comprising a TIM4 PtdSer binding domain; a transmembrane domain comprising a TLR9 transmembrane domain, and an engulfment signaling domain comprising a TLR9 signaling domain (also referred to herein as “CER24”). In certain embodiments, such a CER comprises an amino acid sequence of SEQ ID NO:91. In some embodiments, the CER mature polypeptide sequence comprises an amino acid sequence of SEQ ID NO:91 without the signal peptide sequence (amino acids 1-22 of SEQ ID NO:91).

Another embodiment of a CER including an extracellular domain comprising a binding domain that binds to PtdSer comprises: an extracellular domain comprising a TIM4 PtdSer binding domain; a transmembrane domain comprising a Tim4 transmembrane domain, and an engulfment signaling domain comprising a TLR1 signaling domain (also referred to herein as “CER26”). In certain embodiments, such a CER comprises an amino acid sequence of SEQ ID NO:92. In some embodiments, the CER comprises an amino acid sequence of SEQ ID NO:92 without the signal peptide sequence (amino acids 1-22 of SEQ ID NO:92).

Another embodiment of a CER including an extracellular domain comprising a binding domain that binds to PtdSer comprises: an extracellular domain comprising a TIM4 PtdSer binding domain; a transmembrane domain comprising a Tim4 transmembrane domain, and an engulfment signaling domain comprising a TLR2 signaling domain (also referred to herein as “CER27”). In certain embodiments, such a CER comprises an amino acid sequence of SEQ ID NO:93. In some embodiments, the CER comprises an amino acid sequence of SEQ ID NO:93 without the signal peptide sequence (amino acids 1-22 of SEQ ID NO:93).

Another embodiment of a CER including an extracellular domain comprising a binding domain that binds to PtdSer comprises: an extracellular domain comprising a TIM4 PtdSer binding domain; a transmembrane domain comprising a Tim4 transmembrane domain, and an engulfment signaling domain comprising a TLR7 signaling domain (also referred to herein as “CER28”). In certain embodiments, such a CER comprises an amino acid sequence of SEQ ID NO:94. In some embodiments, the CER comprises an amino acid sequence of SEQ ID NO:94 without the signal peptide sequence (amino acids 1-22 of SEQ ID NO:94).

Another embodiment of a CER including an extracellular domain comprising a binding domain that binds to PtdSer comprises: an extracellular domain comprising a TIM4 PtdSer binding domain; a transmembrane domain comprising a Tim4 transmembrane domain, and an engulfment signaling domain comprising a Traf2 signaling domain (also referred to herein as “CER30”). In certain embodiments, such a CER comprises an amino acid sequence of SEQ ID NO:96. In some embodiments, the CER comprises an amino acid sequence of SEQ ID NO:96 without the signal peptide sequence (amino acids 1-22 of SEQ ID NO:96).

Another embodiment of a CER including an extracellular domain comprising a binding domain that binds to PtdSer comprises: an extracellular domain comprising a TIM4 PtdSer binding domain; a transmembrane domain comprising a Tim4 transmembrane domain, and an engulfment signaling domain comprising a Traf3 signaling domain (also referred to herein as “CER31”). In certain embodiments, such a CER comprises an amino acid sequence of SEQ ID NO:97. In some embodiments, the CER comprises an amino acid sequence of SEQ ID NO:97 without the signal peptide sequence (amino acids 1-22 of SEQ ID NO:97).

Another embodiment of a CER including an extracellular domain comprising a binding domain that binds to PtdSer comprises: an extracellular domain comprising a CD22 specific scFv binding domain; an extracellular spacer domain comprising a mutated IgG4 hinge region; a transmembrane domain comprising a TLR4 transmembrane domain, and an engulfment signaling domain comprising a TLR4 signaling domain (also referred to herein as “CER42”). In certain embodiments, such a CER comprises an amino acid sequence of SEQ ID NO:98. In some embodiments, the CER comprises an amino acid sequence of SEQ ID NO:98 without the signal peptide sequence (amino acids 1-22 of SEQ ID NO:98).

Another embodiment of a CER including an extracellular domain comprising a binding domain that binds to PtdSer comprises: an extracellular domain comprising a Tim4 binding domain, a transmembrane domain comprising a Tim4 transmembrane domain, an engulfment signaling domain comprising a truncated Traf6 signaling domain (also referred to herein as “CER29”). In certain embodiments, such a CER comprises an amino acid sequence of SEQ ID NO: 124. In some embodiments, the CER mature polypeptide sequence comprises an amino acid sequence of SEQ ID NO: 124 without the signal peptide sequence (amino acids 1-22 of SEQ ID NO:124).

Another embodiment of a CER including an extracellular domain comprising a binding domain that binds to PtdSer comprises: a Tim4 binding domain, a transmembrane domain comprising a Tim4 transmembrane domain, and an engulfment signaling domain comprising a primary engulfment signaling domain comprising a truncated Traf6 signaling domain and a secondary engulfment signaling domain comprising a NFAM1 signaling domain (also referred to herein as “CER112”). In certain embodiments, such a CER comprises an amino acid sequence of SEQ ID NO:128. In some embodiments, the CER mature polypeptide sequence comprises an amino acid sequence of SEQ ID NO:128 without the signal peptide sequence (amino acids 1-22 of SEQ ID NO:128).

Another embodiment of a CER including an extracellular domain comprising a binding domain that binds to PtdSer comprises: a Tim4 binding domain, a transmembrane domain comprising a Tim4 transmembrane domain, and an engulfment signaling domain comprising a primary engulfment signaling domain comprising a truncated Traf6 signaling domain and a secondary engulfment signaling domain comprising a DAP12 signaling domain (also referred to herein as “CER110”). In certain embodiments, such a CER comprises an amino acid sequence of SEQ ID NO:125. In some embodiments, the CER mature polypeptide sequence comprises an amino acid sequence of SEQ ID NO:125 without the signal peptide sequence (amino acids 1-22 of SEQ ID NO: 125).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a truncated Traf6 signaling domain, and a secondary engulfment signaling domain comprising a BAFFR signaling domain (also referred to herein as “CER113”). In certain embodiments, such a CER comprises an amino acid sequence of SEQ ID NO:127 or 140. In some embodiments, the CER mature polypeptide sequence comprises an amino acid sequence of SEQ ID NO:127 without the signal peptide sequence (amino acids 1-22 of SEQ ID NO:127).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a truncated Traf6 signaling domain and a secondary engulfment signaling domain comprising a CD79b signaling domain (CD79b 185-213) (also referred to herein as “CER111B”). In certain embodiments, such a CER comprises an amino acid sequence of SEQ ID NO:126. In some embodiments, the CER mature polypeptide sequence comprises an amino acid sequence of SEQ ID NO: 126 without the signal peptide sequence (amino acids 1-22 of SEQ ID NO: 126). Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR8 signaling domain and a secondary engulfment signaling domain comprising a NFAM1 signaling domain (also referred to herein as “CER102”). In certain embodiments, such a CER comprises an amino acid sequence of SEQ ID NO:130. In some embodiments, the CER mature polypeptide sequence comprises an amino acid sequence of SEQ ID NO:130 without the signal peptide sequence (amino acids 1-22 of SEQ ID NO:130).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR8 signaling domain and a secondary engulfment signaling domain comprising a CD79b (185-229) signaling domain (also referred to herein as “CER103A”). In certain embodiments, such a CER comprises an amino acid sequence of SEQ ID NO:131. In some embodiments, the CER mature polypeptide comprises an amino acid sequence of SEQ ID NO:131 without the signal peptide sequence (amino acids 1-22 of SEQ ID NO:131)

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR8 signaling domain and a secondary engulfment signaling domain comprising a CD79b (185-213) signaling domain (also referred to herein as “CER103B”). In certain embodiments, such a CER comprises an amino acid sequence of SEQ ID NO:132. In some embodiments, the CER mature polypeptide comprises an amino acid sequence of SEQ ID NO: 132 without the signal peptide sequence (amino acids 1-22 of SEQ ID NO:132).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR8 signaling domain and a secondary engulfment signaling domain comprising a DAP12 signaling domain (also referred to herein as “CER104”). In certain embodiments, such a CER comprises an amino acid sequence of SEQ ID NO:133. In some embodiments, the CER mature polypeptide comprises an amino acid sequence of SEQ ID NO:133 without the signal peptide sequence (amino acids 1-22 of SEQ ID NO:133).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR8 signaling domain and a secondary engulfment signaling domain comprising a BAFF-R signaling domain (also referred to herein as “CER105”). In certain embodiments, such a CER comprises an amino acid sequence of SEQ ID NO:134. In some embodiments, the CER mature polypeptide comprises an amino acid sequence of SEQ ID NO:134 without the signal peptide sequence (amino acids 1-22 of SEQ ID NO:134).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a NFAM1 signaling domain and a secondary engulfment signaling domain comprising a TLR8 signaling domain (also referred to herein as “CER106”). In certain embodiments, such a CER comprises an amino acid sequence of SEQ ID NO:135. In some embodiments, the CER mature polypeptide comprises an amino acid sequence of SEQ ID NO:135 without the signal peptide sequence (amino acids 1-22 of SEQ ID NO:135).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a CD79b (185-213) signaling domain and a secondary engulfment signaling domain comprising a TLR8 signaling domain (also referred to herein as “CER107”). In certain embodiments, such a CER comprises an amino acid sequence of SEQ ID NO:136. In some embodiments, the CER mature polypeptide comprises an amino acid sequence of SEQ ID NO:136 without the signal peptide sequence (amino acids 1-22 of SEQ ID NO:136).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a DAP12 signaling domain and a secondary engulfment signaling domain comprising a TLR8 signaling domain (also referred to herein as “CER108”). In certain embodiments, such a CER comprises an amino acid sequence of SEQ ID NO:137. In some embodiments, the CER mature polypeptide comprises an amino acid sequence of SEQ ID NO:137 without the signal peptide sequence (amino acids 1-22 of SEQ ID NO:137).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a BAFF-R signaling domain and a secondary engulfment signaling domain comprising a TLR8 signaling domain (also referred to herein as “CER109”). In certain embodiments, such a CER comprises an amino acid sequence of SEQ ID NO:138. In some embodiments, the CER mature polypeptide comprises an amino acid sequence of SEQ ID NO:138 without the signal peptide sequence (amino acids 1-22 of SEQ ID NO:138).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TRAF6 signaling domain and a secondary engulfment signaling domain comprising a CD79b (185-229) signaling domain (also referred to herein as “CER111A”). In certain embodiments, such a CER comprises an amino acid sequence of SEQ ID NO:139. In some embodiments, the CER mature polypeptide comprises an amino acid sequence of SEQ ID NO:139 without the signal peptide sequence (amino acids 1-22 of SEQ ID NO:139).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TRAF6 signaling domain and a secondary engulfment signaling domain comprising a MERTK signaling domain (also referred to herein as “CER114”). In certain embodiments, such a CER comprises an amino acid sequence of SEQ ID NO:141. In some embodiments, the CER mature polypeptide comprises an amino acid sequence of SEQ ID NO:141 without the signal peptide sequence (amino acids 1-22 of SEQ ID NO:141).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a MERTK signaling domain and a secondary engulfment signaling domain comprising TRAF6 signaling domain (also referred to herein as “CER115”). In certain embodiments, such a CER comprises an amino acid sequence of SEQ ID NO:142. In some embodiments, the CER mature polypeptide comprises an amino acid sequence of SEQ ID NO: 142 without the signal peptide sequence (amino acids 1-22 of SEQ ID NO:142).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TRAF6 signaling domain and a secondary engulfment signaling domain comprising a TLR8 signaling domain (also referred to herein as “CER116”). In certain embodiments, such a CER comprises an amino acid sequence of SEQ ID NO:143. In some embodiments, the CER mature polypeptide comprises an amino acid sequence of SEQ ID NO:143 without the signal peptide sequence (amino acids 1-22 of SEQ ID NO:143).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR8 signaling domain and a secondary engulfment signaling domain comprising a TRAF6 signaling domain (also referred to herein as “CER117”). In certain embodiments, such a CER comprises an amino acid sequence of SEQ ID NO:144. In some embodiments, the CER mature polypeptide comprises an amino acid sequence of SEQ ID NO:144 without the signal peptide sequence (amino acids 1-22 of SEQ ID NO:144).

Another embodiment of a CER including an extracellular domain comprising a binding domain that binds to PtdSer comprises a Tim4 binding domain, a transmembrane domain comprising a Tim4 transmembrane domain, an engulfment signaling domain comprising a primary engulfment signaling domain comprising a TLR1 signaling domain and a secondary engulfment signaling domain comprising a NFAM1 signaling domain (also referred to herein as “CER118”). In certain embodiments, such a CER comprises an amino acid sequence of SEQ ID NO:145. In some embodiments, the CER mature polypeptide comprises an amino acid sequence of SEQ ID NO:145 without the signal peptide sequence (amino acids 1-22 of SEQ ID NO:145).

Yet another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR1 signaling domain and a secondary engulfment signaling domain comprising a CD79b (185-229) signaling domain (also referred to herein as “CER119A”). In certain embodiments, such a CER comprises an amino acid sequence of SEQ ID NO:173. In some embodiments, the CER mature polypeptide comprises an amino acid sequence of SEQ ID NO:173 without the signal peptide sequence (amino acids 1-22 of SEQ ID NO:173).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR1 signaling domain and a secondary engulfment signaling domain comprising a CD79b (185-213) signaling domain (also referred to herein as “CER119B”). In certain embodiments, such a CER comprises an amino acid sequence of SEQ ID NO:146. In some embodiments, the CER mature polypeptide comprises an amino acid sequence of SEQ ID NO:146 without the signal peptide sequence (amino acids 1-22 of SEQ ID NO:146).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR1 signaling domain and a secondary engulfment signaling domain comprising a DAP12 signaling domain (also referred to herein as “CER120”). In certain embodiments, such a CER comprises an amino acid sequence of SEQ ID NO:147. In some embodiments, the CER mature polypeptide comprises an amino acid sequence of SEQ ID NO:147 without the signal peptide sequence (amino acids 1-22 of SEQ ID NO:147).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR1 signaling domain and a secondary engulfment signaling domain comprising a TRAF6 signaling domain (also referred to herein as “CER121”). In certain embodiments, such a CER comprises an amino acid sequence of SEQ ID NO: 148. In some embodiments, the CER mature polypeptide comprises an amino acid sequence of SEQ ID NO:148 without the signal peptide sequence (amino acids 1-22 of SEQ ID NO:148).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR2 signaling domain and a secondary engulfment signaling domain comprising a DAP12 signaling domain (also referred to herein as “CER122”). In certain embodiments, such a CER comprises an amino acid sequence of SEQ ID NO:149. In some embodiments, the CER mature polypeptide comprises an amino acid sequence of SEQ ID NO:149 without the signal peptide sequence (amino acids 1-22 of SEQ ID NO:149).

Yet another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR2 signaling domain and a secondary engulfment signaling domain comprising a TRAF6 signaling domain (also referred to herein as “CER123”). In certain embodiments, such a CER comprises an amino acid sequence of SEQ ID NO:150. In some embodiments, the CER mature polypeptide comprises an amino acid sequence of SEQ ID NO:150 without the signal peptide sequence (amino acids 1-22 of SEQ ID NO:150).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR2 signaling domain and a secondary engulfment signaling domain comprising a NFAM1 signaling domain (also referred to herein as “CER124”). In certain embodiments, such a CER comprises an amino acid sequence of SEQ ID NO:151. In some embodiments, the CER mature polypeptide comprises an amino acid sequence of SEQ ID NO:151 without the signal peptide sequence (amino acids 1-22 of SEQ ID NO:151).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR2 signaling domain and a secondary engulfment signaling domain comprising a CD79b (185-229) signaling domain (also referred to herein as “CER125A”). In certain embodiments, such a CER comprises an amino acid sequence of SEQ ID NO:152. In some embodiments, the CER mature polypeptide comprises an amino acid sequence of SEQ ID NO:152 without the signal peptide sequence (amino acids 1-22 of SEQ ID NO:152).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR2 signaling domain and a secondary engulfment signaling domain comprising CD79b (185-213) signaling domain (also referred to herein as “CER125B”). In certain embodiments, such a CER comprises an amino acid sequence of SEQ ID NO:153. In some embodiments, the CER mature polypeptide comprises an amino acid sequence of SEQ ID NO:153 without the signal peptide sequence (amino acids 1-22 of SEQ ID NO:153).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR2 signaling domain and a secondary engulfment signaling domain comprising a TRAF2 signaling domain (also referred to herein as “CER126”). In certain embodiments, such a CER comprises an amino acid sequence of SEQ ID NO:174. In some embodiments, the CER mature polypeptide comprises an amino acid sequence of SEQ ID NO:174 without the signal peptide sequence (amino acids 1-22 of SEQ ID NO:174).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TRAF2 signaling domain and a secondary engulfment signaling domain comprising a TLR2 signaling domain (also referred to herein as “CER127”). In certain embodiments, such a CER comprises an amino acid sequence of SEQ ID NO:175. In some embodiments, the CER mature polypeptide comprises an amino acid sequence of SEQ ID NO:175 without the signal peptide sequence (amino acids 1-22 of SEQ ID NO:175).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TRAF2 signaling domain and a secondary engulfment signaling domain comprising a TLR8 signaling domain (also referred to herein as “CER128”). In certain embodiments, such a CER comprises an amino acid sequence of SEQ ID NO:176. In some embodiments, the CER mature polypeptide comprises an amino acid sequence of SEQ ID NO:176 without the signal peptide sequence (amino acids 1-22 of SEQ ID NO:176).

Another embodiment of a CER including an extracellular domain comprising a binding domain that binds to PtdSer comprises: a Tim4 binding domain, a transmembrane domain comprising a Tim4 transmembrane domain, an engulfment signaling domain comprising a primary engulfment signaling domain comprising a TLR8 signaling domain and a secondary engulfment signaling domain comprising a TRAF2 signaling domain (also referred to herein as “CER129”). In certain embodiments, such a CER comprises an amino acid sequence of SEQ ID NO:177. In some embodiments, the CER mature polypeptide comprises an amino acid sequence of SEQ ID NO: 177 without the signal peptide sequence (amino acids 1-22 of SEQ ID NO:177).

In another aspect, the present disclosure provides a CER comprising a single chain chimeric protein, the single chain chimeric protein comprising: an extracellular domain comprising a binding domain that binds to a pro-engulfment marker or target antigen; an engulfment signaling domain comprising a TLR signaling domain, a Traf2 signaling domain, or a Traf3 signaling domain; and a transmembrane domain positioned between and connecting the extracellular domain and the engulfment signaling domain. Such CERs may provide an inflammatory or immunogenic engulfment phenotype upon binding a target molecule (e.g., pro-engulfment marker or target antigen).

In certain embodiments of a CER including a TLR signaling domain, a Traf2 signaling domain, or a Traf3 signaling domain engulfment signaling domain, the extracellular domain further comprises an extracellular spacer domain positioned between the binding domain and the transmembrane domain.

In yet another aspect, the present disclosure provides a CER comprising a single chain chimeric protein, the single chain chimeric protein comprising: an extracellular domain comprising a binding domain that binds to a pro-engulfment marker or target antigen; an engulfment signaling domain comprising a primary engulfment signaling domain comprising a TLR signaling domain, a Traf6 signaling domain, a Traf2 signaling domain, or a Traf3 signaling domain and a secondary engulfment signaling domain; and a transmembrane domain positioned between and connecting the extracellular domain and the engulfment signaling domain. In some embodiments, the secondary engulfment signaling domain is proinflammatory engulfment signaling domain including an FcR signaling domain (including an FcγR1 signaling domain, an FcγR2A signaling domain, an FcγR2C signaling domain, FcγR2B2 signaling domain, an FcγR3A signaling domain, FcγR2C signaling domain, FcγR3A signaling domain, FcεR1 signaling domain, and FcαR1 signaling domain), a BAFF-R signaling domain, a DAP12 signaling domain, a NFAM1 signaling domain, and a CD79b signaling domain.

In certain embodiments of a CER including an engulfment signaling domain comprising a primary engulfment signaling domain and a secondary engulfment signaling domain, the extracellular domain further comprises an extracellular spacer domain positioned between the binding domain and the transmembrane domain.

In yet another aspect, the present disclosure provides a CER comprising a single chain chimeric protein, the single chain chimeric protein comprising: an extracellular domain comprising an scFv that binds to a pro-engulfment marker or target antigen; an engulfment signaling domain comprising a TLR signaling domain, a Traf6 signaling domain, a Traf2 signaling domain, or a Traf3 signaling domain; and a transmembrane domain positioned between and connecting the extracellular domain and the engulfment signaling domain, wherein the transmembrane domain and engulfment signaling domain are each derived from a different molecule.

In certain embodiments of a CER that includes an extracellular domain comprising a scFv that binds to a pro-engulfment marker or target antigen, the extracellular domain further comprises an extracellular spacer domain positioned between the binding domain and the transmembrane domain.

An embodiment of a CER that includes an extracellular domain comprising an scFv that binds to a pro-engulfment marker or target antigen comprises an extracellular domain comprising a CD19 specific scFv binding domain; an extracellular spacer domain comprising a TLR4 juxtamembrane domain; a transmembrane domain comprising a TLR4 transmembrane domain, and an engulfment signaling domain comprising a TLR4 signaling domain (also referred to herein as “CER43”). In certain embodiments, such a CER comprises an amino acid sequence of SEQ ID NO:122. In some embodiments, the CER comprises an amino acid sequence of SEQ ID NO:122 without the signal peptide sequence (amino acids 1-22 of SEQ ID NO:122).

Another embodiment of a CER that includes an extracellular domain comprising an scFv that binds to a pro-engulfment marker or target antigen comprises an extracellular domain comprising a CD19 specific scFv binding domain; an extracellular spacer domain comprising a modified IgG4 hinge region; a transmembrane domain comprising a TLR4 transmembrane domain, and an engulfment signaling domain comprising a TLR4 signaling domain (also referred to herein as “CER44”). In certain embodiments, such a CER comprises an amino acid sequence of SEQ ID NO:123. In some embodiments, the CER comprises an amino acid sequence of SEQ ID NO:123 without the signal peptide sequence (amino acids 1-22 of SEQ ID NO:123).

Another embodiment of a CER that includes an extracellular domain comprising an scFv that binds to a pro-engulfment marker or target antigen comprises an extracellular domain comprising a mesothelin specific scFv binding domain; an extracellular spacer domain comprising a TLR4 juxtamembrane domain; a transmembrane domain comprising a TLR4 transmembrane domain, and an engulfment signaling domain comprising a TLR4 signaling domain (also referred to herein as “CER51”).

Another embodiment of a CER that includes an extracellular domain comprising an scFv that binds to a pro-engulfment marker or target antigen comprises an extracellular domain comprising a mesothelin specific scFv binding domain; an extracellular spacer domain comprising a modified IgG4 hinge region; a transmembrane domain comprising a TLR4 transmembrane domain, and an engulfment signaling domain comprising a TLR4 signaling domain (also referred to herein as “CER52”).

Another embodiment of a CER that includes an extracellular domain comprising an scFv that binds to a pro-engulfment marker or target antigen comprises an extracellular domain comprising a LGR5 specific scFv binding domain; an extracellular spacer domain comprising a TLR4 juxtamembrane domain; a transmembrane domain comprising a TLR4 transmembrane domain, and an engulfment signaling domain comprising a TLR4 signaling domain (also referred to herein as “CER53”).

Another embodiment of a CER that includes an extracellular domain comprising an scFv that binds to a pro-engulfment marker or target antigen comprises an extracellular domain comprising a LGR5 specific scFv binding domain; an extracellular spacer domain comprising a modified IgG4 hinge region; a transmembrane domain comprising a TLR4 transmembrane domain, and an engulfment signaling domain comprising a TLR4 signaling domain (also referred to herein as “CER54”).

In certain embodiments, following binding of a CER expressed on the surface of a host cell to its cognate target molecule, lateral clustering of CERs occurs on the host cell surface, increasing the local CER concentration. Clustering is driven by the presence of multivalent ligands on the target cell or particle surface.

In certain embodiments, following binding of a CER expressed on the surface of a host cell to its cognate target molecule, dimerization or multimerization of the CERs occurs, bringing together intracellular engulfment signaling domains, which then become targets of intracellular kinases.

In certain embodiments, a CER of the present disclosure when expressed on the surface of a host cell is capable of tethering, internalizing, and processing (degrading) a target molecule or particle (e.g., phagocytosing a target). In other embodiments, a CER of the present disclosure is capable of tethering and internalizing a target molecule or particle (e.g., engulfing a target). In some embodiments, the target cell or particle within the phagosome may be discharged before or during phagosome maturation. Moreover, internalizing may comprise internalizing the whole cell or particle that is bound by the extracellular domain of the CER, or may comprise internalization of a piece or portion of the cell or particle that is bound by the extracellular domain of the CER.

In certain embodiments, a CER of the present disclosure tethers a target molecule or particle without internalization. A host cell expressing a CER may engulf or be tethered to multiple target cells or particles. Without wishing to be bound by theory, even in the absence of internalization and degradation of the target cell or particle, tethering of a target cell or particle by a host cell expressing a CER may result in degradation of the target cell or particle or promote an inflammatory environment, which is desirable in certain therapeutic contexts (e.g., cancer).

Embodiments of CERs according to the present description are illustrated in FIGS. 6, 15, 18, Sequence Listing, Table 1, and the examples.

TABLE 1
Exemplary Chimeric Engulfment Receptors

| | | | First | Second | |
|---|---|---|---|---|---|
| | | | Engulfment | Engulfment | Exemplary |
| CER | Binding | Transmembrane | Signaling | Signaling | Amino Acid |
| Name | Domain | Domain | Domain | Domain | Sequences |
| CER5 | Tim4 | Tim4 | TLR4 | | SEQ ID NO: 81 |
| CER6 | Tim4 | TLR4 | TLR4 | | SEQ ID NO: 82 |
| CER7 | Tim4 + TLR | TLR4 | TLR4 | | SEQ ID NO: 83 |
| | juxtamembrane | | | | |
| | domain | | | | |
| CER17 | Tim4 | Tim4 | TLR_3 | | SEQ ID NO: 84 |
| CER18 | Tim4 | TLR_3 | TLR_3 | | SEQ ID NO: 85 |
| CER19 | Tim4 | Tim4 | TLR_5 | | SEQ ID NO: 86 |
| CER20 | Tim4 | TLR_5 | TLR_5 | | SEQ ID NO: 87 |
| CER21 | Tim4 | Tim4 | TLR_8 | | SEQ ID NO: 88 |
| CER22 | Tim4 | TLR_8 | TLR_8 | | SEQ ID NO: 89 |
| CER23 | Tim4 | Tim4 | TLR_9 | | SEQ ID NO: 90 |
| CER24 | Tim4 | TLR_9 | TLR_9 | | SEQ ID NO: 91 |
| CER26 | Tim4 | Tim4 | TLR_1 | | SEQ ID NO: 92 |
| CER27 | Tim4 | Tim4 | TLR_2 | | SEQ ID NO: 93 |
| CER28 | Tim4 | Tim4 | TLR_7 | | SEQ ID NO: 94 |
| CER29 | Tim4 | Tim4 | TRAF6 | | SEQ ID NO: 124 |
| CER30 | Tim4 | Tim4 | TRAF2 | | SEQ ID NO: 96 |
| CER31 | Tim4 | Tim4 | TRAF3 | | SEQ ID NO: 97 |
| CER102 | Tim4 | Tim4 | TLR8 | NFAM1 | SEQ ID NO: 130 |
| CER103A | Tim4 | Tim4 | TLR8 | CD79b | SEQ ID NO: 131 |
| | | | | (185-229) | |
| CER103B | Tim4 | Tim4 | TLR8 | CD79b | SEQ ID NO: 132 |
| | | | | (185-213) | |
| CER104 | Tim4 | Tim4 | TLR8 | DAP12 | SEQ ID NO: 133 |
| CER105 | Tim4 | Tim4 | TLR8 | Baff-R | SEQ ID NO: 134 |
| CER106 | Tim4 | Tim4 | NFAM1 | TLR8 | SEQ ID NO: 135 |
| CER107 | Tim4 | Tim4 | CD79b | TLR8 | SEQ ID NO: 136 |
| | | | (185-213) | | |
| CER108 | Tim4 | Tim4 | DAP12 | TLR8 | SEQ ID NO: 137 |
| CER109 | Tim4 | Tim4 | Baff-R | TLR8 | SEQ ID NO: 138 |
| CER110 | Tim4 | Tim4 | TRAF6 | DAP12 | SEQ ID NO: 125 |
| CER111A | Tim4 | Tim4 | TRAF6 | CD79b | SEQ ID NO: 139 |
| | | | | (185-229) | |
| CER111B | Tim4 | Tim4 | TRAF6 | CD79b | SEQ ID NO: 126 |
| | | | | (185-213) | |
| CER112 | Tim4 | Tim4 | TRAF6 | NFAM1 | SEQ ID NO: 128 |
| CER113 | Tim4 | Tim4 | TRAF6 | Baff-R | SEQ ID NO: 127 |
| CER114 | Tim4 | Tim4 | TRAF6 | MERTK | SEQ ID NO: 141 |
| CER115 | Tim4 | Tim4 | MERTK | TRAF6 | SEQ ID NO: 142 |
| CER116 | Tim4 | Tim4 | TRAF6 | TLR8 | SEQ ID NO: 143 |
| CER117 | Tim4 | Tim4 | TLR8 | TRAF6 | SEQ ID NO: 144 |
| CER118 | Tim4 | Tim4 | TLR1 | NFAM1 | SEQ ID NO: 145 |
| CER119B | Tim4 | Tim4 | TLR1 | CD79b | SEQ ID NO: 146 |
| | | | | (185-213) | |
| CER119A | Tim4 | Tim4 | TLR1 | CD79b | SEQ ID NO: 173 |
| | | | | (185-229) | |
| CER120 | Tim4 | Tim4 | TLR1 | DAP12 | SEQ ID NO: 147 |
| CER121 | Tim4 | Tim4 | TLR1 | TRAF6 | SEQ ID NO: 148 |
| CER122 | Tim4 | Tim4 | TLR2 | DAP12 | SEQ ID NO: 149 |
| CER123 | Tim4 | Tim4 | TLR2 | TRAF6 | SEQ ID NO: 150 |
| CER124 | Tim4 | Tim4 | TLR2 | NFAM1 | SEQ ID NO: 151 |
| CER125A | Tim4 | Tim4 | TLR2 | CD79b | SEQ ID NO: 152 |
| | | | | (185-229) | |
| CER125B | Tim4 | Tim4 | TLR2 | CD79b | SEQ ID NO: 153 |
| | | | | (185-213_{—} | |
| CER126 | Tim4 | Tim4 | TLR2 | TRAF2 | SEQ ID NO: 174 |
| CER127 | Tim4 | Tim4 | TRAF2 | TLR2 | SEQ ID NO: 175 |
| CER128 | Tim4 | Tim4 | TRAF2 | TLR8 | SEQ ID NO: 176 |
| CER129 | Tim4 | Tim4 | TLR8 | TRAF2 | SEQ ID NO: 177 |

### Nucleic Acids, Vectors, and Host Cells

In certain aspects, the present disclosure provides nucleic acid molecules that encode any one or more of the CERs described herein. The nucleic acid sequences encoding a desired CER can be obtained or produced using recombinant methods known in the art using standard techniques, such as by screening libraries from cells expressing the desired sequence or a portion thereof, by deriving the sequence from a vector known to include the same, or by isolating the sequence or a portion thereof directly from cells or tissues containing the same. Alternatively, the sequence of interest can be produced synthetically, rather than being cloned.

Polynucleotides encoding the CER compositions provided herein may be derived from any animal, such as humans, primates, cows, horses, sheep, dogs, cats, mice, rats, rabbits, guinea pigs, or pigs. In certain embodiments, a polynucleotide encoding the CER is from the same animal species as the host cell into which the polynucleotide is inserted.

Polynucleotides encoding the CER compositions provided herein may also include a sequence encoding a signal peptide (also referred to as leader peptide or signal sequence) at the amino terminal end of the CER for targeting of the precursor protein to the secretory pathway. The signal peptide is optionally cleaved from the N-terminus of the extracellular domain during cellular processing and localization of the CER to the cell membrane. A polypeptide from which a signal peptide sequence has been cleaved or removed may also be called a mature polypeptide. Examples of signal peptides that may be used in the CERs of the present disclosure include signal peptides derived from endogenous secreted proteins, including, e.g., GM-CSF (amino acid sequence of SEQ ID NO:99), Tim4 (amino acid sequence of SEQ ID NO: 100 or amino acids 1-24 of SEQ ID NO:3). In certain embodiments, polynucleotide or polypeptide sequences of CERs of the present disclosure comprise sequences for mature polypeptides. It is understood by persons of skill in the art that for sequences disclosed herein that include a signal peptide sequence, the signal peptide sequence may be replaced with another signal peptide that is capable of trafficking the encoded protein to the extracellular membrane.

In certain embodiments, a nucleic acid molecule encoding a CER of the present disclosure is codon optimized for efficient expression in a target host cell.

Nucleic acid molecules encoding a desired CER can be inserted into an appropriate vector (e.g., viral vector, non-viral plasmid vector, and non-viral vectors, such as lipid-based DNA vectors, modified mRNA (modRNA), self-amplifying mRNA, CELiD, and transposon-mediated gene transfer (PiggyBac, Sleeping Beauty)) for introduction in a host cell of interest (e.g., a T cell, a natural killer cell, a B cell, a lymphocyte precursor cell, an antigen presenting cell, a Langerhans cell, or a myeloid cell). Nucleic acid molecules encoding a CER of the present disclosure can be cloned into any suitable vector, such as an expression vector, a replication vector, a probe generation vector, or a sequencing vector. In certain embodiments, a nucleic acid sequence encoding the extracellular domain, a nucleic acid sequence encoding the transmembrane domain, and a nucleic acid sequence encoding the engulfment signaling domain are joined together in a single polynucleotide and then inserted into a vector. In other embodiments, a nucleic acid sequence encoding the extracellular domain, a nucleic acid sequence encoding the transmembrane domain, and a nucleic acid sequence encoding the engulfment signaling domain may be inserted separately in a vector such that the resulting amino acid sequence produces a functional CER. A vector that encodes a CER is referred to herein as a “CER vector.”

In certain embodiments, a vector comprises a nucleic acid molecule encoding one CER. In other embodiments, a vector comprises one or more nucleic acid molecules encoding two or more CERs. In one embodiment, two or more nucleic acid molecules each encoding a CER may be cloned sequentially into a vector at different multiple cloning sites, with each CER expressed under the regulation of different promoters. In another embodiment, a single nucleic acid molecule encoding multiple CERs is cloned into a cloning site and expressed from a single promoter, with each CER separated from each other by an IRES or viral 2A peptide sequence to allow for co-expression of multiple genes from a single open reading frame (e.g., a multicistronic vector). In certain embodiments, a viral 2A peptide is T2A (SEQ ID NOS: 102, 154, 155, or 156), P2A (SEQ ID NO:101 or 157), E2A (SEQ ID NO:103), or F2A (SEQ ID NO:104).

In some embodiments, vectors that allow long-term integration of a transgene and propagation to daughter cells are utilized. Examples include viral vectors such as, adenovirus, adeno-associated virus, vaccinia virus, herpes viruses, Cytomegalovirus, pox virus, or retroviral vectors, such as lentiviral vectors. Vectors derived from lentivirus can be used to achieve long-term gene transfer and have added advantages over vectors including the ability to transduce non-proliferating cells, such as hepatocytes, and low immunogenicity.

In certain embodiments, a CER vector can be constructed to optimize spatial and temporal control. For example, CER vector can include promoter elements to optimize spatial and temporal control. In some embodiments, a CER vector includes tissue specific promoters or enhancers that enable specific induction of a CER to an organ or a pathologic microenvironment, such as tumor or infected tissue. An “enhancer” is an additional promoter element that can function either cooperatively or independently to activate transcription. In other embodiments, a CER vector includes a constitutive promoter. In still other embodiments, a CER vector includes an inducible promoter.

In further embodiments, a CER vector can include a homing receptor, such as CCR4 or CXCR4, to improve homing and antitumor activity in vivo.

Where temporal control is desired, a CER vector may include an element that allows for inducible depletion of transduced cells. For example, such a vector may include an inducible suicide gene. A suicide gene may be an apoptotic gene or a gene that confers sensitivity to an agent (e.g., drug), such as chemically inducible caspase 9 (iCASP9), chemically inducible Fas, or HSV-TK (confers sensitivity to ganciclovir). In further embodiments, a CER vector can be designed to express a known cell surface antigen that, upon infusion of an associated antibody, enables depletion of transduced cells. Examples of cell surface antigens and their associated antibodies that may be used for depletion of transduced cells include CD20 and Rituximab, RQR8 (combined CD34 and CD20 epitopes, allowing CD34 selection and anti-CD20 deletion) and Rituximab, and EGFR and Cetuximab.

Inducible vector systems, such as the tetracycline (Tet)-On vector system which activates transgene expression with doxycycline (Heinz et al., Hum. Gene Ther. 2011, 22:166-76) may also be used for inducible CER expression. Inducible CER expression may be also accomplished via retention using a selective hook (RUSH) system based on streptavidin anchored to the membrane of the endoplasmic reticulum through a hook and a streptavidin binding protein introduced into the CER structure where addition of biotin to the system leads to the release of the CER from the endoplasmic reticulum (Agaugue et al., 2015, Mol. Ther. 23(Suppl. 1):S88).

As used herein, the term “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications introducing expressible nucleic acid molecules encoding proteins, chimeric proteins or enzymes, or other nucleic acid molecule additions, deletions, substitutions or other functional disruption of a cell's genetic material. Additional modifications include, for example, non-coding regulatory regions in which the modifications alter expression of a gene or operon. In certain embodiments, a cell, such as a T cell, obtained from a subject may be genetically modified into a non-natural or recombinant cell (e.g., a non-natural or recombinant T cell) by introducing a nucleic acid that encodes a CER as described herein and whereby the cell expresses a cell surface located CER.

A vector that encodes a core virus is referred to herein as a “viral vector.” There are a large number of available viral vectors suitable for use with the compositions of the instant disclosure, including those identified for human gene therapy applications (see Pfeifer and Verma, Ann. Rev. Genomics Hum. Genet. 2:177, 2001). Suitable viral vectors include vectors based on RNA viruses, such as retrovirus-derived vectors, e.g., Moloney murine leukemia virus (MLV)-derived vectors, and include more complex retrovirus-derived vectors, e.g., lentivirus-derived vectors. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing chimeric receptor transgenes are known in the art and have been previous described, for example, in U.S. Pat. No. 8,119,772; Walchli et al., PLOS One 6:327930, 2011; Zhao et al., J. Immunol. 174:4415, 2005; Engels et al., Hum. Gene Ther. 14:1155, 2003; Frecha et al., Mol. Ther. 18:1748, 2010; Verhoeyen et al., Methods Mol. Biol. 506:97, 2009. Retroviral and lentiviral vector constructs and expression systems are also commercially available.

In certain embodiments, a viral vector is used to introduce a non-endogenous nucleic acid sequence encoding a CER specific for a target. A viral vector may be a retroviral vector or a lentiviral vector. A viral vector may also include nucleic acid sequences encoding a marker for transduction. Transduction markers for viral vectors are known in the art and include selection markers, which may confer drug resistance, or detectable markers, such as fluorescent markers or cell surface proteins that can be detected by methods such as flow cytometry. In particular embodiments, a viral vector further comprises a gene marker for transduction comprising fluorescent protein (e.g., green, yellow), an extracellular domain of human CD2, or a truncated human EGFR (encoding an amino acid sequence of SEQ ID NO: 105) (huEGFRt; see Wang et al., Blood 118:1255, 2011). When a viral vector genome comprises a plurality of nucleic acid sequences to be expressed in a host cell as separate transcripts, the viral vector may also comprise additional sequences between the two (or more) transcripts allowing bicistronic or multicistronic expression. Examples of such sequences used in viral vectors include internal ribosome entry sites (IRES), furin cleavage sites, viral 2A peptides (e.g., T2A, P2A, E2A, F2A), or any combination thereof.

Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5: 1517, 1998).

Other viral vectors recently developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors). In some embodiments, a viral or plasmid vector further comprises a gene marker for transduction (e.g. green fluorescent protein, huEGFRt (encoding an amino acid sequence of SEQ ID NO:105).

In certain embodiments, gene editing methods are used to modify the host cell genome to comprise a polynucleotide encoding a CER of the present disclosure. Gene editing, or genome editing, is a method of genetic engineering wherein DNA is inserted, replaced, or removed from a host cell's genome using genetically engineered endonucleases. The nucleases create specific double-stranded breaks at targeted loci in the genome. The host cell's endogenous DNA repair pathways then repair the induced break(s), e.g., by non-homologous ending joining (NHEJ) and homologous recombination. Exemplary endonucleases useful in gene editing include a zinc finger nuclease (ZFN), a transcription activator-like effector (TALE) nuclease, a clustered regularly interspaced short palindromic repeats (CRISPR)/Cas nuclease system (e.g., CRISPR-Cas9), a meganuclease, or combinations thereof. Methods of disrupting or knocking out genes or gene expression in immune cells including B cells and T cells, using gene editing endonucleases are known in the art and described, for example, in PCT Publication Nos. WO 2015/066262; WO 2013/074916; WO 2014/059173; Cheong et al., Nat. Comm. 2016 7:10934; Chu et al., Proc. Natl. Acad. Sci. USA 2016 113:12514-12519; methods from each of which are incorporated herein by reference in their entirety.

In certain embodiments, B cells, lymphoid precursor cells, including common lymphocyte precursor cells, antigen presenting cells, including dendritic cells, Langerhans cells, a myeloid precursor cell, or mature myeloid cells are modified to comprise a non-endogenous nucleic acid molecule that encodes a CER of this disclosure.

In some embodiments, B cells are genetically modified to express one or more CERs. B cells possess certain properties that may be advantageous as host cells, including: trafficking to sites of inflammation (e.g., lymph nodes, tumors), capable of internalizing and presenting antigen, capable of costimulating T cells, highly proliferative, and self-renewing (persist for life). In certain embodiments, CER modified B cells are capable of digesting an engulfed target cell or engulfed target particle into smaller peptides and presenting them to T cells via an MHC molecule. Antigen presentation by CER modified B cells may contribute to antigen spreading of the immune response to non-targeted antigens. B cells include progenitor or precursor cells committed to the B cell lineage (e.g., pre-pro-B cells, pro-B cells, and pre-B cells); immature and inactivated B cells or mature and functional or activated B cells. In certain embodiments, B cells may be naïve B cells, plasma cells, regulatory B cells, marginal zone B cells, follicular B cells, lymphoplasmacytoid cell, plasmablast cell, memory B cells, or any combination thereof. Memory B cells may be distinguished from naïve B cells by expression of CD27, which is absent on naïve B cells. In certain embodiments, the B cells can be primary cells or cell lines derived from human, mouse, rat, or other mammals. B cell lines are well known in the art. If obtained from a mammal, a B cell can be obtained from numerous sources, including blood, bone marrow, spleen, lymph node, or other tissues or fluids. In certain embodiments, a B cell is isolated from a tumor site (tumor infiltrating B cell). A B cell composition may be enriched or purified.

In certain embodiments, expression of an endogenous gene of the host B cell is inhibited, knocked down, or knocked out. Examples of endogenous genes that may be inhibited, knocked down, or knocked out in a B cell include a B cell receptor (BCR) gene (e.g., CD79b, IGH, IGK, IGA, or any combination thereof), an immune checkpoint molecule (e.g., PD-L1, PD-L2, CD80, CD86, B7-H3, B7-H4, HVEM, adenosine, GAL9, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, PVRIG/CD112R, or any combination thereof), or any combination thereof. Expression of a BCR gene, an immune checkpoint molecule gene, or both may be inhibited, knocked down, or knocked out at the gene level, transcriptional level, or translational level, or a combination thereof. Methods of inhibiting, knocking down, or knocking out a BCR gene, immune checkpoint molecule gene, or both may be accomplished, for example, by RNA interference agents (e.g., siRNA, shRNA, miRNA, etc.) or engineered endonucleases (e.g., CRISPR/Cas nuclease system, a zinc finger nuclease (ZFN), a Transcription Activator Like Effector nuclease (TALEN), a meganuclease, or any combination thereof). In some embodiments, an endogenous gene (e.g., a BCR gene or an immune checkpoint molecule gene) is knocked out by insertion of a polynucleotide encoding a CER of the present disclosure into the locus of the endogenous B cell gene, such as via an engineered endonuclease.

In some embodiments, cells capable of expressing a CER of this disclosure on the cell surface are T cells, including CD4⁺, CD8⁺, naïve (CD45 RA+, CCR7+, CD62L+, CD27+, CD45RO−), central memory (CD45RO⁺, CD62L⁺, CD8⁺), effector memory (CD45RA+, CD45RO−, CCR7−, CD62L−, CD27−), virus-specific, mucosal-associated invariant, γδ (gd), tissue resident T cells, and natural killer T cells. In certain embodiments, the T cells can be primary cells or cell lines derived from human, mouse, rat, or other mammals. If obtained from a mammal, a T cell can be obtained from numerous sources, including blood, bone marrow, lymph node, thymus, or other tissues or fluids. In certain embodiments, a T cell is isolated from a tumor site (tumor infiltrating T cell). A T cell composition may be enriched or purified. T cell lines are well known in the art, some of which are described in Sandberg et al., Leukemia 21:230, 2000. In certain embodiments, T cells that lack endogenous expression of TCRα and β chains are used. Such T cells may naturally lack endogenous expression of TCRα and β chains or may have been modified to block expression (e.g., T cells from a transgenic mouse that does not express TCR α and β chains or cells that have been manipulated to inhibit expression of TCR α and β chains) or to knockout TCRα chain, TCRβ chain, or both genes. In certain embodiments, cells capable of expressing a chimeric protein of this disclosure on the cell surface are not T cells or cells of a T cell lineage, but cells that are progenitor cells, stem cells or cells that have been modified to express cell surface anti-CD3.

In certain embodiments, CER modified T cells are capable of digesting an engulfed target cell or engulfed target particle into smaller peptides and presenting them to T cells via an MHC molecule. Antigen presentation by CER modified T cells may contribute to antigen spreading of the immune response to non-targeted antigens.

In certain embodiments, a host T cell transfected to express a CER of this disclosure is a functional T cell, such as a virus-specific T cell, a tumor antigen specific cytotoxic T cell, a naïve T cell, a memory stem T cell, a central or effector memory T cell, or a CD4+ CD25+ regulatory T cell.

In certain embodiments, expression of an endogenous gene of the host T cell is inhibited, knocked down, or knocked out. Examples of endogenous genes that may be inhibited, knocked down, or knocked out in a T cell include a TCR gene (TRA, TRB, or both), HLA gene (HLA class I gene, HLA class II gene, or both), an immune checkpoint molecule (PD-L1, PD-L2, CD80, CD86, B7-H3, B7-H4, HVEM, adenosine, GAL9, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, PVRIG/CD112R, or any combination thereof), or any combination thereof. Expression of a TCR gene, an HLA gene, an immune checkpoint molecule gene, or any combination thereof may be inhibited, knocked down, or knocked out at the gene level, transcriptional level, or translational level, or any combination thereof. Methods of inhibited, knocked down, or knocked out a TCR gene, an HLA gene, immune checkpoint molecule gene, or any combination thereof may be accomplished, for example, by RNA interference agents (e.g., siRNA, shRNA, miRNA, etc.) or engineered endonucleases (e.g., CRISPR/Cas nuclease system, a zinc finger nuclease (ZFN), a Transcription Activator Like Effector nuclease (TALEN), a meganuclease, or any combination thereof). In some embodiments, an endogenous gene (e.g., a TCR gene, an HLA gene, or an immune checkpoint molecule gene) is knocked out by insertion of a polynucleotide encoding a CER of the present disclosure into the locus of the endogenous T cell gene, such as via an engineered endonuclease.

In certain embodiments, a host cell comprising a CER that comprises an extracellular domain comprising a binding domain that binds to phosphatidylserine (PtdSer) according to any of the embodiments described herein is a T cell, a natural killer cell, a B cell, a lymphoid precursor cell, including common lymphocyte precursor cells, an antigen presenting cell, including dendritic cells, a Langerhans cell, a myeloid precursor cell, or a mature myeloid cell.

In other embodiments, a host cell comprising a CER that comprises an extracellular domain comprising a binding domain (e.g., a scFv) that binds to a target antigen according to any of the embodiments described herein is a B cell.

In yet other embodiments a host cell comprising a CER that comprises an extracellular domain comprising a binding domain that binds to a pro-engulfment marker or target antigen according to any of the embodiments described herein is a cell that does not naturally exhibit an engulfment phenotype. In a particular embodiment, the host cell is a T cell, a natural killer cell, a B cell, or a lymphoid precursor cell, including common lymphocyte precursor cells. In a particular embodiment, the host cell is a cell that does not naturally exhibit an engulfment phenotype towards a mammalian cell.

In certain embodiments, a host cell may be genetically modified to express one type of CER. In other embodiments, a host cell may express at least two or more different CERs.

In certain embodiments, a population of host cells that are modified to express one or more CERs may be a population of B cells, a population of T cells, a population of natural killer cells, a population of lymphoid precursor cells, including common lymphocyte precursor cells, a population of antigen presenting cells, including dendritic cells, Langerhans cells, a population of myeloid precursor cells, a population of mature myeloid cells, or any combination thereof. In a particular embodiment, the population of host cells that are modified to express one or more CERs is a population of B cells, a population of T cells, or both.

In certain embodiments, each host cell within a population of host cells expresses the same CER or set of CERs. In other embodiments, a population of host cells comprises a mixture of two or more subpopulation of host cells, wherein each subpopulation expresses a different CER or set of CERs.

In certain embodiments, when preparing host cells, e.g., B cells or T cells, that express a CER as described herein, one or more growth factor cytokines that promote proliferation of the host cells, e.g., B cells or T cells, may be added to the cell culture. The cytokines may be human or non-human. Exemplary growth factor cytokines that may be used to promote T cell proliferation include IL-2, IL-15, or the like. Exemplary growth factor cytokines that may be used to promote B cell proliferation include CD40L, IL-2, IL-4, IL-15, IL-21, BAFF, or the like.

In further embodiments, selective gene transfer is used to localize the CER vector to a specific region or organ. In some embodiments, selective gene transfer is used to localize the CER vector to the liver or the lungs of a subject.

Prior to genetic modification of the host cells with a CER vector, a source of host cells (e.g., T cells, B cells, natural killer cells, etc.) is obtained from a subject (e.g., whole blood, peripheral blood mononuclear cells, bone marrow, lymph node tissue, cord blood, thymus tissue, tissue from a site of infection, ascites, pleural effusion, spleen tissue), from which host cells are isolated using methods known in the art. Specific host cell subsets can be collected in accordance with known techniques and enriched or depleted by known techniques, such as affinity binding to antibodies, flow cytometry and/or immunomagnetic selection. After enrichment and/or depletion steps and introduction of a CER, in vitro expansion of the desired modified host cells can be carried out in accordance with known techniques, or variations thereof that will be apparent those skilled in the art.

In certain embodiments, a host cell, including a T cell, a natural killer cell, a B cell, a lymphoid precursor cell, an antigen presenting cell, dendritic cell, a Langerhans cell, a myeloid precursor cell, and a mature myeloid cell, comprising a CER according to any of the embodiments described herein has a phagocytic index of about 20 to about 1,500 for a target cell. A “phagocytic index” is a measure of phagocytic activity of the transduced host cell as determined by counting the number of target cells ingested per CER modified host cell during a set period of incubation of a suspension of target cells and CER modified host cells in media. Phagocytic index may be calculated by multiplying [total number of engulfed target cells/total number of counted CER modified cells (e.g., phagocytic frequency)]×[average area of target cell staining per CER+ Ba/F3 cell×100 (e.g., hybrid capture)] or [total number of engulfed particles/total number of counted CER modified host cells]×[number of CER modified host cells containing engulfed particles/total number of counted CER cells]×100. In certain embodiments, a CER modified cell has a phagocytic index of about 30 to about 1,500; about 40 to about 1,500; about 50 to about 1,500; about 75 to about 1,500; about 100 to about 1,500; about 200 to about 1,500; about 300 to about 1,500; about 400 to about 1,500; about 500 to about 1,500; about 20 to about 1,400; about 30 to about 1,400; about 40 to about 1,400; about 50 to about 1,400; about 100 to about 1,400; about 200 to about 1,400; about 300 to about 1,400; about 400 to about 1,400; about 500 to about 1,400; about 20 to about 1,300; about 30 to about 1,300; about 40 to about 1,300; about 50 to about 1,300; about 100 to about 1,300; about 200 to about 1,300; about 300 to about 1,300; about 400 to about 1,300; about 500 to about 1,300; about 20 to about 1,200; about 30 to about 1,200; about 40 to about 1,200; about 50 to about 1,200; about 100 to about 1,200; about 200 to about 1,200; about 300 to about 1,200; about 400 to about 1,200; about 500 to about 1,200; about 20 to about 1,100; about 30 to about 1,100; about 40 to about 1,100; about 50 to about 1,100; about 100 to about 1,100; about 200 to about 1,100; about 300 to about 1,100; about 400 to about 1,100; or about 500 to about 1,100; about 20 to about 1,000; about 30 to about 1,000; about 40 to about 1,000; about 50 to about 1,000; about 100 to about 1,000; about 200 to about 1,000; about 300 to about 1,000; about 400 to about 1,000; or about 500 to about 1,000; about 20 to about 750; about 30 to about 750; about 40 to about 750; about 50 to about 750; about 100 to about 750; about 200 to about 750; about 300 to about 750; about 400 to about 750; or about 500 to about 750; about 20 to about 500; about 30 to about 500; about 40 to about 500; about 50 to about 500; about 100 to about 500; about 200 to about 500; or about 300 to about 500. In further embodiments, the incubation time is from about 2 hours to about 4 hours, about 2 hours, about 3 hours, or about 4 hours. In yet further embodiments, a CER modified cell exhibits phagocytic index that is statistically significantly higher than a cell transduced with truncated EGFR control. Phagocytic index may be calculated using methods known in the art and as further described in the Examples, including quantification by flow cytometry or fluorescence microscopy.

In certain embodiments, a host cell that has been modified to express a CER according to one of the embodiments described herein exhibits: cytolytic activity towards a target cell, i.e., capable of lysing a target cell expressing a target antigen on its surface; exhibits enhanced activation (e.g., enhanced cytokine production, such as IFNγ); exhibits enhanced cell proliferation; exhibits enhanced cell expansion; exhibits enhanced persistence; exhibits enhanced memory formation; exhibits antigen presentation activity; exhibits induction of antigen-specific phagocytic signaling or enhanced antigen-specific phagocytic signaling; exhibits degradation of an engulfed target cell; or any combination thereof as compared to a host cell that does not express the CER. In certain embodiments, a CER modified host cell is capable of inducing antigenic spread via its antigen presenting activities.

Host cells may be from an animal, such as a primate, cow, horse, sheep, dog, cat, mouse, rat, rabbit, guinea pig, or pig. In a preferred embodiment, the animal is a human. Host cells may be obtained from a healthy subject or a subject having a disease associated with expression of an antigen.

### Uses of CERs and Cells Modified to Express CERs

The present disclosure provides methods for altering the engulfment phenotype of a host cell. In one aspect, the present disclosure provides methods for producing a population of cells exhibiting an engulfment phenotype comprising introducing into a population of host cells that do not naturally exhibit an engulfment phenotype a nucleic acid molecule encoding at least one CER or a vector comprising at least one CER according to any of the embodiments described herein; and expressing the at least one CER in the population of host cells. In certain embodiments, the engulfment phenotype is phagocytosis. In certain embodiments, the population of host cells expressing the at least one CER is capable of antigen-specific phagocytic signaling activity. Induction of an antigen-specific phagocytic signaling cascade may comprise activation of CDC42, Rac1, or both. In certain embodiments, the population of host cells expressing the at least one CER is capable of degrading engulfed target cells.

In another aspect, the present disclosure provides methods for altering the engulfment phenotype of a population of cells comprising introducing into a population of host cells a nucleic acid molecule encoding at least one CER or a vector comprising at least one CER according to any of the embodiments described herein; and expressing the at least one CER in the population of host cells, wherein the at least one CER confers an engulfment phenotype specific to a pro-engulfment marker or antigenic marker (target antigen) that is not naturally targeted by the host cells. In certain embodiments, the engulfment phenotype is phagocytosis.

In yet another aspect, the present disclosure provides methods for enhancing the engulfment phenotype of a population of cells comprising introducing into a population of host cells a nucleic acid molecule encoding at least one CER or a vector comprising at least one CER according to any of the embodiments described herein; and expressing the at least one CER in the population of host cells, wherein the at least one CER is specific to a pro-engulfment marker or antigenic marker (target antigen) that is naturally targeted by the host cells and expression of the at least one CER by the host cells enhances the engulfment by the host cells of cells, microbes, or particles exhibiting the targeted pro-engulfment or antigenic marker.

In further embodiments of the methods of producing a population of host cells exhibiting an engulfment phenotype, altering the engulfment phenotype in a population of cells, or enhancing the engulfment phenotype in a population of cells, expression of at least one CER by the population of host cells enhances proliferative capacity of the population of cells, enhances activation of the population of cells (e.g., enhanced cytokine production, such as IFNγ), enhances expansion of the population of host cells, enhances persistence of the population of host cells, enhances memory formation of the population of cells, confers antigen presenting activity to the population of host cells, confers or enhances cytolytic activity of the population of host cells, or any combination thereof. In certain embodiments, a population of CER modified host cell is capable of inducing antigenic spread via the antigen presenting activities.

CERs, nucleic acid molecules encoding CERs, vectors comprising CERs, and host cells that express CERs according to any of the embodiments described herein may also be used in a method of treating a subject suffering from a disease, disorder or undesired condition. Embodiments of these methods include administering to a subject a therapeutically effective amount of a pharmaceutical composition including one or more CERs, nucleic acid molecules encoding one or more CERs, vectors comprising one or more CERs, or a population of host cells genetically modified to express one or more CERs according to the present description.

Diseases that may be treated with cells expressing a CER as described in the present disclosure include cancer, infectious diseases (viral, bacterial, fungal, protozoan infections), inflammatory, or immune diseases (e.g., autoimmune diseases, inflammatory bowel diseases, multiple sclerosis), degenerative disease (e.g., joint and cartilage), and neurodegenerative diseases (e.g., Alzheimer's disease). Adoptive immune and gene therapies are promising treatments for various types of cancer (Morgan et al., Science 314:126, 2006; Schmitt et al., Hum. Gene Ther. 20:1240, 2009; June, J. Clin. Invest. 117:1466, 2007) and infectious disease (Kitchen et al., PLOS One 4:38208, 2009; Rossi et al., Nat. Biotechnol. 25:1444, 2007; Zhang et al., PLOS Pathog. 6:e1001018, 2010; Luo et al., J. Mol. Med. 89:903, 2011).

Subjects that can be treated by the compositions and methods of the present disclosure include animals, such as humans, primates, cows, horses, sheep, dogs, cats, mice, rats, rabbits, guinea pigs, or pigs. The subject may be male or female, and can be any suitable age, including infant, juvenile, adolescent, adult, and geriatric subjects.

A wide variety of cancers, including solid tumors and leukemias are amenable to the compositions and methods disclosed herein. Exemplary types of cancer that may be treated include adenocarcinoma of the breast, prostate, and colon; all forms of bronchogenic carcinoma of the lung; myeloid leukemia; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, Merkel cell, mucinous, non-small cell lung, oat cell, papillary, scirrhous, bronchiolar, bronchogenic, squamous cell, and transitional cell). Additional types of cancers that may be treated include histiocytic disorders; malignant histiocytosis; leukemia; Hodgkin's disease; immunoproliferative small; non-Hodgkin's lymphoma; plasmacytoma; multiple myeloma; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. The types of cancers that may be treated also include angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

Exemplifying hyperproliferative disorders amenable to CER therapy are B-cell cancers, including B-cell lymphomas (such as various forms of Hodgkin's disease, non-Hodgkins lymphoma (NHL) or central nervous system lymphomas), leukemias (such as acute lymphoblastic leukemia (ALL), chronic lymphocytic leukemia (CLL), Hairy cell leukemia, B cell blast transformation of chronic myeloid leukemia) and myelomas (such as multiple myeloma). Additional B cell cancers include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt's lymphoma/leukemia, B-cell proliferations of uncertain malignant potential, lymphomatoid granulomatosis, and post-transplant lymphoproliferative disorder.

Inflammatory and autoimmune diseases include arthritis, rheumatoid arthritis, juvenile rheumatoid arthritis, osteoarthritis, polychondritis, psoriatic arthritis, psoriasis, dermatitis, polymyositis/dermatomyositis, inclusion body myositis, inflammatory myositis, toxic epidermal necrolysis, systemic scleroderma and sclerosis, CREST syndrome, inflammatory bowel disease, Crohn's disease, ulcerative colitis, respiratory distress syndrome, adult respiratory distress syndrome (ARDS), meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), subacute cutaneous lupus erythematosus, discoid lupus, lupus myelitis, lupus cerebritis, juvenile onset diabetes, multiple sclerosis, allergic encephalomyelitis, neuromyelitis optica, rheumatic fever, Sydenham's chorea, immune responses associated with acute and delayed hypersensitivity mediated by cytokines and T-lymphocytes, tuberculosis, sarcoidosis, granulomatosis including Wegener's granulomatosis and Churg-Strauss disease, agranulocytosis, vasculitis (including hypersensitivity vasculitis/angiitis, ANCA and rheumatoid vasculitis), aplastic anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, central nervous system (CNS) inflammatory disorders, Alzheimer's disease, multiple organ injury syndrome, myasthenia gravis, antigen-antibody complex mediated diseases, anti-glomerular basement membrane disease, anti-phospholipid antibody syndrome, allergic neuritis, Behcet disease, Castleman's syndrome, Goodpasture's syndrome, Lambert-Eaton Myasthenic Syndrome, Reynaud's syndrome, Sjorgen's syndrome, Stevens-Johnson syndrome, solid organ transplant rejection, graft versus host disease (GVHD), bullous pemphigoid, pemphigus, autoimmune polyendocrinopathies, seronegative spondyloarthropathies, Reiter's disease, stiff-man syndrome, giant cell arteritis, immune complex nephritis, IgA nephropathy, IgM polyneuropathies or IgM mediated neuropathy, idiopathic thrombocytopenia purpura (ITP), thrombotic throbocytopenic purpura (TTP), Henoch-Schonlein purpura, autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, lymphoid interstitial pneumonitis (HIV), bronchiolitis obliterans (non-transplant), non-specific interstitial pneumonia (NSIP), Guillain-Barré Syndrome, large vessel vasculitis (including polymyalgia rheumatica and giant cell (Takayasu's) arteritis), medium vessel vasculitis (including Kawasaki's disease and polyarteritis nodosa), polyarteritis nodosa (PAN) ankylosing spondylitis, Berger's disease (IgA nephropathy), rapidly progressive glomerulonephritis, primary biliary cirrhosis, Celiac sprue (gluten enteropathy), cryoglobulinemia, cryoglobulinemia associated with hepatitis, amyotrophic lateral sclerosis (ALS), coronary artery disease, familial Mediterranean fever, microscopic polyangiitis, Cogan's syndrome, Whiskott-Aldrich syndrome and thromboangiitis obliterans. In certain embodiments, in the context of treating an inflammatory disease, it may be preferable to design a CER with a homeostatic (non-inflammatory) engulfment signaling domain.

Infectious diseases include those associated with infectious agents and include any of a variety of bacteria (e.g., pathogenic E. coli, S. typhimurium, P. aeruginosa, B. anthracis, C. botulinum, C. difficile, C. perfringens, H. pylori, V. cholerae, Listeria spp., Rickettsia spp., Chlamydia spp., and the like), mycobacteria, and parasites (including any known parasitic member of the Protozoa). Infectious viruses include eukaryotic viruses, such as adenovirus, bunyavirus, herpesvirus, papovavirus, papillomavirus (e.g., HPV), paramyxovirus, picornavirus, rhabdovirus (e.g., Rabies), orthomyxovirus (e.g., influenza), poxvirus (e.g., Vaccinia), reovirus, retrovirus, lentivirus (e.g., HIV), flavivirus (e.g., HCV, HBV) or the like. In certain embodiments, a composition comprising a CER according to the present disclosure is used for treating infection with a microbe capable of establishing a persistent infection in a subject.

Neurodegenerative diseases include Lewy body disease, postpoliomyelitis syndrome, Shy-Draeger syndrome, olivopontocerebellar atrophy, Parkinson's disease, multiple system atrophy, striatonigral degeneration, frontotemporal lobar degeneration with ubiquitinated inclusions (FLTD-U), tauopathies (including, but not limited to, Alzheimer disease and supranuclear palsy), prion diseases (also known as transmissible spongiform encephalopathies, including, but not limited to, bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease (including Amyotrophic lateral sclerosis (Lou Gehrig's disease)), and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g., of the CNS and/or brain, including brain metastases resulting from cancer elsewhere in the body). Many neurodegenerative diseases, including Alzheimer's disease, Parkinson's disease, Huntington's disease, Amyotrophic lateral sclerosis (Lou Gehrig's disease) and prion diseases, share a neuropathological signature, the aberrant accumulation of proteins, such as amyloid-β or tau in Alzheimer's disease; α-synuclein in Parkinson's disease (PD), dementia with Lewy bodies, multiple system atrophy, or Alzheimer's disease; huntingtin in Huntington's disease, SOD1 in Amyotrophic lateral sclerosis, proteins with polyglutamine (polyQ) repeats in Huntington's disease or Amyotrophic lateral sclerosis; TDP-43 in Amyotrophic lateral sclerosis or FLTD-U; or prion protein (e.g., Prp^{Sc}) in prion diseases. Thus, in certain embodiments, CER therapy may be designed to target the disease-associated protein in order to reduce or prevent aberrant protein accumulation, thereby slowing or preventing progression of the neurodegenerative disease.

A CER of this disclosure may be administered to a subject in cell-bound form (e.g., gene therapy of target cell population (mature T cells (e.g., CD8⁺ or CD4⁺ T cells) or other cells of T cell lineage)). Thus, for example, a CER of the present disclosure may be administered to a subject expressed on the surface of T cells, Natural Killer Cells, Natural Killer T cells, B cells, lymphoid precursor cells, antigen presenting cells, dendritic cells, Langerhans cells, myeloid precursor cells, mature myeloid cells, including subsets thereof, or any combination thereof. In certain embodiments, methods of treating a patient include administering an effective amount of CER modified cells (i.e., recombinant cells that express one or more CERs). In such embodiments, the CER modified cells are xenogeneic, syngeneic, allogeneic, or autologous cells of T cell lineage, Natural Killer cell lineage, Natural Killer T cell lineage, B cell lineage, lymphoid precursor cell lineage, dendritic cell lineage, Langerhans cell lineage, myeloid cell lineage, or any combination thereof.

Pharmaceutical compositions including a CER modified cells may be administered in a manner appropriate to the disease or condition to be treated (or prevented) as determined by persons skilled in the medical art. An appropriate dose, suitable duration, and frequency of administration of the compositions will be determined by such factors as the condition of the patient, size, weight, body surface area, age, sex, type and severity of the disease, particular therapy to be administered, particular form of the active ingredient, time and the method of administration, and other drugs being administered concurrently. The present disclosure provides pharmaceutical compositions comprising CER modified cells and a pharmaceutically acceptable carrier, diluent, or excipient. Suitable excipients include water, saline, dextrose, glycerol, or the like and combinations thereof. Other suitable infusion medium can be any isotonic medium formulation, including saline, Normosol R (Abbott), Plasma-Lyte A (Baxter), 5% dextrose in water, or Ringer's lactate.

A treatment effective amount of cells in a pharmaceutical composition is at least one cell (for example, one CER modified B cell) or is more typically greater than 10² cells, for example, up to 10⁶, up to 10⁷, up to 10⁸ cells, up to 10⁹ cells, up to 10¹⁰ cells, or up to 10¹¹ cells or more. In certain embodiments, the cells are administered in a range from about 10⁶ to about 10¹⁰ cells/m², preferably in a range of about 10⁷ to about 10⁹ cells/m². The number of cells will depend upon the ultimate use for which the composition is intended as well the type of cells included therein. For example, a composition comprising cells modified to contain a CER specific for a particular antigen will comprise a cell population containing from about 5% to about 95% or more of such cells. In certain embodiments, a composition comprising CER modified cells comprises a cell population comprising at least 5%, 10%, 15%, 20%, 25%, 30%, 35%, 40%, 45%, 50%, 55%, 60%, 65%, 70%, 75%, 80%, 85%, 90%, 95% or more of such cells. For uses provided herein, the cells are generally in a volume of a liter or less, 500 mls or less, 250 mls or less, or 100 mls or less. Hence the density of the desired cells is typically greater than 10⁴ cells/ml and generally is greater than 10⁷ cells/ml, generally 10⁸ cells/ml or greater. The cells may be administered as a single infusion or in multiple infusions over a range of time. Repeated infusions of CER modified cells may be separated by days, weeks, months, or even years if relapses of disease or disease activity are present. A clinically relevant number of immune cells can be apportioned into multiple infusions that cumulatively equal or exceed 10⁶, 10⁷, 10⁸, 10⁹, 10¹⁰, or 10¹¹ cells. A preferred dose for administration of a host cell comprising a recombinant expression vector as described herein is about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m². In certain embodiments, a composition of CER modified B cells and a composition of CER modified T cells are both administered, which administration may be simultaneous, concurrent or sequential.

In some embodiments, a composition as described herein is administered intravenously, intraperitoneally, intratumorally, into the bone marrow, into the lymph node, and/or into cerebrospinal fluid. In some embodiments, chimeric engulfment receptor engineered compositions are delivered to the site of the tumor.

In some embodiments, CER modified cells are administered to a subject in conjunction or combination with one or more additional therapies. In such embodiments, the one or more additional therapies may be one or more of radiation therapy, genetically engineered cellular immunotherapy (e.g., T cell, dendritic cell, natural killer cell, macrophage, chimeric antigen receptor (CAR) therapy), antibody therapy, immune checkpoint molecule inhibitor therapy, or a pharmaceutical therapy, such as a chemotherapeutic, a therapeutic peptide, a hormonal therapy, antibiotic, anti-viral agent, anti-fungal agent, anti-inflammatory agent, UV light therapy, electric pulse therapy, high intensity focused ultrasound therapy, oncolytic virus therapy, or a small molecule therapy. In such embodiments, the CER modified cells may clear apoptotic, dead, dying, damaged, infected, or necrotic cells displaying pro-apoptotic markers induced in the setting of the one or more additional therapies. In certain embodiments where CER modified cells are administered in combination with one or more additional therapies, the one or more additional therapies may be administered at a subtherapeutic dose due to an additive or synergistic effect of the combination with CER therapy. Combination therapy includes administration of a CER before an additional therapy (e.g., 1 day to 30 days or more before the additional therapy), concurrently with an additional therapy (on the same day), or after an additional therapy (e.g., 1 day-30 days or more after the additional therapy). In certain embodiments, the CER modified cells are administered after administration of the one or more additional therapies. In further embodiments, the CER modified cells are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administration of the one or more additional therapies. In still further embodiments, the CER modified cells are administered within 4 weeks, within 3 weeks, within 2 weeks, or within 1 week after administration of the one or more additional therapies. Where the one or more additional therapies involves multiple doses, the CER modified cells may be administered after the initial dose of the one or more additional therapies, after the final dose of the one or more additional therapies, or in between multiple doses of the one or more additional therapies.

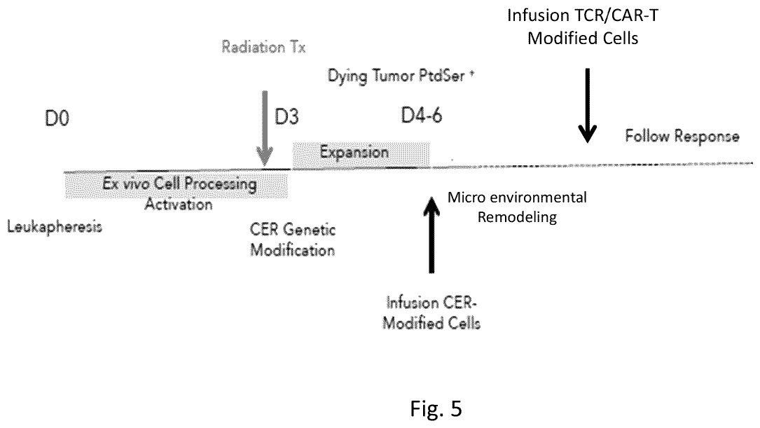

An example of a triple combination therapy (radiation+CER+CAR/or TCR) regimen is shown in FIG. 5. Following radiation therapy, tumor antigen specific, CER modified host cells (e.g., comprising a binding domain that binds to a tumor antigen) according to the present disclosure are administered to a subject to promote an anti-tumor immune response and recruit immune activating cells into the tumor microenvironment. In certain embodiments, CERs traffic to local, irradiated tumors and render the tumor tissue permissive for immune infiltration and destruction (e.g., via expression of inflammatory cytokines, activation of effector T cells, activation of dendritic cells, inhibition of regulatory T cells), thereby sensitizing the tumor microenvironment for subsequent adoptive T cell immunotherapy (e.g., CAR or TCR immunotherapy). In certain embodiments, the CER modified cells are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administration of the radiation therapy. In further embodiments, the CER modified cells are administered within 4 weeks, within 3 weeks, within 2 weeks, or within 1 week after administration of the radiation therapy. In certain embodiments, the CAR or TCR immunotherapy is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administration of the CER therapy or within 4 weeks, within 3 weeks, within 2 weeks, or within 1 week after administration of the CER therapy. In certain embodiments, the radiation therapy, the CAR or TCR immunotherapy, or both are administered at subtherapeutic levels.

Examples of radiation therapy that may be used in combination with CER therapy include external beam radiation therapy (e.g., conventional external beam radiation therapy, stereotactic radiation, 3-dimensional conformal radiation therapy, intensity-modulated radiation therapy, volumetric modulated arc therapy, particle therapy, proton therapy, auger therapy), brachytherapy, systemic radioisotope therapy, intraoperative radiotherapy, or any combination thereof. In certain embodiments, a lower or dose of radiation therapy than the typical dose or a subtherapeutic dose is used in combination with CER therapy. Low or subtherapeutic dose radiation therapy may be sufficient to cause sub-lytic membrane damage to the cells but not necessarily be cytolytic. The sub-lytic membrane damage is sufficient to expose pro-engulfment markers (e.g., phosphatidylserine) that can be targeted by CER therapy.

Examples of immune checkpoint molecules that may be targeted in combination with CER therapy include PD-L1, PD-L2, CD80, CD86, B7-H3, B7-H4, HVEM, adenosine, GAL9, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, PVRIG/CD112R, or any combination thereof. In certain embodiments, an immune checkpoint molecule inhibitor is an antibody, a peptide, an RNAi agent, or a small molecule. An antibody specific for CTLA-4 may be ipilimumab or tremelimumab. An antibody specific for PD-1 may be pidilizumab, nivolumab, or pembrolizumab. An antibody specific for PD-L1 may be durvalumab, atezolizumab, or avelumab.

A chemotherapeutic includes non-specific cytotoxic agents that inhibit mitosis or cell division, as well as molecularly targeted therapy that blocks the growth and spread of cancer cells by targeting specific molecules that are involved in tumor growth, progression, and metastasis (e.g., oncogenes). Exemplary non-specific chemotherapeutics include an alkylating agent, a platinum based agent, a cytotoxic agent, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor.

Examples of chemotherapeutic agents considered for use in combination therapies include vemurafenib, dabrafenib, trametinib, cobimetinib, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Exemplary alkylating agents include nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary platinum based agents include carboplatin, cisplatin, oxaliplatin, nedaplatin, picoplatin, satraplatin, phenanthriplatin, and triplatin tetranitrate.

Exemplary angiogenesis inhibitors include, without limitation A6 (Angstrom Pharmaceuticals), ABT-510 (Abbott Laboratories), ABT-627 (Atrasentan) (Abbott Laboratories/Xinlay), ABT-869 (Abbott Laboratories), Actimid (CC4047, Pomalidomide) (Celgene Corporation), AdGVPEDF.11D (GenVec), ADH-1 (Exherin) (Adherex Technologies), AEE788 (Novartis), AG-013736 (Axitinib) (Pfizer), AG3340 (Prinomastat) (Agouron Pharmaceuticals), AGX1053 (AngioGenex), AGX51 (AngioGenex), ALN-VSP (ALN-VSP 02) (Alnylam Pharmaceuticals), AMG 386 (Amgen), AMG706 (Amgen), Apatinib (YN968D1) (Jiangsu Hengrui Medicine), AP23573 (Ridaforolimus/MK8669) (Ariad Pharmaceuticals), AQ4N (Novavea), ARQ 197 (ArQule), ASA404 (Novartis/Antisoma), Atiprimod (Callisto Pharmaceuticals), ATN-161 (Attenuon), AV-412 (Aveo Pharmaceuticals), AV-951 (Aveo Pharmaceuticals), Avastin (Bevacizumab) (Genentech), AZD2171 (Cediranib/Recentin) (AstraZeneca), BAY 57-9352 (Telatinib) (Bayer), BEZ235 (Novartis), BIBF1120 (Boehringer Ingelheim Pharmaceuticals), BIBW 2992 (Boehringer Ingelheim Pharmaceuticals), BMS-275291 (Bristol-Myers Squibb), BMS-582664 (Brivanib) (Bristol-Myers Squibb), BMS-690514 (Bristol-Myers Squibb), Calcitriol, CCI-779 (Torisel) (Wyeth), CDP-791 (ImClone Systems), Ceflatonin (Homoharringtonine/HHT) (ChemGenex Therapeutics), Celebrex (Celecoxib) (Pfizer), CEP-7055 (Cephalon/Sanofi), CHIR-265 (Chiron Corporation), NGR-TNF, COL-3 (Metastat) (Collagenex Pharmaceuticals), Combretastatin (Oxigene), CP-751,871(Figitumumab) (Pfizer), CP-547,632 (Pfizer), CS-7017 (Daiichi Sankyo Pharma), CT-322 (Angiocept) (Adnexus), Curcumin, Dalteparin (Fragmin) (Pfizer), Disulfiram (Antabuse), E7820 (Eisai Limited), E7080 (Eisai Limited), EMD 121974 (Cilengitide) (EMD Pharmaceuticals), ENMD-1198 (EntreMed), ENMD-2076 (EntreMed), Endostar (Simcere), Erbitux (ImClone/Bristol-Myers Squibb), EZN-2208 (Enzon Pharmaceuticals), EZN-2968 (Enzon Pharmaceuticals), GC1008 (Genzyme), Genistein, GSK1363089 (Foretinib) (GlaxoSmithKline), GW786034 (Pazopanib) (GlaxoSmithKline), GT-111 (Vascular Biogenics Ltd.), IMC-1121B (Ramucirumab) (ImClone Systems), IMC-18F1 (ImClone Systems), IMC-3G3 (ImClone LLC), INCB007839 (Incyte Corporation), INGN 241 (Introgen Therapeutics), Iressa (ZD1839/Gefitinib), LBH589 (Faridak/Panobinostst) (Novartis), Lucentis (Ranibizumab) (Genentech/Novartis), LY317615 (Enzastaurin) (Eli Lilly and Company), Macugen (Pegaptanib) (Pfizer), MEDI522 (Abegrin) (MedImmune), MLN518 (Tandutinib) (Millennium), Neovastat (AE941/Benefin) (Aeterna Zentaris), Nexavar (Bayer/Onyx), NM-3 (Genzyme Corporation), Noscapine (Cougar Biotechnology), NPI-2358 (Nereus Pharmaceuticals), OSI-930 (OSI), Palomid 529 (Paloma Pharmaceuticals, Inc.), Panzem Capsules (2ME2) (EntreMed), Panzem NCD (2ME2) (EntreMed), PF-02341066 (Pfizer), PF-04554878 (Pfizer), PI-88 (Progen Industries/Medigen Biotechnology), PKC412 (Novartis), Polyphenon E (Green Tea Extract) (Polypheno E International, Inc.), PPI-2458 (Praecis Pharmaceuticals), PTC299 (PTC Therapeutics), PTK787 (Vatalanib) (Novartis), PXD101 (Belinostat) (CuraGen Corporation), RAD001 (Everolimus) (Novartis), RAF265 (Novartis), Regorafenib (BAY73-4506) (Bayer), Revlimid (Celgene), Retaane (Alcon Research), SN38 (Liposomal) (Neopharm), SNS-032 (BMS-387032) (Sunesis), SOM230 (Pasireotide) (Novartis), Squalamine (Genaera), Suramin, Sutent (Pfizer), Tarceva (Genentech), TB-403 (Thrombogenics), Tempostatin (Collard Biopharmaceuticals), Tetrathiomolybdate (Sigma-Aldrich), TG100801 (TargeGen), Thalidomide (Celgene Corporation), Tinzaparin Sodium, TKI258 (Novartis), TRC093 (Tracon Pharmaceuticals Inc.), VEGF Trap (Aflibercept) (Regeneron Pharmaceuticals), VEGF Trap-Eye (Regeneron Pharmaceuticals), Veglin (VasGene Therapeutics), Bortezomib (Millennium), XL184 (Exelixis), XL647 (Exelixis), XL784 (Exelixis), XL820 (Exelixis), XL999 (Exelixis), ZD6474 (AstraZeneca), Vorinostat (Merck), and ZSTK474.

Exemplary molecularly targeted inhibitors include angiogenesis inhibitors (e.g., a VEGF pathway inhibitors), tyrosine kinase inhibitors (e.g., an EGF pathway inhibitors), receptor tyrosine kinase inhibitors, growth factor inhibitors, GTPase inhibitors, serine/threonine kinase inhibitors, transcription factor inhibitors, B-Raf inhibitors, MEK inhibitors, mTOR inhibitors, EGFR inhibitors, ALK inhibitors, ROS1 inhibitors, BCL-2 inhibitors, PI3K inhibitors, VEGFR inhibitors, BCR-ABL inhibitors, MET inhibitors, MYC inhibitors, ABL inhibitors, HER2 inhibitors, BTK inhibitors, H-RAS inhibitors, K-RAS inhibitors, and PDGFR inhibitors. In certain embodiments, use of molecularly targeted therapy comprises administering a molecularly targeted therapy specific for the molecular target to a subject identified as having a tumor that possesses the molecular target (e.g., driver oncogene). In certain embodiments, the molecular target has an activating mutation. In certain embodiments, use of CER modified cells in combination with a molecularly targeted inhibitor increases the magnitude of anti-tumor response, the durability of anti-tumor response, or both. In certain embodiments, a lower than typical dose or a subtherapeutic dose of molecularly targeted therapy is used in combination with CER modified cells.

Exemplary Vascular Endothelial Growth Factor (VEGF) receptor inhibitors include, but are not limited to, Bevacizumab (Avastin®), axitinib (Inlyta®); Brivanib alaninate (BMS-582664, (S) ((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®).

Exemplary EGF pathway inhibitors include, without limitation tyrphostin 46, EKB-569, erlotinib (Tarceva®), gefitinib (Iressa®), erbitux, nimotuzumab, lapatinib (Tykerb®), cetuximab (anti-EGFR mAb), ¹⁸⁸Re-labeled nimotuzumab (anti-EGFR mAb), and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980. Exemplary EGFR antibodies include, but are not limited to, Cetuximab (Erbitux®); Panitumumab (Vectibix®); Matuzumab (EMD-72000); Trastuzumab (Herceptin®); Nimotuzumab (hR3); Zalutumumab; TheraCIM h-R3; MDX0447 (CAS 339151-96-1); and ch806 (mAb-806, CAS 946414-09-1). Exemplary Epidermal growth factor receptor (EGFR) inhibitors include, but not limited to, Erlotinib hydrochloride (Tarceva®); brigatinib; osimeritinib; icotinib; Gefitnib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (BIBW2992); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα, 5β, 6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PKI166, CAS 187724-61-4).

Exemplary mTOR inhibitors include, without limitation, rapamycin (Rapamune®), and analogs and derivatives thereof; SDZ-RAD; Temsirolimus (Torisel®; also known as CCI-779); Ridaforolimus (formally known as deferolimus, (1R,2R,4S)-4-[(2R)-2[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04.9]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-[[4-(4-oxo-8-phenyl-4H-1-benzopyran-2-yl)morpholinium-4-yl]methoxy]butyl]-L-arginylglycyl-L-α-aspartyIL-serine-, inner salt (SF1126, CAS 936487-67-1).

Exemplary Phosphoinositide 3-kinase (PI3K) inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806); 4-(trifluoromethyl)-5-(2,6-dimorpholinopyrimidin-4-yl)pyridin-2-amine (also known as BKM120 or NVP-BKM120, and described in PCT Publication No. WO2007/084786); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); and 8-Phenyl-2-(morpholin-4-yl)-chromen-4-one (LY294002, CAS 154447-36-6). Exemplary Protein Kinase B (PKB) or AKT inhibitors include, but are not limited to. 8-[4-(1-Aminocyclobutyl)phenyl]-9-phenyl-1,2,4-triazolo[3,4-f][1,6]naphthyridin-3(2H)-one (MK-2206, CAS 1032349-93-1); Perifosine (KRX0401); 4-Dodecyl-N-1,3,4-thiadiazol-2-yl-benzenesulfonamide (PHT-427, CAS 1191951-57-1); 4-[2-(4-Amino-1,2,5-oxadiazol-3-yl)-1-ethyl-7-[(3S)-3-piperidinylmethoxy]-1H-imidazo[4,5-c]pyridin-4-yl]-2-methyl-3-butyn-2-ol (GSK690693, CAS 937174-76-0); 8-(1-Hydroxyethyl)-2-methoxy-3-[(4-methoxyphenyl)methoxy]-6H-dibenzo[b,d]pyran-6-one (palomid 529, P529, or SG-00529); Tricirbine (6-Amino-4-methyl-8-(β-D-ribofuranosyl)-4H,8H-pyrrolo[4,3,2-de]pyrimido[4,5-c]pyridazine); (αS)-α-[[[5-(3-Methyl-1H-indazol-5-yl)-3-pyridinyl]oxy]methyl]-benzeneethanamine (A674563, CAS 552325-73-2); 4-[(4-Chlorophenyl)methyl]-1-(7H-pyrrolo[2,3-d]pyrimidin-4-yl)-4-piperidinamine (CCT128930, CAS 885499-61-6); 4-(4-Chlorophenyl)-4-[4-(1H pyrazol-4-yl)phenyl]-piperidine (AT7867, CAS 857531-00-1); and Archexin (RX-0201, CAS 663232-27-7).

In certain embodiments, a tyrosine kinase inhibitor used in combination with CER modified cells is an anaplastic lymphoma kinase (ALK) inhibitor. Exemplary ALK inhibitors include crizotinib, ceritinib, alectinib, brigatinib, dalantercept, entrectinib, and lorlatinib.

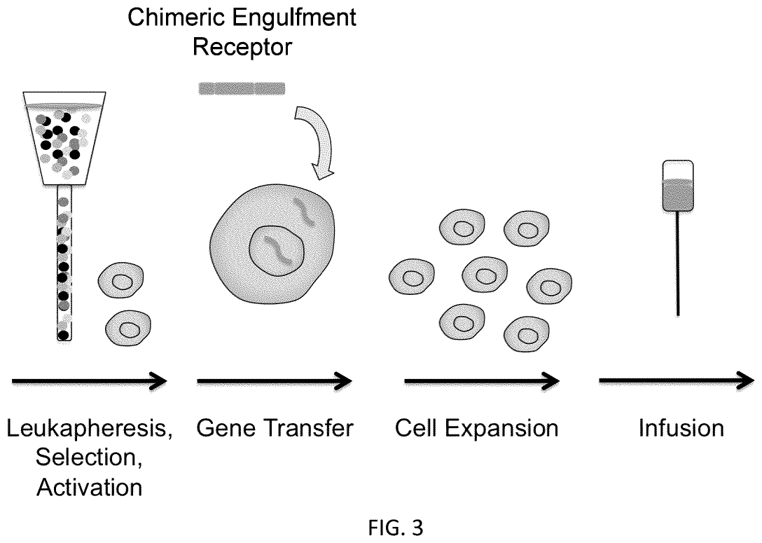

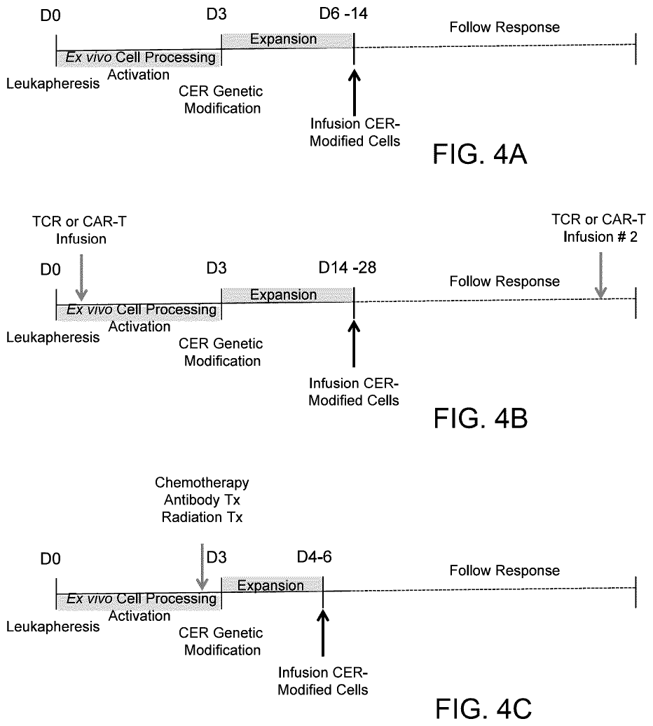

FIGS. 3-5 illustrate embodiments of regimens that utilize CER modified cells. As shown in FIGS. 3 and 4A, following leukapheresis, cells can be processed and activated ex vivo, undergoing genetic modification and expansion in preparation for infusion into a subject. FIG. 4B shows an illustrative treatment scheme for CER-modified cells used in combination with conventional T cell based therapies (e.g., CAR or TCR). An initial infusion of engineered T cells induces tumor cell apoptosis indicative of an anti-tumor effect. CER modified cells are then infused. The CER modified cells clear tumor cells displaying a pro-engulfment (e.g., PtdSer), which facilitates tumor regression while also bypassing the T cell suppressive tumor microenvironment. Alteration of the tumor microenvironment then re-sensitizes the tumor to T cell therapy, allowing a second infusion of T cells. Another embodiment of a therapeutic method is shown in FIG. 4C. The treatment scheme shown in FIG. 4C utilizes CER modified cells in combination with a monoclonal antibody therapy. Infusion of tumor-specific antibodies, such as Cetuximab targeting EGFR or Rituximab targeting CD20 may trigger cell death or induce a targeting moiety that is bound by CER modified cells. Subsequently, a subject receives CER modified cells that bind to and clear antibody bound cells. In such an embodiment, the CER extracellular domain may include an FcR binding domain, a PtdSer binding domain, or other antigen binding domain.

In another scenario, a CER modified cell can be combined with small molecule inhibitors such as a BTK inhibitor, a MEK inhibitor, an adenosine pathway inhibitor A2AR antagonist, an IDO1 inhibitor, IMiDs such as Lenalidomide, PI3K8 inhibitors, a BRAF inhibitor, or a BCR-ABL inhibitor.

In certain embodiments, methods of the present disclosure include a depletion step. A depletion step to remove CERs from the subject may occur after a sufficient amount of time for therapeutic benefit in order to mitigate toxicity to a subject. In such embodiments, the CER vector includes an inducible suicide gene, such as iCASP9, inducible Fas, or HSV-TK. Similarly, a CER vector may be designed for expression of a known cell surface antigen such as CD20 or truncated EGFR (SEQ ID NO:105) that facilitates depletion of transduced cells through infusion of an associated monoclonal antibody (mAb), for example, Rituximab for CD20 or Cetuximab for EGFR. Alemtuzumab, which targets CD52 present on the surface of mature lymphocytes, may also be used to deplete transduced B cells, T cells, or natural killer cells.

In further embodiments, cells expressing CER of the instant disclosure may be used in diagnostic methods or imaging methods, including methods used in relation to the indications or conditions identified herein.

### Example 1

### Construction of TIM4-TLR4 CER “CER05”

The extracellular domain of the phosphatidylserine binding protein Tim4 (encoding amino acid sequence of SEQ ID NO:106), including the signal peptide (amino acids 1-22 of SEQ ID NO:106) and transmembrane domain (encoding amino acid sequence of SEQ ID NO:108), were fused to the intracellular signaling domain of the TLR4 (encoding amino acid sequence of SEQ ID NO:51) to create a chimeric engulfment receptor “CER05” (Tim4-TLR CER having an amino acid sequence of SEQ ID NO:81). The TLR4 signaling domain transduces a signal for engulfment, and Tim4 is a phosphatidylserine binding receptor. The Tim4-TLR4 (CER05) chimeric engulfment receptor nucleotide sequence was then inserted into the pLenti lentiviral vector along with truncated EGFR (EGFRt or tEGFR) (encoding amino acid sequence of SEQ ID NO: 105) as a transduction marker, separated by T2A sequence (see, FIG. 6). Murine Ba/F3 B-cells were cultured in RMPI 1640 media supplemented with 10% fetal bovine serum, 1% penicillin-streptomycin, and 10 ng/ml murine IL-3 (Peprotech Catalog #213-13) in a 12 well plate at a density of 0.5 million cells/ml. Under normal conditions, the Ba/F3 murine B-cell line lacks the capacity to engulf target cells and was therefore selected to establish an assay system for engulfment. To transduce Ba/F3 cells, 100 μl of viral vector expressing Tim4-TLR4 (CER05) and 5 μl TRANSDUX™ transduction reagent were diluted in 0.5 ml Complete Cell Growth Media and added to the Ba/F3 cells. The Ba/F3 cells were then centrifuged at 270×g rpm for 1 hour in a 32° C. pre-warmed centrifuge. The Ba/F3 cells were incubated for 24 hours at 37° C. Ba/F3 cells were expanded for another 48 hours in Complete Cell Growth Media. Positive Ba/F3 cell transductants were sorted using fluorescence activated cell sorting (FACs) (Sony Sorter SH800) by either staining with a labeled EGFR-specific antibody (Cetixumab). Post sorting, purified, transduced Ba/F3 cells comprising the Tim4-TLR4-T2A-transduction marker containing viral vector were rested for 48 hours prior to being utilized for phagocytic assays.

### Phagocytic Activity Against Primary Apoptotic Thymocytes

One day prior to phagocytic assay, primary thymocytes were isolated from a C3H mouse (Charles River Laboratories International, Inc.). Thymocytes were cultured in complete RPMI 1640 growth media supplemented with 10% fetal bovine serum and 1% penicillin-streptomycin in a 6-well plate. To induce apoptosis and phosphatidylserine expression on the cell surface, thymocytes were treated with 1 μM dexamethasone for 24 hours. Untreated thymocytes were used as a negative control. Thymocytes were collected from the 6-well plates, washed once with sterile 1×PBS, and then stained with 1 ng/μl pH sensitive pHrodo™ Red dye (ThermoFisher Scientific, Catalog #P36600) in PBS at room temperature for 15 minutes. Labeling target cells with pHrodo Red dye permits visualization of cells that are engulfed and transported into lysosomes due to their increased light emission in the acidic lysosomal environment (Miksa et al., 2009, Immunol. Methods 342:71-7). The cells were then supplemented with growth media and washed one more time to remove any excess pHrodo Red. pHrodo Red stained thymocytes were plated on a flat bottom 96 well plate at 250,000 cells/well in RMPI 1640 complete media.

Ba/F3 CER01⁺ tEGFR⁺ cells made as described above were washed once with 1×PBS and stained with 1 μM CELLTRACE™ Violet dye (ThermoFisher Scientific, Catalog #C34557) in PBS for 10 minutes at 37° C. Stained, transduced Ba/F3 cells were supplemented with growth media, washed once with 1×PBS to remove excess CELLTRACE™ Violet, and plated on the same flat bottom 96 well plate at approximately 25,000 cells/well in RPMI 1640 complete media.

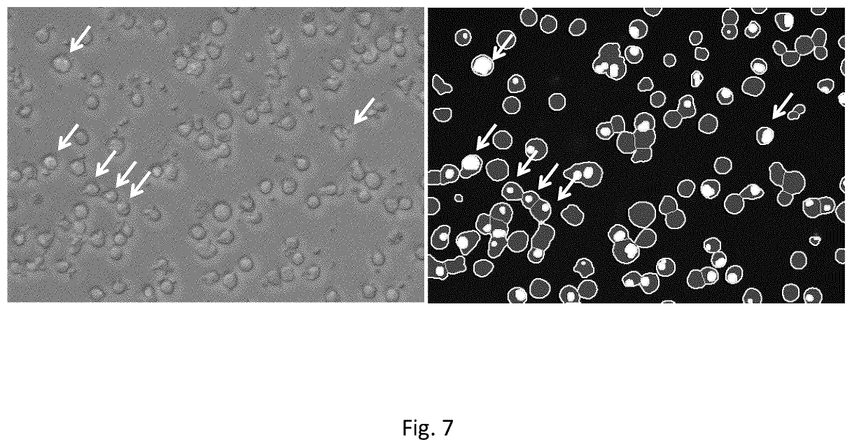

Target thymocytes were co-cultured with stained, Ba/F3 CER05⁺ cells at a ratio of 10:1 (target cell:effector cell) for 3 hours or overnight (˜14 hours) at 37° C. After incubation, the plate was centrifuged and the media replaced with PBS supplemented with 2% fetal bovine serum, pH 9. The 96 well plate was then viewed using KEYENCE BZ-X710 fluorescence microscope, 20× objective. Ba/F3 cells transduced with pLenti vector expressing truncated EGFR were used as a negative control. Fluorescent microscopy showed that CER05⁺ Ba/F3 cells engulf dexamethasone-treated thymocytes (white arrows indicate engulfment events) (see, FIG. 7).

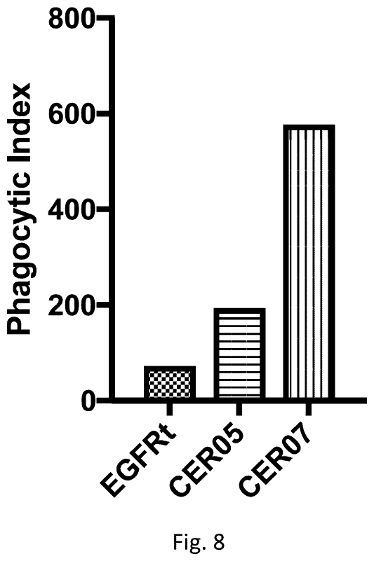

A phagocytic index was calculated by multiplying [mean of total number of engulfed target cells/total number of counted CER modified cells (e.g., phagocytic frequency)] by [average area of target cell staining per CER+ Ba/F3 cell×100 (e.g., hybrid capture)] as compared to control EGFRt+ Ba/F3 cells (see, FIG. 8).

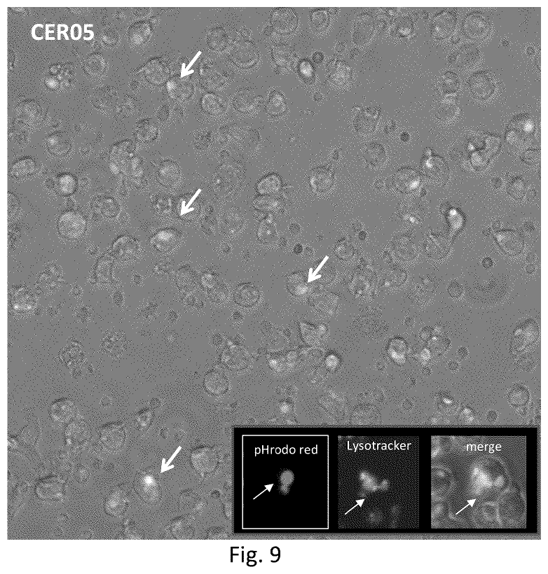

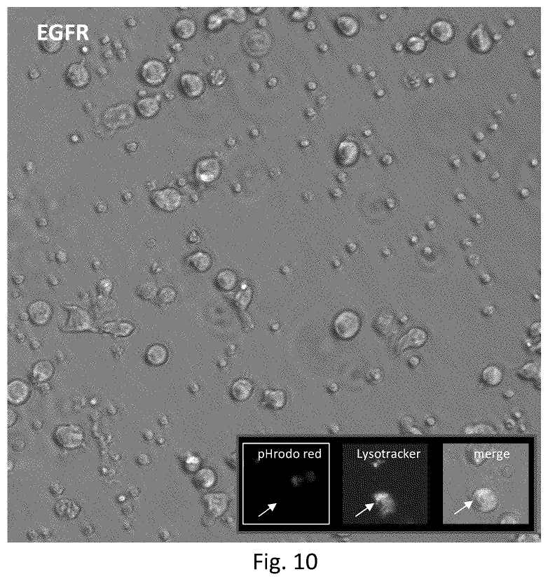

A duplicate plate of co-cultured Ba/F3 CER05⁺ cells and dexamethasone treated thymocytes was incubated for 6 hours in media containing IL-3. 50 nM LysoTracker green, which stains acidic compartments in live cells green (e.g., lysosomes) was added 5 minutes prior to the end of incubation period. Co-localization of internalized pHrodo red labeled thymocytes with LysoTracker green vesicles can be visualized by the overlay of these 2 images. Co-localization of red and green fluorescence gives rise to yellow/orange fluorescence in the merged images, indicating pHrodo-labeled target cells have been internalized into lysosomes, leading to rapid acidification and killing of the ingested cell (see, FIG. 9; white arrows indicate co-localization of pHrodo red labeled thymocytes with LysoTracker green vesicles). Fluorescent microscope image of co-cultured control Ba/F3 cells transduced with truncated EGFR and dexamethasone treated thymocytes is shown in FIG. 10.

### Phagocytic Activity Against Murine Cell Lines

One day prior to the phagocytosis assay, CT26 murine colon carcinoma cells were cultured in complete RPMI 1640 growth media supplemented with 10% fetal bovine serum and 1% penicillin-streptomycin in a 6-well plate and treated with 1 mM staurosporine (STS) for 12 hours to induce apoptosis. Untreated CT26 cells were used as a negative control.

On the day of the phagocytosis assay, CT26 cells were collected, washed twice with 1×PBS to remove excess staurosporine and then stained with 1 ng/μl pHrodo Red in PBS at room temperature for 15 minutes. The CT26 cells were supplemented with growth media, washed once to remove excess pHrodo Red, and plated onto a flat bottom, 96 well plate at 250,000 cells/well in RPMI 1640 complete media.

Ba/F3 CER05⁺ EGFR⁺ cells made as described above were washed once with 1×PBS and stained with 1 μM CELLTRACE™ Violet dye (ThermoFisher Scientific, Catalog #C34557) in PBS for 10 minutes at 37° C. Stained, transduced Ba/F3 cells were supplemented with growth media, washed once with 1×PBS to remove excess CELLTRACE™ Violet, and plated on the same flat bottom 96 well plate at approximately 50,000 cells/well in RPMI 1640 complete media.

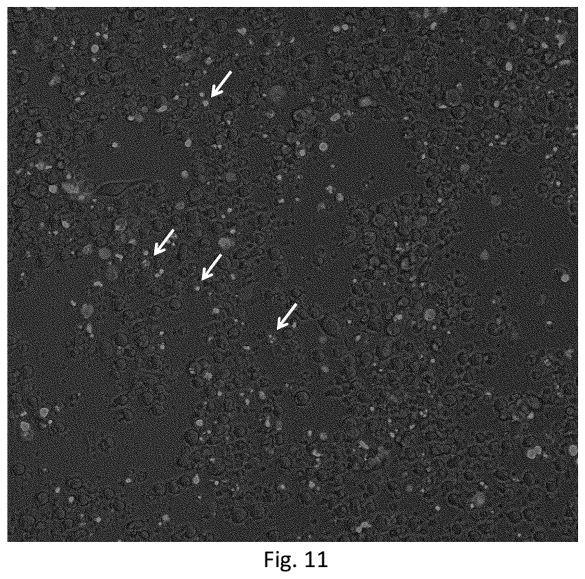

Target CT26 cells were co-cultured with stained, CER05⁺ tEGFR⁺ cells at a ratio of 5:1 (target cell:effector cell) for 3 hours at 37° C. After incubation, the plate was centrifuged and the media replaced with PBS supplemented with 2% fetal bovine serum, pH 9. The 96 well plate was then viewed using KEYENCE BZ-X710 fluorescence microscope, 20× objective. Ba/F3 cells transduced with pLenti vector expressing truncated EGFR were used as negative control. Fluorescent micrograph showing in vitro phagocytosis is shown in FIG. 11 (white arrows show phagocytosis events). CT26 cells labeled with pHrodo Red fluoresced inside the low pH compartments of lysosomes when engulfed (outlined in pink).

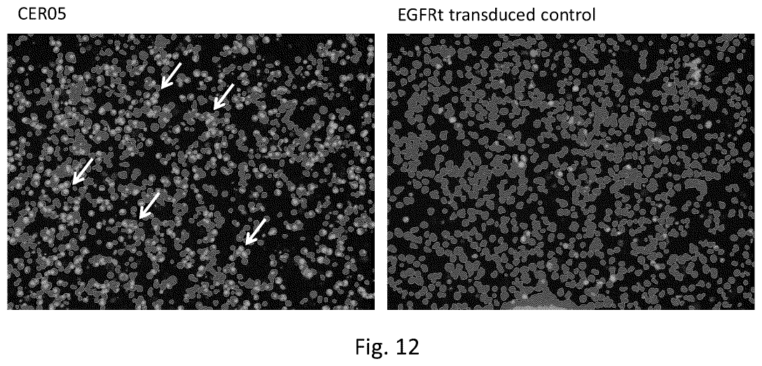

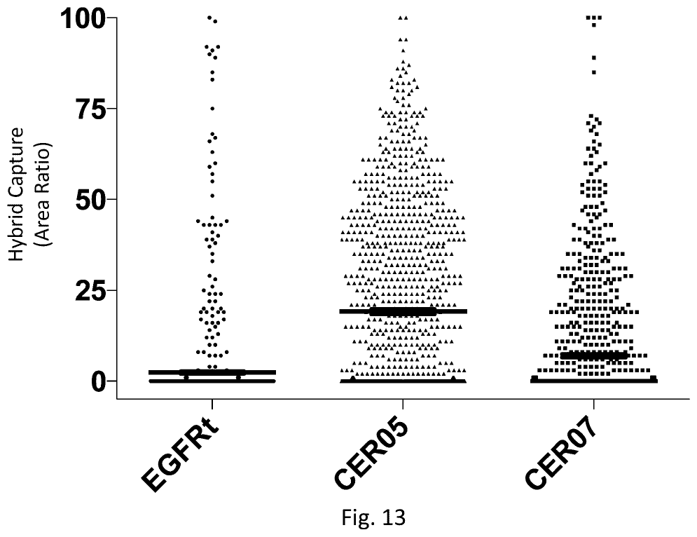

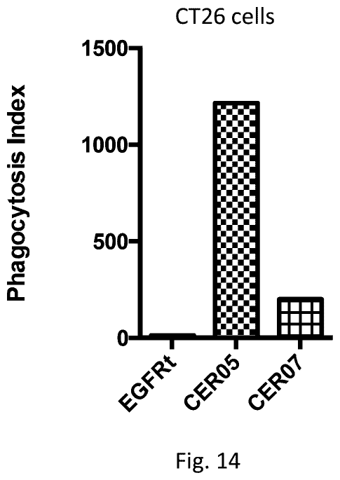

A hybrid capture algorithm that detects fluorescence of pHrodo Red within CELLTRACE Violet staining area was applied to fluorescent images to quantify the area of engulfed target cells/area of CER⁺ B cells or control tEGFR+ B cells (see, FIG. 12). FIG. 13 shows a scatterplot of hybrid cell counts extracting CT26 target cell area within Ba/F3 cells transduced with CER05⁺ EGFR⁺ or EGFR⁺ control. The area ratio represents the co-localization area of CT26 cells within Ba/F3 cells. A phagocytic index for CER05+ Ba/F3 cells as compared to EGFRt transduced Ba/F3 control cells is shown in FIG. 14.

### Example 2

### Construction of TIM4-TLR4 (TLR4 TMD) CER “CER07”

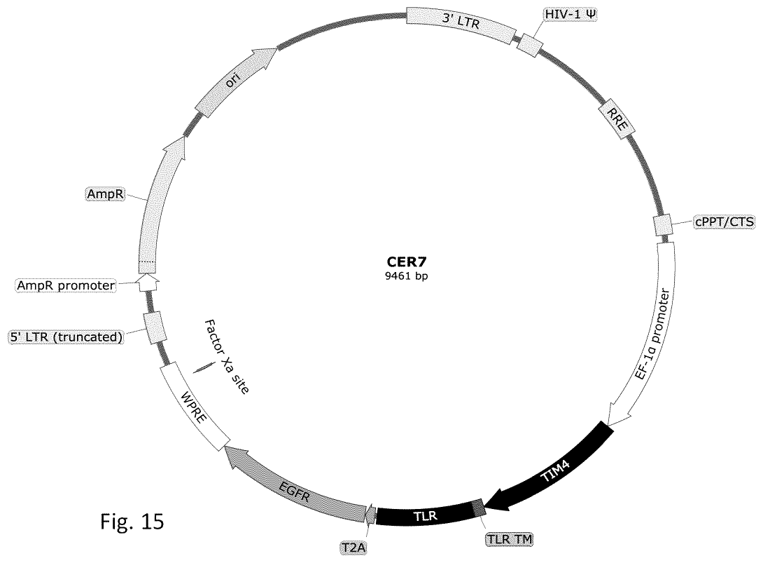

The extracellular domain of the phosphatidylserine binding protein Tim4 (amino acid sequence of SEQ ID NO: 106), including the signal peptide (amino acids 1-22 of SEQ ID NO:106), was fused to a TLR4 transmembrane domain (amino acid sequence of SEQ ID NO:34) and an intracellular signaling domain of TLR4 (SEQ ID NO:51) to create a chimeric engulfment receptor “CER07” (Tim4-Tyro3 CER having an amino acid sequence of SEQ ID NO:83). The TLR4 signaling domain transduces a signal for engulfment, and Tim4 is a phosphatidylserine binding receptor. The Tim4-TLR4-TLR4 (CER07) chimeric engulfment receptor nucleotide sequence was then inserted into the pLenti lentiviral vector along with truncated EGFR as a transduction marker, separated by T2A sequence (see, FIG. 15). Murine Ba/F3 B-cells were transduced with pLenti vector expressing Tim4-TLR4-TLR4 (CER07) and EGFRt, expanded, sorted by FACs, and used for in vitro studies as described in Example 1.

### Phagocytic Activity Against Primary Apoptotic Thymocytes

Primary C3H mouse thymocytes were isolated, treated with dexamethasone, and stained with pHrodo Red as described in Example 1. Ba/F3 CER07⁺ tEGFR⁺ cells were labeled with CELLTRACE™ Violet dye as described in Example 1. Co-culture experiments were carried out at a 10:1 target cell to effector cell ratio, and Ba/F3 CER07⁺ tEGFR⁺ cells were quantified for phagocytosis by fluorescence microscopy and FACs as described in Example 1. Ba/F3 cells transduced with pLenti vector expressing truncated EGFR were used as a negative control.

Fluorescent microscopy showed that CER07⁺ Ba/F3 cells engulfed dexamethasone-treated thymocytes as compared to EGFRt transduced Ba/F3 control cells.

A phagocytic index was calculated by multiplying [mean of total number of engulfed target cells/total number of counted CER modified cells (e.g., phagocytic frequency)] by [average area of target cell staining per CER+ Ba/F3 cell×100 (e.g., hybrid capture)] as compared to EGFRt transduced Ba/F3 control cells (see, FIG. 8).

### Phagocytic Activity Against Murine Cell Lines

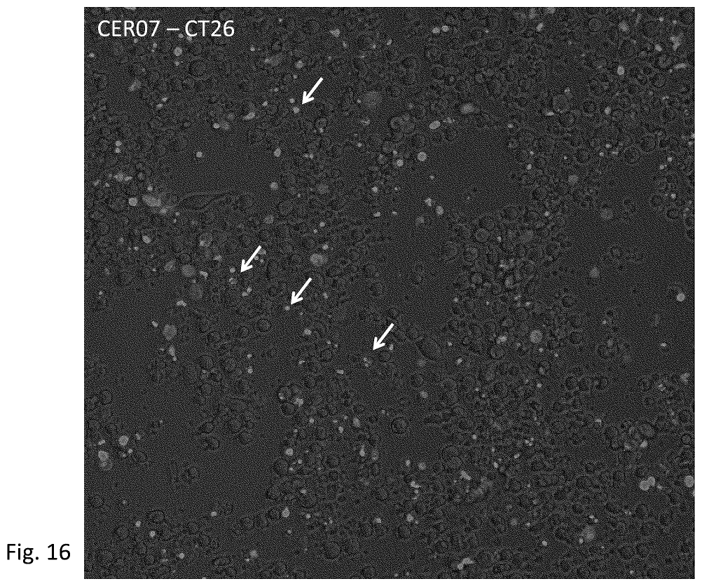

CER07+ Ba/F3 cells were co-cultured with CT26 murine colon carcinoma cells as described in Example 1. Fluorescent microscopy showed that CER07⁺ Ba/F3 cells engulfed staurosporine treated CT26 cells (see, FIG. 16, white arrows indicate phagocytosis). Ba/F3 cells transduced with EGFRt was used as a control.

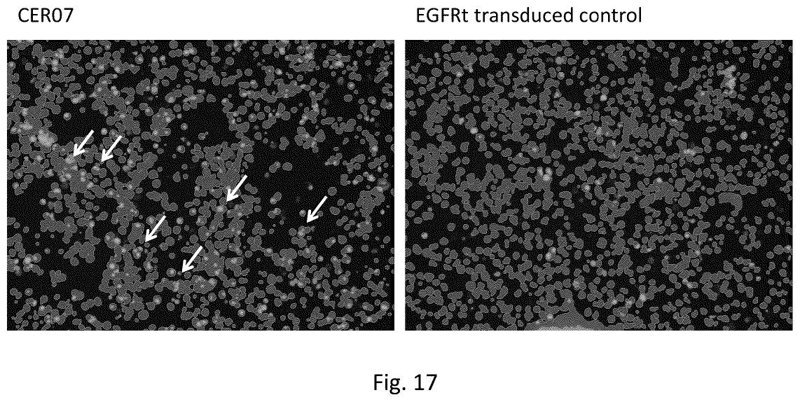

A hybrid capture algorithm that detects fluorescence of pHrodo Red within CELLTRACE Violet staining area was applied to fluorescent images to quantify the area of engulfed target cells/area of CER⁺ B cells (see, FIG. 17). FIG. 13 shows a scatterplot of hybrid cell counts extracting CT26 target cell area within Ba/F3 cells transduced with CER07⁺ EGFR⁺ or EGFR⁺ control. The area ratio represents the co-localization area of CT26 cells within Ba/F3 cells. A phagocytic index for CER07+ Ba/F3 cells as compared to EGFRt transduced Ba/F3 control cells is shown in FIG. 14.

### Example 3

### Construction of TIM4-TLR8 (TLR4 TMD and spacer) CER “CER21” and TIM4-TLR5) CER “CER19”

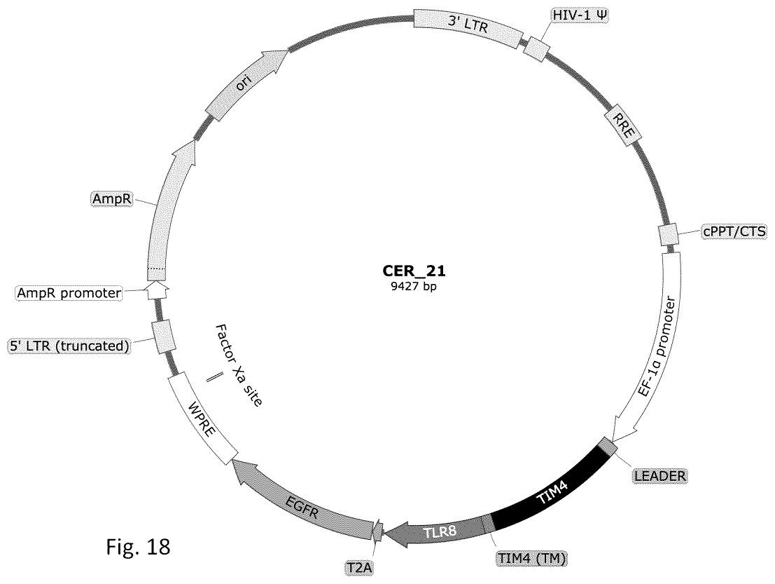

The extracellular domain of the phosphatidylserine binding protein Tim4 (amino acid sequence of SEQ ID NO: 106), including the signal peptide (amino acids 1-22 of SEQ ID NO:106), and Tim4 transmembrane domain (amino acid sequence of SEQ ID NO:108), were fused to the intracellular signaling domain of TLR8 (SEQ ID NO:55) to create a chimeric engulfment receptor “CER21” (Tim4-TLR8 CER having an amino acid sequence of SEQ ID NO:88). The TLR8 signaling domain transduces a signal for engulfment, and Tim4 is a phosphatidylserine binding receptor. The Tim4-TLR8 (CER21) chimeric engulfment receptor nucleotide sequence was then inserted into the pLenti lentiviral vector along with truncated EGFR as a transduction marker, separated by T2A sequence (see, FIG. 18). Human primary B cells were transduced with pLenti vector expressing Tim4-TLR8 (CER21) and EGFRt, expanded, sorted by FACS, and used for in vitro studies as described in Example 1.

The extracellular domain of the phosphatidylserine binding protein Tim4 (amino acid sequence of SEQ ID NO: 106), including the signal peptide (amino acids 1-22 of SEQ ID NO:106), and Tim4 transmembrane domain (amino acid sequence of SEQ ID NO:108), were fused to the intracellular signaling domain of TLR5 (SEQ ID NO:52) to create a chimeric engulfment receptor “CER19” (Tim4-TLR5 CER having an amino acid sequence of SEQ ID NO:86). The TLR5 signaling domain transduces a signal for engulfment, and Tim4 is a phosphatidylserine binding receptor. The Tim4-TLR5 (CER19) chimeric engulfment receptor nucleotide sequence was then inserted into the pLenti lentiviral vector along with truncated EGFR as a transduction marker, separated by T2A sequence. Human primary B cells were transduced with pLenti vector expressing Tim4-TLR8 (CER19) and EGFRt, expanded, sorted by FACS, and used for in vitro studies as described in Example 1.

### Phagocytic Activity of Human CER21+ B Cells Against Human Cell Line

One day prior to setting up the phagocytosis assay, Jurkat human B lymphocytes were cultured in complete RPMI 1640 growth media supplemented with 10% fetal bovine serum and 1% penicillin-streptomycin in a 6 well plate and treated with 1 mM staurosporine for three hours to induce apoptosis. Jurkat cells were washed twice in 1×PBS to remove excess staurosporine and then stained with pHrodo Red (1 ng/μl in PBS) for 15 minutes at room temperature. The Jurkat cells were supplemented with growth media, washed once to remove excess pHrodo Red, and plated on flat bottom 96 well plates at approximately 250,000 cells/well in RPMI 1640 complete media.

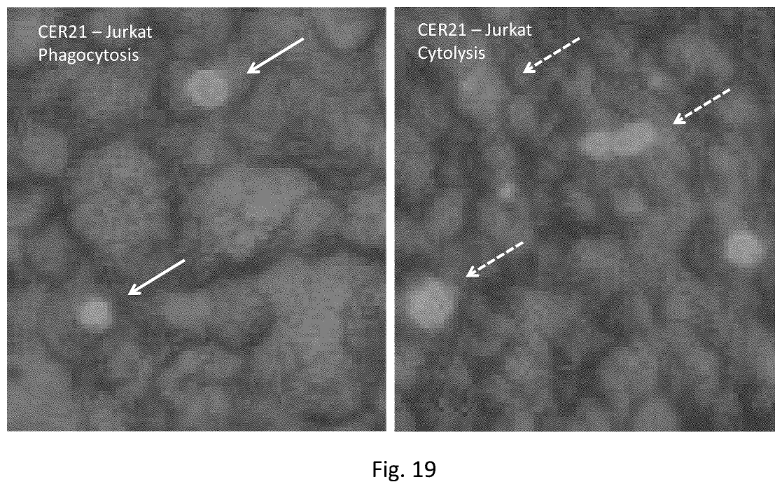

Transduced CER21+ human primary B cells were washed once with 1×PBS and stained with 1 μM CELLTRACE Violet in PBS for 10 minutes at 37° C. The CER21+ human primary B cells were supplemented with growth media, washed once with 1×PBS to remove excess CELLTRACE Violet, and plated onto 96 well plate at approximately 50,000 cells/well in RPMI 1640 complete media. Human CER21+ primary B cells and Jurkat cells were co-cultured at a target cell to effector cell ratio of 5:1 at 37° C. for 3 hours. After incubation, the co-culture plate was then centrifuged, and the media replaced with PBS supplemented with 2% fetal bovine serum, pH 9. Phagocytic events were quantified by fluorescent microscopy (KEYENCE BZ-X710 fluorescence microscope, 20× objective). Fluorescent microscope image showing in vitro phagocytosis is shown in FIG. 19A for CER021⁺ B cells co-cultured with Jurkat cells. Solid arrows show engulfment activity. Fluorescent microscope image showing in vitro cytolysis of Jurkat cells engulfed by CER21+ B cells is shown in FIG. 19B. Dashed arrows show cytolytic activity.

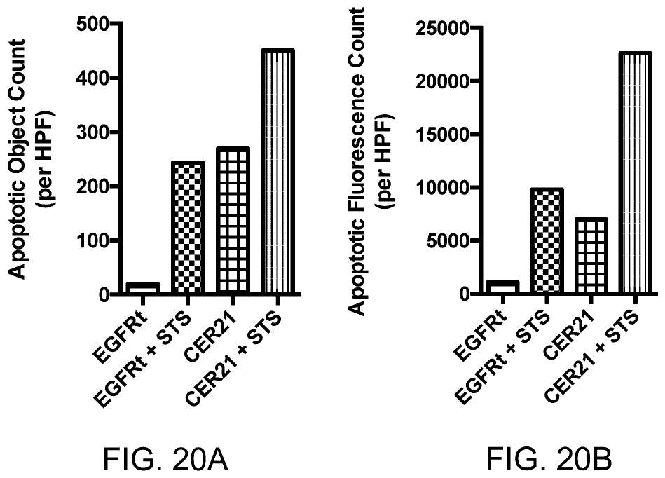

Apoptotic cells were quantified from fluorescent images using automated software, and the number of apoptotic cells per high power view (FIG. 20A) and total fluorescence emission per high power view (FIG. 20B) were calculated using pH indicator dye. CER21+ B cells co-cultured with staurosporine treated (subtherapeutic dose) Jurkat cells exhibited enhanced killing of target cells.

### Enhanced Killing by Human CER21⁺ B Cells Against Chemotherapy-Treated Human Cell Line

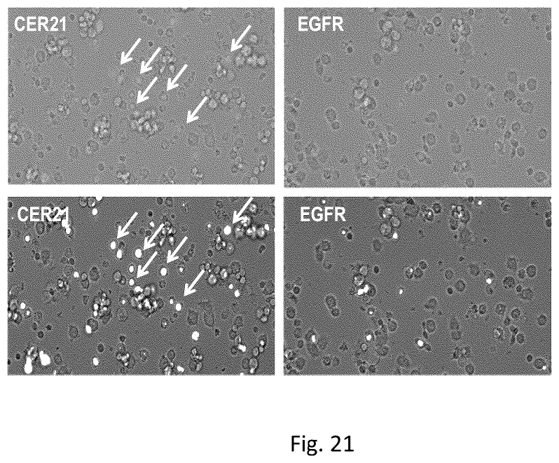

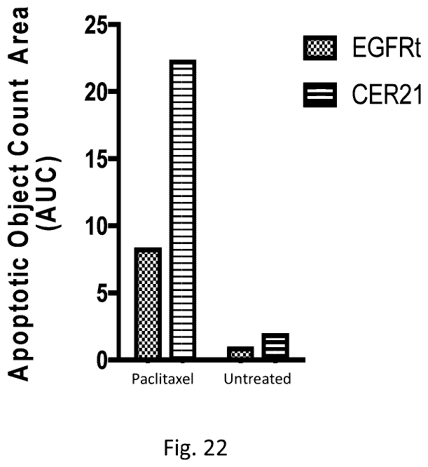

Human primary B cells were transduced with pLenti Tim4-TLR8 (CER21) lentivirus expressing truncated EGFR as a transduction marker and stained with CELLTRACE Violet as described in Example 1. One day prior to setting up a co-culture assay, H1703 non-small cell lung cancer cells were incubated with phosphatidylserine inducing chemotherapy Paclitaxel (30 μM) in serum-free media for 24 hours. Floating and adherent H1703 cells were collected, centrifuged, incubated with pHrodo red (1 ng/μL) for 15 minutes at room temperature in PBS, washed, and then plated in a non-adherent 96 well plate. Human CER21+ B cells and H1703 cells were co-cultured at a target cell to effector cell ratio of 5:1 at 37° C. for 3 hours. B cells transduced with truncated EGFR were used as control. The plate was then imaged using a 20× objective, Keyence BZ-X710 microscope (FIG. 21). Cells undergoing apoptosis show increasing Red fluorescence as the intracellular pH drops in the earliest stages of apoptosis (FIG. 21, top row). Adjacent cells not induced by Paclitaxel treatment remain only dimly fluorescent. Apoptotic measurements in the presence of CER21+ B cells were quantified as the Area under the Curve of red fluorescent objects and outlined in blue using automated software (FIG. 21, bottom row; FIG. 22), for each high powered field. White arrows show apoptosis events. CER21+ B cells were found to enhance killing of target cells at sub-therapeutic dose of chemotherapy.

### Enhanced Proliferation Capacity of CER19+ and CER21+ B Cells

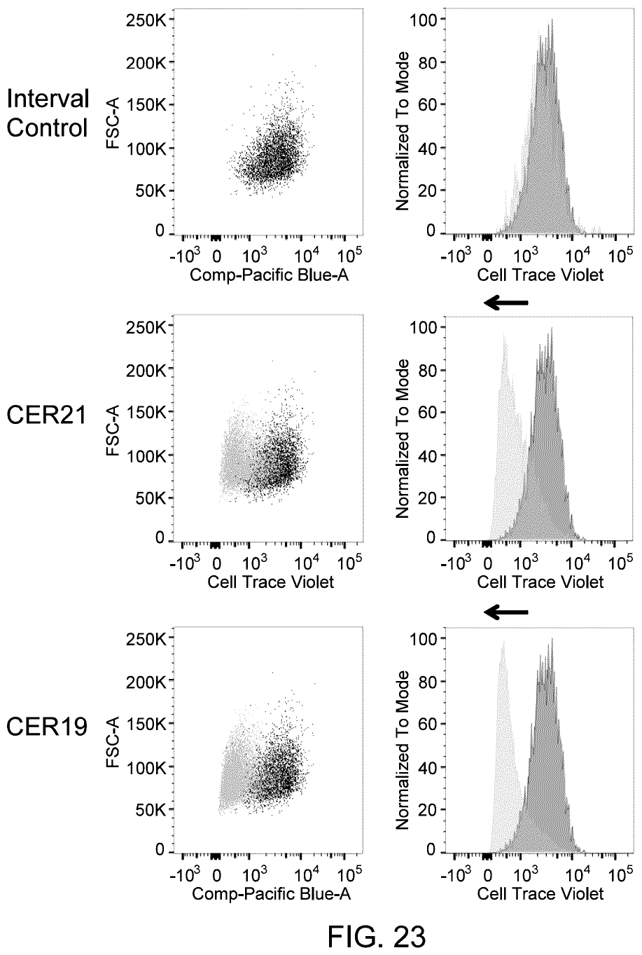

The proliferation capacity of CER19+ and CER21+ human primary B cells was evaluated by co-culture with paclitaxel (30 μM) treated Jurkat lymphoma cells. CER-transduced B cells were labeled with CELLTRACE Violet and then co-cultured with paclitaxel treated Jurkat cells a target cell to effector cell ratio of 5:1 at 37° C. in the absence of exogenous cytokines for 5 days. Proliferation of CER19+ or CER21+ B cells was assessed by flow cytometry by measuring dilution of CELLTRACE Violet (FIG. 23). Both CER19+ and CER21+ B cells increased approximately 10-fold after 5 days of co-culture in the absence of exogenous cytokines. In contrast, cells transduced with a control vector displayed no increase in cell numbers.

### Enhanced Activation State of CER21+ B Cells

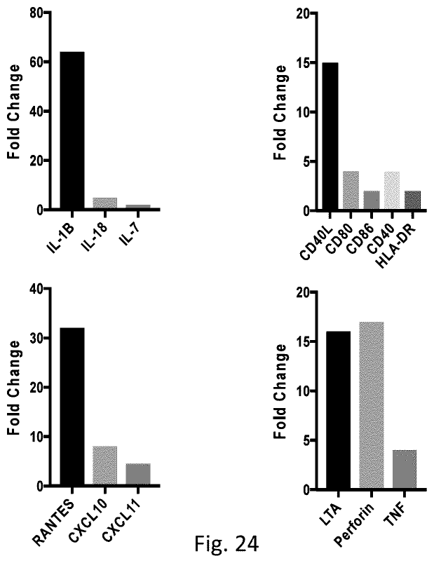

To evaluate the activation state of CER21+ human primary B cells, gene expression profiles were examined from transduced B cell populations. In agreement with previous reports linking activation of the TLR family to expression of pro-inflammatory IL-1 cytokines (e.g., IL-1B and IL-18) and elevation in co-stimulatory molecules (e.g., CD80, CD86), CER21 also promoted B cell activation molecules and survival factors (e.g., CD40, CD40L), lymphocyte chemo-attractants (RANTES, CXCL10, and CXCL11), and expresses molecules involved in lymph node tissue remodeling that facilitate the development of tumor-specific, adaptive immune responses, such as LTα and TNFα (see, FIG. 24; bar graphs show fold change in B cell mRNA levels compared to transduced control B cells).

### Example 4

### Construction of FMC63 scFv-TLR4 CER “CER43” and FMC 63 scFv-IgG4-TLR4 CER “CER44”

An anti-CD19 single chain fragment variable (scFv) (encoding amino acid sequence of SEQ ID NO: 109) derived from the FMC63 mouse IgG2a mouse monoclonal antibody and comprising a GM-CSF derived signal peptide (amino acids 1-22 of SEQ ID NO:109) was fused to a TLR4 juxtamembrane domain (SEQ ID NO:17), a TLR4 transmembrane domain (amino acid sequence of SEQ ID NO:34) and to an intracellular signaling domain of TLR4 (SEQ ID NO:51) to create a chimeric engulfment receptor “CER43” (FMC63 scFv-TLR4 CER having an amino acid sequence of SEQ ID NO:122). The TLR4 signaling domain transduces a signal for engulfment, and FMC63 scFv binds to CD19. The FMC63 scFv-TLR4 (CER43) chimeric engulfment receptor nucleotide sequence was then inserted into the pLenti lentiviral vector along with truncated EGFR (EGFRt) as a transduction marker, separated by T2A sequence. Murine Ba/F3 B-cells were transduced with pLenti vector expressing FMC63 scFv-TLR4 (CER43) and EGFRt, expanded, sorted by FACs, and used for in vitro studies as described in Example 1.

An anti-CD19 single chain fragment variable (scFv) (encoding amino acid sequence of SEQ ID NO:109) derived from the FMC63 mouse IgG2a mouse monoclonal antibody and comprising a GM-CSF derived signal peptide (amino acids 1-22 of SEQ ID NO: 109) was fused to a modified IgG4 hinge extracellular spacer domain comprising (SEQ ID NO:16), a TLR4 transmembrane domain (amino acid sequence of SEQ ID NO:34), and an intracellular signaling domain of TLR4 (SEQ ID NO:51) to create a chimeric engulfment receptor “CER44” (FMC63 scFv-IgG4-TLR4 CER having an amino acid sequence of SEQ ID NO:123). The TLR4 signaling domain transduces a signal for engulfment, and FMC63 scFv binds to CD19. The FMC63 scFv-IgG4-TLR4 (CER44) chimeric engulfment receptor nucleotide sequence was then inserted into the pLenti lentiviral vector along with truncated EGFR as a transduction marker, separated by T2A sequence. Murine Ba/F3 B-cells were transduced with pLenti vector expressing FMC63 scFv-IgG4-TLR4 (CER44) and EGFRt, expanded, sorted by FACs, and used for in vitro studies as described in Example 1.

### Phagocytic Activity Against Human Lymphoma Cell Line

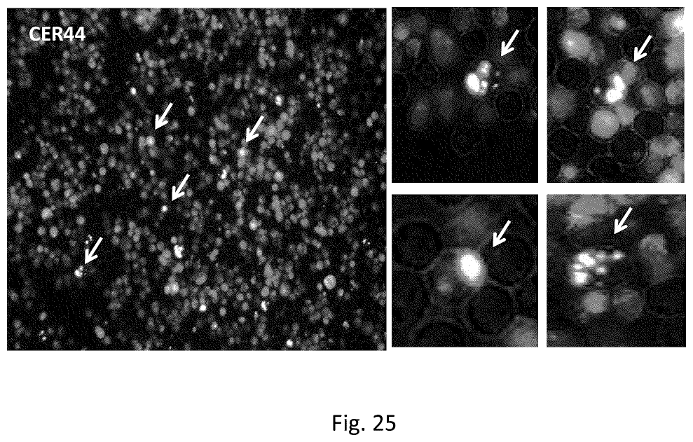

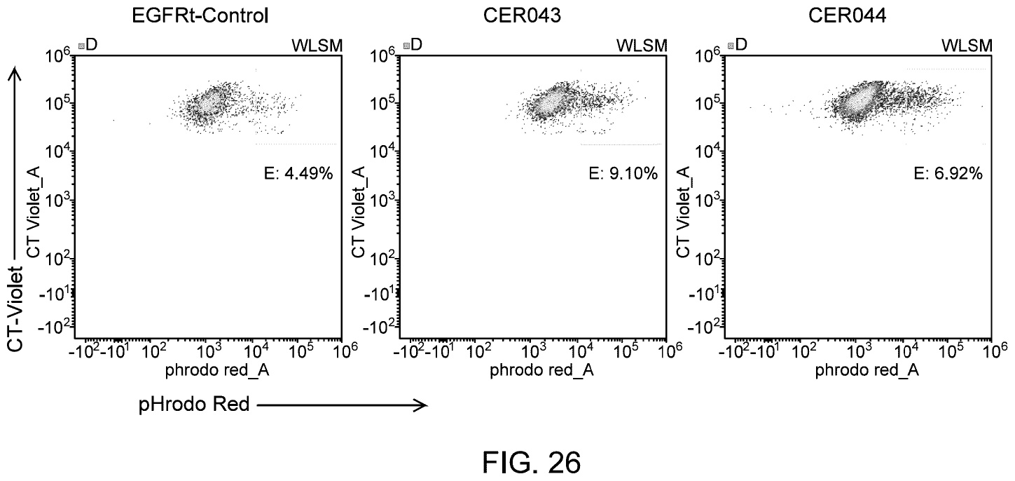

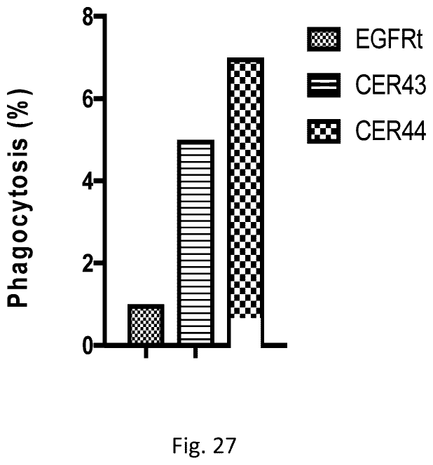

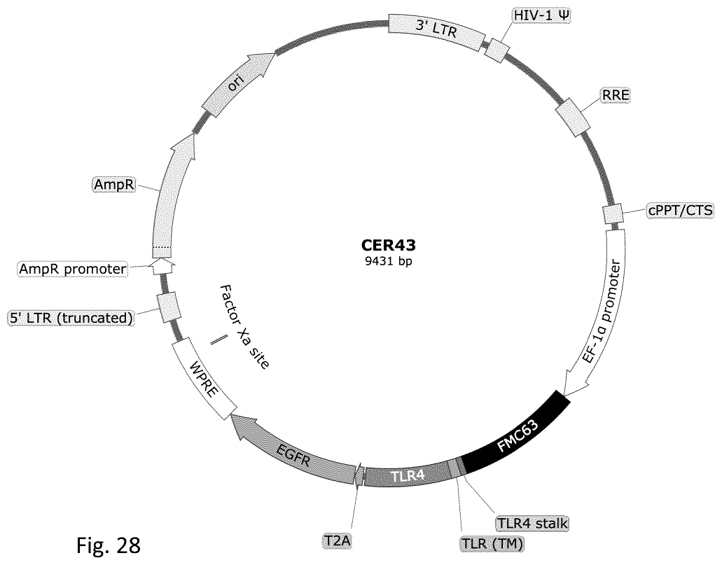

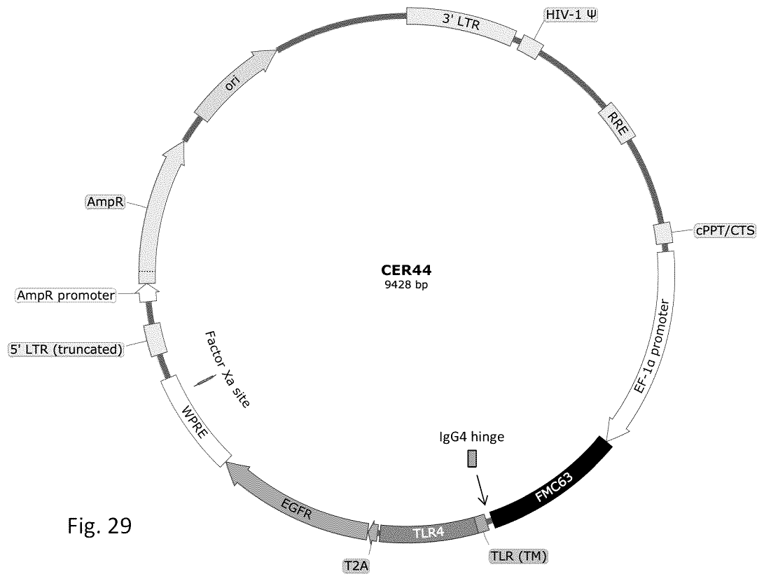

Raji human Burkitt B-cell lymphoma cells were labeled with 1 μM of pHrodo Red dye and used as target cells for phagocytosis assays. CER43+ or CER44+ modified Ba/F3 cells were stained with CELLTRACE Violet as described in Example 1. Co-culture experiments with genetically modified CD19-targeted CER43+ or CER44+ Ba/F3 cells and CD19+ Raji cells were carried out as described in Example 1. Ba/F3 cells transduced with truncated EGFR were used as control. CER43+ or CER44+ Ba/F3 cells and Raji cells were co-cultured at a target cell to effector cell ratio of 5:1 at 37° C. for 3 hours. Phagocytic events were quantified by fluorescent microscopy (KEYENCE BZ-X710 fluorescence microscope, 20× objective). FIG. 25 shows engulfment of Raji cells by CD19 specific CER44 expressing Ba/F3 cells (white arrows indicate engulfment events). The frequency of phagocytosis was quantified as the cell population staining double positive for pHrodo Red and CELLTRACE Violet as detected by FACS. FIG. 26 shows FACS plots showing the double positive cell population for CER43+ Ba/F3 cells (9.10%), CER44+ Ba/F3 cells (6.92%), or control EGFRt+ Ba/F3 cells (4.49%) co-cultured with Raji cells. Frequency of phagocytosis for CER43+, CER44+, or control EGFRt+ Ba/F3 cells co-cultured with Raji cells is also shown in the bar graph of FIG. 27. Ba/F3 cells transduced with lentivirus vector expressing CD19 specific CER43 or CER44 exhibited enhanced phagocytic uptake of Raji lymphoma cells.

### Example 5

### Construction of CERs, TCRs, and Modified T Cells for Cellular Immunotherapy Combinations

A polynucleotide comprising the extracellular domain of the phosphatidylserine binding protein Tim4 and Tim4 transmembrane domain was fused to the intracellular signaling domain of TLR4 to create chimeric engulfment receptor “CER5” encoding an amino acid sequence of SEQ ID NO:81.

A polynucleotide comprising the extracellular domain of the phosphatidylserine binding protein Tim4 and Tim4 transmembrane domain was fused to the intracellular signaling domain of TLR3 to create chimeric engulfment receptor “CER17” encoding an amino acid sequence of SEQ ID NO:84.

A polynucleotide comprising the extracellular domain of the phosphatidylserine binding protein Tim4 and Tim4 transmembrane domain was fused to the intracellular signaling domain of TLR5 to create chimeric engulfment receptor “CER19” encoding an amino acid sequence of SEQ ID NO:86.

A polynucleotide comprising the extracellular domain of the phosphatidylserine binding protein Tim4 was Tim4 transmembrane domain and TLR8 intracellular signaling domain to create chimeric engulfment receptor “CER21” encoding an amino acid sequence of SEQ ID NO:88.

A polynucleotide comprising the extracellular domain of the phosphatidylserine binding protein Tim4 and Tim4 transmembrane domain was fused to the intracellular signaling domain of TLR9 to create chimeric engulfment receptor “CER23” encoding an amino acid sequence of SEQ ID NO:90.

A polynucleotide comprising the extracellular domain of the phosphatidylserine binding protein Tim4 and Tim4 transmembrane domain was fused to the intracellular signaling domain of TLR1 to create chimeric engulfment receptor “CER26” encoding an amino acid sequence of SEQ ID NO:92.

A polynucleotide comprising the extracellular domain of the phosphatidylserine binding protein Tim4 and Tim4 transmembrane domain was fused to the intracellular signaling domain of TLR2 to create chimeric engulfment receptor “CER27” encoding an amino acid sequence of SEQ ID NO:93.

A polynucleotide comprising the extracellular domain of the phosphatidylserine binding protein Tim4 and Tim4 transmembrane domain was fused to the intracellular signaling domain of TLR8 and a truncated intracellular signaling domain of CD79b to create chimeric engulfment receptor “CER103B” encoding an amino acid sequence of SEQ ID NO: 132.

A polynucleotide comprising the extracellular domain of the phosphatidylserine binding protein Tim4 and Tim4 transmembrane domain was fused to the intracellular signaling domain of TLR8 and the intracellular signaling domain of DAP12 to create chimeric engulfment receptor “CER104” encoding an amino acid sequence of SEQ ID NO: 133.

A polynucleotide comprising the extracellular domain of the phosphatidylserine binding protein Tim4 and Tim4 transmembrane domain was fused to the intracellular signaling domain of TLR8 and the intracellular signaling domain of BAFF-R to create chimeric engulfment receptor “CER105” encoding an amino acid sequence of SEQ ID NO: 134.

A polynucleotide comprising the extracellular domain of the phosphatidylserine binding protein Tim4 and Tim4 transmembrane domain was fused to the intracellular signaling domain of NFAM1 and the intracellular signaling domain of TLR8 to create chimeric engulfment receptor “CER106” encoding an amino acid sequence of SEQ ID NO: 135.

A polynucleotide comprising the extracellular domain of the phosphatidylserine binding protein Tim4 and Tim4 transmembrane domain was fused to the intracellular signaling domain of Traf6 and the intracellular signaling domain of TLR8 to create chimeric engulfment receptor “CER116” encoding an amino acid sequence of SEQ ID NO:143.

A polynucleotide encoding a TCRβ chain and a polynucleotide encoding a TCRα of a HPV16 E7 specific TCR (see, PCT Publication No. WO2015/184228) were fused using a sequence encoding a P2A self-cleaving peptide there between. The TCR Vα domain comprises an amino acid sequence of SEQ ID NO:162, and the TCR Vβ region comprises an amino acid sequence of SEQ ID NO:160. The Cα domain comprises a cysteine substitution and LVL substitutions at positions 12, 14, and 15 and comprises an amino acid sequence of SEQ ID NO: 163. The Cβ also comprises a cysteine substitution and comprises an amino acid sequence of SEQ ID NO:161. The encoded HPV16 E7 specific TCR comprises an amino acid sequence of SEQ ID NO: 158.

A selected CER polynucleotide and the HPV16 E7 TCR polynucleotide were each inserted into a pLenti lentiviral vector. Peripheral blood was collected by venipuncture from a human donor, and human peripheral blood mononuclear cells (PBMCs) were isolated by density gradient centrifugation using lymphocyte separation media. CD8+ or CD4+ T cells were enriched from PBMCs using a commercially available isolation kit and activated with anti-CD3 and anti-CD28 in Complete Cell Growth Media. 50 μl of viral vector expressing the HPV16 E7 TCR were diluted in 0.5 ml Complete Cell Growth Media and added to the CD8+ T cells. 50 μl of viral vector expressing the a selected CER were diluted in 0.5 ml Complete Cell Growth Media and added to the CD4+ T cells. The transduced T cells were then centrifuged at 270×g rpm for 1 hour in a 32° C. pre-warmed centrifuge. The T cells were incubated for 24 hours at 37° C. T cells were expanded for another 72 hours in Complete Cell Growth Media, de-beaded, and allowed to expand ×5 days prior to being utilized for functional assays. Transduced CD4 and CD8 T cells were combined at a 1:1 ratio for functional assays.

### Combinations of CD8 T Cell-TCR+CD4 T Cell-CER Exhibit Enhanced Antigen Specific Cytolytic Activity and Phagocytic Activity

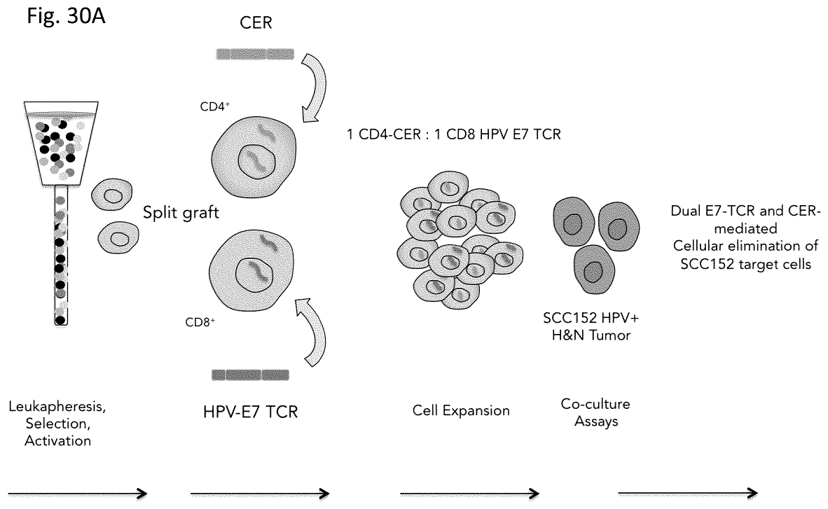

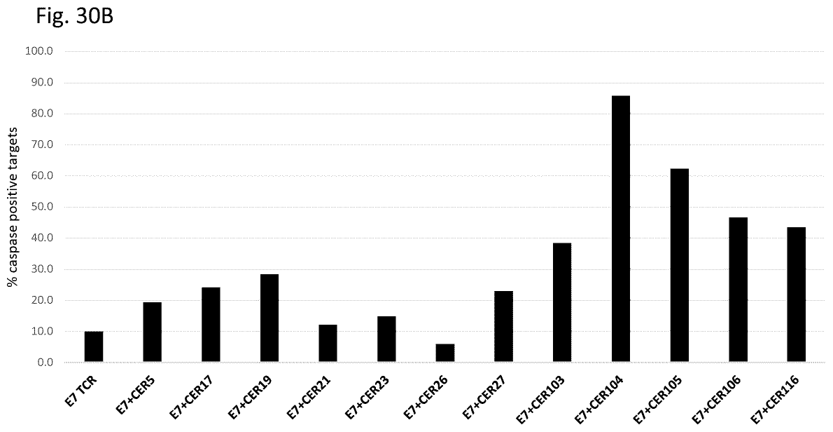

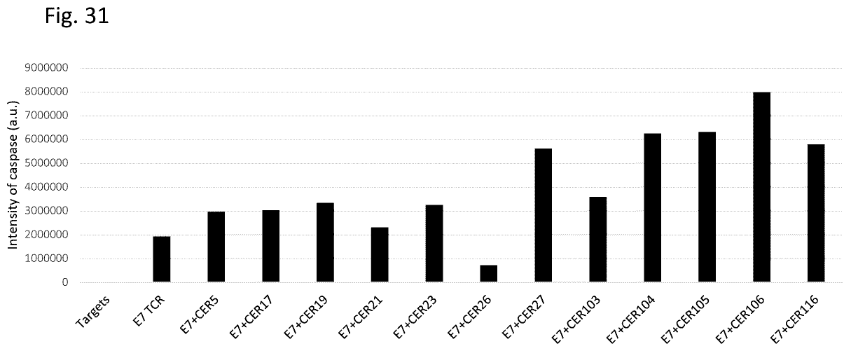

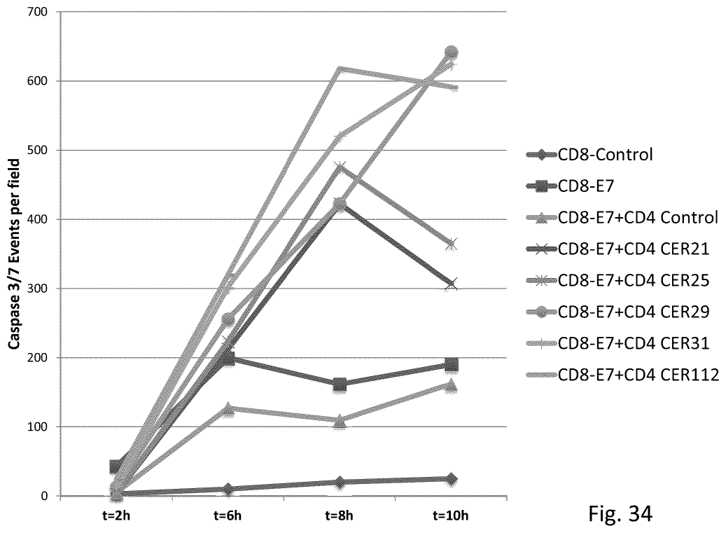

Dual HPV16 E7 TCR and CER-mediated elimination of target SCC152 cells was detected using cytotoxicity and phagocytosis assays (see, FIG. 30A). SCC152 cells are HPV+ cells from a squamous cell carcinoma of the hypopharynx. Cytotoxic activity of CD8+ T cells transduced with HPV16 E7 specific TCR was detected using a caspase 3/7 apoptosis reagent (IncuCyte®) that couples the activated caspase 3/7 recognition motif with a red reagent that fluoresces upon cleavage. The fluorescent signal was measured using fluorescent microscopy. HPV16 E7 TCR transduced CD8+ T cells and selected CER transduced CD4+ T cells were mixed at a 1:1 ratio and co-cultured with HPV16 E7+ head and neck squamous cell carcinoma cells (SCC152) at a 1:1 ratio, and caspase 3/7 apoptosis reagent was added to the co-culture. Cytotoxic activity was measured over time by measuring fluorescence. Control samples were CD8 T cells transduced with HPV16 E7 TCR alone. As shown in the graphs of FIGS. 30B, 31, and 34, and fluorescent micrographs (data not shown), addition of CD4+ T cells transduced with most of the CERs tested to CD8 T cells transduced with the HPV16 E7 TCR enhanced cytolytic activity over mono-treatment with CD8 T cells transduced with HPV16 E7 TCR.

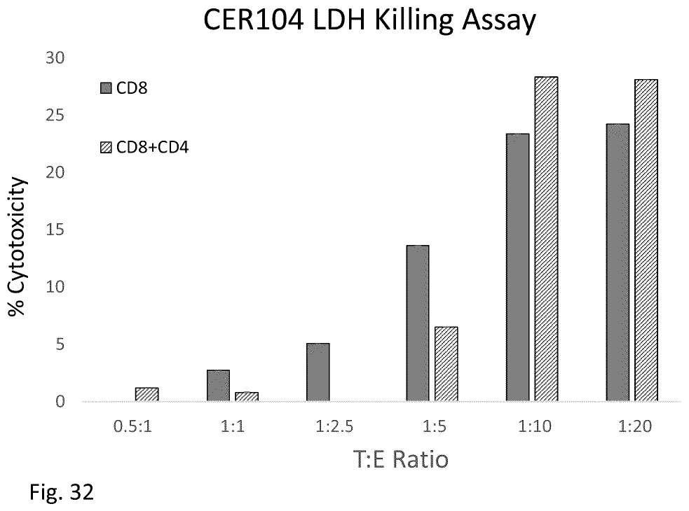

The enhanced cytolytic activity of CD4 T cell transduced with CER104+CD8 T cells transduced with HPV16 E7 TCR was observed when measured using a lactate dehydrogenase (LDH) cytoxicity assay (see, FIG. 32). LDH is a cytosolic enzyme that is released by a cell into cell culture media when the plasma membrane is damaged. Thus, LDH's presence in culture medium is a marker for cell death. LDH assays are capable of detecting low level damage to cell membrane which cannot be detected using other methods. LDH may be detected using colorimetric or fluorometric methods.

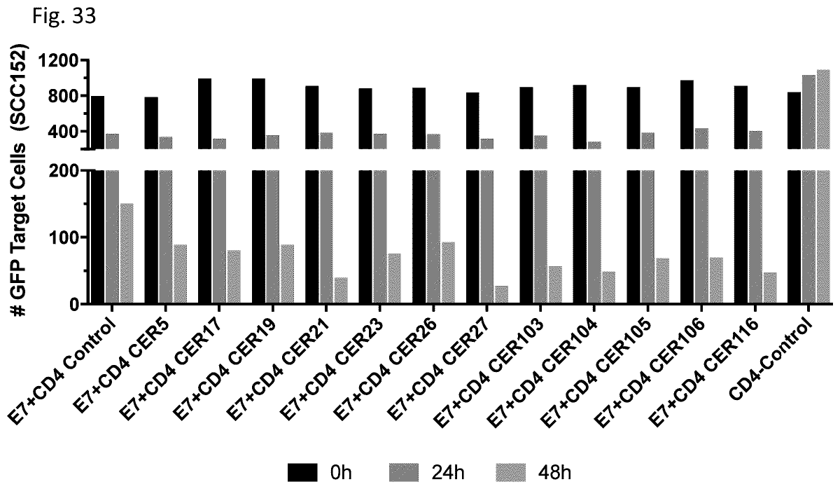

Elimination of target SCC152 cells was also detected by quantifying green fluorescent protein expression by SCC152 cells over time (0 hr, 24 hr, 48 hr) during co-incubation with CD8+ T cells transduced with HPV16 E7 specific TCR+CD4 T cells transduced with selected CER (see, FIG. 33). By 48 hrs, all of the CD4 T cell/CER+CD8 T cell/HPV16 E7 TCR combination co-cultures showed enhanced elimination of SCC152 cells compared to controls. Time lapse imaging of co-culture experiments similarly showed enhanced elimination of SCC152 cells by CD4 T cell/CER+CD8 T cell/HPV16 E7 TCR combination co-cultures compared to controls (data not shown).

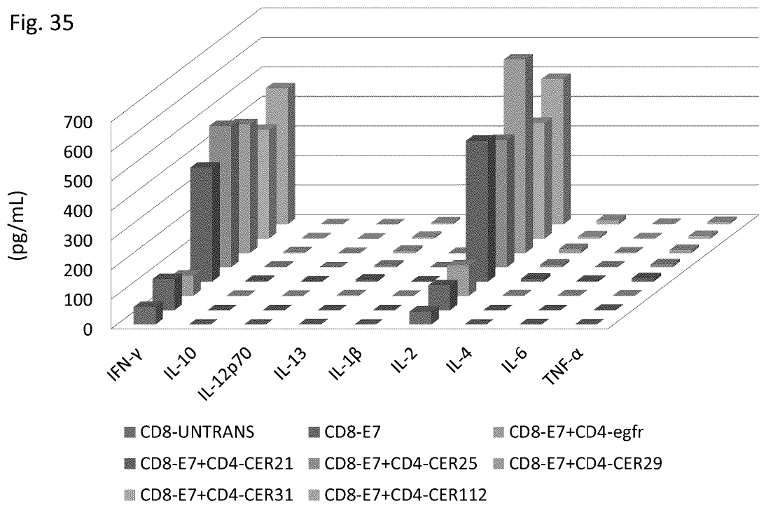

Cytokine response of co-culture experiments was measured by sampling the cellular supernatants using a mesoscale multi-array cytokine plate. The following cytokines were measured: IFNγ, IL-2, TNFα, IL-4, IL-6, IL-12b, IL-13, IL-1b, and IL-10. Enhanced cytokine production indicative of activated profile (e.g., IFNγ, IL-2) were elicited in co-cultures with CD4 T cell/CER+CD8 T cell/HPV16 E7 TCR combinations compared to controls (see, FIG. 35).

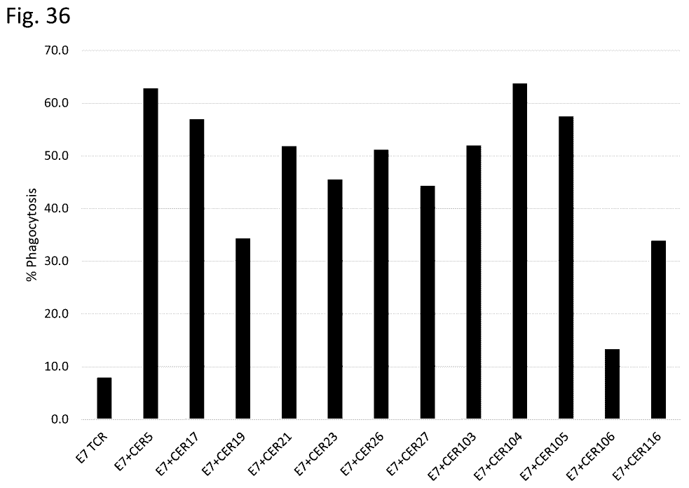

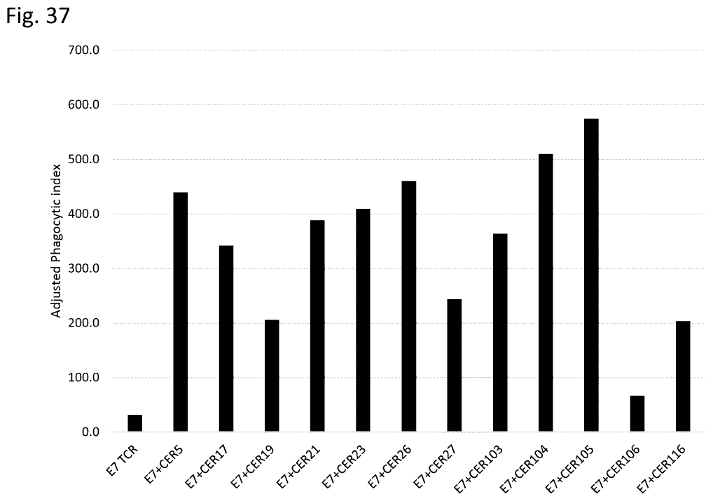

Phagocytic activity of CD4 T cell/CER+CD8 T cell/HPV16 E7 TCR combinations co-cultured with SCC152 cells was visualized and quantified using KEYENCE BZ-X710 fluorescence microscope, 20× objective and hybrid capture software. FIGS. 36-37 show that CD4+ T cells transduced with various CERs used in co-culture with CD8 T cells/HPV E7 TCR exhibited enhanced engulfment of SCC152 target cells over co-culture with control CD8 T cell/HPV16 E7 TCR alone.

### Example 6

### Construction of Tandem Expression Cassettes

A polynucleotide comprising the extracellular domain of the phosphatidylserine binding protein Tim4 and Tim4 transmembrane domain was fused to the intracellular signaling domain of TLR4 to create chimeric engulfment receptor “CER5” encoding an amino acid sequence of SEQ ID NO:81. A polynucleotide comprising the extracellular domain of the phosphatidylserine binding protein Tim4 and Tim4 transmembrane domain was fused to the intracellular signaling domain of TLR5 to create chimeric engulfment receptor “CER19” encoding an amino acid sequence of SEQ ID NO:98. A polynucleotide comprising the extracellular domain of the phosphatidylserine binding protein Tim4 was Tim4 transmembrane domain and TLR8 intracellular signaling domain to create chimeric engulfment receptor “CER21” encoding an amino acid sequence of SEQ ID NO:86. A polynucleotide comprising the extracellular domain of the phosphatidylserine binding protein Tim4 and Tim4 transmembrane domain was fused to the intracellular signaling domain of NFAM1 to create chimeric engulfment receptor “CER25” encoding an amino acid sequence of SEQ ID NO: 159. A polynucleotide comprising the extracellular domain of the phosphatidylserine binding protein Tim4 and Tim4 transmembrane domain was fused to the intracellular signaling domain of TLR2 to create chimeric engulfment receptor “CER27” encoding an amino acid sequence of SEQ ID NO:93. A polynucleotide comprising the extracellular domain of the phosphatidylserine binding protein Tim4 and Tim4 transmembrane domain was fused to the intracellular signaling domain of Traf6 to create chimeric engulfment receptor “CER29” encoding an amino acid sequence of SEQ ID NO:102. A polynucleotide comprising the extracellular domain of the phosphatidylserine binding protein Tim4 and Tim4 transmembrane domain was fused to the intracellular signaling domain of Traf3 to create chimeric engulfment receptor “CER31” encoding an amino acid sequence of SEQ ID NO: 124.

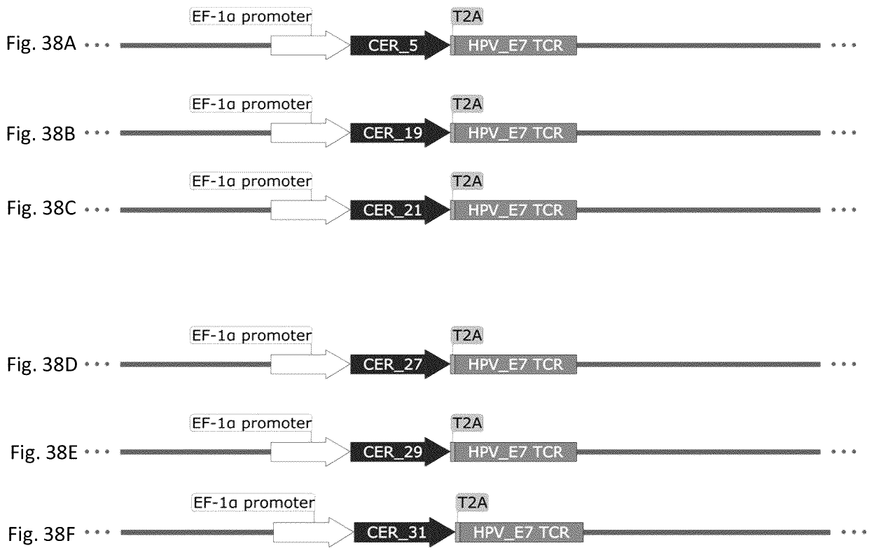

A polynucleotide encoding a TCRβ chain and a polynucleotide encoding a TCRα of an HPV16 E7 specific TCR (see, PCT Publication No. WO2015/184228) were fused using a sequence for P2A self-cleaving peptide there between. The TCR Vα domain comprises an amino acid sequence of SEQ ID NO: 162, and the TCR Vβ region comprises an amino acid sequence of SEQ ID NO:160. The Cα domain comprises a cysteine substitution and LVL substitutions at positions 12, 14, and 15 and comprises an amino acid sequence of SEQ ID NO:163. The Cβ also comprises a cysteine substitution and comprises an amino acid sequence of SEQ ID NO:161. The encoded HPV16 E7 specific TCR comprises an amino acid sequence of SEQ ID NO:158. Amino acid sequences for the tandem expression constructs described in this example are provided in Table 2 (see, also FIGS. 38A-38F).

TABLE 2
Exemplary Tandem Expression Cassettes

| Name | Amino Acid Sequence | SEQ ID NO:# |
|---|---|---|
| CER5_T2A_HPV16 E7 | MSKGLLLLWLVTELWWLYLTPAASEDTII | SEQ ID NO: 164 |
| TCR | GFLGQPVTLPCHYLSWSQSRNSMCWGKGS | |
| | CPNSKCNAELLRTDGTRIISRKSTKYTLLG | |
| | KVQFGEVSLTISNTNRGDSGVYCCRIEVPG | |
| | WFNDVKKNVRLELRRATTTKKPTTTTRPT | |
| | TTPYVTTTTPELLPTTVMTTSVLPTTTPPQT | |
| | LATTAFSTAVTTCPSTTPGSFSQETTKGSAF | |
| | TTESETLPASNHSQRSMMTISTDIAVLRPTG | |
| | SNPGILPSTSQLTTQKTTLTTSESLQKTTKS | |
| | HQINSRQTILIIACCVGFVLMVLLFLAFLKF | |
| | YFHLMLLAGCIKYGRGENIYDAFVIYSSQD | |
| | EDWVRNELVKNLEEGVPPFQLCLHYRDFIP | |
| | GVAIAANIIHEGFHKSRKVIVVVSQHFIQSR | |
| | WCIFEYEIAQTWQFLSSRAGIIFIVLQKVEK | |
| | TLLRQQVELYRLLSRNTYLEWEDSVLGRHI | |
| | FWRRLRKALLDGKSWNPEGTVGTGCNWQ | |
| | EATSILEGGGEGRGSLLTCGDVEENPGPMA | |
| | PGLLCWALLCLLGAGLVDAGVTQSPTHLI | |
| | KTRGQQVTLRCSPKSGHDTVSWYQQALG | |
| | QGPQFIFQYYEEEERQRGNFPDRFSGHQFP | |
| | NYSSELNVNALLLGDSALYLCASSLGWRG | |
| | GRYNEQFFGPGTRLTVLEDLRNVTPPKVSL | |
| | FEPSKAEIANKQKATLVCLARGFFPDHVEL | |
| | SWWVNGKEVHSGVCTDPQAYKESNYSYC | |
| | LSSRLRVSATFWHNPRNHFRCQVQFHGLS | |
| | EEDKWPEGSPKPVTQNISAEAWGRADCGI | |
| | TSASYQQGVLSATILYEILLGKATLYAVLV | |
| | STLVVMAMVKRKNSRAKRSGSGATNFSLL | |
| | KQAGDVEENPGPMWGVFLLYVSMKMGG | |
| | TTGQNIDQPTEMTATEGAIVQINCTYQTSG | |
| | FNGLFWYQQHAGEAPTFLSYNVLDGLEEK | |
| | GRFSSFLSRSKGYSYLLLKELQMKDSASYL | |
| | CASVDGNNRLAFGKGNQVVVIPNIQNPEP | |
| | AVYQLKDPRSQDSTLCLFTDFDSQINVPKT | |
| | MESGTFITDKCVLDMKAMDSKSNGAIAWS | |
| | NQTSFTCQDIFKETNATYPSSDVPCDATLT | |
| | EKSFETDMNLNFQNLLVIVLRILLLKVAGF | |
| | NLLMTLRLWSS | |
| CER19_T2A_HPV16 E7 | MSKGLLLLWLVTELWWLYLTPAASEDTII | SEQ ID NO: 165 |
| TCR | GFLGQPVTLPCHYLSWSQSRNSMCWGKGS | |
| | CPNSKCNAELLRTDGTRIISRKSTKYTLLG | |
| | KVQFGEVSLTISNTNRGDSGVYCCRIEVPG | |
| | WFNDVKKNVRLELRRATTTKKPTTTTRPT | |
| | TTPYVTTTTPELLPTTVMTTSVLPTTTPPQT | |
| | LATTAFSTAVTTCPSTTPGSFSQETTKGSAF | |
| | TTESETLPASNHSQRSMMTISTDIAVLRPTG | |
| | SNPGILPSTSQLTTQKTTLTTSESLQKTTKS | |
| | HQINSRQTILIIACCVGFVLMVLLFLAFLTK | |
| | FRGFCFICYKTAQRLVFKDHPQGTEPDMY | |
| | KYDAYLCFSSKDFTWVQNALLKHLDTQYS | |
| | DQNRFNLCFEERDFVPGENRIANIQDAIWN | |
| | SRKIVCLVSRHFLRDGWCLEAFSYAQGRC | |
| | LSDLNSALIMVVVGSLSQYQLMKHQSIRGF | |
| | VQKQQYLRWPEDFQDVGWFLHKLSQQIL | |
| | KKEKEKKKDNNIPLQTVATISLEGGGEGRG | |
| | SLLTCGDVEENPGPMAPGLLCWALLCLLG | |
| | AGLVDAGVTQSPTHLIKTRGQQVTLRCSP | |
| | KSGHDTVSWYQQALGQGPQFIFQYYEEEE | |
| | RQRGNFPDRFSGHQFPNYSSELNVNALLLG | |
| | DSALYLCASSLGWRGGRYNEQFFGPGTRL | |
| | TVLEDLRNVTPPKVSLFEPSKAEIANKQKA | |
| | TLVCLARGFFPDHVELSWWVNGKEVHSG | |
| | VCTDPQAYKESNYSYCLSSRLRVSATFWH | |
| | NPRNHFRCQVQFHGLSEEDKWPEGSPKPV | |
| | TQNISAEAWGRADCGITSASYQQGVLSATI | |
| | LYEILLGKATLYAVLVSTLVVMAMVKRK | |
| | NSRAKRSGSGATNFSLLKQAGDVEENPGP | |
| | MWGVFLLYVSMKMGGTTGQNIDQPTEMT | |
| | ATEGAIVQINCTYQTSGFNGLFWYQQHAG | |
| | EAPTFLSYNVLDGLEEKGRFSSFLSRSKGY | |
| | SYLLLKELQMKDSASYLCASVDGNNRLAF | |
| | GKGNQVVVIPNIQNPEPAVYQLKDPRSQDS | |
| | TLCLFTDFDSQINVPKTMESGTFITDKCVL | |
| | DMKAMDSKSNGAIAWSNQTSFTCQDIFKE | |
| | TNATYPSSDVPCDATLTEKSFETDMNLNFQ | |
| | NLLVIVLRILLLKVAGFNLLMTLRLWSS | |
| CER21_T2A_HPV16 E7 | MSKGLLLLWLVTELWWLYLTPAASEDTII | SEQ ID NO: 166 |
| TCR | GFLGQPVTLPCHYLSWSQSRNSMCWGKGS | |
| | CPNSKCNAELLRTDGTRIISRKSTKYTLLG | |
| | KVQFGEVSLTISNTNRGDSGVYCCRIEVPG | |
| | WFNDVKKNVRLELRRATTTKKPTTTTRPT | |
| | TTPYVTTTTPELLPTTVMTTSVLPTTTPPQT | |
| | LATTAFSTAVTTCPSTTPGSFSQETTKGSAF | |
| | TTESETLPASNHSQRSMMTISTDIAVLRPTG | |
| | SNPGILPSTSQLTTQKTTLTTSESLQKTTKS | |
| | HQINSRQTILIIACCVGFVLMVLLFLAFLHH | |
| | LFYWDVWFIYNVCLAKVKGYRSLSTSQTF | |
| | YDAYISYDTKDASVTDWVINELRYHLEES | |
| | RDKNVLLCLEERDWDPGLAIIDNLMQSINQ | |
| | SKKTVFVLTKKYAKSWNFKTAFYLALQRL | |
| | MDENMDVIIFILLEPVLQHSQYLRLRQRIC | |
| | KSSILQWPDNPKAEGLFWQTLRNVVLTEN | |
| | DSRYNNMYVDSIKQYLEGGGEGRGSLLTC | |
| | GDVEENPGPMAPGLLCWALLCLLGAGLV | |
| | DAGVTQSPTHLIKTRGQQVTLRCSPKSGHD | |
| | TVSWYQQALGQGPQFIFQYYEEEERQRGN | |
| | FPDRFSGHQFPNYSSELNVNALLLGDSALY | |
| | LCASSLGWRGGRYNEQFFGPGTRLTVLED | |
| | LRNVTPPKVSLFEPSKAEIANKQKATLVCL | |
| | ARGFFPDHVELSWWVNGKEVHSGVCTDP | |
| | QAYKESNYSYCLSSRLRVSATFWHNPRNH | |
| | FRCQVQFHGLSEEDKWPEGSPKPVTQNISA | |
| | EAWGRADCGITSASYQQGVLSATILYEILL | |
| | GKATLYAVLVSTLVVMAMVKRKNSRAKR | |
| | SGSGATNFSLLKQAGDVEENPGPMWGVFL | |
| | LYVSMKMGGTTGQNIDQPTEMTATEGAIV | |
| | QINCTYQTSGFNGLFWYQQHAGEAPTFLS | |
| | YNVLDGLEEKGRFSSFLSRSKGYSYLLLKE | |
| | LQMKDSASYLCASVDGNNRLAFGKGNQV | |
| | VVIPNIQNPEPAVYQLKDPRSQDSTLCLFT | |
| | DFDSQINVPKTMESGTFITDKCVLDMKAM | |
| | DSKSNGAIAWSNQTSFTCQDIFKETNATYP | |
| | SSDVPCDATLTEKSFETDMNLNFQNLLVIV | |
| | LRILLLKVAGFNLLMTLRLWSS | |
| CER25_T2A_HPV16 E7 | MSKGLLLLWLVTELWWLYLTPAASEDTII | SEQ ID NO: 167 |
| TCR | GFLGQPVTLPCHYLSWSQSRNSMCWGKGS | |
| | CPNSKCNAELLRTDGTRIISRKSTKYTLLG | |
| | KVQFGEVSLTISNTNRGDSGVYCCRIEVPG | |
| | WFNDVKKNVRLELRRATTTKKPTTTTRPT | |
| | TTPYVTTTTPELLPTTVMTTSVLPTTTPPQT | |
| | LATTAFSTAVTTCPSTTPGSFSQETTKGSAF | |
| | TTESETLPASNHSQRSMMTISTDIAVLRPTG | |
| | SNPGILPSTSQLTTQKTTLTTSESLQKTTKS | |
| | HQINSRQTILIIACCVGFVLMVLLFLAFLLW | |
| | NKKRMRGPGKDPTRKCPDPRSASSPKQHP | |
| | SESVYTALQRRETEVYACIENEDGSSPTAK | |
| | QSPLSQERPHRFEDDGELNLVYENLLEGGG | |
| | EGRGSLLTCGDVEENPGPMAPGLLCWALL | |
| | CLLGAGLVDAGVTQSPTHLIKTRGQQVTL | |
| | RCSPKSGHDTVSWYQQALGQGPQFIFQYY | |
| | EEEERQRGNFPDRFSGHQFPNYSSELNVNA | |
| | LLLGDSALYLCASSLGWRGGRYNEQFFGP | |
| | GTRLTVLEDLRNVTPPKVSLFEPSKAEIAN | |
| | KQKATLVCLARGFFPDHVELSWWVNGKE | |
| | VHSGVCTDPQAYKESNYSYCLSSRLRVSA | |
| | TFWHNPRNHFRCQVQFHGLSEEDKWPEGS | |
| | PKPVTQNISAEAWGRADCGITSASYQQGV | |
| | LSATILYEILLGKATLYAVLVSTLVVMAM | |
| | VKRKNSRAKRSGSGATNFSLLKQAGDVEE | |
| | NPGPMWGVFLLYVSMKMGGTTGQNIDQP | |
| | TEMTATEGAIVQINCTYQTSGFNGLFWYQ | |
| | QHAGEAPTFLSYNVLDGLEEKGRFSSFLSR | |
| | SKGYSYLLLKELQMKDSASYLCASVDGNN | |
| | RLAFGKGNQVVVIPNIQNPEPAVYQLKDPR | |
| | SQDSTLCLFTDFDSQINVPKTMESGTFITDK | |
| | CVLDMKAMDSKSNGAIAWSNQTSFTCQDI | |
| | FKETNATYPSSDVPCDATLTEKSFETDMNL | |
| | NFQNLLVIVLRILLLKVAGFNLLMTLRLWS | |
| | S | |
| CER27_T2A_HPV16 E7 | MSKGLLLLWLVTELWWLYLTPAASEDTII | SEQ ID NO: 168 |
| TCR | GFLGQPVTLPCHYLSWSQSRNSMCWGKGS | |
| | CPNSKCNAELLRTDGTRIISRKSTKYTLLG | |
| | KVQFGEVSLTISNTNRGDSGVYCCRIEVPG | |
| | WFNDVKKNVRLELRRATTTKKPTTTTRPT | |
| | TTPYVTTTTPELLPTTVMTTSVLPTTTPPQT | |
| | LATTAFSTAVTTCPSTTPGSFSQETTKGSAF | |
| | TTESETLPASNHSQRSMMTISTDIAVLRPTG | |
| | SNPGILPSTSQLTTQKTTLTTSESLQKTTKS | |
| | HQINSRQTILIIACCVGFVLMVLLFLAFLHR | |
| | FHGLWYMKMMWAWLQAKRKPRKAPSRN | |
| | ICYDAFVSYSERDAYWVENLMVQELENFN | |
| | PPFKLCLHKRDFIPGKWIIDNIIDSIEKSHKT | |
| | VFVLSENFVKSEWCKYELDFSHFRLFDEN | |
| | NDAAILILLEPIEKKAIPQRFCKLRKIMNTK | |
| | TYLEWPMDEAQREGFWVNLRAAIKSLEG | |
| | GGEGRGSLLTCGDVEENPGPMAPGLLCWA | |
| | LLCLLGAGLVDAGVTQSPTHLIKTRGQQV | |
| | TLRCSPKSGHDTVSWYQQALGQGPQFIFQ | |
| | YYEEEERQRGNFPDRFSGHQFPNYSSELNV | |
| | NALLLGDSALYLCASSLGWRGGRYNEQFF | |
| | GPGTRLTVLEDLRNVTPPKVSLFEPSKAEIA | |
| | NKQKATLVCLARGFFPDHVELSWWVNGK | |
| | EVHSGVCTDPQAYKESNYSYCLSSRLRVS | |
| | ATFWHNPRNHFRCQVQFHGLSEEDKWPE | |
| | GSPKPVTQNISAEAWGRADCGITSASYQQ | |
| | GVLSATILYEILLGKATLYAVLVSTLVVMA | |
| | MVKRKNSRAKRSGSGATNFSLLKQAGDV | |
| | EENPGPMWGVFLLYVSMKMGGTTGQNID | |
| | QPTEMTATEGAIVQINCTYQTSGFNGLFW | |
| | YQQHAGEAPTFLSYNVLDGLEEKGRFSSFL | |
| | SRSKGYSYLLLKELQMKDSASYLCASVDG | |
| | NNRLAFGKGNQVVVIPNIQNPEPAVYQLK | |
| | DPRSQDSTLCLFTDFDSQINVPKTMESGTFI | |
| | TDKCVLDMKAMDSKSNGAIAWSNQTSFT | |
| | CQDIFKETNATYPSSDVPCDATLTEKSFET | |
| | DMNLNFQNLLVIVLRILLLKVAGFNLLMTL | |
| | RLWSS | |
| CER29_T2A_HPV16 E7 | MSKGLLLLWLVTELWWLYLTPAASEDTII | SEQ ID NO: 169 |
| TCR | GFLGQPVTLPCHYLSWSQSRNSMCWGKGS | |
| | CPNSKCNAELLRTDGTRIISRKSTKYTLLG | |
| | KVQFGEVSLTISNTNRGDSGVYCCRIEVPG | |
| | WFNDVKKNVRLELRRATTTKKPTTTTRPT | |
| | TTPYVTTTTPELLPTTVMTTSVLPTTTPPQT | |
| | LATTAFSTAVTTCPSTTPGSFSQETTKGSAF | |
| | TTESETLPASNHSQRSMMTISTDIAVLRPTG | |
| | SNPGILPSTSQLTTQKTTLTTSESLQKTTKS | |
| | HQINSRQTILIIACCVGFVLMVLLFLAFLMS | |
| | LLNCENSCGSSQSESDCCVAMASSCSAVT | |
| | KDDSVGGTASTGNLSSSFMEEIQGYDVEFD | |
| | PPLESKYECPICLMALREAVQTPCGHRFCK | |
| | ACIIKSIRDAGHKCPVDNEILLENQLFPDNF | |
| | AKREILSLMVKCPNEGCLHKMELRHLEDH | |
| | QAHCEFALMDCPQCQRPFQKFHINIHILKD | |
| | CPRRQVSCDNCAASMAFEDKEIHDQNCPL | |
| | ANVICEYCNTILIREQMPNHYDLDCPTAPIP | |
| | CTFSTFGCHEKMQRNHLARHLQENTQSHM | |
| | RMLALEGGGEGRGSLLTCGDVEENPGPMA | |
| | PGLLCWALLCLLGAGLVDAGVTQSPTHLI | |
| | KTRGQQVTLRCSPKSGHDTVSWYQQALG | |
| | QGPQFIFQYYEEEERQRGNFPDRFSGHQFP | |
| | NYSSELNVNALLLGDSALYLCASSLGWRG | |
| | GRYNEQFFGPGTRLTVLEDLRNVTPPKVSL | |
| | FEPSKAEIANKQKATLVCLARGFFPDHVEL | |
| | SWWVNGKEVHSGVCTDPQAYKESNYSYC | |
| | LSSRLRVSATFWHNPRNHFRCQVQFHGLS | |
| | EEDKWPEGSPKPVTQNISAEAWGRADCGI | |
| | TSASYQQGVLSATILYEILLGKATLYAVLV | |
| | STLVVMAMVKRKNSRAKRSGSGATNFSLL | |
| | KQAGDVEENPGPMWGVFLLYVSMKMGG | |
| | TTGQNIDQPTEMTATEGAIVQINCTYQTSG | |
| | FNGLFWYQQHAGEAPTFLSYNVLDGLEEK | |
| | GRFSSFLSRSKGYSYLLLKELQMKDSASYL | |
| | CASVDGNNRLAFGKGNQVVVIPNIQNPEP | |
| | AVYQLKDPRSQDSTLCLFTDFDSQINVPKT | |
| | MESGTFITDKCVLDMKAMDSKSNGAIAWS | |
| | NQTSFTCQDIFKETNATYPSSDVPCDATLT | |
| | EKSFETDMNLNFQNLLVIVLRILLLKVAGF | |
| | NLLMTLRLWSS | |
| CER31_T2A_HPV16 E7 | MSKGLLLLWLVTELWWLYLTPAASEDTII | SEQ ID NO: 170 |
| TCR | GFLGQPVTLPCHYLSWSQSRNSMCWGKGS | |
| | CPNSKCNAELLRTDGTRIISRKSTKYTLLG | |
| | KVQFGEVSLTISNTNRGDSGVYCCRIEVPG | |
| | WFNDVKKNVRLELRRATTTKKPTTTTRPT | |
| | TTPYVTTTTPELLPTTVMTTSVLPTTTPPQT | |
| | LATTAFSTAVTTCPSTTPGSFSQETTKGSAF | |
| | TTESETLPASNHSQRSMMTISTDIAVLRPTG | |
| | SNPGILPSTSQLTTQKTTLTTSESLQKTTKS | |
| | HQINSRQTILIIACCVGFVLMVLLFLAFLME | |
| | SSKKMDSPGALQTNPPLKLHTDRSAGTPVF | |
| | VPEQGGYKEKFVKTVEDKYKCEKCHLVL | |
| | CSPKQTECGHRFCESCMAALLSSSSPKCTA | |
| | CQESIVKDKVFKDNCCKREILALQIYCRNE | |
| | SRGCAEQLMLGHLLVHLKNDCHFEELPCV | |
| | RPDCKEKVLRKDLRDHVEKACKYREATCS | |
| | HCKSQVPMIALQKHEDTDCPCVVVSCPHK | |
| | CSVQTLLRSELSAHLSECVNAPSTCSFKRY | |
| | GCVFQGTNQQIKAHEASSAVQHVNLLKE | |
| | WSNSLEKKVLEGGGEGRGSLLTCGDVEEN | |
| | PGPMAPGLLCWALLCLLGAGLVDAGVTQ | |
| | SPTHLIKTRGQQVTLRCSPKSGHDTVSWY | |
| | QQALGQGPQFIFQYYEEEERQRGNFPDRFS | |
| | GHQFPNYSSELNVNALLLGDSALYLCASSL | |
| | GWRGGRYNEQFFGPGTRLTVLEDLRNVTP | |
| | PKVSLFEPSKAEIANKQKATLVCLARGFFP | |
| | DHVELSWWVNGKEVHSGVCTDPQAYKES | |
| | NYSYCLSSRLRVSATFWHNPRNHFRCQVQ | |
| | FHGLSEEDKWPEGSPKPVTQNISAEAWGR | |
| | ADCGITSASYQQGVLSATILYEILLGKATL | |
| | YAVLVSTLVVMAMVKRKNSRAKRSGSGA | |
| | TNFSLLKQAGDVEENPGPMWGVFLLYVS | |
| | MKMGGTTGQNIDQPTEMTATEGAIVQINC | |
| | TYQTSGFNGLFWYQQHAGEAPTFLSYNVL | |
| | DGLEEKGRFSSFLSRSKGYSYLLLKELQMK | |
| | DSASYLCASVDGNNRLAFGKGNQVVVIPN | |
| | IQNPEPAVYQLKDPRSQDSTLCLFTDFDSQI | |
| | NVPKTMESGTFITDKCVLDMKAMDSKSNG | |
| | AIAWSNQTSFTCQDIFKETNATYPSSDVPC | |
| | DATLTEKSFETDMNLNFQNLLVIVLRILLL | |
| | KVAGFNLLMTLRLWSS | |

A selected CER polynucleotide and the HPV16 E7 TCR polynucleotide were inserted into the same pLenti lentiviral vector with a T2A sequence (encoding an amino acid sequence of SEQ ID NO:156) there between. (see, FIGS. 1A-1G). Peripheral blood was collected by venipuncture from a human donor, and human peripheral blood mononuclear cells (PBMCs) were isolated by density gradient centrifugation using lymphocyte separation media. CD8+ T cells were enriched from PBMCs using a commercially available isolation kit and activated with anti-CD3 and anti-CD28 in Complete Cell Growth Media. 50 μl of viral vector expressing the CER-HPV16 E7 TCR combination were diluted in 0.5 ml Complete Cell Growth Media and added to the CD8+ T cells. The transduced T cells were then centrifuged at 270×g rpm for 1 hour in a 32° C. pre-warmed centrifuge. The T cells were incubated for 24 hours at 37° C. T cells were expanded for another 72 hours in Complete Cell Growth Media, de-beaded, and allowed to expand ×5 days prior to being utilized for functional assays.

CD8 T Cells Transduced with CER-TCR Tandem Expression Cassette Exhibit Antigen Specific Cytolytic and Phagocytic Activity

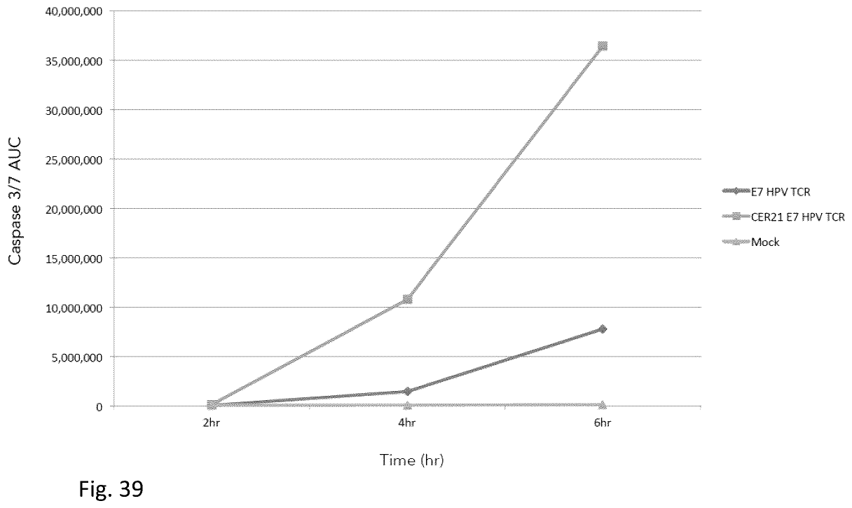

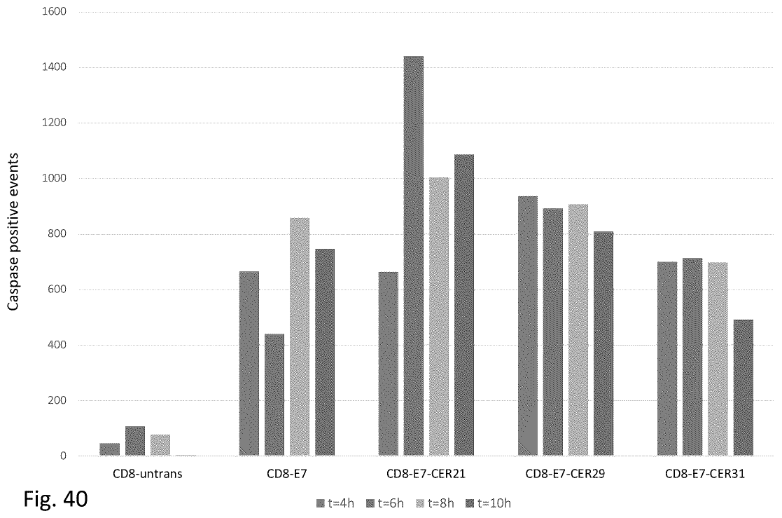

Cytotoxic activity of tandem expression cassette transduced CD8+ T cells was detected using a caspase 3/7 apoptosis reagent (IncuCyte®) that couples the activated caspase 3/7 recognition motif with a red reagent that fluoresces upon cleavage. The fluorescent signal was measured using fluorescent microscopy. Transduced CD8+ T cells were co-cultured with HPV16 E7+ head and neck squamous cell carcinoma cells (SCC152) at a 1:1 ratio, and caspase 3/7 apoptosis reagent was added to the co-culture. CD8+ T cells comprising CER21-HPV16 E7 TCR tandem expression cassette exhibit cytotoxic activity toward SCC152 cells. The cytotoxic response by the CD8+ T cells transduced with CER21-HPV16 E7 TCR tandem expression cassette appears to be exponentially higher than the CD8+ T cells comprising HPV16 E7 TCR alone by 6 hours (see, FIG. 39). CD8+ T cells transduced with CER21-HPV16 E7 TCR tandem expression cassette, CER29-HPV16 E7 TCR tandem expression cassette, or CER31-HPV16 E7 TCR tandem expression cassette were co-cultured with SCC152 cells at a target:effector cell ratio of 1:1. The caspase 3/7 apoptosis reagent was added to the co-culture, and cytotoxic activity was measured over time by measuring fluorescence (see, FIG. 40). Control samples were CD8 T cells transduced with HPV16 E7 TCR alone or mock transduced T cells.

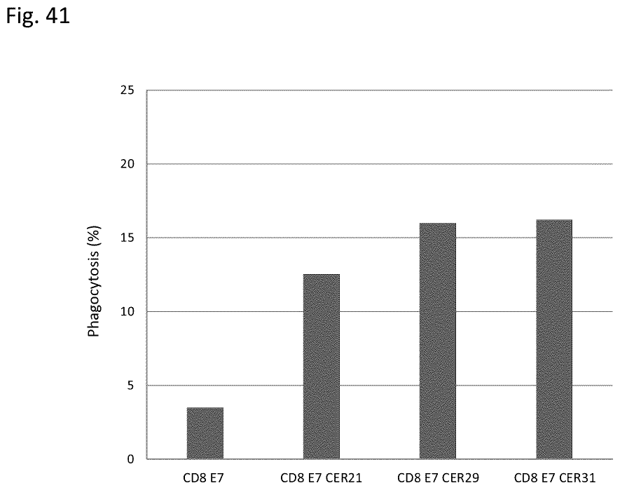

Phagocytic activity of tandem expression cassette transduced CD8+ T cells was detected by co-culturing tandem expression cassette transduced CD8+ T cells with SCC152 cells for 6 hours at a 1:1 ratio. Phagocytic events were visualized and quantified using KEYENCE BZ-X710 fluorescence microscope, 20× objective and hybrid capture software. CD8+ T cells transduced with CER21-HPV16 E7 TCR, CER29-HPV16 E7 TCR, or CER31-HPV16 E7 TCR tandem cassettes were capable of phagocytosing SCC152 cells (see, FIG. 41). Rac1 inhibitor NSC23766 (50 μM) was also added to co-culture experiments and in vitro phagocytosis was measured. Treatment with Rac1 inhibitor revealed that the engulfment of SCC152 cells by the CER21-HPV16 E7 TCR, CER29-HPV16 E7 TCR, or CER31-HPV16 E7 TCR transduced T cells occurred in a Rac1-dependent manner (data not shown). CD8+ T cells transduced with CER21-HPV16 E7 TCR tandem expression cassette engulfed streptavidin coated latex beads, to which were coated with biotin-conjugated phosphatidylserine (data not shown). After about 30 minutes of incubation, the phosphatidylserine coated beads could be visualized inside the CER21-HPV16 E7 TCR+ T cells.

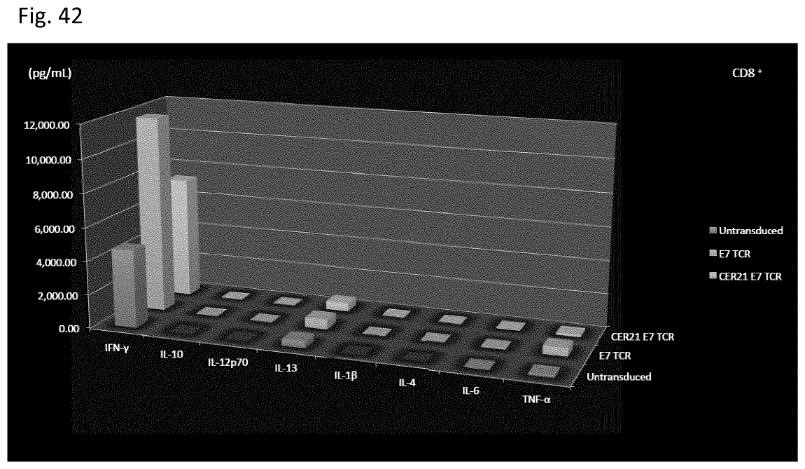

Cytokine response of CD8+ T cells transduced with CER21-HPV16 E7 TCR tandem expression cassette was measured during co-culture experiments with SCC152 cells by sampling the cellular supernatants and showed that CER21-HPV16 E7 TCR+ T cells exhibit antigen specific effector function as measured by IFNγ response (see, FIG. 42).

### Example 7

### CER Enhancement of Molecularly Targeted Cancer Therapy

This example describes approaches to utilize molecularly targeted therapy in combination with CER-expressing cells, for the treatment of cancer. In this scenario, a small molecule inhibitor targeting a first molecule, e.g., a driver-oncogene, induces expression or membrane exposure of a second target molecule, which is recognized by a CER-expressing cell. This drug-inducible target may be a pro-engulfment marker (e.g., phosphatidylserine). Upon recognition and binding of the induced second target molecule, CER-expressing cells elicit anti-tumor activity via activation of phagocytic signal transduction cascades. This approach can be utilized to enhance molecular targeted therapy for hematologic and solid tumors.

### CER Enhancement of EGFR Inhibitors

˜30-40% of non-small cell lung cancer (NSCLC) in Japanese patients and ˜15% of harbor an epidermal growth factor (EGFR)-activating mutation. For the treatment of EGFR-mutated NSCLC, EGFR-tyrosine kinase inhibitors (EGFR-TKIs) have been developed that inhibit EGFR-induced downstream signaling pathways. Clinical studies show improved prognoses with EGFR inhibitors in patients with EGFR-mutated lung cancer, extending overall survival of advanced NSCLC from ˜1 year to 2-3 years. EGFR inhibitors may be used in the treatment of other cancers possessing an activating EGFR mutation, including colorectal cancer, breast cancer, ovarian cancer, pancreatic cancer, and glioblastoma. Clinical oncology studies demonstrate that even with the most potent targeted therapeutics the vast majority of patients that receive drugs designed to interfere with a specific gene or protein eventually relapse, often with new tumors that no longer respond to therapy.

CER-modified cells that were engineered to recognize pro-engulfment marker phosphatidylserine were administered in conjunction with various EGFR inhibitors, Osimeritinib, Brigatinib, Erlotinib, and Gefitinib, to determine whether CER therapy could enhance EGFR targeted therapy.

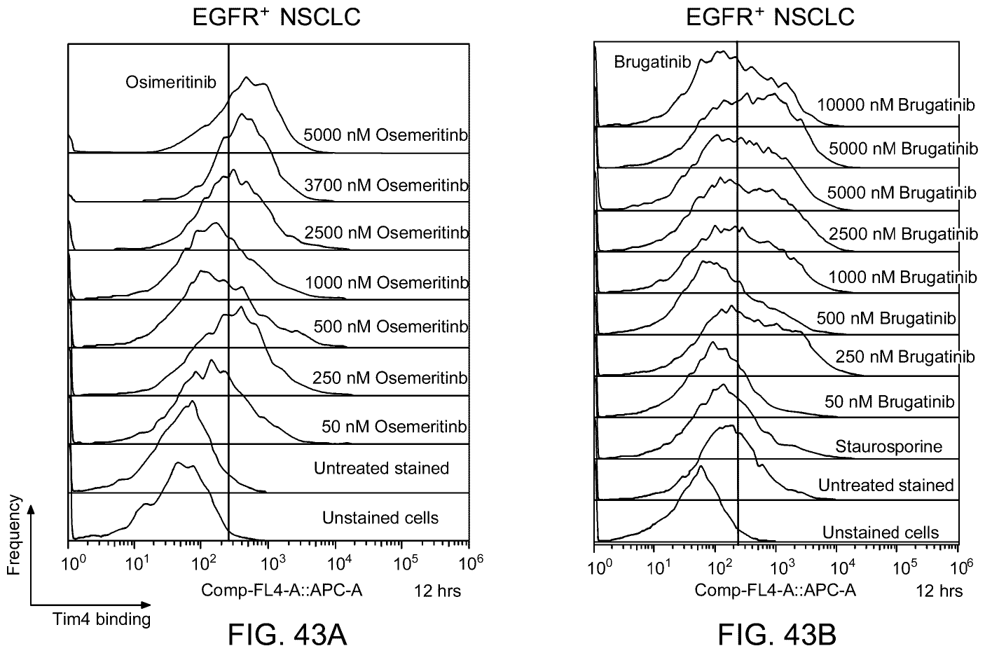

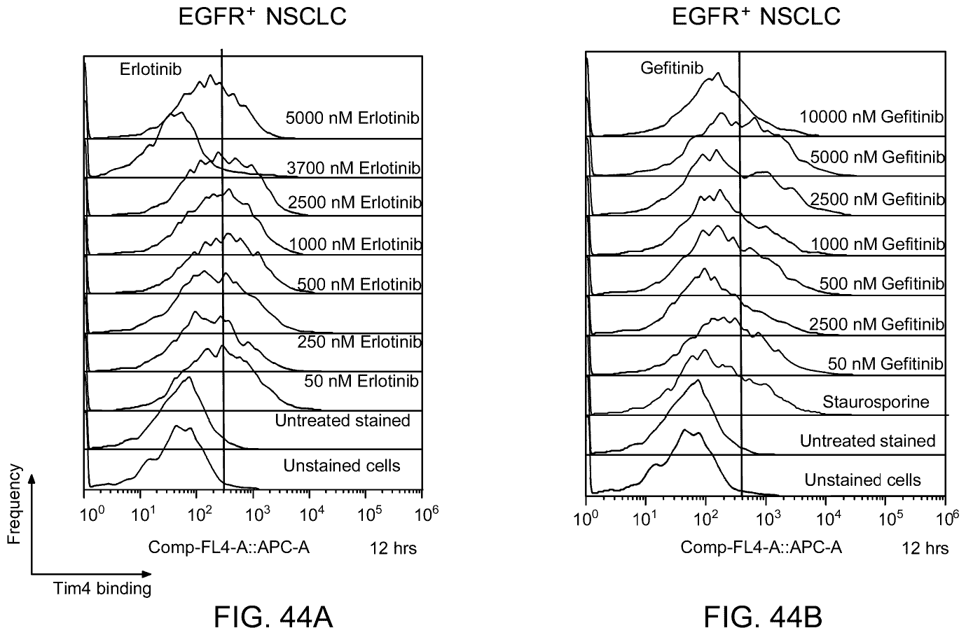

HCC159 lung adenocarcinoma cells harbor an EGFR mutation and are sensitive to EGFR inhibition. HCC159 cells were treated for 12 hours in the presence of EGFR kinase inhibitor Osimeritinib, Brigatinib, Erlotinib, or Gefitinib at increasing concentrations (50 nM, 250 nM, 500 nM, 1000 nM, 2500 nM, 3700 nM, or 5000 nM for Osimeritinib, Brigatinib, and Erlotinib; 50 nM, 250 nM, 500 nM, 1000 nM, 2500 nM, 5000 nM, 10000 nM for Brigatinib) and incubated with a Tim4-IgG1 Fc recombinant fusion protein to evaluate for pro-engulfment marker (phosphatidylserine) exposure on target cells following EGFR inhibitor treatment. Increasing concentrations of Osimeritinib (FIG. 43A), Brigatinib (FIG. 43B), Erlotinib (FIG. 44A), and Gefitinib (FIG. 44B) enhanced surface staining with Tim4-IgG1 Fc fusion protein, indicating that exposure of the phosphatidylserine target molecule is EGFR drug inducible.

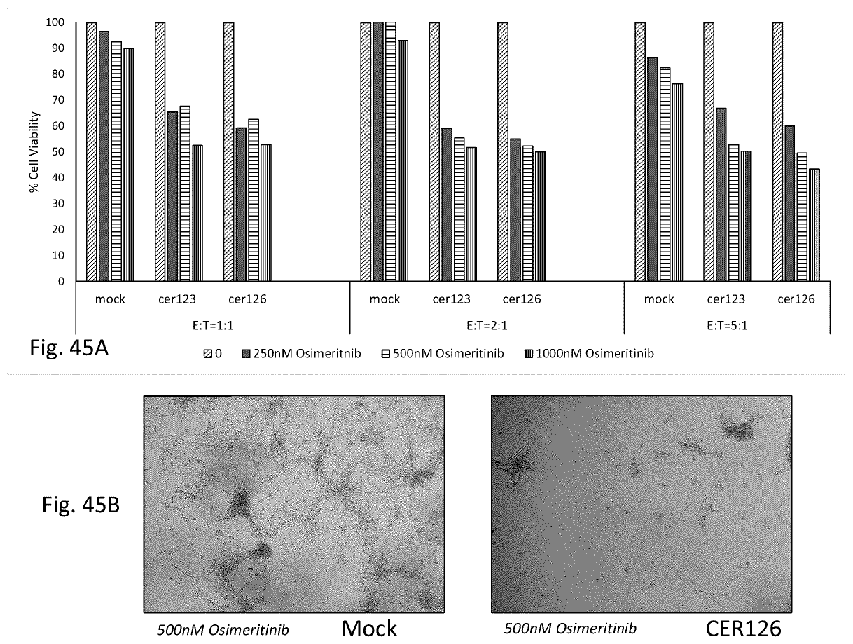

H1975 lung adenocarcinoma cells harboring an EGFR mutation were co-cultured with CER123- or CER126-modified or Mock-transduced (vector only) CD4+ T cells with increasing concentrations of Osimeritinib. CER123 has a polypeptide sequence as set forth in SEQ ID NO: 150 and comprises a Tim4 binding domain, Tim4 transmembrane domain, and an engulfment signaling domain comprising a primary engulfment signaling domain comprising a TLR2 signaling domain and a secondary engulfment signaling domain comprising a TRAF6 signaling domain. CER126 has a polypeptide sequence as set forth in SEQ ID NO:174 and comprises a Tim4 binding domain, Tim4 transmembrane domain, and an engulfment signaling domain comprising a primary engulfment signaling domain comprising a TLR2 signaling domain and a secondary engulfment signaling domain comprising a TRAF2 signaling domain. CD4 T cells were transduced with a lentiviral vector comprising CER123 or CER126 nucleic acid and truncated EGFR (transduction marker) nucleic acid. Assays were performed using CER modified T cells purified by FACS using an EGFR specific antibody 7 days after activation using CD3 & CD28 microbeads. CER modified T cells and H1975 cells were co-cultured at effector:target cell ratio (E:T) of 1:1, 2:1, or 5:1. After 48 hours of co-culture in the presence of Osimeritinib (0, 250 nM, 500 nM, or 1000 nM), T cells were washed away, and the number of viable H1975 cells were quantified using a calorimetric MTT assay. Cell viability experiments were performed in triplicate and presented as % of control (FIG. 45A). Bright field images of co-culture experiments demonstrate loss of H1975 cells in the presence of Osimeritinib (500 nM)+CER126-modified T cells (FIG. 45B, right panel) compared to control T cells (vector alone) (FIG. 45B, left panel). Thus, in the presence of an EGFR kinase inhibitor, CER-expressing T cells targeting a pro-engulfment marker demonstrate dose-dependent cell killing responses.

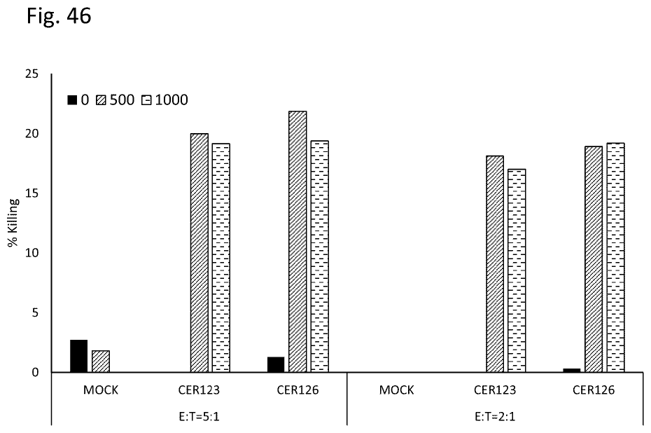

H1975 NSCLC cells were co-cultured with CER123- or CER126-expressing CD4+ T cells with increasing concentrations of Osimeritnib (0, 500 nM, or 1000 nM). Mock-transduced (vector only) T cells were used as control. Assays were performed using CER modified T cells purified by FACS using an EGFR specific antibody 7 days after activation using CD3 & CD28 microbeads. CER modified T cells and H1975 cells were co-cultured at effector:target cell ratios of 2:1 or 5:1. After 18 hours of co-culture, bulk supernatants were evaluated using LDH-based cytotoxicity assay. In the presence of Osimeritinib, CER123- or CER126-expressing T cells demonstrate inducible cell killing responses (FIG. 46).

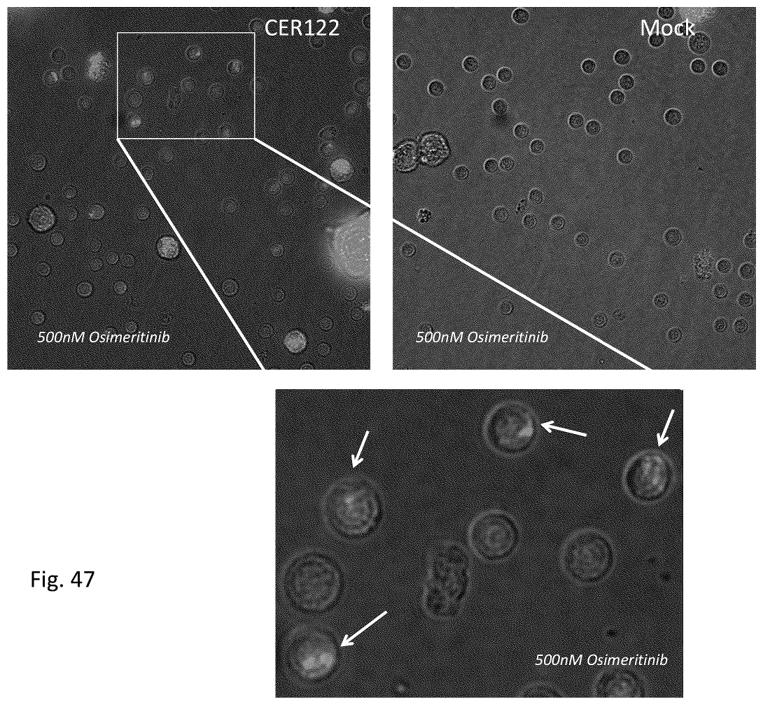

H1975 NSCLC cells were treated with 500 nM Osimeritinib and then labeled with pH-rodo red, a pH sensing dye, to indicate localization to low-pH retaining endosomes. CD4+ T cells were transduced with a lentiviral vector comprising a CER122 nucleic acid and tEGFR nucleic acid (transduction marker). CER122 has a polypeptide sequence as set forth in SEQ ID NO:149 and comprises a Tim4 binding domain, Tim4 transmembrane domain, and an engulfment signaling domain comprising a primary engulfment signaling domain comprising a TLR2 signaling domain and a secondary engulfment signaling domain comprising a DAP12 signaling domain. CER122-transduced T cells labeled with CELLTRACE Violet were co-cultured with Osimeritinib-treated H1975 NSCLC cells. Fluorescent microscopy images (40×) were obtained 12 hours after co-culture of H1975 cells and CER-expressing T cells (FIG. 47, top left panel). Mock-transduced CD4+ T cells (vector alone) were used as control and exhibit no phagocytic activity (FIG. 47, top right panel). Phagocytic events can be visualized as pHrodo red targets within CELLTRACE Violet labeled T cells. An enlargement of phagocytosis of pHrodo red labeled H1975 cells by CELLTRACE Violet labeled CER122-modified T cells is shown in the bottom panel of FIG. 47, with white arrows indicating phagocytic events.

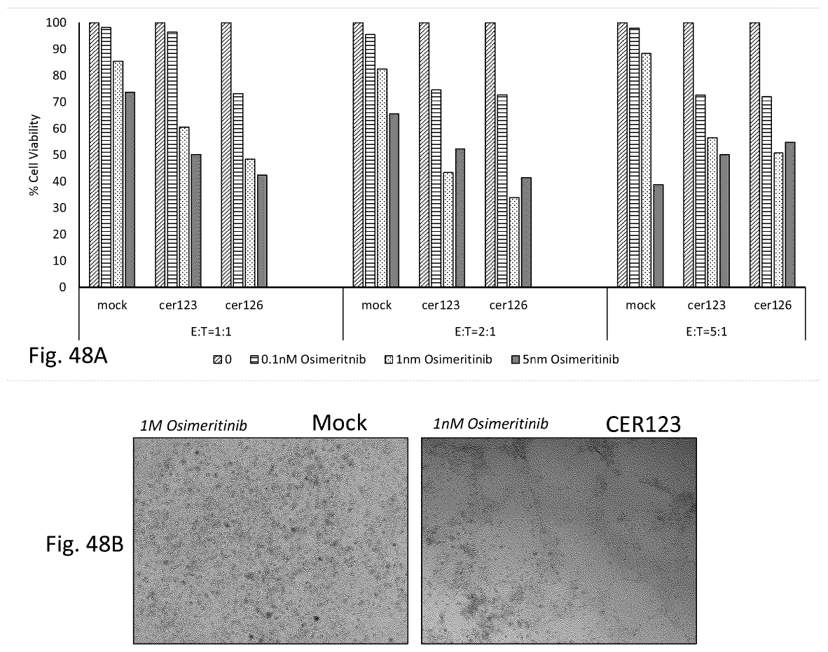

HCC159 lung adenocarcinoma cells harboring an EGFR mutation were co-cultured with CER123- or CER126-expressing CD4+ T cells with increasing concentrations of Osimeritinib (0.1 nM, 1 nM, or 5 nM). Mock transduced (vector only) T cells were used as control. After 48 hours of co-culture at effector:target cell ratios at 1:1, 2:1 or 5:1 in the presence of drug, T cells were washed away, and the number of viable HCC159 cells was quantified using a calorimetric MTT assay. Cell viability experiments were performed in triplicates and presented as % of control with (FIG. 48A). In the presence of an EGFR kinase inhibitor, CER123- and CER126-expressing cells demonstrate dose-dependent target cell killing responses. Bright field images demonstrate loss of HCC159 cells in the presence of 1 nM Osimeritinib and CER123-expressing T cells (FIG. 48B, right panel) compared to control T cells (vector alone) (FIG. 48B, left panel).

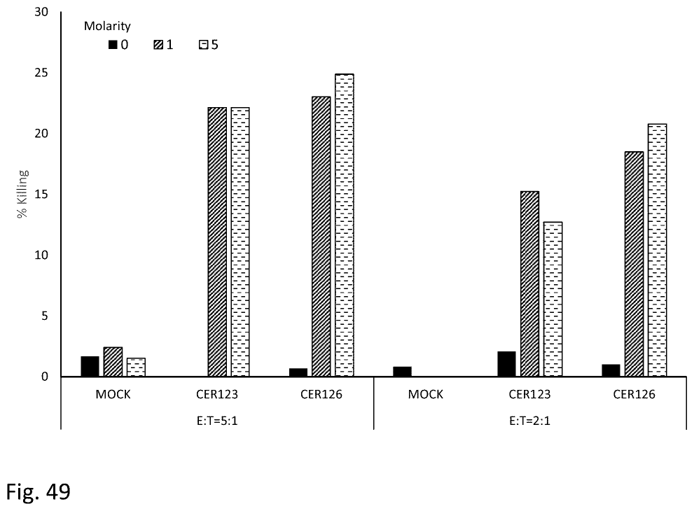

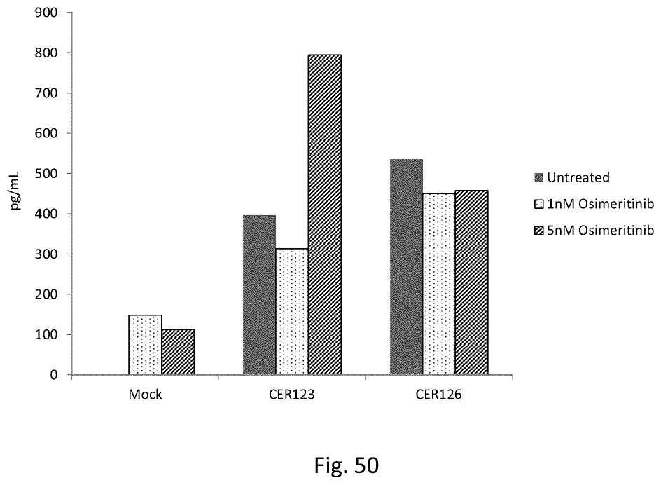

HCC159 lung adenocarcinoma cells were co-cultured with CER123- or CER126-expressing CD4+ T cells with increasing concentrations of Osimeritnib (0, 1 nM, 5 nM). After 18 hours of co-culture, bulk supernatants were evaluated using a LDH-based cytotoxicity assay. T cells assays were performed using purified, EGFR (transduction marker), CER modified T cells 7 days after activation using CD3 & CD28 microbeads at effector:target cell ratios of 2:1 or 5:1. Control “Mock” T cells were transduced with vector alone. In the presence of Osimeritinib, CER123- and CER126-expressing cells demonstrate inducible cell killing responses (FIG. 49). Bulk supernatants were also analyzed after 18 hours of co-culture for Interferon Gamma (IFNγ) secretion (FIG. 50). Control “Mock” T cells were transduced with vector alone. In the presence of Osimeritinib, CER123- demonstrates inducible cytokine secretion.

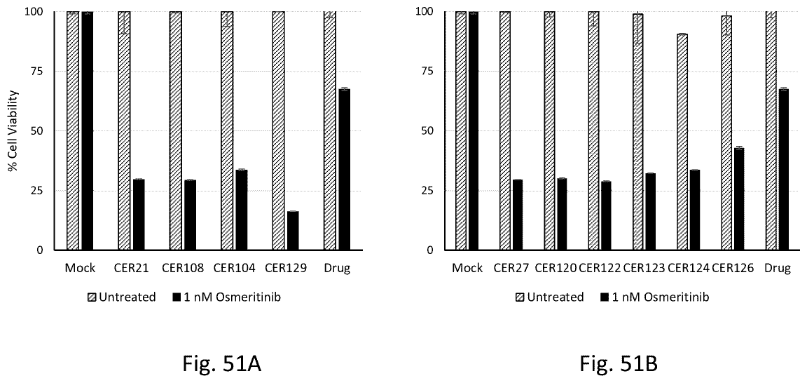

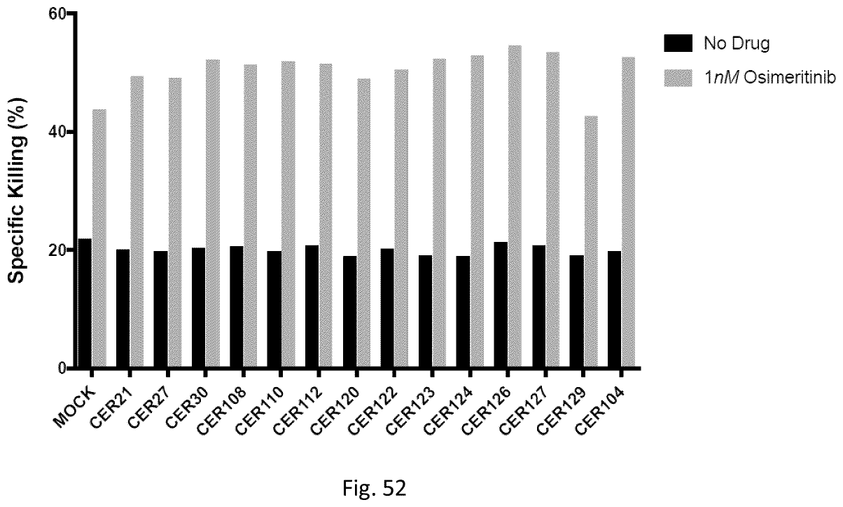

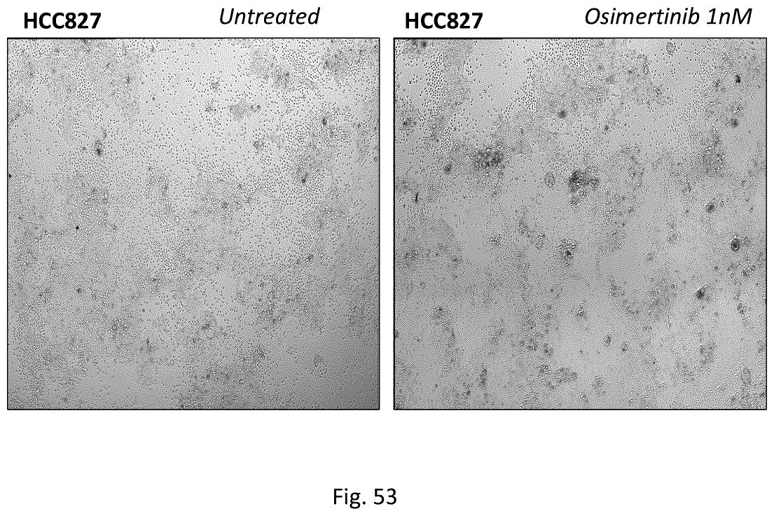

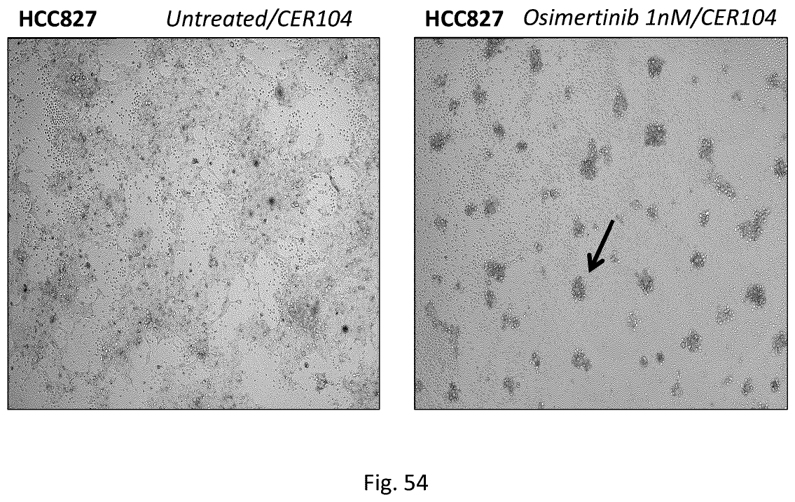

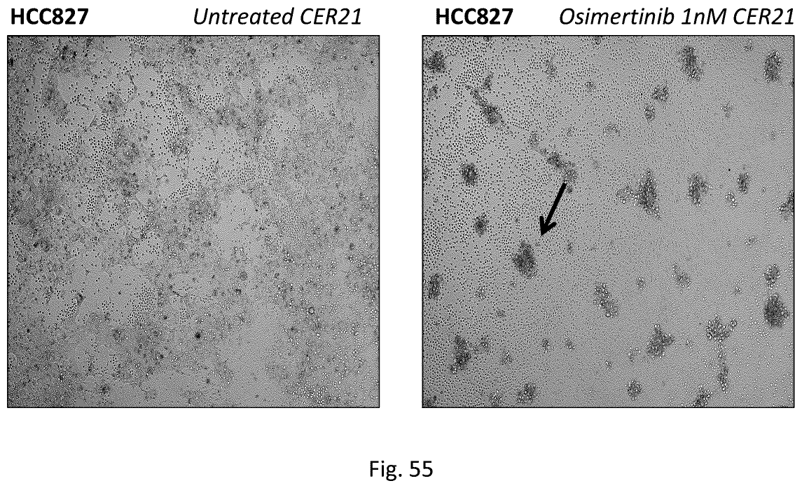

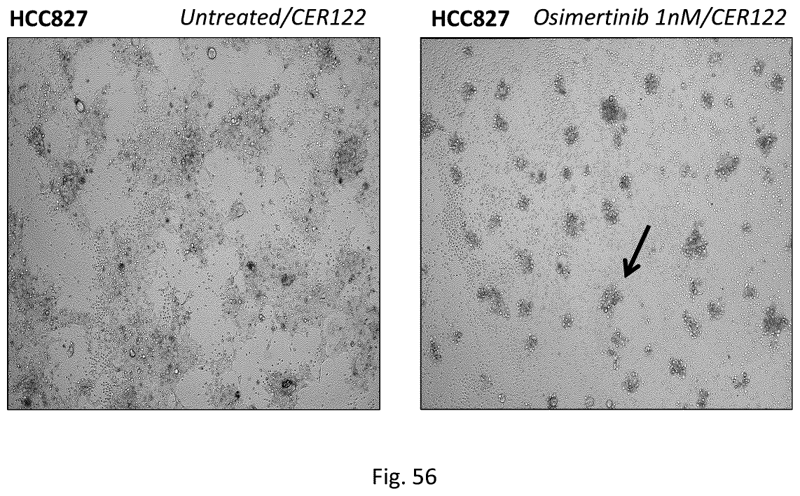

CD4+ T cells were transduced with lentiviral vectors encoding various CERs (CER21, CER108, CER104, CER129, CER27, CER120, CER122, CER123, CER124, or CER126). Mock transduced (vector only) CD4+ T cells were used as control. CER21 comprises a Tim4 binding domain, Tim4 transmembrane domain, and an engulfment signaling domain comprising a primary engulfment signaling domain comprising a TLR8 signaling domain. CER21 has a polypeptide sequence as set forth in SEQ ID NO:88. CER108 has a polypeptide sequence as set forth in SEQ ID NO:137 and comprises a Tim4 binding domain, Tim4 transmembrane domain, and an engulfment signaling domain comprising a primary engulfment signaling domain comprising a DAP12 signaling domain and a secondary engulfment signaling domain comprising a TLR8 signaling domain. CER104 has a polypeptide sequence as set forth in SEQ ID NO: 133 and comprises a Tim4 binding domain, Tim4 transmembrane domain, and an engulfment signaling domain comprising a primary engulfment signaling domain comprising a TLR8 signaling domain and a secondary engulfment signaling domain comprising a DAP12 signaling domain. CER129 has a polypeptide sequence as set forth in SEQ ID NO: 177 and comprises a Tim4 binding domain, Tim4 transmembrane domain, and an engulfment signaling domain comprising a primary engulfment signaling domain comprising a TLR8 signaling domain and a secondary engulfment signaling domain comprising a TRAF2 signaling domain. CER27 comprises a Tim4 binding domain, Tim4 transmembrane domain, and an engulfment signaling domain comprising a primary engulfment signaling domain comprising a TLR2 signaling domain. CER27 has a polypeptide sequence as set forth in SEQ ID NO:93. CER120 has a polypeptide sequence as set forth in SEQ ID NO: 147 and comprises a Tim4 binding domain, Tim4 transmembrane domain, and an engulfment signaling domain comprising a primary engulfment signaling domain comprising a TLR1 signaling domain and a secondary engulfment signaling domain comprising a DAP12 signaling domain. CER122 has a polypeptide sequence as set forth in SEQ ID NO:149 and comprises a Tim4 binding domain, Tim4 transmembrane domain, and an engulfment signaling domain comprising a primary engulfment signaling domain comprising a TLR2 signaling domain and a secondary engulfment signaling domain comprising a DAP12 signaling domain. CER123 has a polypeptide sequence as set forth in SEQ ID NO: 150 and comprises a Tim4 binding domain, Tim4 transmembrane domain, and an engulfment signaling domain comprising a primary engulfment signaling domain comprising a TLR2 signaling domain and a secondary engulfment signaling domain comprising a TRAF6 signaling domain. CER126 has a polypeptide sequence as set forth in SEQ ID NO: 174 and comprises a Tim4 binding domain, Tim4 transmembrane domain, and an engulfment signaling domain comprising a primary engulfment signaling domain comprising a TLR2 signaling domain and a secondary engulfment signaling domain comprising a TRAF2 signaling domain. CER124 has a polypeptide sequence as set forth in SEQ ID NO:151 and comprises a Tim4 binding domain, Tim4 transmembrane domain, and an engulfment signaling domain comprising a primary engulfment signaling domain comprising a TLR2 signaling domain and a secondary engulfment signaling domain comprising a NFAM1 signaling domain. HCC827 lung adenocarcinoma cells harboring an EGFR mutation were co-cultured with CER-expressing T cells or mock-transduced T cells at a 1:1 effector:target cell ratio with 1 nM Osimeritinib for 48 hours. T cells were washed away, and the number of viable HCC827 cells was quantified using a calorimetric MTT assay. Cell viability experiments were performed in triplicate and presented as % of control. CER-expressing T cells demonstrate synergistic killing of HCC827 cells upon EGFR inhibition (FIGS. 51A-B). HCC827 cells harboring an EGFR mutation were co-cultured with CER-expressing or mock-transduced (vector only) CD4+ T cells with 1 nM of Osimeritinib. After 48 hours of co-culture at a 1:1 effector:target cell ratio, T cells were washed away, and the number of viable HCC827 cells was quantified using a calorimetric LDH assay (FIG. 52). Cell viability experiments were performed in triplicate and presented as % of control. Bright field microscopy images from CER+HCC827±1 nM Osimeritinib co-culture experiments were obtained at 48 hours (FIGS. 53-56).

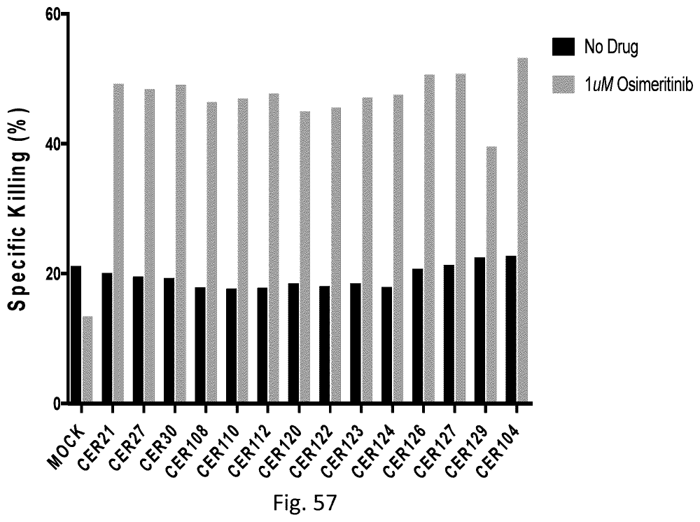

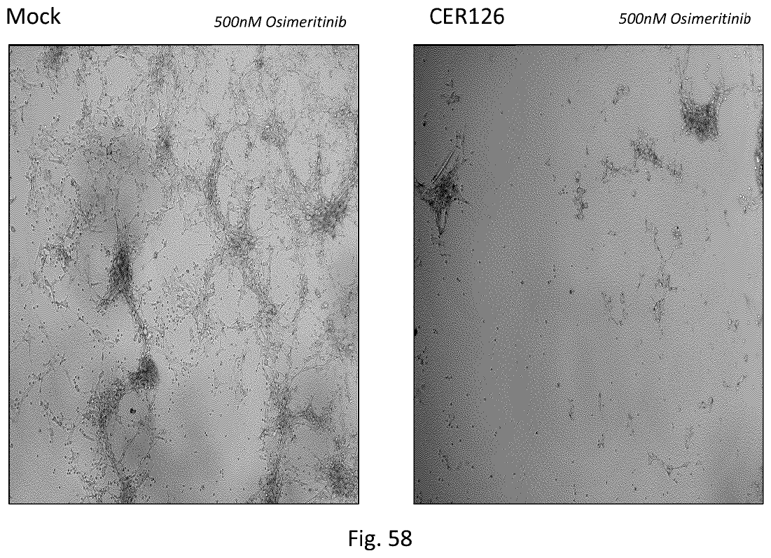

CER-expressing (CER21, CER27, CER30, CER108, CER110, CER112, CER120, CER122, CER123, CER124, CER126, CER127, CER129, or CER104) CD4+ T cells also demonstrated synergistic killing of H1975 lung adenocarcinoma cells upon EGFR inhibition (FIG. 57). CER30 comprises a Tim4 binding domain, Tim4 transmembrane domain, and an engulfment signaling domain comprising a primary engulfment signaling domain comprising a TRAF2 signaling domain. CER30 has a polypeptide sequence as set forth in SEQ ID NO:96. CER110 has a polypeptide sequence of SEQ ID NO:125 and comprises a Tim4 binding domain, Tim4 transmembrane domain, and an engulfment signaling domain comprising a primary engulfment signaling domain comprising a TRAF6 signaling domain and a secondary engulfment signaling domain comprising a DAP12 signaling domain. CER112 has a polypeptide sequence of SEQ ID NO:128 and comprises a Tim4 binding domain, Tim4 transmembrane domain, and an engulfment signaling domain comprising a primary engulfment signaling domain comprising a TRAF6 signaling domain and a secondary engulfment signaling domain comprising a NFAM1 signaling domain. CER127 has a polypeptide sequence of SEQ ID NO: 175 and comprises a Tim4 binding domain, Tim4 transmembrane domain, and an engulfment signaling domain comprising a primary engulfment signaling domain comprising a TRAF2 signaling domain and a secondary engulfment signaling domain comprising a TLR2 signaling domain. H1975 cells are more resistant to EGFR inhibitors, so Osimeritinib was used at slightly higher concentration. H1975 lung adenocarcinoma cells harboring an EGFR mutation were co-cultured with CER-expressing or mock-transduced (vector only) CD4+ T cells with 1 μM of Osimeritinib. After 48 hours of co-culture at a 1:1 effector:target cell ratio, T cells were washed away, and the number of viable H1975 cells was quantified using a calorimetric LDH assay (FIG. 57). Cell viability experiments were performed in triplicate and presented as % of control. FIG. 58 shows bright field images from co-culture of H1975 cells with mock-transduced T cells (left image) or CER126+ T cells (right image) treated with Osimeritinib (500 nM) for 48 hours.

### CER Enhancement of ALK Inhibitors

Anaplastic Lymphoma Kinase (ALK) gene rearrangements account for ˜7% of NSCLC patients. Selective inhibitors of ALK have been developed for the treatment of NSCLC. While clinical studies demonstrate superior efficacy and lower toxicity in the primary treatment of ALK-positive NSCLC with an ALK inhibitor, responses are typically incomplete and temporary.

CER-modified cells engineered to recognize pro-engulfment marker phosphatidylserine were administered in conjunction with various ALK inhibitors, Alectinib and Crizotinib, to determine whether CER therapy could enhance ALK targeted therapy.

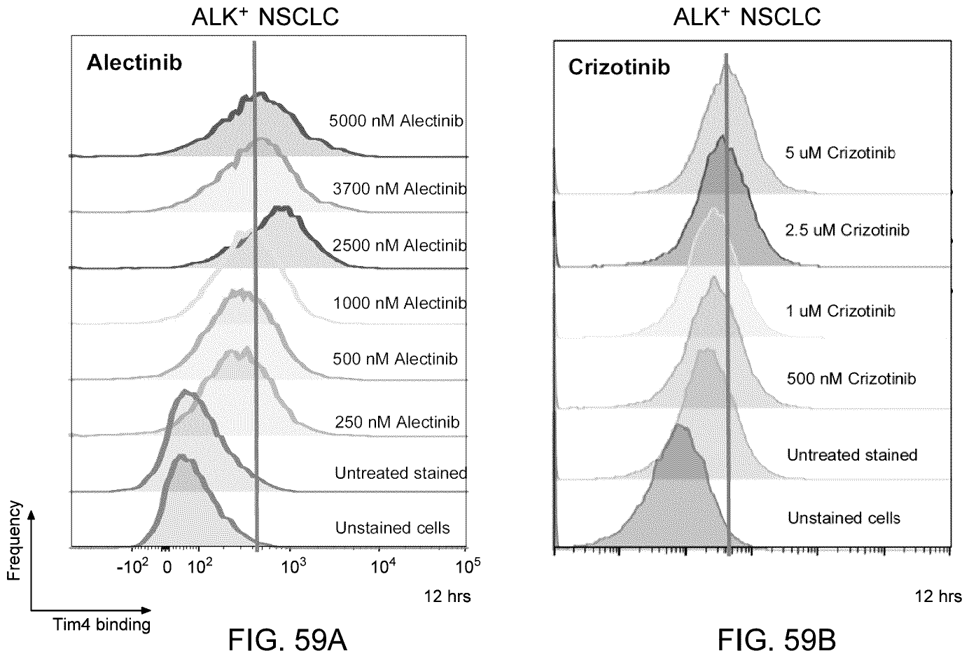

An ELM4-ALK fusion translocation was introduced into A549 lung adenocarcinoma cells. The A549 cells were then treated for 12 hours in the presence of increasing concentrations of ALK kinase inhibitors Alectinib (250 nM, 500 nM, 1000 nM, 2500 nM, 3700 nM, or 5000 nM) or Crizotinib (500 nM, 1 μM, 2.5 μM, or 5 μM) and stained with a Tim4-IgG1 Fc recombinant fusion protein to evaluate for pro-engulfment marker (phosphatidylserine) exposure on target cells following ALK inhibitor treatment. Increasing concentrations of Alectinib (FIG. 59A) and Crizotinib (FIG. 59B) enhance surface staining with Tim4-IgG1 Fc fusion protein, indicating that exposure of the phosphatidylserine target molecule is ALK inhibitor drug inducible.

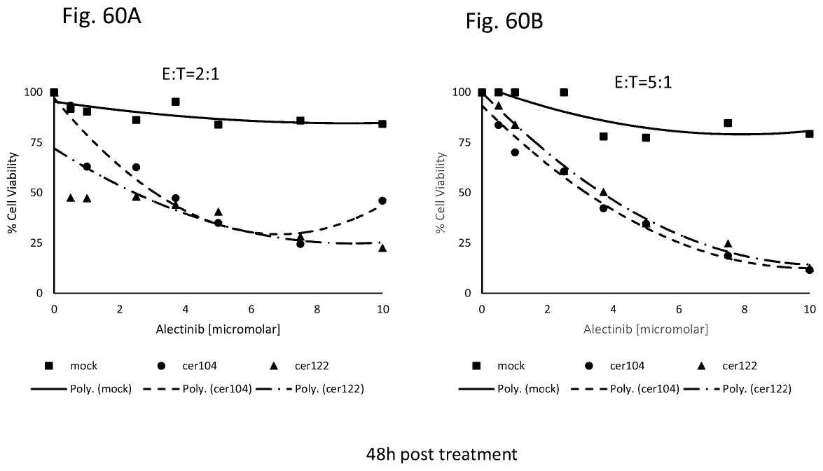

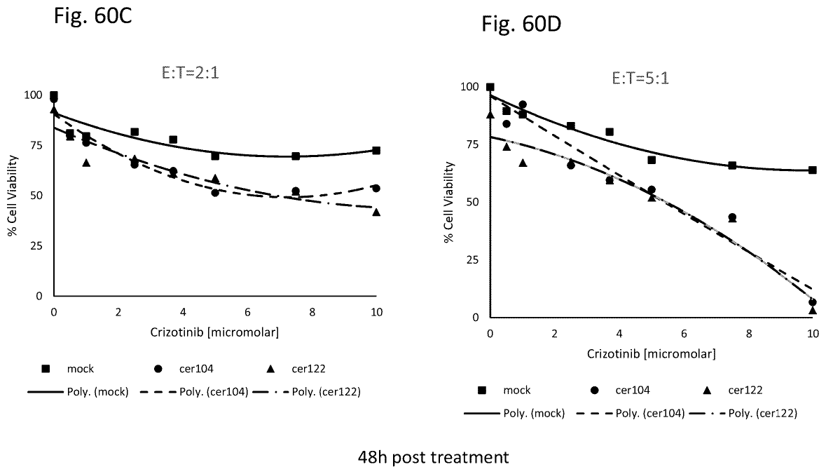

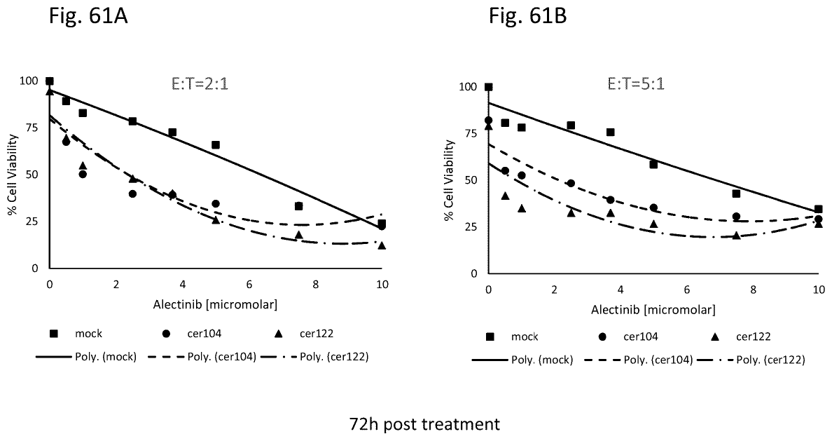

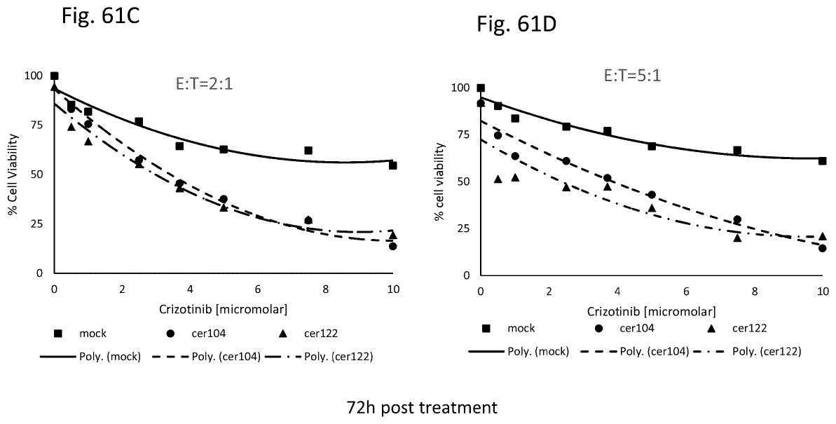

An ELM4-ALK translocation was introduced into A549 cells. The A549 cells were then co-cultured with CER104- or CER122-expressing T cells or Mock-transduced (vector only) T cells with increasing concentrations of ALK inhibitor drug Alectinib (250 nM, 500 nM, 1 μM, 2.5 μM, 3.7 μM, 7 μM, or 10 μM) or Crizotinib (250 nM, 500 nM, 1 μM, 2.5 μM, 3.7 μM, 7 μM, or 10 μM). CER104 has a polypeptide sequence set forth in SEQ ID NO: 133 and comprises a Tim4 binding domain, Tim4 transmembrane domain, and an engulfment signaling domain comprising a primary engulfment signaling domain comprising a TLR8 signaling domain and a secondary engulfment signaling domain comprising a DAP12 signaling domain. CER122 has a polypeptide sequence set forth in SEQ ID NO: 149 and comprises a Tim4 binding domain, Tim4 transmembrane domain, and an engulfment signaling domain comprising a primary engulfment signaling domain comprising a TLR2 signaling domain and a secondary engulfment signaling domain comprising a DAP12 signaling domain. Effector:target (E:T) cell ratio for co-culture was 2:1 or 5:1. After 48 hours and 72 hours of co-culture in the presence of ALK inhibitor drug, T cells were washed away, and the number of viable A549 cells were quantified using a calorimetric MTT assay. % cell viability for CER+Alectinib treated A549 cells at 48 hours post-treatment are shown in FIG. 60A (Effector:Target cell ratio of 2:1) and FIG. 60B (Effector:Target cell ratio of 5:1). % cell viability for CER+Crizotinib treated A549 cells at 48 hours post-treatment are shown in FIG. 60C (Effector:Target cell ratio of 2:1) and FIG. 60D (Effector:Target cell ratio of 5:1). % cell viability for CER+Alectinib treated A549 cells at 72 hours post-treatment are shown in FIG. 61A (Effector:Target cell ratio of 2:1) and FIG. 61B (Effector:Target cell ratio of 5:1). % cell viability for CER+Crizotinib treated A549 cells at 72 hours post-treatment are shown in FIG. 61C (Effector:Target cell ratio of 2:1) and FIG. 61D (Effector:Target cell ratio of 5:1). Cell viability experiments were performed in triplicates and presented as % of control. Best-fit curves were generated from raw data using linear regression models. In the presence of ALK kinase inhibitors, CER-expressing cells demonstrate dose-dependent cell killing responses.

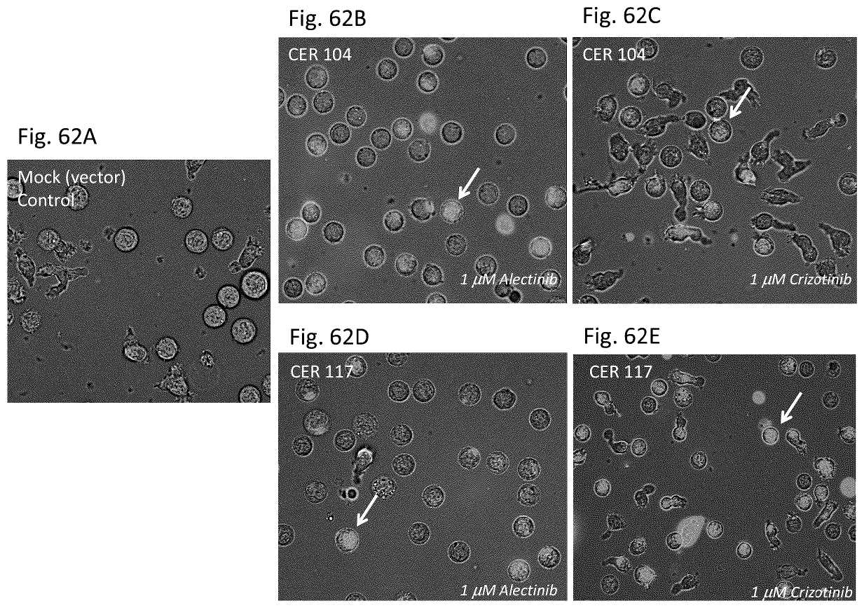

An ELM4-ALK translocation was introduced into A549 cells. ALK+ A549 cells were treated with 1 μM Alectinib or 1 μM Crizotinib and labeled with pHrodo red, a pH sensitive dye, to indicate localization in low-pH retaining endosomes. CD4 T cells are modified to express CER104 or CER117 and labeled with CELLTRACE Violet. CER117 has a polypeptide sequence set forth in SEQ ID NO:144 and comprises a Tim4 binding domain, a Tim4 transmembrane domain, and an engulfment signaling domain comprising a primary engulfment signaling domain comprising a TLR8 signaling domain and a secondary engulfment signaling domain comprising a TRAF6 signaling domain. The ALK+ A549 cells were then co-cultured with CER104- or CER117-expressing CD4+ T cells or Mock-transduced (vector only) T cells for 12 hours and images were obtained by fluorescent microscopy at 40× magnification (FIGS. 62A-E). White arrows indicate exemplary phagocytic events (pH rodo red cell targets within CELLTRACE Violet labeled CD4+ T cells). CER-expressing T cells phagocytosed ALK+ A549 cells treated with Alectinib or Crizotinib (FIGS. 62B-E). Mock-transduced (vector only) controls exhibit no phagocytic activity (FIG. 62A).

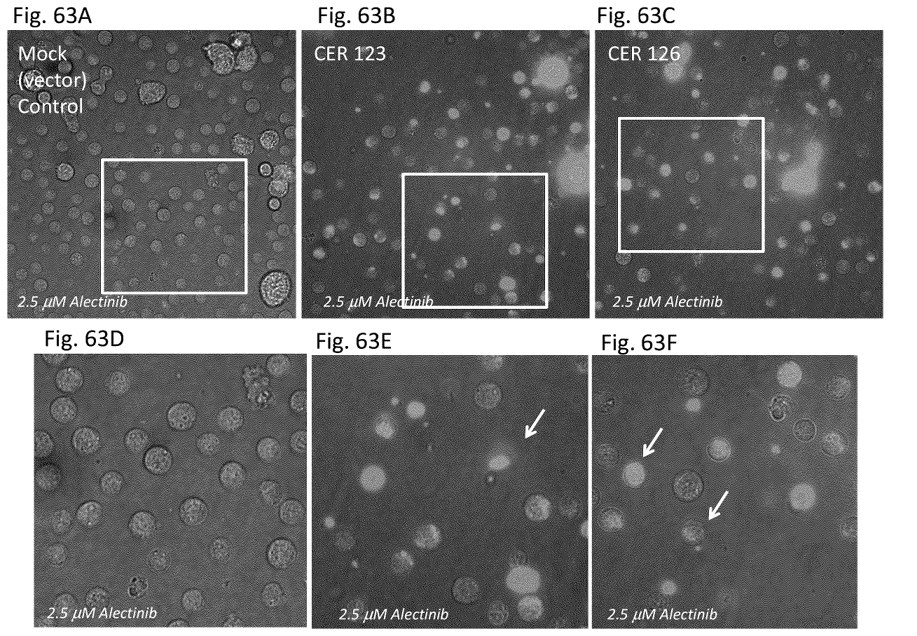

Similarly, CER123- and CER126-expressing CD4+ T cells exhibited phagocytic elimination of ALK+ A549 cells treated with 2.5 μM Alectinib (FIGS. 63B, 63C, 63E, 63F). ALK+ A549 cells were treated with 2.5 μM Alectinib and then labeled with pH-rodo red, a pH sensing dye, to indicate localization to low-pH retaining endosomes. CER123- and CER126-transduced CD4+ and CD8+ T cells were labeled with CELLTRACE Violet and co-cultured with the A549 ALK-positive cells. Co-cultured cells were imaged by fluorescent microscopy at 63× magnification after 12 hours (FIGS. 63A-F). White arrows indicate exemplary phagocytic events (pHrodo red targets within CELLTRACE Violet-labeled CD4 T cells). CER-expressing T cells phagocytosed ALK+ A549 cells treated with Alectinib (FIGS. 63B, 63C, 63E, and 63F). Mock-transduced (vector only) controls exhibit no phagocytic activity (FIGS. 63A, 63D).

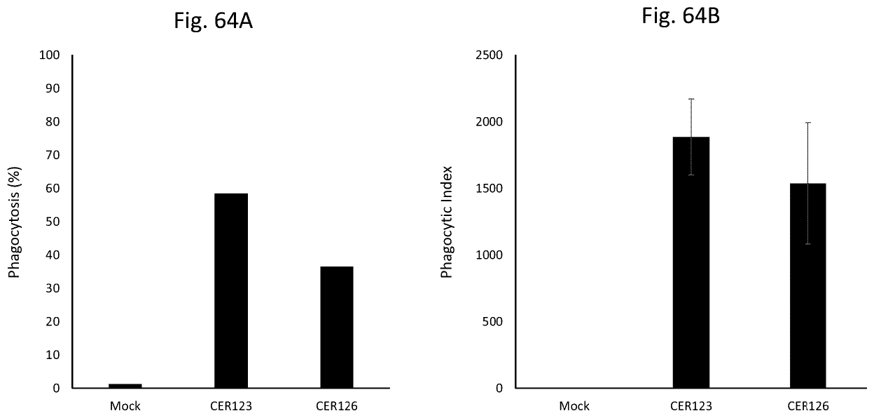

Phagocytosis of Alectinib-treated ALK+ A549 cells by CER-expressing T cells was also quantified. A549 cells were treated with Alectinib at 1 μM for 12 hours and co-cultured with CER123- or CER126-expressing T cells at a 1:1 ratio for 12 hours. Bar graph in FIG. 64A represents quantification of percentage phagocytosis calculated as ((number of phagocytic target events)/(total number of effector cells))*100. Events were calculated from fluorescent 3×3 stitched images at 40× resolution after 12 hours of co-culture. Bar graph in FIG. 64B represents quantification of adjusted phagocytic index calculated as (median area ratio of target events in effector cells*% phagocytosis). Events calculated from fluorescent 3×3 stitched images at 40× resolution after 12 hours of co-culture.

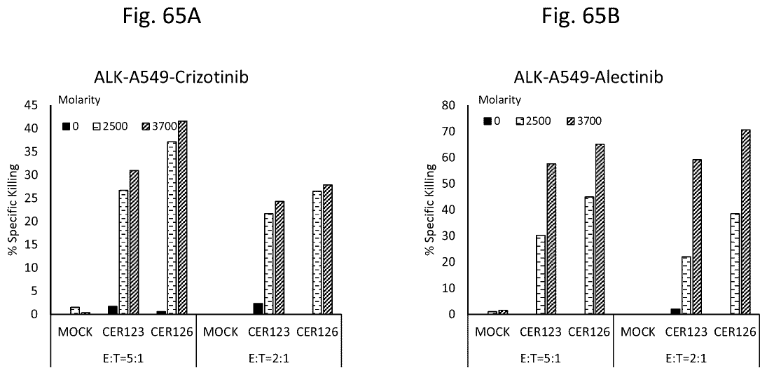

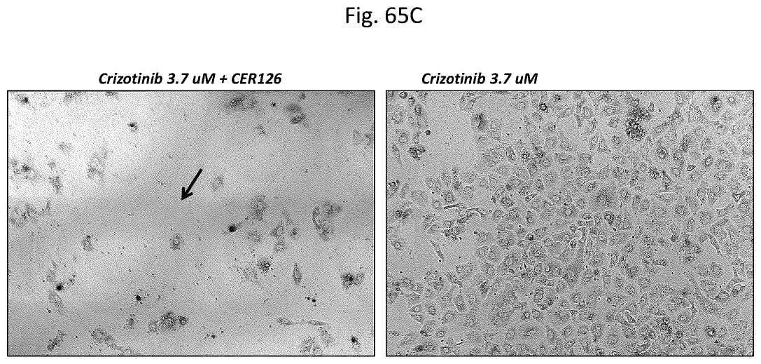

In the presence of ALK kinase inhibitors Crizotinib and Alectinib, CER123- or CER126-expressing T cells demonstrate dose-dependent inducible cytokine secretion and cell killing responses. An ELM4-ALK translocation was introduced into A549 cells. ALK+ A549 cells were co-cultured with CER-expressing T cells with increasing concentrations of ALK inhibitor drugs Crizotinib or Alectinib (0, 2500 nM, or 3700 nM). Assays were performed with purified tEGFR⁺ (transduction marker), CER-expressing T cells 7 days after activation using CD3 & CD28 microbeads at effector:target cell ratios of 2:1 or 5:1. Mock T cells were transduced with vector alone and used as control. After 18 hours of co-culture, bulk supernatants were evaluated using a LDH cytoxicity assay indicating cell killing (FIGS. 65A-B). Micrograph images from co-culture experiments demonstrate near complete loss of Crizotinib-treated A549 cells from wells in the presence of a CER126-transduced cell (FIG. 65C, left panel).

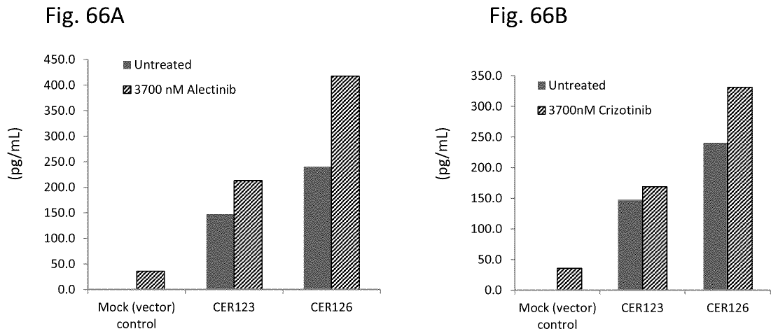

In the presence of ALK kinase inhibitors Crizotinib and Alectinib, CER123- or CER126-expressing T cells demonstrate dose-dependent inducible cytokine secretion (FIGS. 66A-B). A549 cells introduced with an ELM4-ALK translocation were co-cultured with CER123- or CER126-expressing T cells with ALK inhibitor Crizotinib (3700 nM) or Alectinib (3700 nM). After 18 hours of co-culture, bulk supernatants were evaluated for Interferon Gamma secretion (FIGS. 66A-B). Assays were performed using purified, tEGFR⁺ (transduction marker), CER-expressing T cells 7 days after activation using CD3 & CD28 microbeads at effector:target ratios of (2:1 & 5:1). Control “Mock” T cells were transduced with vector alone.

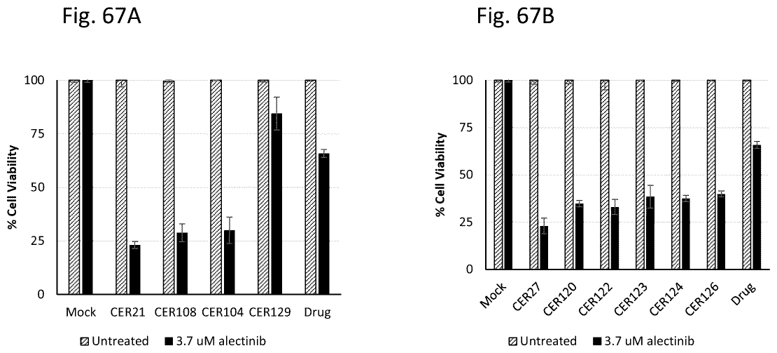

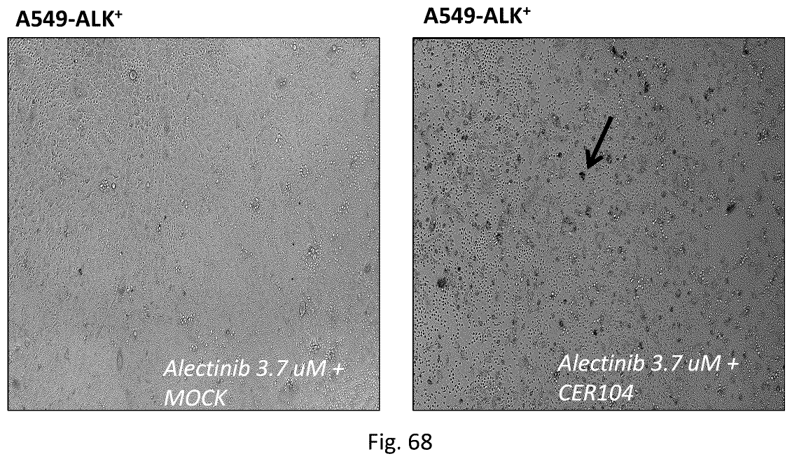

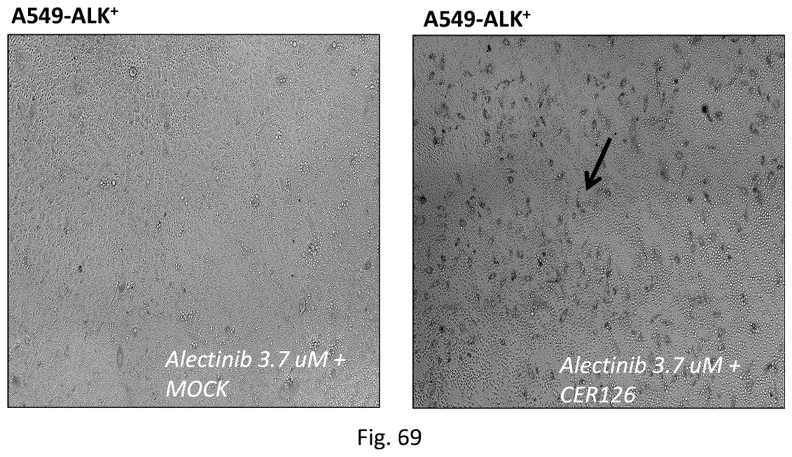

CER21-, CER108-, CER104-, and CER129-transduced CD4+ T cells were tested for their ability to kill A549 ALK+ cells in the presence of ALK inhibitor. CD4+ T cells were transduced with a lentiviral vector comprising a CER21, CER108, CER104, CER129, CER27, CER120, CER122, CER123, CER124, or CER126 nucleic acid. A549 lung adenocarcinoma cells introduced with an ELM4-Alk translocation were co-cultured with CER-transduced or mock-transduced (vector only) CD4+ T cells at 1:1 effector:target cell ratio with 3.7 μM Alectinib for 48 hours. After 48 hours of co-culture, T cells were washed away and the number of viable A549 ALK+ cells was quantified using a calorimetric MTT assay (FIGS. 67A-B). Cell viability experiments were performed in triplicates and presented as % of control. Bright field microscopy images were also obtained from A549 ALK+co-culture experiments. FIG. 68 shows A549 ALK+ cells treated with Alectinib (3.7 μM) for 48 hours and co-cultured with mock-transduced T cells (left image) or CER104-transduced T cells (right image). Arrow indicates cluster of dead A549 ALK+ cells surrounded by phagocytic CER104+ T cells. FIG. 69 shows A549 ALK+ cells treated with Alectinib (3.7 μM) for 48 hours and co-cultured with mock-transduced T cells (left image) or CER126-transduced T cells (right image). Arrow indicates cluster of dead A549 ALK+ cells surrounded by phagocytic CER104+ T cells.

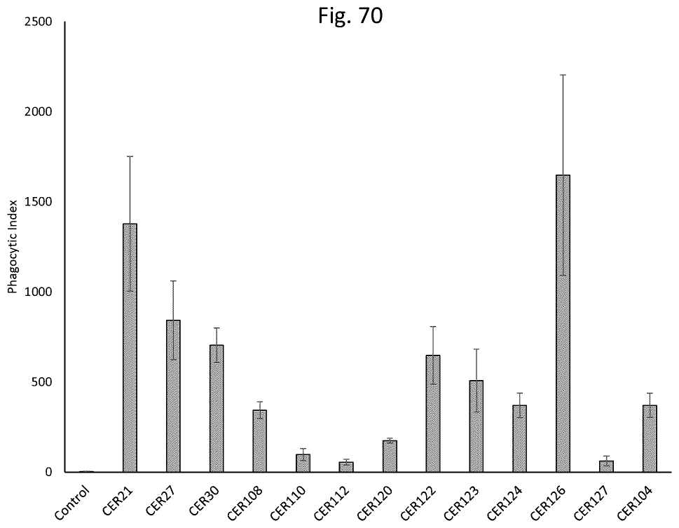

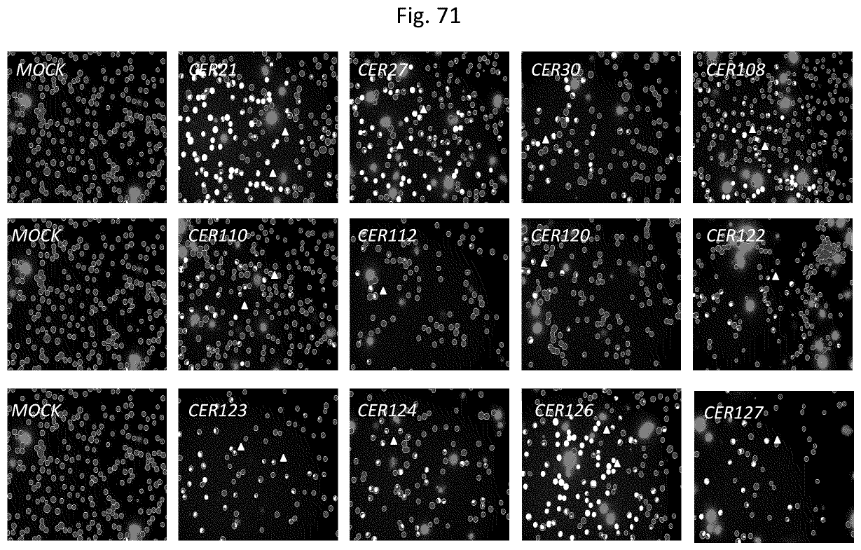

Phagocytosis of alectinib treated A549 lung adenocarcinoma cells by various CER-transduced CD4+ T cells was also quantified. A549 cells were treated with Alectinib (1 μM) for 12 hours. CER21-, CER27-, CER30-, CER108-, CER110-, CER112-, CER120-, CER123-, CER124-, CER126-, CER127-, or CER104-transduced CD4+ T cells were co-cultured with Alectinib-treated A549 cells at a 1:1 ratio for 12 hours. Phagocytic events were calculated from 3×3 stitched fluorescent microscopy images at 40× resolution. FIG. 70 shows an adjusted phagocytic index calculated as (median area ratio of target events in effector cells*% phagocytosis). FIG. 71 shows fluorescent micrographs of Alectinib-treated A549 ALK+ cells that were labeled with pHrodo red and co-cultured with CER-transduced T cells labeled with CELL TRACE violet. Images were obtained after 12 hours of co-culture. Yellow triangles indicate exemplary phagocytic events (pHrodo red targets within CELLTRACE violet labeled CD4+ T cells). Mock-transduced (vector only) T cells exhibit no phagocytic activity.

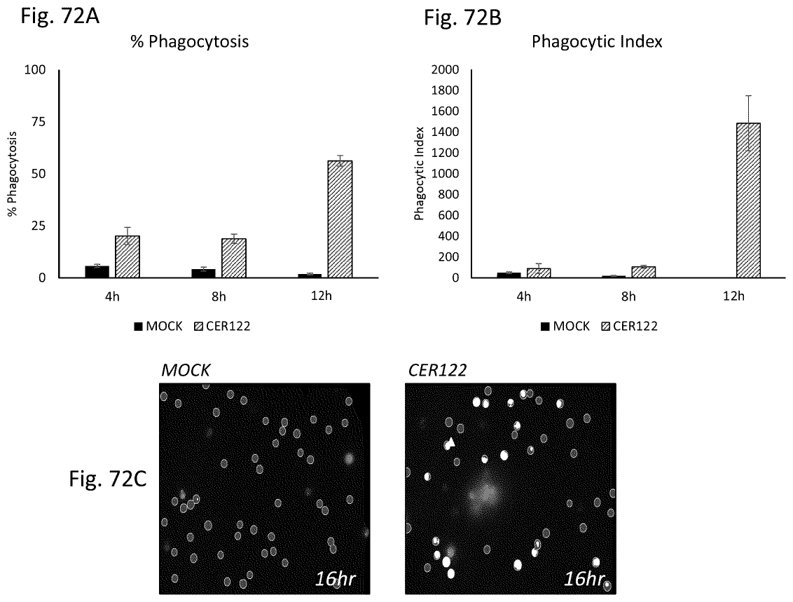

A time course study of phagocytic uptake of tumor cells by CER-transduced T cells was performed. CD4+ T cells were transduced with a lentiviral vector comprising CER122 nucleic acid. Mock-transduced (vector only) CD4+ T cells were used as control. CER122-transduced T cells were labeled with CELLTRACE violet and co-cultured with pHrodo red labeled A549 ALK+ lung adenocarcinoma cells. Fluorescent microscopy images were obtained at 4, 8, and 16 hours co-culture (FIG. 72C), and % phagocytosis (FIG. 72A) and phagocytic index (FIG. 72B) were calculated. Yellow triangles indicate exemplary phagocytic events by CER122-transduced T cells (pH rodo red targets within CELLTRACE violet labeled CD4+ T cells) (right image of FIG. 72C). Mock-transduced T cells exhibit no phagocytic activity (FIGS. 72C, left image)

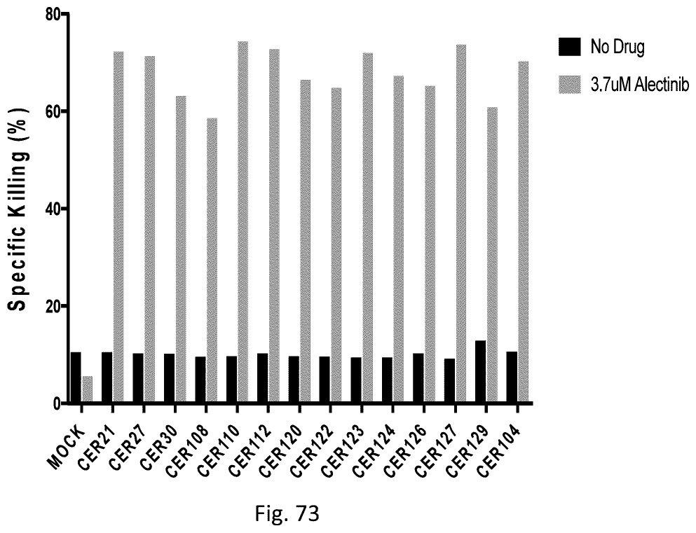

Testing of additional CER types showed synergistic killing of A549 lung adenocarcinoma cells harboring ALK rearrangement upon ALK inhibition (FIG. 73). A549 cells introduced with an ELM4-ALK translocation were co-cultured with mock-transduced (vector only) CD4+ T cells or CER-transduced CD4+ T cells (CER21, CER27, CER30, CER108, CER110, CER112, CER120, CER122, CER123, CER124, CER126, CER127, CER129, or CER104) in combination with Alectinib (3.7 μM). Effector:target cell ratio for co-culture was 1:1. After 18 hours of co-culture, bulk supernatants were evaluated by LDH cytotoxicity assay to measure cell killing (FIG. 73). CER-expressing T cells demonstrated synergistic killing responses upon ALK inhibition.

### CER Enhancement of ALK Inhibitor In Vivo

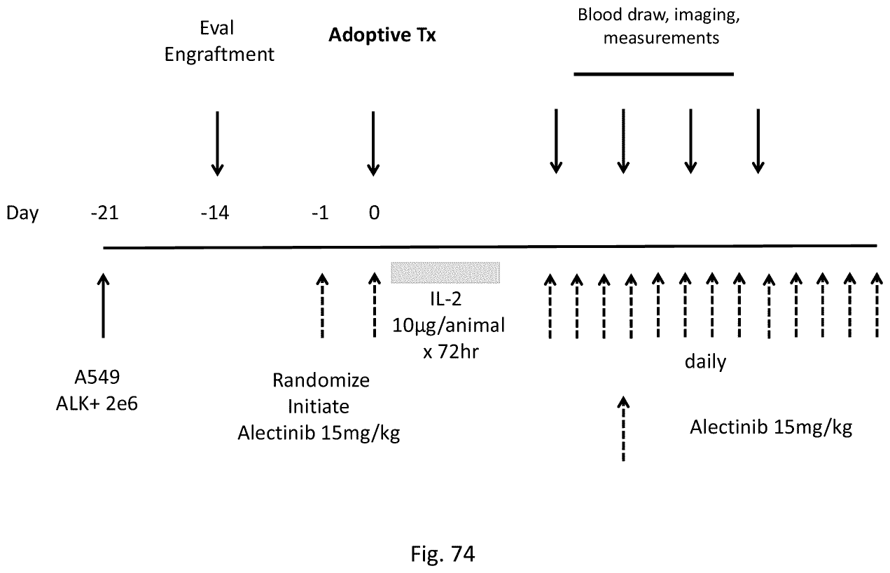

The combination of CER therapy and ALK inhibitor therapy was also evaluated in vivo. FIG. 74 shows a schematic depicting details of adoptive cell therapy experiments using CER-expressing T cells in combination with ALK inhibitor therapy. A549 ALK+ Luciferase⁺ cells were engrafted into immunodeficient (NSG) mice 21 days prior to initiation of study and evaluated for engraftment by bioluminescent emission and tumor volume seven days later. The day prior to CER adoptive transfer (day −1), animals with established tumors were randomized into groups and treated with Alectinib 15 mg/kg (intraperitoneally) and then infused retro-orbitally with 10e6 CER-transduced human T cells. Animals received 10 μg of systemic IL-2 every 24 hours×3 days following cell infusion and then monitored for tumor progression and cell expansion and persistence thereafter.

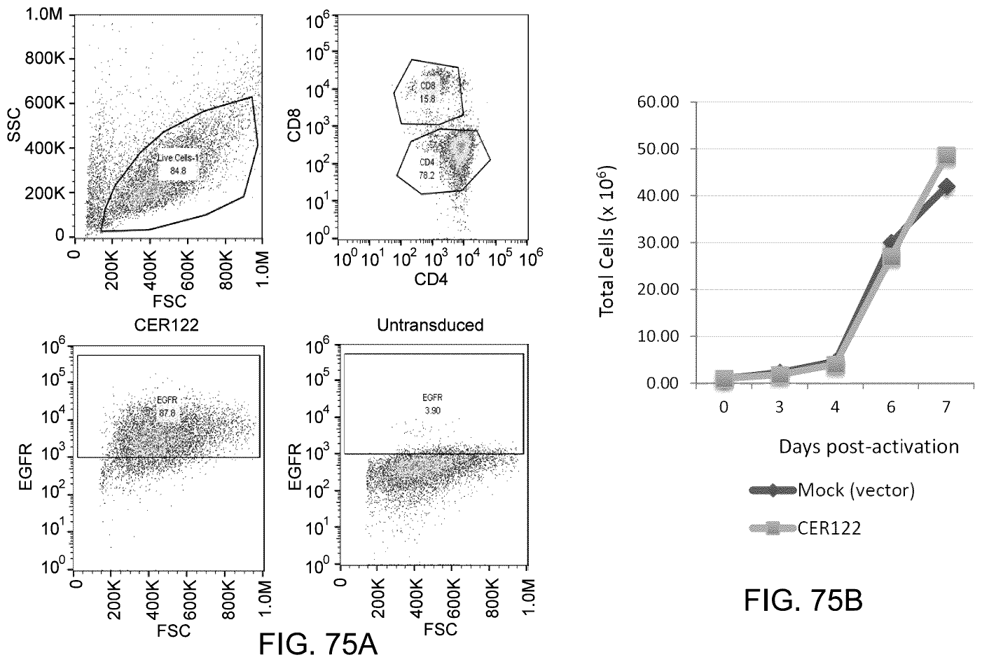

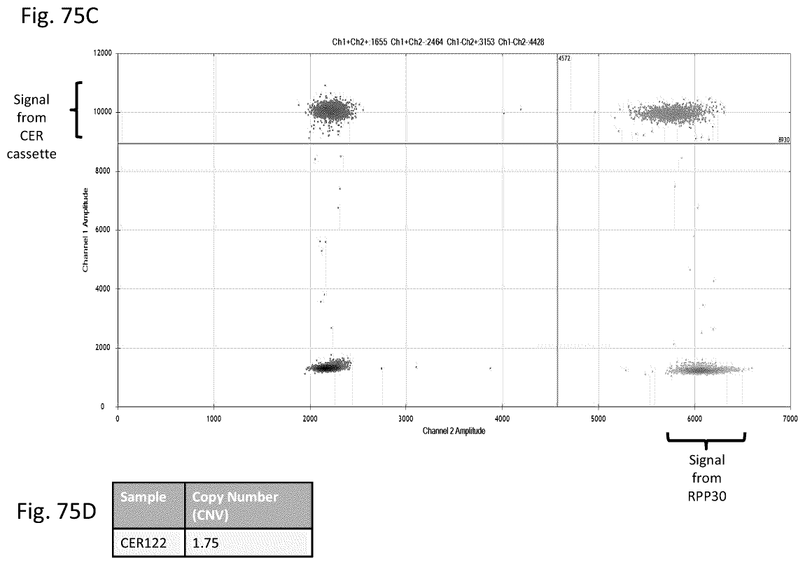

CER-expression T cells can be expanded ex vivo. T cells were enriched, activated, and transduced with a CER122-T2A-tEGFR lentiviral construct and phenotyped for surface EGFR and T-cell markers CD4, CD8 by FACS (FIG. 75A). The total number of transduced and control T cells (transduced with vector only) in unselected cultures was determined after CD3 and CD28 bead activation (FIG. 75B). 2-D fluorescence droplet digital PCR was performed on CER122-transduced T cell genomic DNA and demonstrates amplification of a region from the CER cassette (FIG. 75C). Copy number value for CER122-transduced T cells was determined from digital droplet PCR (FIG. 75D).

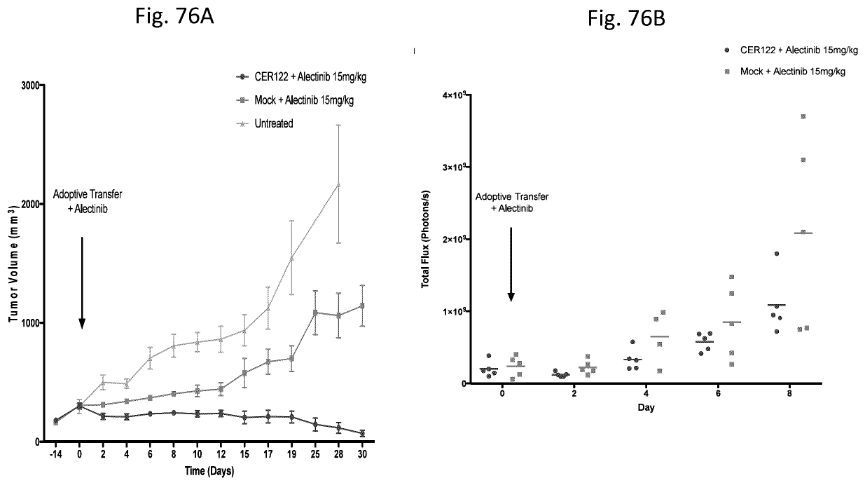

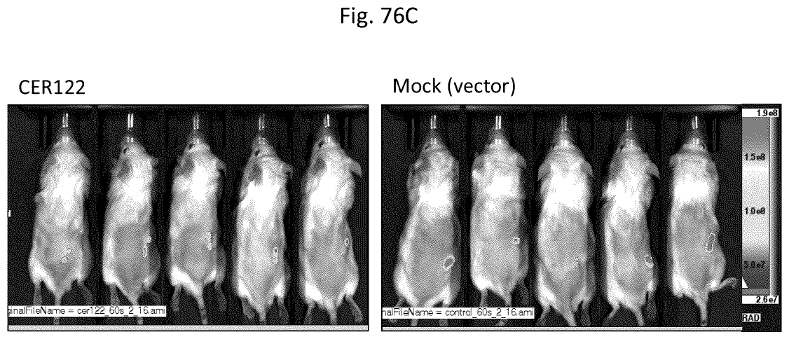

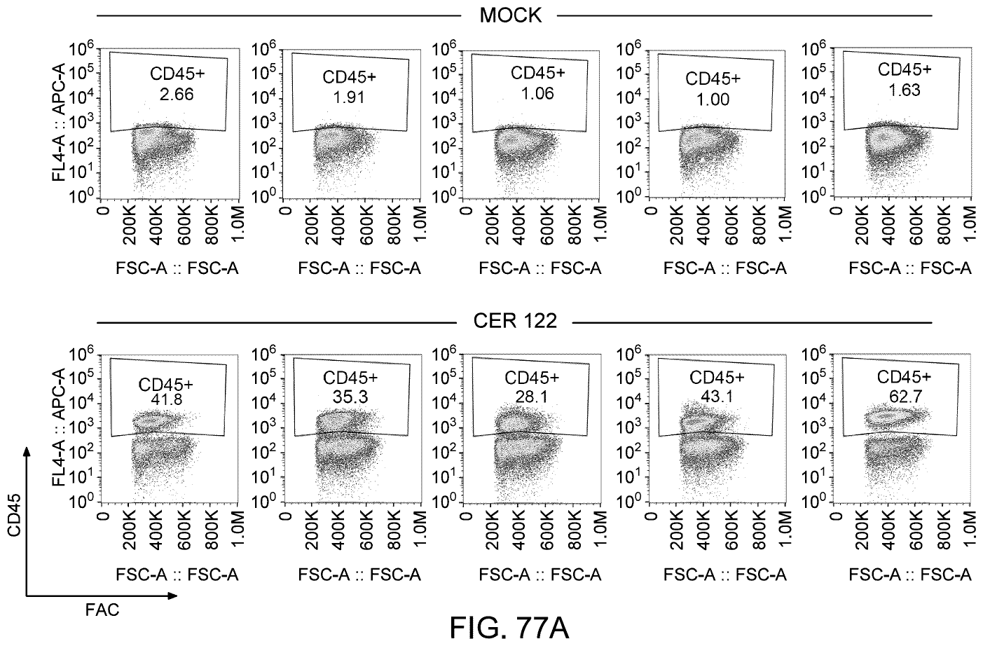

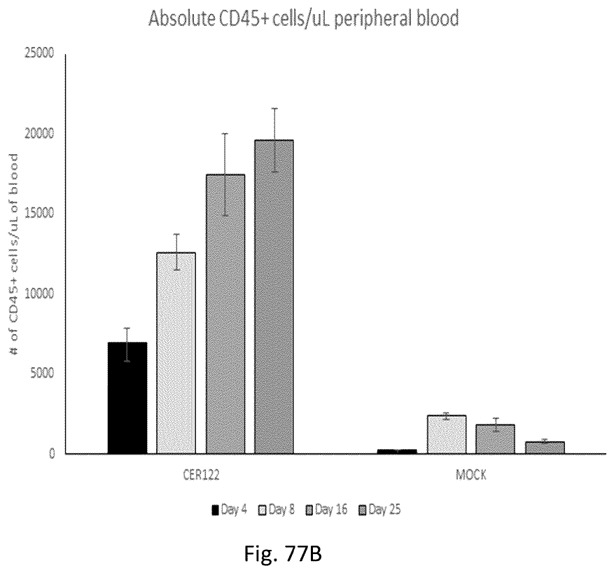

FIG. 76A shows tumor volume measurements from −14 to 30 days post-adoptive transfer in untreated, Alectinib only, and Alectinib+CER122-transduced T cells (n=5 per group). FIG. 76B shows growth of A549-luciferase+ ALK-positive cells in NSG mice from 0 to 8 days post-adoptive transfer, as evaluated by bioluminescence imaging. FIG. 76C shows bioluminescence image of A549 ALK-positive tumor burden at day 8 post-adoptive transfer. Combination of CER122 therapy with alectinib treatment enhanced anti-tumor response to ALK+ NSCLC in vivo. CER-transduced T cells exhibited early expansion post-adoptive transfer. FACS plots demonstrate early expansion of CD45⁺ human cells in peripheral blood of animals post-adoptive cell treatment (FIG. 77A). Peripheral blood sample was stained with anti-human CD45-APC-conjugated antibody 8 days following cell infusion. Each FACS plot indicates a single animal. A bar graph depicting frequency of human CD45+ cells/μL of peripheral blood at days 4, 8, 16, and 25 post-adoptive transfer of CER122+ T cells (FIG. 77B). Peripheral blood was obtained by retro-orbital bleeding and examined for evidence of T cell engraftment by FACS. The frequency of human-CD45 cells/μL of peripheral blood was determined by FACs using TRUCOUNT (BD Biosciences) beads.

### CER Enhancement of EGFR Inhibitor In Vivo

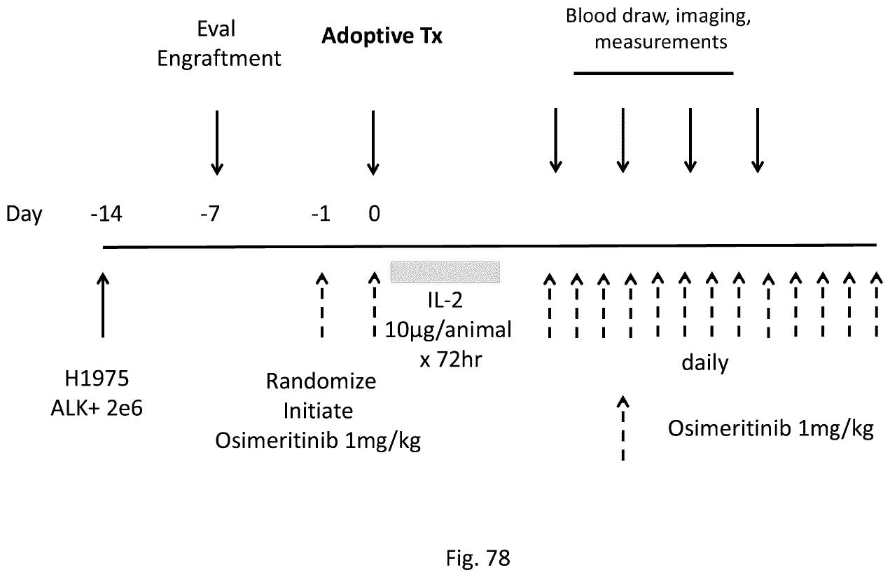

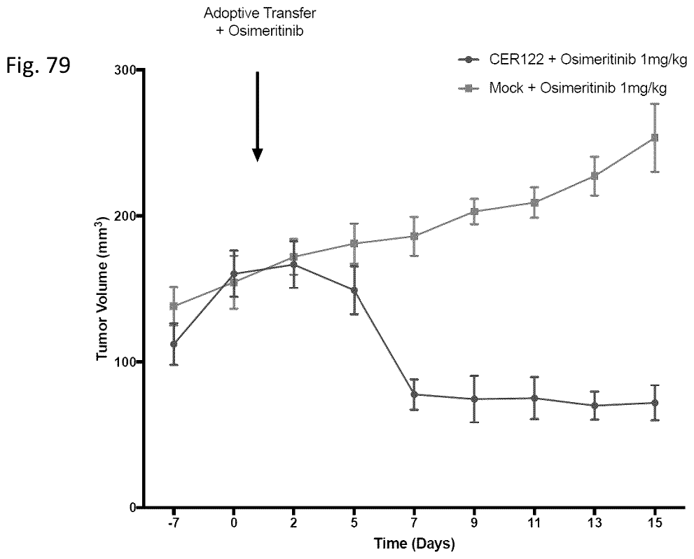

The combination of CER therapy and EGFR inhibitor therapy was also evaluated in vivo. FIG. 78 shows a schematic depicting details of adoptive cell therapy experiments using CER-expressing cells in combination with EGFR inhibitor therapy. H1975 EGFR+/Luciferase⁺ cells were engrafted into immunodeficient (NSG) mice 14 days prior to initiation of study and evaluated for engraftment by bioluminescent emission and tumor volume seven days later. The day prior to CER adoptive transfer (day −1), animals with established tumors were randomized into groups and treated with Osimeritinib 1 mg/kg (intraperitoneally) and then infused by tail vein with 10e6 CER-transduced human T cells. Animals received 10 μg of systemic IL-2 every 24 hours×3 days following cell infusion and then monitored for tumor progression and cell expansion and persistence thereafter. Tumor volume measurements were obtained post-adoptive transfer in untreated, Osimeritinib treated (1 mg/kg)+mock-transduced T cells (vector only), and Osimeritinib (1 mg/kg)+CER122-transduced T cells (n=5/group) (FIG. 79). CER122-expressing T cells combined with Osimeritinib enhanced anti-tumor responses in EGFR+ NSCLC model in vivo.

### Example 8

### Characterization of CER Modified CD4 T Cells

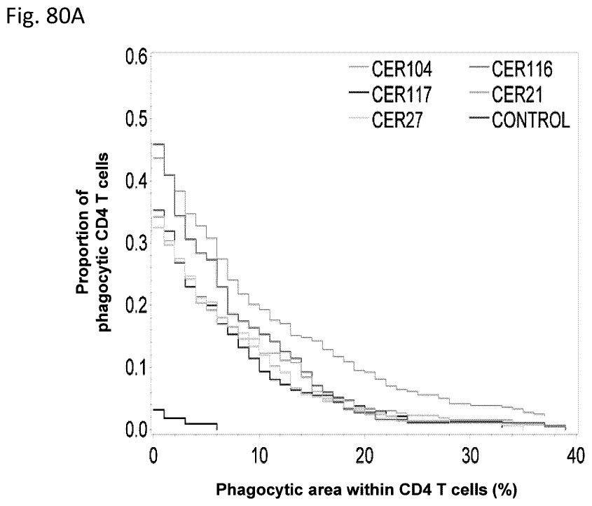

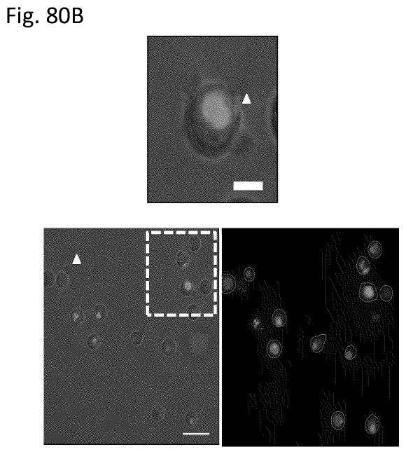

Various CER-modified CD4+ T cells were also evaluated for breadth of response to determine whether a particular CER confers a broad phagocytic response of low magnitude (e.g., 10% engulfment in 90% of cells) or a less frequent but strong phagocytic response (e.g., 90% engulfment in 10% of cells) in the host cells. CD8+ T cells were transduced with HPV16 E7 specific TCR as described in Example 5. CD4+ T cells were transduced with lentiviral vectors comprising a CER21, CER27, CER104, CER116, or CER117 nucleic acid. Mock-transduced (vector alone) CD4+ T cell were used as control. CD4+/CER+ and CD8+/E7 TCR+ T cells were stained with CELLTRACE violet. HPV16 E7+ head and neck squamous cell carcinoma cells (SCC152) were stained with pHrodo red. HPV16 E7 TCR transduced CD8+ T cells and selected CER transduced CD4+ T cells were mixed at a 1:1 ratio and co-cultured with SCC152 cells at a 1:1 ratio for 8 hours. Phagocytosis of target SCC152 cells by CER-transduced CD4+ T cells was analyzed by fluorescence microscopy. FIG. 80A shows a magnitude breadth curve for phagocytosis by CER type. The horizontal axis represents the % area of CER-transduced CD4+ T cells having engulfment or % area of the CER-transduced CD4+ T cells taken up by target SCC152 cells. This measure was rarely above 40% across CER types tested. The vertical axis represents the proportion of CER-transduced CD4+ T cells that were phagocytic. For CER104, about 20% of CER104-transduced CD4+ T cells have more than 10% engulfment. For CER117-transduced CD4+ T cells, less than 10% have more than 10% engulfment. FIG. 80B shows fluorescent micrograph images of SCC152 target cells engulfed by CER126-transduced CD4+ T cells.

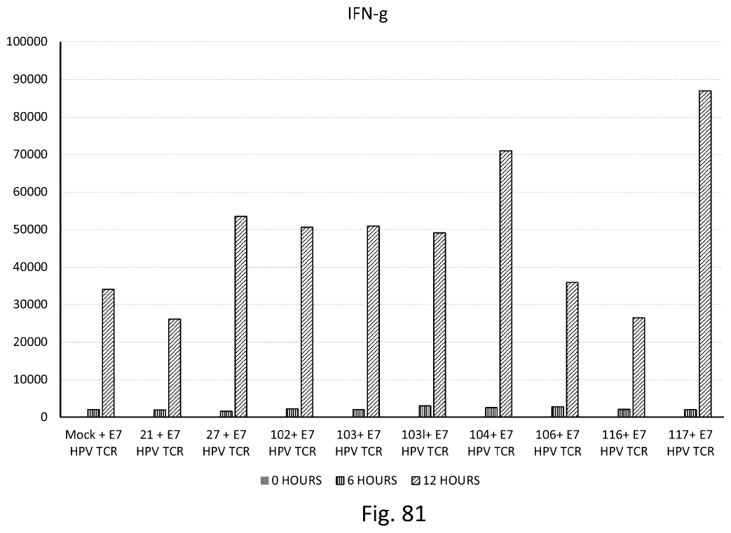

CD4+ T cells were transduced with lentiviral vectors comprising a CER21, CER27, CER102, CER103A, CER103B, CER104, CER106, CER116, or CER117 nucleic acid. Mock-transduced (vector alone) CD4+ T cell were used as control. CD8+ T cells were transduced with HPV16 E7 specific TCR. HPV16 E7 TCR transduced CD8+ T cells and selected CER transduced CD4+ T cells were mixed at a 1:1 ratio and co-cultured with SCC152 cells at a 1:1 ratio for 10 hours. Supernatants were then collected and analyzed for bulk cytokine secretion. As shown in FIG. 81, addition of a CER-expressing CD4+ T cell to E7 TCR-transduced CD8+ T cells enhanced levels of IFNγ secretion.

### Example 9

### Marker Analysis of CER Modified CD4 T Cells

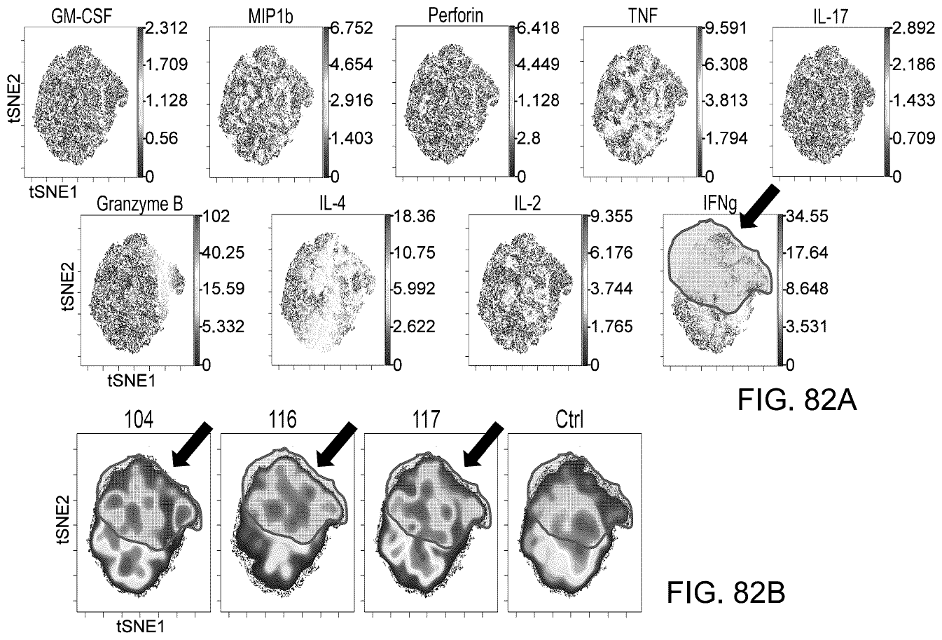

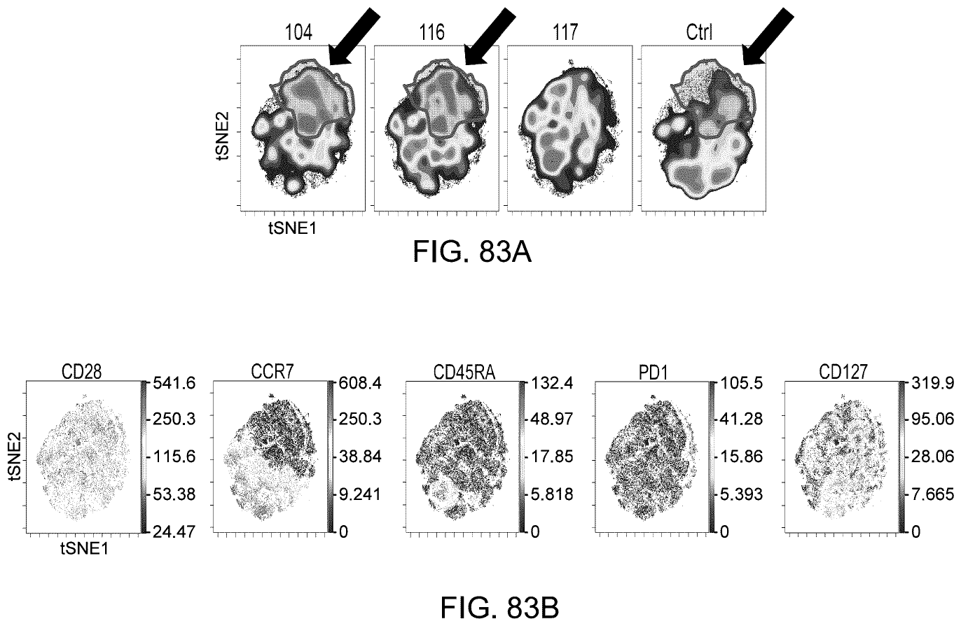

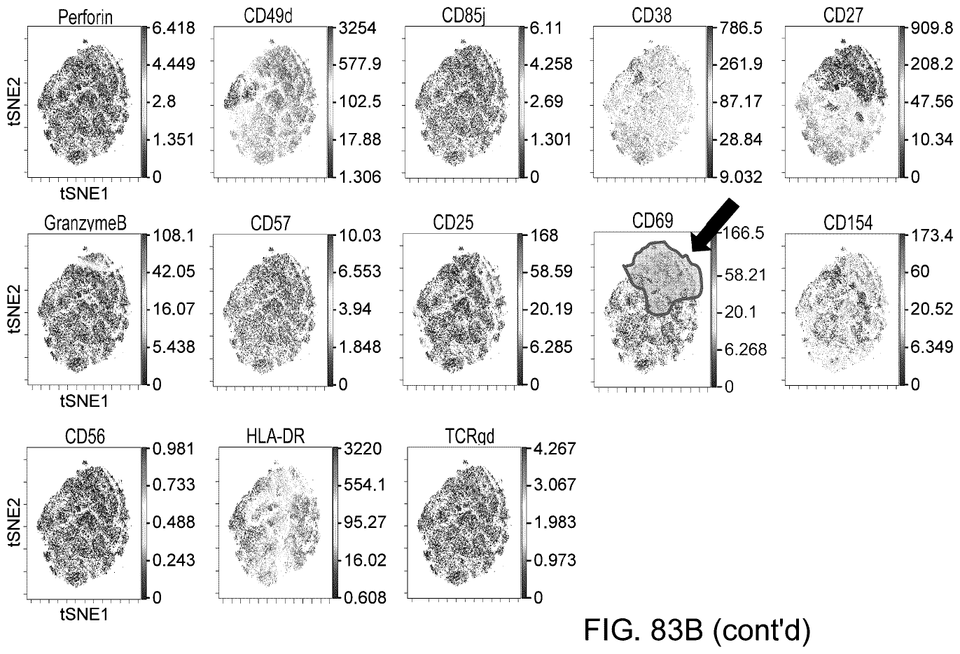

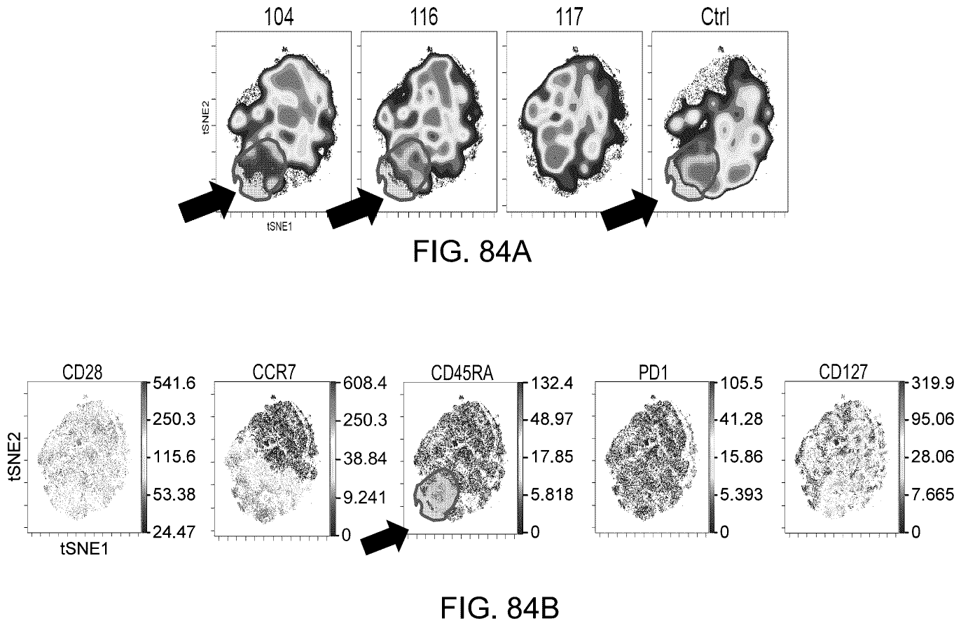

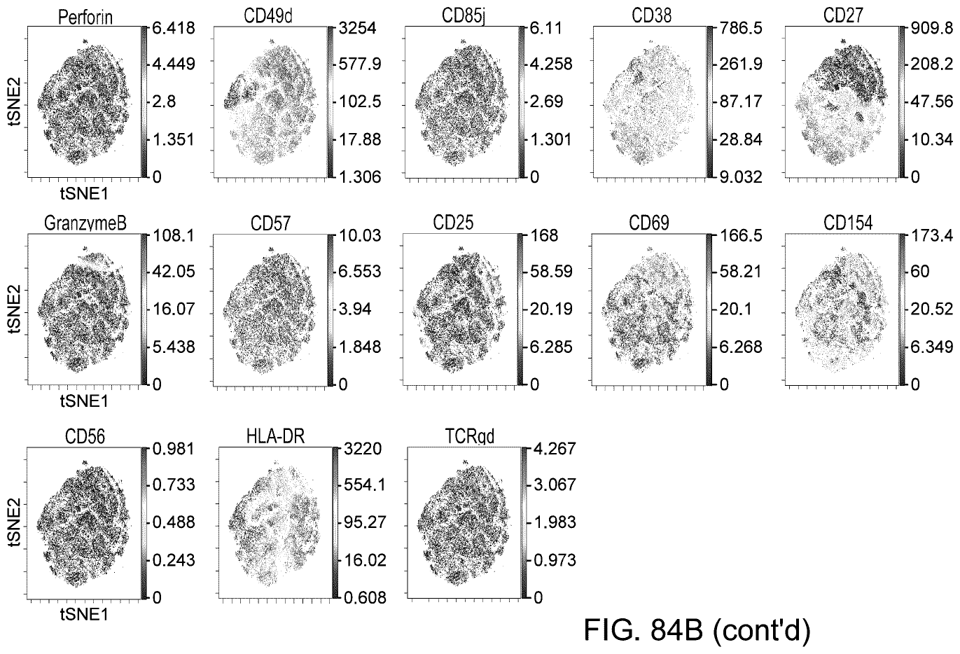

CD4+ T cells were transduced with lentiviral vector comprising CER104, CER116, or CER117 nucleic acid. CER104 (SEQ ID NO:133) comprises a Tim4 binding domain, Tim4 transmembrane domain, and an engulfment signaling domain comprising a primary engulfment signaling domain comprising a TLR8 signaling domain and a secondary engulfment signaling domain comprising a DAP12 signaling domain. CER116 (SEQ ID NO:143) comprises a Tim4 binding domain, Tim4 transmembrane domain, and an engulfment signaling domain comprising a primary engulfment signaling domain comprising a TRAF6 signaling domain and a secondary engulfment signaling domain comprising a TLR8 signaling domain. CER117 (SEQ ID NO:144) comprises a Tim4 binding domain, Tim4 transmembrane domain, and an engulfment signaling domain comprising a primary engulfment signaling domain comprising a TLR8 signaling domain and a secondary engulfment signaling domain comprising a TRAF6 signaling domain. CER-transduced CD4+ T cells were co-cultured with E7 TCR-transduced CD8+ T cells and HPV+ SCC152 target cells and interrogated by mass cytometry (CyTOF) with viSNE for visualization of high dimensional single cell data (FIGS. 82-84). Intact CER-transduced CD4+ T cells are shown in plots displaying tSNE1 (horizontal) and tSNE2 (vertical) axes. 27 intracellular markers were used for the viSNE analysis. Each dot represents a single cell. Coloring the plots by a few of the measured markers (GM-CSF, MIP1b, Perforin, TNF, IL-17, Granzyme B, IL-4, IL-2, and IFNγ) shows the phenotype across viSNE ‘islands’ (FIG. 82A). Red represents high expression and blue represents low expression for each marker. Populations of CD4 T cells were generated using a clustering algorithm from all 27 markers and overlaid onto the viSNE map. Arrows indicate enrichment of islands expressing the intracellular marker IFNγ in samples containing CER104, CER116, and CER117 (FIG. 82B). Populations of CD4 T cells were generated using a clustering algorithm from all 18 markers and overlaid onto the viSNE map (FIG. 83A). Arrows indicate enrichment of islands expressing the T cell activation marker CD69 in samples containing CER104- and CER116-transduced CD4+ T cells. Color plots by 18 intracellular markers (CD28, CCR7, CD45RA, PD1, CD127, Perforin, CD49d, CD85j, CD38, CD27, Granzyme B, CD57, CD25, CD69, CD154, CD56, HLA-DR, and TCRγδ) show the phenotype across viSNE ‘islands’ (FIG. 83B). Red represents high expression and blue represents low expression for each marker. Highlighted region with arrow indicates cells expressing T cell activation marker CD69. Populations of CD4 T cells were generated using a clustering algorithm from 18 intracellular markers (CD28, CCR7, CD45RA, PD1, CD127, Perforin, CD49d, CD85j, CD38, CD27, Granzyme B, CD57, CD25, CD69, CD154, CD56, HLA-DR, and TCRγδ) and overlaid onto the viSNE map. Arrows indicate loss of islands expressing the naïve T cell marker CD45RA within the CCR7 population among CER104 and CER116 samples compared to controls (FIG. 84A). Color plots by the 18 intracellular markers show the phenotype across viSNE ‘islands’ (FIG. 84B). Red represents high expression and blue represents low expression for each marker. Highlighted region with arrow indicates cells expressing the naïve T cell marker CD45RA. Thus, this data show that CER104 and CER116-transduced CD4+ T cells are associated with memory formation after antigen encounter.

### Example 10

### Phagocytic Signaling Transduction in CER Modified Cells

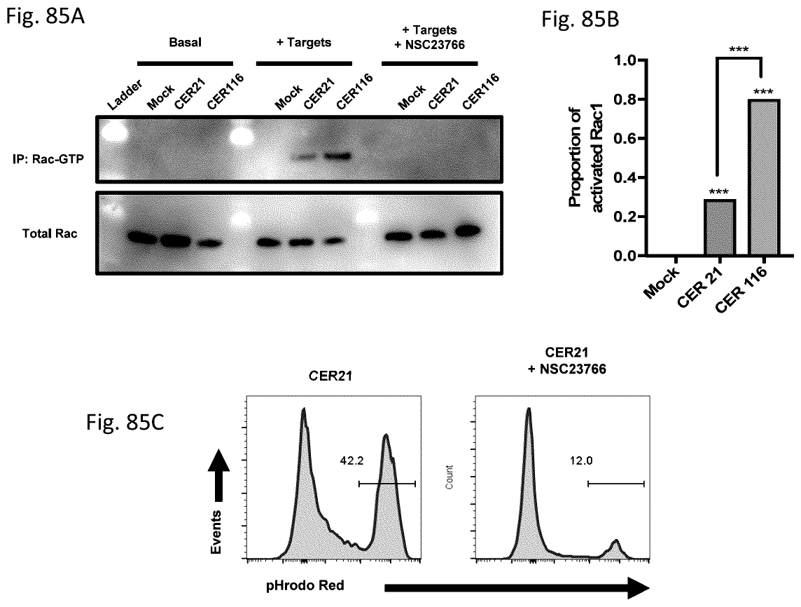

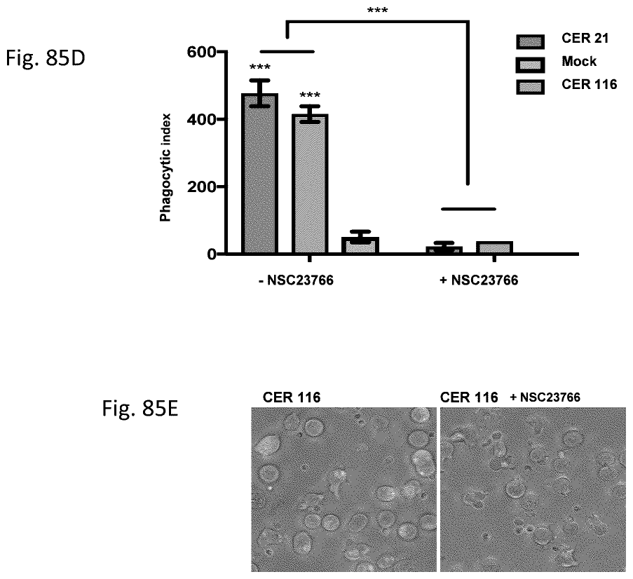

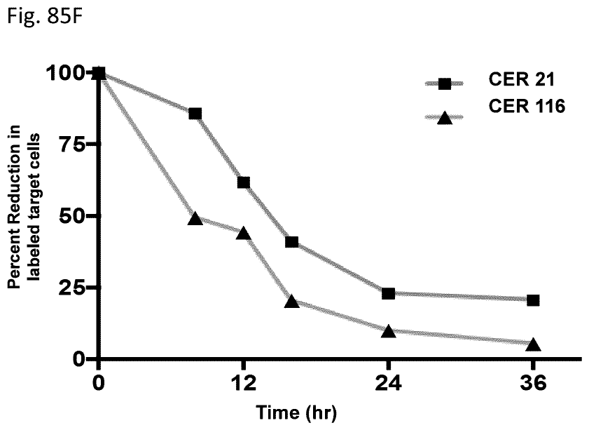

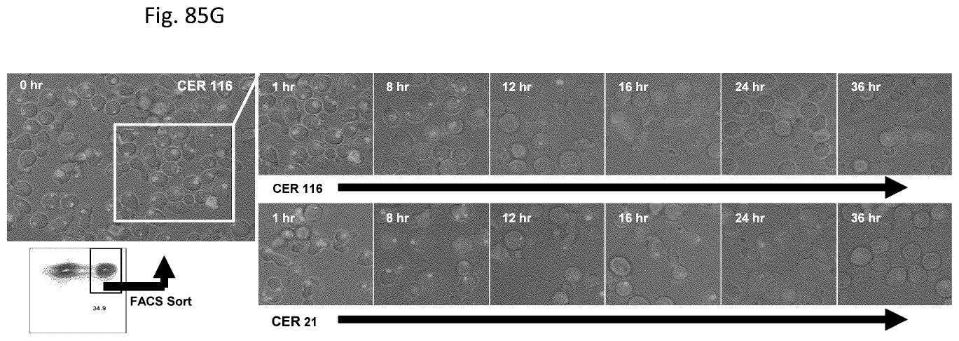

The Rho family of GTPases play essential roles in actin assembly during phagocytosis. Inhibition of either Rho GTPase Rac1 or CDC42 in macrophages results in a complete blockade of FcR-mediated phagocytosis due to defective actin assembly. To investigate induction of phagocytic signal transduction cascades, CER-expressing cells were interrogated for the activation of CDC42 and Rac1. The activation of either Rho GTPase involves the transition from an inactive GDP-bound form to an active GTP-bound form, catalyzed by guanine nucleotide-exchange factors (GEFs). Ba/F3 cells were transduced with lentiviral vectors comprising CER21 nucleic acid, CER116 nucleic acid, or vector alone (mock). Target thymocyte cells were stained with pHrodo red. CER-transduced Ba/F3 cells were co-cultured with dexamethasone pre-treated thymocytes for 2 hours. The Rac1 inhibitor NSC23766 (Selleck Chem) was added during the co-culture in appropriate wells. Cells were then collected, solubilized in lysis buffer, and immunoprecipitation was performed on protein lysates to detect phospho-Rac1 using PAK-PBD agarose beads (Cytoskeleton, Inc.). Immunoprecipitates were eluted and 25 μg of protein was loaded onto SDS-PAGE gradient gels and then probed with monoclonal, mouse anti-Rac1 primary antibody (Cytoskeleton Inc.) overnight at 4° C., washed, and hybridized with secondary, anti-mouse antibody conjugated to horseradish peroxidase (Jackson Labs) (FIG. 85A). Prior to immunoprecipitation, Some protein lysate sample was reserved for total protein estimation and total Rac1 estimation. Basal samples comprise CER-expressing cells cultured without target cells. Protein gel bands were quantified using ImageJ image processing program, and the proportion of activated Rac1 was quantified (FIG. 85B). CER21 (SEQ ID NO:88) comprises a Tim4 binding domain, a Tim4 transmembrane domain, and a TLR8 signaling domain. CER116 (SEQ ID NO:143) comprises a Tim4 binding domain, a Tim4 transmembrane domain, an engulfment signaling domain comprising a primary engulfment signaling domain comprising a TRAF6 signaling domain and a secondary engulfment signaling domain comprising a TLR8 signaling domain. The addition of a TRAF6 signaling domain enhanced Rac1 signaling in CER116-transduced Ba/F3 cells compared to CER21-transduced Ba/F3 cells. FIG. 85C shows representative FACs profiles of pHrodo in CER21-transduced Ba/F3 cells after 6 hour co-culture. CER21-transduced Ba/F3 cells shows considerable pHrodo+ signal indicating phagocytosis of target thymocytes (see, FIG. 85C left image). The addition of a specific Rac1 small molecule inhibitor abolishes phagocytosis (FIG. 85C, right image). The numbers associated with each peak indicates the percentage (phagocytosis) of pHrodo thymocytes in CER21+ Ba/F3 cells. Phagocytic indices (FIG. 85D) were calculated from fluorescent imaging shown in FIG. 85E. Representative fluorescent micrographs of phagocytosis assays of CER116-harboring Ba/F3 cells in presence or absence of Rac1 inhibitor NSC23766 (FIG. 85E). Engulfed thymocytes stained with pHrodo red are observed inside CELLTRACE violet stained CER116+ Ba/F3 cells. Rac1 inhibition abolishes CER116 mediated phagocytosis of thymocytes.

Luminal degradation in CER-transduced cells was also evaluated following engulfment. Ba/F3 cells transduced with CER21 or CER116 were co-cultured with pHrodo-red labeled, dexamethasone treated thymocytes overnight and subsequently purified by FACS. Target cell destruction was visualized by time-lapse imaging and quantified over time (FIG. 85F). The addition of a TRAF6 signaling domain enhanced CER116 luminal content degradation over time as compared to CER21, with near complete resolution of luminal contents by 36 hours (FIG. 85F). Time lapse imaging of CER116+ Ba/F3 cells (FIG. 85G, top row images) and CER21+ Ba/F3 cells (FIG. 85G, bottom row images) demonstrates destruction of luminal contents. pHrodo-red labeled contents are broken down over-time in both CER116- and CER21-modified cells, but appears to be more rapid in CER116-modified cells. CER116-harboring Ba/F3 cells (top) catabolize target cells, allowing cells to return to homeostasis and resume immune responsiveness. The addition of a TRAF6 signaling domain to the CER construct promotes rapid destruction of engulfed material.

### Example 11

### Antigen Presentation by CER Modified T Cells

One strategy to enhance tumor cell killing by cytotoxic CD8+ T cells (CTLs) is to utilize antigen presenting cells (APCs), which have the unique capacity to “cross-present” exogenous antigen on MHC I molecules. Broadening tumor-specific CTL responses has the potential to induce effective immune responses against tumors. In this example the viral HPV E6 and E7 oncoproteins were used as model antigens to characterize the antigen processing and presenting capacity of chimeric engulfment receptor (CER)-expressing cells.

CD4+ and CD8+ CER-expressing T cell lines were established from human PBMCs. Purified T cells were transduced with lentivirus encoding CER123 (SEQ ID NO:150) and truncated EGFR (transduction marker), after activation with CD3 & CD28 microbeads, and then expanded in medium containing IL-7, IL-15, and IL-2 for 5 days. The percentage of tEGFR+ T cells ranged between 40-60%.

A Jurkat cell line with a stable integration of an NFAT-inducible Luciferase reporter construct was utilized to study T cell responses. Human E6- and E7-specific engineered TCRs were transduced into Jurkat NFAT reporter cell lines to characterize NFAT activation upon co-culture with engineered CERs.

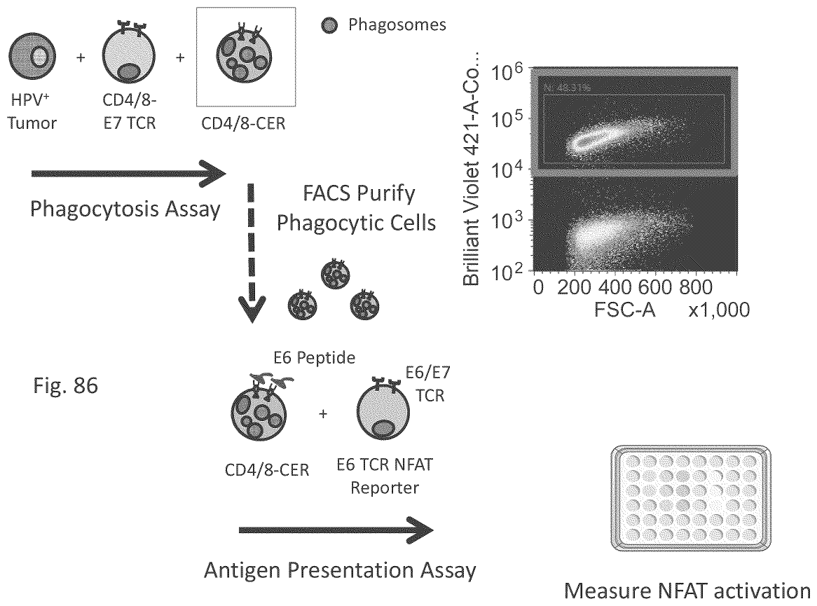

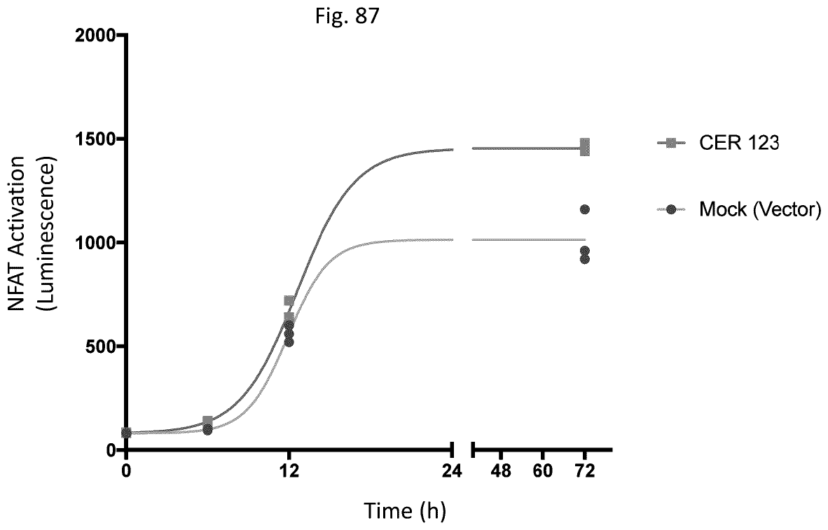

For assessing MHC-I cross-presentation, SCC152 HPV⁺ cells were co-cultured overnight with CER123-expressing CD4+ and CD8+ T cells or mock-transduced (vector only) T cells in the presence of T cells expressing an E7-specific TCR. Following overnight co-culture, CER123-expressing T cells or Mock-transduced T cells were purified using FACS, washed, and subsequently cultured with E6/E7-specific human TCR/NFAT reporter cell line at a 1:1 ratio. NFAT activation was assessed at serial time points (0, 6, 12, 24, and 72 hrs) by measuring luciferase activity in cell culture supernatants. A schematic of this assay is provided in FIG. 86. Cells were cultured in RPMI/10% FCS in 96-well round-bottom plates. CER123-expressing CD4⁺ and CD8⁺ T cell lines, after phagocytosing HPV⁺ tumors, were co-cultured overnight with Jurkat T cells expressing a E7₁₁₋₁₉-specific TCR and an NFAT reporter. Induction of E7₁₁₋₁₉-specific Jurkat T cells were quantified by luminescence of NFAT signaling at indicated time points and compared to Mock (vector-alone) transduced T cells (FIG. 87). CER123-expressing T cells demonstrated enhanced cross-presentation efficiency of HPV E7 oncoproteins following phagocytosis of HPV⁺ tumor cells.

Additional definitions are provided throughout the present disclosure.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application Nos. 62/563,615, 62/649,529, and 62/652,822 are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1.-69. (canceled)

70. A method of treating a subject with cancer, the method comprising administering to the subject an effective amount of a population of T cells comprising a nucleic acid molecule encoding a chimeric engulfment receptor (CER) comprising a single chain chimeric protein, the single chain chimeric protein comprising:
(a) an extracellular domain comprising a Tim4 binding domain that binds to phosphatidylserine (PtdSer);
(b) an engulfment signaling domain that comprises a toll-like receptor (TLR) signaling domain, a Traf2 signaling domain, or a Traf3 signaling domain; and
(c) a transmembrane domain positioned between and connecting the extracellular domain and the engulfment signaling domain;
thereby treating the subject.

71.-74. (canceled)

75. The method of claim 70, wherein the cell is an autologous cell or allogeneic cell.

76. (canceled)

77. The method of claim 70, further comprising administration of a second therapeutic agent to the subject.

78. The method of claim 77, wherein the second therapeutic agent is an antibody, radiation therapy, chemotherapeutic agent, cellular immunotherapy, antibiotic, anti-fungal agent, or anti-viral agent.

79. The method of claim 77, wherein the population of T cells comprising the nucleic acid encoding the CER is administered concurrently with or subsequent to administration of the second therapeutic agent.

80. The method claim 77, wherein the second therapeutic agent is administered at a subtherapeutic dose.

81. The method of claim 70, wherein the TIM4 domain comprises the amino acid sequence of SEQ ID NO:3 or amino acids 25-314 of SEQ ID NO:3.

82. The method of claim 70, wherein the extracellular domain further comprises an extracellular spacer domain positioned between the binding domain and transmembrane domain.

83. The method of claim 70, wherein the extracellular spacer domain comprises an immunoglobulin hinge region, a hinge region of a type 1 membrane protein, a stalk region of a type II C-lectin, an immunoglobulin constant domain, a juxtamembrane region of a toll-like receptor, or a fragment thereof.

84. The method of claim 83, wherein the extracellular spacer domain comprises:
(a) an IgG1 hinge region, IgG2 hinge region, IgG3 hinge region, IgG4 hinge region, IgA hinge region, or IgD hinge region;
(b) a modified IgG4 hinge region comprising the amino acid sequence of SEQ ID NO:16;
(c) a TLR1 juxtamembrane region, TLR2 juxtamembrane region, TLR3 juxtamembrane region, TLR4 juxtamembrane region, TLR5 juxtamembrane region, TLR6 juxtamembrane region, TLR7 juxtamembrane region, TLR8 juxtamembrane region, or TLR9 juxtamembrane region; or
(d) a TLR4 juxtamembrane region comprising the amino acid sequence of SEQ ID NO:17.

85. The method of claim 70, wherein the transmembrane domain comprises a Tim1 transmembrane domain, Tim4 transmembrane domain, Tim3 transmembrane domain, FcγR1 transmembrane domain, FcγR2A transmembrane domain, FcγR2B2 transmembrane domain, FcγR2C transmembrane domain, FcγR3A transmembrane domain, FcεR1 transmembrane domain, FcαR1 transmembrane domain, CD8a transmembrane domain, CD28 transmembrane domain, MERTK transmembrane domain, Axl transmembrane domain, MRC1 transmembrane domain, Tyro3 transmembrane domain, BAI1 transmembrane domain, CD4 transmembrane domain, DAP12 transmembrane domain, TLR1 transmembrane domain, TLR2 transmembrane domain, TLR3 transmembrane domain, TLR4 transmembrane domain, TLR5 transmembrane domain, TLR6 transmembrane domain, TLR7 transmembrane domain, TLR8 transmembrane domain, or TLR9 transmembrane domain.

86. The method of claim 85, wherein the transmembrane domain comprises a Tim1 transmembrane domain comprising the amino acid sequence of SEQ ID NO: 18, a Tim4 transmembrane domain comprising the amino acid sequence of SEQ ID NO: 19, an FcγRI transmembrane domain comprising the amino acid sequence of SEQ ID NO:20, a CD8a transmembrane domain comprising the amino acid sequence of SEQ ID NO:22, a MERTK transmembrane domain comprising the amino acid sequence of SEQ ID NO:23, an Axl transmembrane domain comprising the amino acid sequence of SEQ ID NO:24, a MRC1 transmembrane domain comprising the amino acid sequence of SEQ ID NO:30, a Tyro3 transmembrane domain comprising the amino acid sequence of SEQ ID NO:25, a CD28 transmembrane domain of SEQ ID NO:26, a BAI1 transmembrane domain of SEQ ID NO:29, a CD4 transmembrane domain comprising the amino acid sequence of SEQ ID NO:27, a FcεRIγ transmembrane domain comprising the amino acid sequence of SEQ ID NO:21, a DAP12 transmembrane domain comprising the amino acid sequence of SEQ ID NO:28, a TLR1 transmembrane domain comprising the amino acid sequence of SEQ ID NO:31, a TLR2 transmembrane domain comprising the amino acid sequence of SEQ ID NO:32, a TLR3 transmembrane domain comprising the amino acid sequence of SEQ ID NO:33, a TLR4 transmembrane domain comprising the amino acid sequence of SEQ ID NO:34, a TLR5 transmembrane domain comprising the amino acid sequence of SEQ ID NO:35, a TLR6 transmembrane domain comprising the amino acid sequence of SEQ ID NO:36, a TLR7 transmembrane domain comprising the amino acid sequence of SEQ ID NO:37, a TLR8 transmembrane domain comprising the amino acid sequence of SEQ ID NO:38, or a TLR9 transmembrane domain comprising the amino acid sequence of SEQ ID NO:39.

87. The method of claim 70, wherein the TLR signaling domain is a TLR1 signaling domain, TLR2 signaling domain, TLR3 signaling domain, TLR4 signaling domain, TLR5 signaling domain, TLR6 signaling domain, TLR7 signaling domain, TLR8 signaling domain, or TLR9 signaling domain.

88. The method of claim 87, wherein the engulfment signaling domain comprises a TLR1 signaling domain comprising the amino acid sequence of SEQ ID NO:48, a TLR2 signaling domain comprising the amino acid sequence of SEQ ID NO:49, a TLR3 signaling domain comprising the amino acid sequence of SEQ ID NO:50, a TLR4 signaling domain comprising the amino acid sequence of SEQ ID NO:51, a TLR5 signaling domain comprising the amino acid sequence of SEQ ID NO:52, a TLR6 signaling domain comprising the amino acid sequence of SEQ ID NO:53, a TLR7 signaling domain comprising the amino acid sequence of SEQ ID NO:54, a TLR8 signaling domain comprising the amino acid sequence of SEQ ID NO:55, a TLR9 signaling domain comprising the amino acid sequence of SEQ ID NO:56, a Traf2 signaling domain comprising the amino acid sequence of SEQ ID NO:72, or a Traf3 signaling domain comprising the amino acid sequence of SEQ ID NO:73.

89. The method of claim 70, wherein the engulfment signaling domain comprises a primary engulfment signaling domain and a secondary engulfment signaling domain, wherein the primary engulfment signaling domain is a TLR signaling domain, a Traf2 signaling domain, or a Traf3 signaling domain.

90. The method of claim 89, wherein the secondary engulfment signaling domain is a FcγR1 signaling domain, FcγR2A signaling domain, FcγR2B2 signaling domain, FcγR2C signaling domain, FcγR3A signaling domain, FcεR1 signaling domain, FcαR1 signaling domain, BAFF-R signaling domain, DAP12 signaling domain, NFAM1 signaling domain, CD79b signaling domain, TLR1 signaling domain, TLR2 signaling domain, TLR3 signaling domain, TLR4 signaling domain, TLR5 signaling domain, TLR6 signaling domain, TLR7 signaling domain, TLR8 signaling domain, TLR9 signaling domain, Traf2 signaling domain, or Traf3 signaling domain.

91. The method of claim 90, wherein the secondary engulfment signaling domain is a FcγR1 signaling domain comprising the amino acid sequence of SEQ ID NO:63, an FcγR2A signaling domain comprising the amino acid sequence of SEQ ID NO:64, an FcγR2C signaling domain comprising the amino acid sequence of SEQ ID NO:65, an FcγR3A signaling domain comprising the amino acid sequence of SEQ ID NO:66, a FcεRIγ signaling domain comprising the amino acid sequence of SEQ ID NO:62, a BAFF-R signaling domain comprising the amino acid sequence of SEQ ID NO:67, a DAP12 signaling domain comprising the amino acid sequence of SEQ ID NO:68, a NFAM1 signaling domain comprising the amino acid sequence of SEQ ID NO:69, a truncated NFAM1 signaling domain comprising the amino acid sequence of SEQ ID NO:70, a CD79b signaling domain comprising the amino acid sequence of SEQ ID NO:71, a TLR1 signaling domain comprising the amino acid sequence of SEQ ID NO:48, a TLR2 signaling domain comprising the amino acid sequence of SEQ ID NO:49, a TLR3 signaling domain comprising the amino acid sequence of SEQ ID NO:50, a TLR4 signaling domain comprising the amino acid sequence of SEQ ID NO:51, a TLR5 signaling domain comprising the amino acid sequence of SEQ ID NO:52, a TLR6 signaling domain comprising the amino acid sequence of SEQ ID NO:53, a TLR7 signaling domain comprising the amino acid sequence of SEQ ID NO:54, a TLR8 signaling domain comprising the amino acid sequence of SEQ ID NO:55, a TLR9 signaling domain comprising the amino acid sequence of SEQ ID NO:56, a Traf2 signaling domain comprising the amino acid sequence of SEQ ID NO:72, or a Traf3 signaling domain comprising the amino acid sequence of SEQ ID NO:73.
